(19) United States
(12) Patent Application Publication
(10) Pub. No.: US 2010/0286360 A1
(43) Pub. Date: Nov. 11, 2010

(54) POLYMER COMPOUND AND POLYMER LIGHT EMITTING DEVICE USING THE SAME

(75) Inventors: Satoshi Kobayashi, Ibaraki (JP); Shigeya Kobayashi, Ibaraki (JP)

Correspondence Address: SUGHRUE MION, PLLC, 2100 PENNSYLVANIA AVENUE, N.W., SUITE 800, WASHINGTON, DC, 20037, US

(73) Assignee: Sumitomo Chemical Company, Limited, Chuo-ku, Tokyo (JP)

(21) Appl. No.: 11/997,568

(22) PCT Filed: Aug. 10, 2006
(86) PCT No.: PCT/JP06/16128

§ 371 Date: Feb. 1, 2008

(30) Foreign Application Priority Data
Aug. 12, 2005 (JP) 2005-234318

(51) Int. Cl. C08G 75/00; C08G 73/06; C07D 265/34; C07D 279/22; B41J 2/01

(52) U.S. Cl. 528/377; 528/403; 544/100; 544/38; 347/1

(57) ABSTRACT

A polymer compound comprising a residue of a compound of the following formula (1):

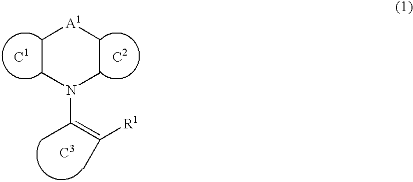

(wherein, a ring C¹, ring C² and ring C³ represent each independently an aromatic hydrocarbon ring or hetero ring. A¹ represents a di-valent group containing one or more atoms selected from a boron atom, carbon atom, nitrogen atom, oxygen atom, phosphorus atom, sulfur atom and selenium atom. R¹ represents an alkyl group, alkoxy group, alkylthio group, aryl group, aryloxy group, arylthio group, arylalkyl group, arylalkyloxy group, arylalkylthio group, alkenyl group, alkynyl group, di-substituted amino group, tri-substituted silyl group, acyl group, acyloxy group, imine residue, amide group, acid imide group, mono-valent heterocyclic group, substituted carboxyl group, heteroaryloxy group or heteroarylthio group, or is connected to an atom adjacent to an atom on the ring C³ to which R¹ is connected, to form a ring.).

### TECHNICAL FIELD

The present invention relates to a polymer compound, a method for producing the polymer compound, a compound used as a synthesis raw material of the polymer compound, a solution containing the polymer compound, a thin film containing the polymer compound, and a polymer light emitting device containing the polymer compound.

### BACKGROUND ART

Light emitting materials and charge transporting materials of high molecular weight are under various investigations since these materials are soluble in solvents and capable of forming an organic layer in a light emitting device by an application method, and known as examples thereof are polymer compounds having phenoxazine as a repeating unit on the main chain skeleton (Patent document 1: Japanese Patent Application Laid-Open (JP-A) No. 2003-165829) and blue electric field light emitting polymers having a phenoxazine unit introduced in the polyarylene main chain (Patent document 2: JP-A No. 2004-137456).

The above-described polymer compounds, however, had problems that when used in a polymer light emitting device (polymer LED), its light emission wavelength is long, and device properties such as chromaticity when used as a blue light emitting material and life when used as a light emitting material of blue, green, red or white color and the like are not necessarily sufficient.

### DISCLOSURE OF THE INVENTION

An object of the present invention is to provide a polymer compound which is useful as a light emitting material or a charge transporting material, shows short light emission wavelength when used in a polymer light emitting device, and excellent in device properties such as chromaticity when used as a blue light emitting material and life when used as a light emitting material of blue, green, red or white color and the like.

That is, the present invention provides a polymer compound comprising a residue of a compound of the following formula (1):

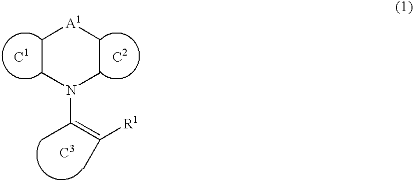

(wherein, a ring C¹, ring C² and ring C³ represent each independently an aromatic hydrocarbon ring or hetero ring. A¹ represents a di-valent group containing one or more atoms selected from a boron atom, carbon atom, nitrogen atom, oxygen atom, phosphorus atom, sulfur atom and selenium atom. R¹ represents an alkyl group, alkoxy group, alkylthio group, aryl group, aryloxy group, arylthio group, arylalkyl group, arylalkyloxy group, arylalkylthio group, alkenyl group, alkynyl group, di-substituted amino group, tri-substituted silyl group, acyl group, acyloxy group, imine residue, amide group, acid imide group, mono-valent heterocyclic group, substituted carboxyl group, heteroaryloxy group or heteroarylthio group, or is connected to an atom adjacent to an atom on the ring C³ to which R¹ is connected, to form a ring.).

The residue of a compound of the above-described formula (1) is believed to function as a light emitting part in a polymer LED, and it is hypothesized that the ring C³ is in twisted position against a plane of the rings C¹ and C² due to steric hindrance of R¹, to shorten light emission wavelength. Further, it is hypothesized that by making regions near a N atom to be sterically bulky by introduction of R¹, cleavage of a carbon-nitrogen bond having smaller bond energy as compared with a carbon-carbon bond is suppressed, thereby, the life of a polymer LED can be improved.

### BEST MODES FOR CARRYING OUT THE INVENTION

The polymer compound of the present invention contains a residue of a compound of the above-described formula (1).

In the formula (1), a ring C¹, ring C² and ring C³ represent each independently an aromatic hydrocarbon ring or hetero ring, and these rings optionally have a substituent. Here, the aromatic hydrocarbon ring has a carbon number of about 6 to 30, preferably about 6 to 15, and represents a benzene ring or a condensed aromatic hydrocarbon ring. The carbon number of the aromatic hydrocarbon group does not include the carbon number of the substituent. Specifically exemplified are a benzene ring, naphthalene ring, anthracene ring, phenanthrene ring, phenalene ring, naphthacene ring, triphenylene ring, pyrene ring, chrysene ring, pentacene ring, perylene ring, pentalene ring, indene ring, azulene ring, biphenylene ring, fluorene ring, acenaphthylene ring and the like.

The hetero ring has a carbon number of about 2 to 30, preferably about 2 to 15. The carbon number of the hetero ring group does not include the carbon number of the substituent. Here, the hetero ring refers to organic compounds having a cyclic structure in which elements constituting the ring include not only a carbon atom, but also a hetero atom such as oxygen, sulfur, nitrogen, phosphorus, boron and the like contained in the ring.

Among the hetero rings, aromatic hetero rings are preferable. Specifically exemplified are a furan ring, thiophene ring, pyrrole ring, imidazole ring, pyrazole ring, oxazole ring, thiazole ring, pyridine ring, pyrazine ring, pyrimidine ring, pyridazine ring, benzofuran ring, benzothiophene ring, indole ring, quinoline ring, quinoxaline ring, dibenzofuran ring, dibenzothiophene ring, carbazole ring, acridine ring and the like.

When the ring C³ is an aromatic hydrocarbon ring or aromatic hetero ring, a double bond in the ring C³ constitutes a conjugate structure together with other unsaturated bond in the ring. When the ring C³ is a hetero ring which is not an aromatic hetero ring, a bond between carbon on the ring C³ to which a N atom is connected and a carbon atom to which R¹ is connected is a double bond.

The aromatic hydrocarbon ring or hetero ring is preferably a benzene ring or monocyclic hetero ring, and more preferably a benzene ring.

Exemplified as substituents on the ring C¹, ring C² or ring C³ are halogen atoms, alkyl groups, alkoxy groups, alkylthio groups, aryl groups, aryloxy groups, arylthio groups, arylalkyl groups, arylalkoxy groups, arylalkylthio groups, alkenyl groups, alkynyl groups, di-substituted amino groups, tri-substituted silyl groups, acyl groups, acyloxy groups, imine residues, amide groups, acid imide groups, monovalent heterocyclic groups, substituted carboxyl groups, heteroaryloxy groups or heteroarylthio groups.

Here, exemplified as the halogen atom are a fluorine atom, chlorine atom, bromine atom and iodine atom.

The alkyl group may be any of linear, branched or cyclic, the carbon number thereof is usually about 1 to 30, and from the standpoint of solubility in a solvent, preferably about 3 to 15, and mentioned as specific examples thereof are a methyl group, ethyl group, propyl group, i-propyl group, butyl group, i-butyl group, t-butyl group, pentyl group, isoamyl group, hexyl group, cyclohexyl group, heptyl group, octyl group, 2-ethylhexyl group, nonyl group, decyl group, 3,7-dimethyloctyl group, lauryl group, trifluoromethyl group, pentafluoroethyl group, perfluorobutyl group, perfluorohexyl group, perfluorooctyl group and the like, and preferable according to balance between heat resistance and the standpoints of solubility in an organic solvent, device properties, easiness of synthesis, and the like are a pentyl group, isoamyl group, hexyl group, octyl group, 2-ethylhexyl group, decyl group and 3,7-dimethyloctyl group.

The alkoxy group may be any of linear, branched or cyclic, the carbon number thereof is usually about 1 to 30, and from the standpoint of solubility in a solvent, preferably about 3 to 15, and mentioned as specific examples thereof are a methoxy group, ethoxy group, propyloxy group, i-propyloxy group, butoxy group, i-butoxy group, t-butoxy group, pentyloxy group, hexyloxy group, cyclohexyloxy group, heptyloxy group, octyloxy group, 2-ethylhexyloxy group, nonyloxy group, decyloxy group, 3,7-dimethyloctyloxy group, lauryloxy group, trifluoromethoxy group, pentafluoroethoxy group, perfluorobutoxy group, perfluorohexyl group, perfluorooctyl group, methoxymethyloxy group, 2-methoxyethyloxy group and the like, and preferable according to balance between heat resistance and the standpoints of solubility in an organic solvent, device properties, easiness of synthesis, and the like are a pentyloxy group, hexyloxy group, octyloxy group, 2-ethylhexyloxy group, decyloxy group and 3,7-dimethyloctyloxy group.

The alkylthio group may be any of linear, branched or cyclic, the carbon number thereof is usually about 1 to 30, and from the standpoint of solubility in a solvent, preferably about 3 to 15, and mentioned as specific examples thereof are a methylthio group, ethylthio group, propylthio group, i-propylthio group, butylthio group, i-butylthio group, t-butylthio group, pentylthio group, hexylthio group, cyclohexylthio group, heptylthio group, octylthio group, 2-ethylhexylthio group, nonylthio group, decylthio group, 3,7-dimethyloctylthio group, laurylthio group, trifluoromethylthio group and the like are mentioned, and preferable according to balance between heat resistance and the standpoints of solubility in an organic solvent, device properties, easiness of synthesis, and the like are a pentylthio group, hexylthio group, octylthio group, 2-ethylhexylthio group, decylthio group and 3,7-dimethyloctylthio group.

The aryl group is an atom group obtained by removing one hydrogen atom from an aromatic hydrocarbon, and includes also those having a condensed ring and those obtained by bonding of two or more independent benzene rings or condensed rings directly or via a group such as vinylene and the like. The aryl group has a carbon number of usually about 6 to 60, preferably about 6 to 48, and exemplified as specific examples thereof are a phenyl group, C₁ to C₁₂ alkoxyphenyl groups (C₁ to C₁₂ means a carbon number of 1 to 12. Applicable also in the later descriptions), C₁ to C₁₂ alkylphenyl groups, 1-naphthyl group, 2-naphthyl group, 1-anthracenyl group, 2-anthracenyl group, 9-anthracenyl group, pentafluorophenyl group and the like, and preferable from the standpoints of solubility in an organic solvent, device properties, easiness of synthesis, and the like are C₁ to C₁₂ alkoxyphenyl groups and C₁ to C₁₂ alkylphenyl groups. Specifically exemplified as the C₁ to C₁₂ alkoxy are methoxy, ethoxy, propyloxy, i-propyloxy, butoxy, i-butoxy, t-butoxy, pentyloxy, hexyloxy, cyclohexyloxy, heptyloxy, octyloxy, 2-ethylhexyloxy, nonyloxy, decyloxy, 3,7-dimethyloctyloxy, lauryloxy and the like.

Specifically exemplified as the C₁ to C₁₂ alkylphenyl group are a methylphenyl group, ethylphenyl group, dimethylphenyl group, propylphenyl group, mesityl group, methylethylphenyl group, 1-propylphenyl group, butylphenyl group, 1-butylphenyl group, t-butylphenyl group, pentylphenyl group, isoamylphenyl group, hexylphenyl group, heptylphenyl group, octylphenyl group, nonylphenyl group, decylphenyl group, dodecylphenyl group and the like.

The aryloxy group has a carbon number of usually about 6 to 60, preferably about 6 to 30, and as specific examples thereof, a phenoxy group, C₁ to C₁₂ alkoxyphenoxy groups, C₁ to C₁₂ alkylphenoxy groups, 1-naphthyloxy group, 2-naphthyloxy group, pentafluorophenyloxy group and the like are exemplified, and preferable from the standpoints of solubility in an organic solvent, device properties, easiness of synthesis, and the like are C₁ to C₁₂ alkoxyphenoxy groups and C₁ to C₁₂ alkylphenoxy groups.

Specifically exemplified as the C₁ to C₁₂ alkoxy are methoxy, ethoxy, propyloxy, i-propyloxy, butoxy, i-butoxy, t-butoxy, pentyloxy, hexyloxy, cyclohexyloxy, heptyloxy, octyloxy, 2-ethylhexyloxy, nonyloxy, decyloxy, 3,7-dimethyloctyloxy, lauryloxy and the like.

Specifically exemplified as the C₁ to C₁₂ alkylphenoxy group are a methylphenoxy group, ethylphenoxy group, dimethylphenoxy group, propylphenoxy group, 1,3,5-trimethylphenoxy group, methylethylphenoxy group, i-propylphenoxy group, butylphenoxy group, i-butylphenoxy group, t-butylphenoxy group, pentylphenoxy group, isoamylphenoxy group, hexylphenoxy group, heptylphenoxy group, octylphenoxy group, nonylphenoxy group, decylphenoxy group, dodecylphenoxy group and the like.

The arylthio group has a carbon number of usually about 6 to 60 carbon atoms, preferably about 6 to 30. As specific examples thereof, a phenylthio group, C₁ to C₁₂ alkoxyphenylthio groups, C₁ to C₁₂ alkylphenylthio groups, 1-naphthylthio group, 2-naphthylthio group, pentafluorophenylthio group and the like are exemplified, and preferable from the standpoints of solubility in an organic solvent, device properties, easiness of synthesis, and the like are C₁ to C₁₂ alkoxyphenylthio groups and C₁ to C₁₂ alkylphenylthio groups.

The arylalkyl group has a carbon number of usually about 7 to 60, preferably about 7 to 30, and as specific examples thereof, phenyl-C₁ to C₁₂ alkyl groups, C₁ to C₁₂ alkoxyphenyl-C₁ to C₁₂ alkyl groups, C₁ to C₁₂ alkylphenyl-C₁ to C₁₂ alkyl groups, 1-naphthyl-C₁ to C₁₂ alkyl groups, 2-naphthyl-C₁ to C₁₂ alkyl groups and the like are exemplified, and preferable from the standpoints of solubility in an organic solvent, device properties, easiness of synthesis, and the like are C₁ to C₁₂ alkoxyphenyl-C₁ to C₁₂ alkyl groups and C₁ to C₁₂ alkylphenyl-C₁ to C₁₂ alkyl groups.

The arylalkoxy group has a carbon number of usually about 7 to 60, preferably about 7 to 30, and as specific examples thereof, phenyl-C₁ to C₁₂ alkoxy groups such as a phenylmethoxy group, phenylethoxy group, phenylbutoxy group, phenylpentyloxy group, phenylhexyloxy group, phenylheptyloxy group, phenyloctyloxy group and the like, C₁ to C₁₂ alkoxyphenyl-C₁ to C₁₂ alkoxy groups, C₁ to C₁₂ alkylphenyl-C₁ to C₁₂ alkoxy groups, 1-naphthyl-C₁ to C₁₂ alkoxy groups, 2-naphthyl-C₁ to C₁₂ alkoxy groups and the like are exemplified, and preferable from the standpoints of solubility in an organic solvent, device properties, easiness of synthesis, and the like are C₁ to C₁₂ alkoxyphenyl-C₁ to C₁₂ alkoxy groups and C₁ to C₁₂ alkylphenyl-C₁ to C₁₂ alkoxy groups.

The arylalkylthio group has a carbon number of usually about 7 to 60, preferably about 7 to 30, and as specific examples thereof, phenyl-C₁ to C₁₂ alkylthio groups, C₁ to C₁₂ alkoxyphenyl-C₁ to C₁₂ alkylthio groups, C₁ to C₁₂ alkylphenyl-C₁ to C₁₂ alkylthio groups, 1-naphthyl-C₁ to C₁₂ alkylthio groups, 2-naphthyl-C₁ to C₁₂ alkylthio groups and the like are exemplified, and preferable from the standpoints of solubility in an organic solvent, device properties, easiness of synthesis, and the like are C₁ to C₁₂ alkoxyphenyl-C₁ to C₁₂ alkylthio groups and C₁ to C₁₂ alkylphenyl-C₁ to C₁₂ alkylthio groups.

The alkenyl group has a carbon number of about 2 to 30, preferably about 2 to 15. Specifically, a vinyl group, 1-propylenyl group, 2-propylenyl group, butenyl group, pentenyl group, hexenyl group, heptenyl group, octenyl group and cyclohexenyl group are exemplified, and also dienyl groups and trienyl groups such as a 1,3-butadienyl group, cyclohexa-1,3-dienyl group, 1,3,5-hexatrienyl group and the like are included.

The alkynyl group has a carbon number of about 2 to 30, preferably about 2 to 15. Specifically, an ethynyl group, 1-propynyl group, 2-propylenyl group, butynyl group, pentynyl group, hexynyl group, heptynyl group, octynyl group, cyclohexylethynyl group and the like are exemplified, and also diynyl groups such as a 1,3-butadiynyl group and the like are included.

The di-substituted amino group includes amino groups substituted with two groups selected from alkyl groups, aryl groups, arylalkyl groups or mono-valent heterocyclic groups, and the alkyl group, aryl group, arylalkyl group or mono-valent heterocyclic group optionally has a substituent. The carbon number of the di-substituted amino group is usually about 2 to 60, preferably about 2 to 30 excluding the carbon number of the substituent.

Specifically exemplified are a dimethylamino group, diethylamino group, dipropylamino group, diisopropylamino group, dibutylamino group, diisobutylamino group, dipentylamino group, dihexylamino group, diheptylamino group, dioctylamino group, di-2-ethylhexylamino group, dinonylamino group, didecylamino group, di-3,7-dimethyloctylamino group, dilaurylamino group, dicyclopentylamino group, dicyclohexylamino group, pyrrolidyl group, piperidyl group, ditrifluoromethylamino group, phenylamino group, diphenylamino group, di(C₁ to C₁₂ alkoxyphenyl)amino groups, di(C₁ to C₁₂ alkylphenyl)amino groups, di-1-naphthylamino group, di-2-naphthylamino group, dipentafluorophenylamino group, dipyridylamino group, dipyridazinylamino group, dipyrimidylamino group, dipyrazylamino group, ditriazylamino group, di(phenyl-C₁ to C₁₂ alkyl)amino groups, di(C₁ to C₁₂ alkoxyphenyl-C₁ to C₁₂ alkyl)amino groups, di(C₁ to C₁₂ alkylphenyl-C₁ to C₁₂ alkyl)amino groups and the like.

The tri-substituted silyl group includes silyl groups substituted with three groups selected from alkyl groups, aryl groups, arylalkyl groups or mono-valent heterocyclic groups. The carbon number of the substituted silyl group is usually about 3 to 90, preferably about 3 to 45. The alkyl group, aryl group, arylalkyl group or mono-valent heterocyclic group optionally has a substituent.

Specifically exemplified are a trimethylsilyl group, triethylsilyl group, tripropylsilyl group, tri-1-propylsilyl group, dimethyl-1-propylsilyl group, diethyl-1-propylsilyl group, t-butylsilyldimethylsilyl group, pentyldimethylsilyl group, hexyldimethylsilyl group, heptyldimethylsilyl group, octyldimethylsilyl group, 2-ethylhexyl-dimethylsilyl group, nonyldimethylsilyl group, decyldimethylsilyl group, 3,7-dimethyloctyl-dimethylsilyl group, lauryldimethylsilyl group, phenyl-C₁ to C₁₂ alkylsilyl groups, C₁ to C₁₂ alkoxyphenyl-C₁ to C₁₂ alkylsilyl groups, C₁ to C₁₂ alkylphenyl-C₁ to C₁₂ alkylsilyl groups, 1-naphthyl-C₁ to C₁₂ alkylsilyl groups, 2-naphthyl-C₁ to C₁₂ alkylsilyl groups, phenyl-C₁ to C₁₂ alkyldimethylsilyl groups, triphenylsilyl group, tri-p-xylylsilyl group, tribenzylsilyl group, diphenylmethylsilyl group, t-butyldiphenylsilyl group, dimethylphenylsilyl group and the like.

The acyl group has a carbon number of usually about 2 to 30, preferably about 2 to 15, and as specific examples thereof, an acetyl group, propionyl group, butyryl group, isobutyryl group, pivaloyl group, benzoyl group, trifluoroacetyl group, pentafluorobenzoyl group and the like are exemplified.

The acyloxy group has a carbon number of usually about 2 to 30, preferably about 2 to 15, and as specific examples thereof, an acetoxy group, propionyloxy group, butyryloxy group, isobutyryloxy group, pivaloyloxy group, benzoyloxy group, trifluoroacetyloxy group, pentafluorobenzoyloxy group and the like are exemplified.

The imine residue has a carbon number of usually about 2 to 30, preferably about 2 to 15, and as specific examples thereof, groups of the following structural formulae, and the like, are exemplified.

A wavy line represents syn or anti, and both syn and anti are permissible.

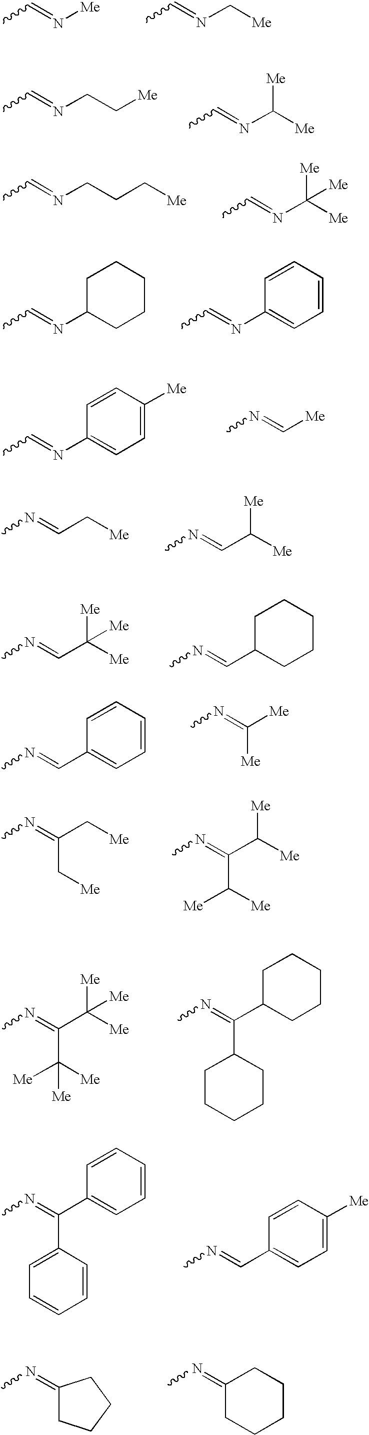

The amide group has a carbon number of usually about 2 to 30, preferably about 2 to 15, and as specific examples thereof, a formamide group, acetamide group, propioamide group, butyroamide group, benzamide group, trifluoroacetamide group, pentafluorobenzamide group, diformamide group, diacetamide group, dipropioamide group, dibutyroamide group, dibenzamide group, ditrifluoroacetamide group, dipentafluorobenzamide group and the like are exemplified.

As the acid imide group, residues obtained by removing a hydrogen atom bonded to its nitrogen atom from an acid imide are mentioned, and the carbon number thereof is about 4 to 30, preferably about 4 to 15. Specifically, the following groups and the like are exemplified.

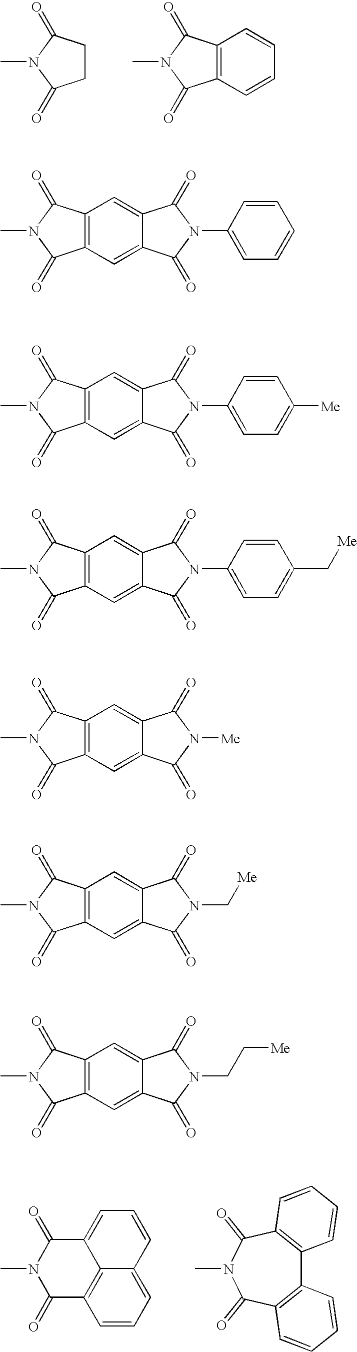

The mono-valent heterocyclic group means an atomic group remaining after removing one hydrogen atom from a heterocyclic compound, and the carbon number is usually about 2 to 30, preferably about 2 to 15. The heterocyclic group may carry thereon a substituent, and the number carbon thereof does not include the carbon number of the substituent. Here, the heterocyclic compound refers to organic compounds having a cyclic structure in which elements constituting the ring include not only a carbon atom, but also a hetero atom such as oxygen, sulfur, nitrogen, phosphorus, boron and the like contained in the ring. Specifically exemplified are a thienyl group, C₁ to C₁₂ alkylthienyl groups, pyrrolyl group, furyl group, pyridyl group, C₁ to C₁₂ alkylpyridyl groups, piperidyl group, quinolyl group, isoquinolyl group and the like, and preferable are a thienyl group, C₁ to C₁₂ alkylthienyl groups, pyridyl group and C₁ to C₁₂ alkylpyridyl groups.

As the substituted carboxyl group, carboxyl groups substituted with an alkyl group, aryl group, arylalkyl group or mono-valent heterocyclic group are mentioned, and the carbon number is usually about 2 to 30, preferably about 2 to 15, and specific examples thereof include a methoxycarbonyl group, ethoxycarbonyl group, propoxycarbonyl group, i-propoxycarbonyl group, butoxycarbonyl group, i-butoxycarbonyl group, t-butoxycarbonyl group, pentyloxycarbonyl group, hexyloxycarbonyl group, cyclohexyloxycarbonyl group, heptyloxycarbonyl group, octyloxycarbonyl group, 2-ethylhexyloxycarbonyl group, nonyloxycarbonyl group, decyloxycarbonyl group, 3,7-dimethyloctyloxycarbonyl group, dodecyloxycarbonyl group, trifluoromethoxycarbonyl group, pentafluoroethoxycarbonyl group, perfluorobutoxycarbonyl group, perfluorohexyloxycarbonyl group, perfluorooctyloxycarbonyl group, phenoxycarbonyl group, naphthoxycarbonyl group, pyridyloxycarbonyl group, and the like. The alkyl group, aryl group, arylalkyl group or mono-valent heterocyclic group optionally has a substituent. The carbon number of the substituted carboxyl group does not include the carbon number of the substituent.

The heteroaryloxy group (group represented by Q¹-O—, Q¹ represents a mono-valent heterocyclic group) has a carbon number of usually about 2 to 30, preferably about 2 to 15. The heterocyclic group may carry thereon a substituent, and the number carbon thereof does not include the carbon number of the substituent. As specific examples thereof, a thienyloxy group, C₁ to C₁₂ alkylthienyloxy groups, pyrrolyloxy group, furyloxy group, pyridyloxy group, C₁ to C₁₂ alkylpyridyloxy groups, imidazolyloxy group, pyrazolyloxy group, triazolyloxy group, oxazolyloxy group, thiazoleoxy group, thiadiazoleoxy group and the like are exemplified. Q¹ is preferably a mono-valent aromatic heterocyclic group.

The heteroarylthio group (group represented by Q²-S—, Q² represents a mono-valent heterocyclic group) has a carbon number of usually about 2 to 30, preferably about 2 to 15. The heterocyclic group may carry thereon a substituent, and the number carbon thereof does not include the carbon number of the substituent. As specific examples thereof, a thienylmercapto group, C₁ to C₁₂ alkylthienylmercapto groups, pyrrolylmercapto group, furylmercapto group, pyridylmercapto group, C₁ to C₁₂ alkylpyridylmercapto groups, imidazolylmercapto group, pyrazolylmercapto group, triazolylmercapto group, oxazolylmercapto group, thiazolemercapto group, thiadiazolemercapto group and the like are exemplified. Q² is preferably a mono-valent aromatic heterocyclic group.

In the above-described formula (1), A¹ represents a di-valent group containing one or more atoms selected from a boron atom, carbon atom, nitrogen atom, oxygen atom, phosphorus atom, sulfur atom and selenium atom.

Among the di-valent groups represented by A¹, those forming a 6-membered ring or 7-membered ring together with the N atom, ring C¹ and ring C² are preferable, and those forming a 6-membered ring are more preferable.

Specifically, the following groups are exemplified.

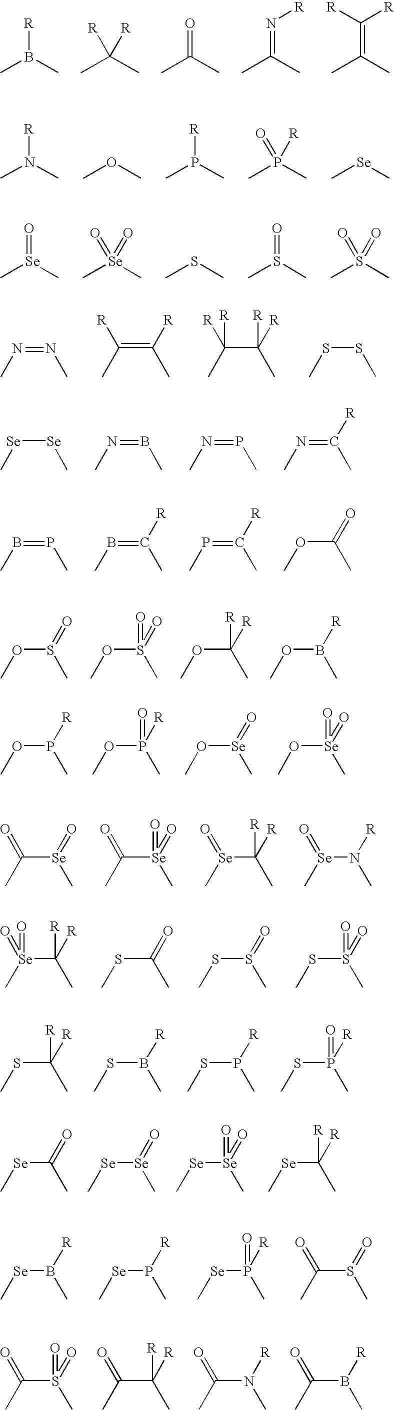

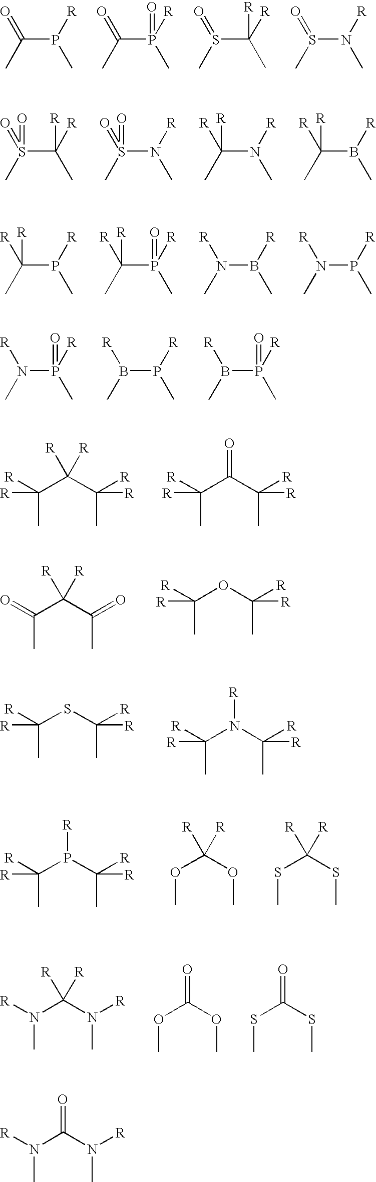

(wherein, Rs represent each independently a hydrogen atom, halogen atom, alkyl group, alkoxy group, alkylthio group, aryl group, aryloxy group, arylthio group, arylalkyl group, arylalkyloxy group, arylalkylthio group, alkenyl group, alkynyl group, heteroaryloxy group or heteroarylthio group.

Definitions and examples of the halogen atom, alkyl group, alkoxy group, alkylthio group, aryl group, aryloxy group, arylthio group, arylalkyl group, arylalkyloxy group, arylalkylthio group, alkenyl group, alkynyl group, heteroaryloxy group or heteroarylthio group represented by R are the same as the descriptions for the above-mentioned substituents on the ring C¹, C² or C³.

As the di-valent group represented by A¹, those represented by —C(R)₂—, —O—, —S—, —S(═O)—, —S(═O)₂—, —Se—, —Se(═O)— and —Se(═O)₂— are preferable, and those represented by —C(R)₂—, —O— and —S— are more preferable, from the standpoint of stability of the compound.

In the above-described formula (1), R¹ represents an alkyl group, alkoxy group, alkylthio group, aryl group, aryloxy group, arylthio group, arylalkyl group, arylalkyloxy group, arylalkylthio group, alkenyl group, alkynyl group, di-substituted amino group, tri-substituted silyl group, acyl group, acyloxy group, imine residue, amide group, acid imide group, mono-valent heterocyclic group, substituted carboxyl group, heteroaryloxy group or heteroarylthio group, or is connected to an atom adjacent to an atom on the ring C³ to which R¹ is connected, to form a ring.

R¹ is preferably an alkyl group, aryl group, arylalkyl group or mono-valent heterocyclic group, more preferably an alkyl group, and most preferably a methyl group.

Definitions and examples of the alkyl group, alkoxy group, alkylthio group, aryl group, aryloxy group, arylthio group, arylalkyl group, arylalkyloxy group, arylalkylthio group, alkenyl group, alkynyl group, di-substituted amino group, tri-substituted silyl group, acyl group, acyloxy group, imine residue, amide group, acid imide group, mono-valent heterocyclic group, substituted carboxyl group, heteroaryloxy group and heteroarylthio group represented by R¹ are the same as the descriptions for substituents on the above-mentioned ring C¹, C² or C³.

When R¹ is connected to an atom adjacent to an atom on the ring C³ to which R¹ is connected, to form a ring, the following structures are exemplified for the ring C³.

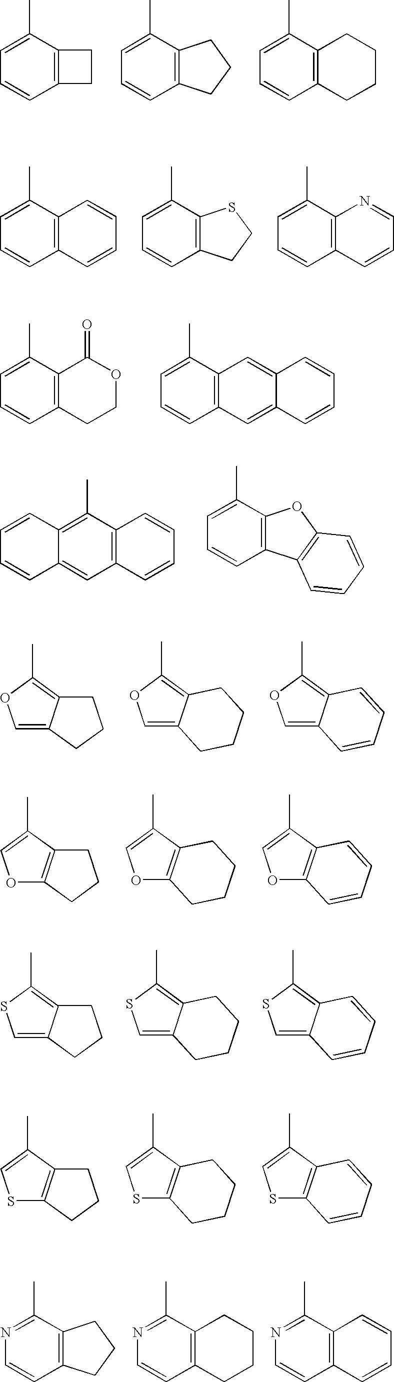

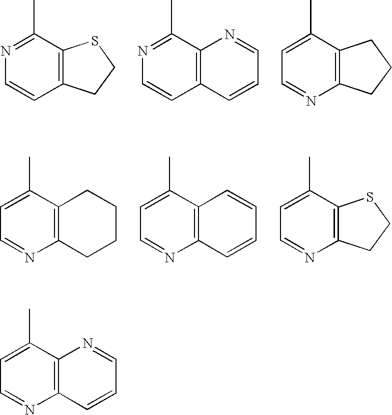

In the formulae, a connecting bond represents a connecting bond to a nitrogen atom. Further, condensed rings may carry thereon a substituent selected from halogen atoms, alkyl groups, alkoxy groups, alkylthio groups, aryl groups, aryloxy groups, arylthio groups, arylalkyl groups, arylalkyloxy groups, arylalkylthio groups, alkenyl groups, alkynyl groups, di-substituted amino groups, tri-substituted silyl groups, acyl groups, acyloxy groups, imine residues, amide groups, acid imide groups, monovalent heterocyclic groups, substituted carboxyl groups, heteroaryloxy groups and heteroarylthio groups.

Among the residues of compounds of the above-described formula (1), those in which the ring C¹ and ring C² are a benzene ring or monocyclic hetero cycle are preferable from the standpoint of stability of the compound. Those in which the ring C¹ and ring C² are a 6-membered ring are more preferable, and those in which the ring C¹ and ring C² are a benzene ring are further preferable.

Among residues of compounds of the above-described formula (1), those in which the ring C³ is an aromatic hydrocarbon ring are preferable, those in which the ring C³ is a benzene ring are more preferable, and those of the following formula are most preferable.

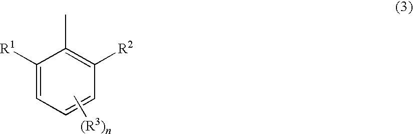

In the formula, R¹ has the same meaning as described above. R² and R³ represent each independently an alkyl group, alkoxy group, alkylthio group, aryl group, aryloxy group, arylthio group, arylalkyl group, arylalkyloxy group, arylalkylthio group, alkenyl group, alkynyl group, di-substituted amino group, tri-substituted silyl group, acyl group, acyloxy group, imine residue, amide group, acid imide group, mono-valent heterocyclic group, substituted carboxyl group, heteroaryloxy group or heteroarylthio group, and when R³ is adjacent to R¹ or R², it may be connected to R¹ or R² to form a ring.

R² is preferably an alkyl group, aryl group, arylalkyl group or mono-valent heterocyclic group, more preferably an alkyl group, and most preferably a methyl group.

Definitions and examples of the alkyl group, alkoxy group, alkylthio group, aryl group, aryloxy group, arylthio group, arylalkyl group, arylalkyloxy group, arylalkylthio group, alkenyl group, alkynyl group, di-substituted amino group, tri-substituted silyl group, acyl group, acyloxy group, imine residue, amide group, acid imide group, mono-valent heterocyclic group, substituted carboxyl group, heteroaryloxy group and heteroarylthio group represented by R² and R³ are the same as the descriptions for substituents on the above-mentioned ring C¹, C² or C³.

n represents 0, 1, 2 or 3. When n is 2 or more, plural R³s may be the same or different.

As the substituent represented by the above-described formula (3), the following structures are exemplified.

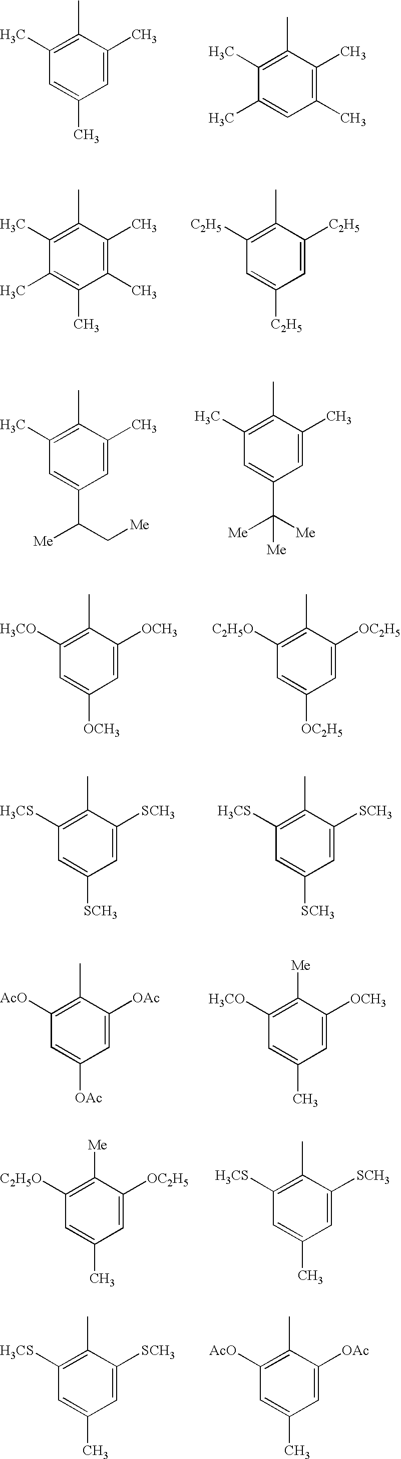

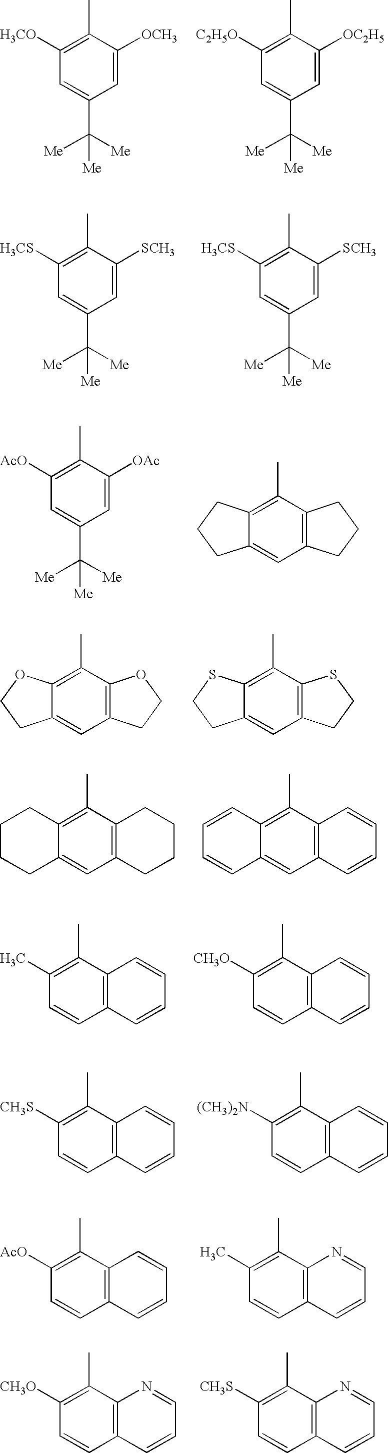

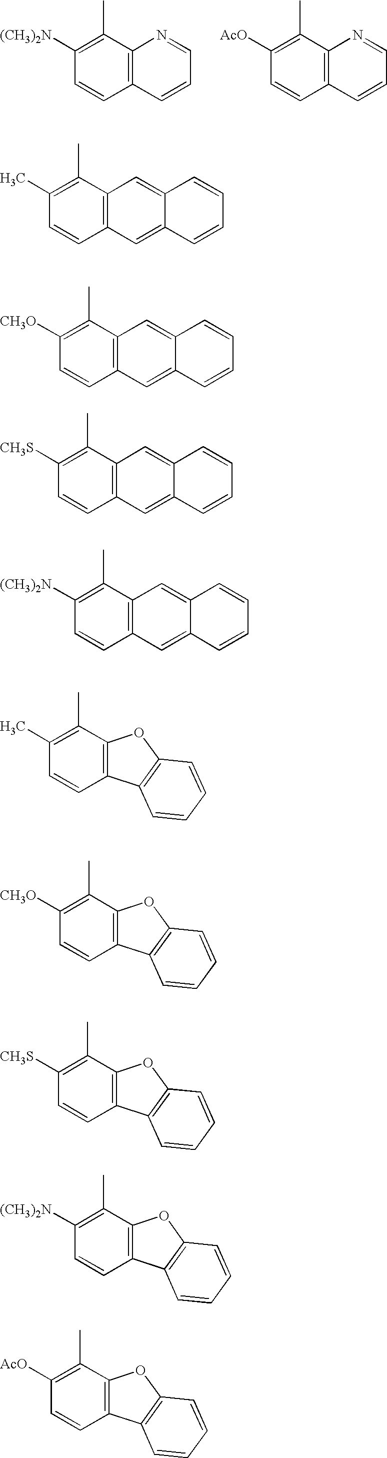

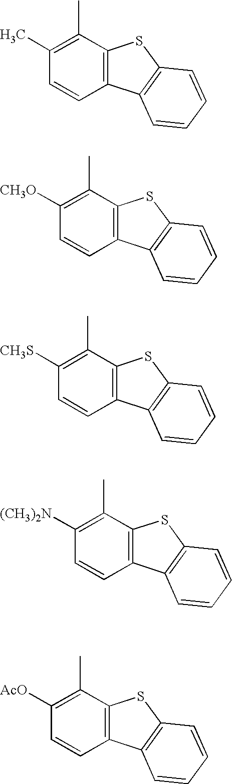

Of them, structures of the following formula (3-1) are preferable, and structures of the following formula (3-2) are more preferable.

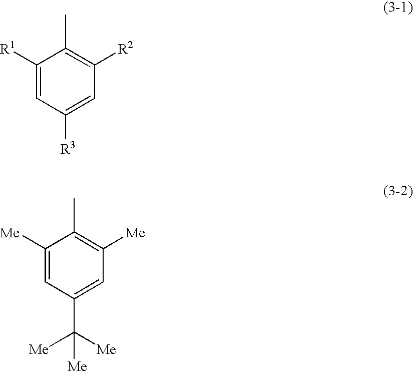

In the formulae, R¹, R² and R³ have the same meanings as described above.

Among residues of compounds of the above-described formula (1), those in which A¹ is an oxygen atom, sulfur atom, —S(═O)—, —S(═O)₂—, selenium atom, —Se(═O)— or —Se(═O)₂— are preferable, those in which A¹ is an oxygen atom, sulfur atom or selenium atom are more preferable, and those in which A¹ is an oxygen atom or sulfur atom are most preferable, from the standpoint of stability of the compound.

Examples of structural units containing residues of compounds of the above-described formula (1) include those containing a residue of the compound on the main chain, those containing a residue of the compound on the end of the main chain, and those containing a residue of the compound on the side chain.

As those containing a residue of the compound on the main chain, structural units of the following formula (2) are exemplified. As the polymer compound of the present invention, those having structural units of the following formula (2) are mentioned.

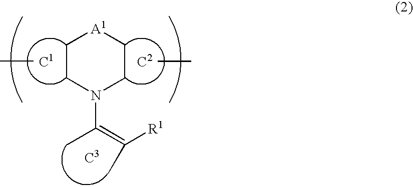

In the formula, the ring C¹, ring C², ring C³, A¹ and R¹ have the same meanings as described above.

Specifically, the following moieties, and those having a substituent on a benzene ring or hetero ring of the following moieties, and the like, are mentioned.

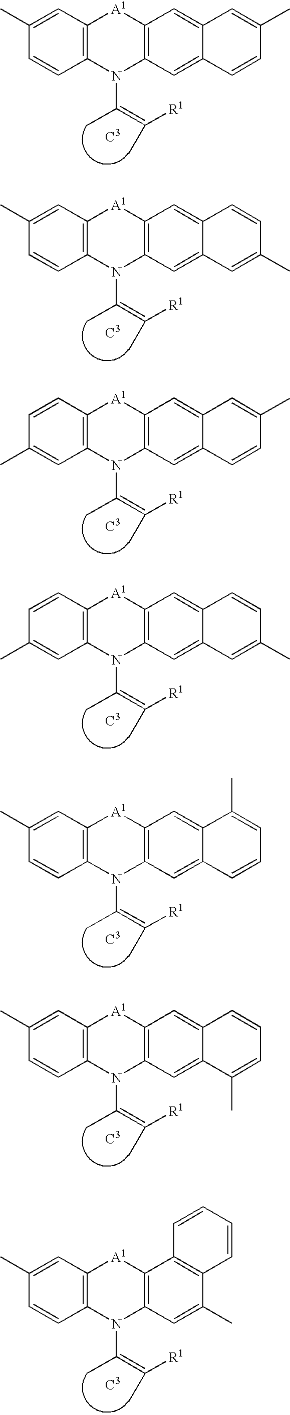

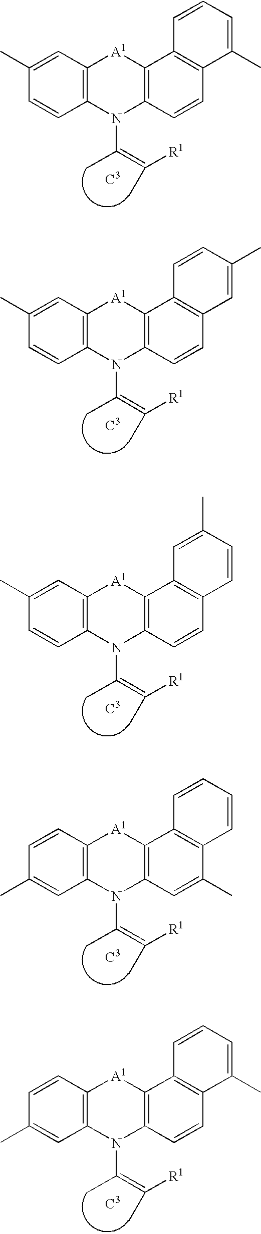

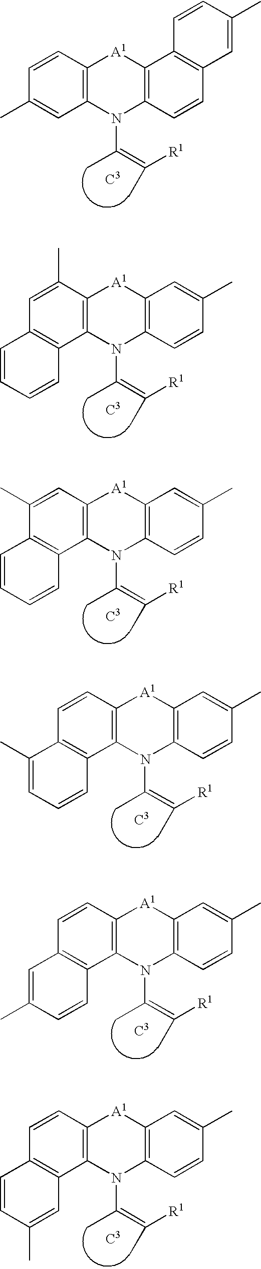

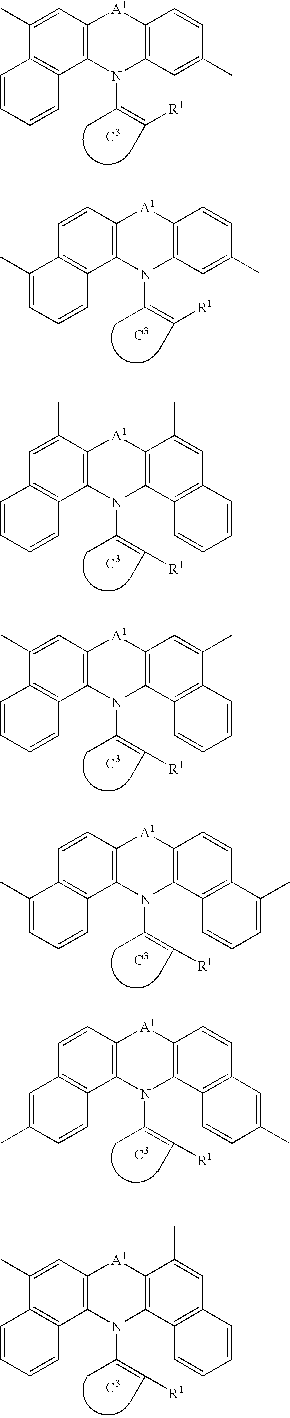

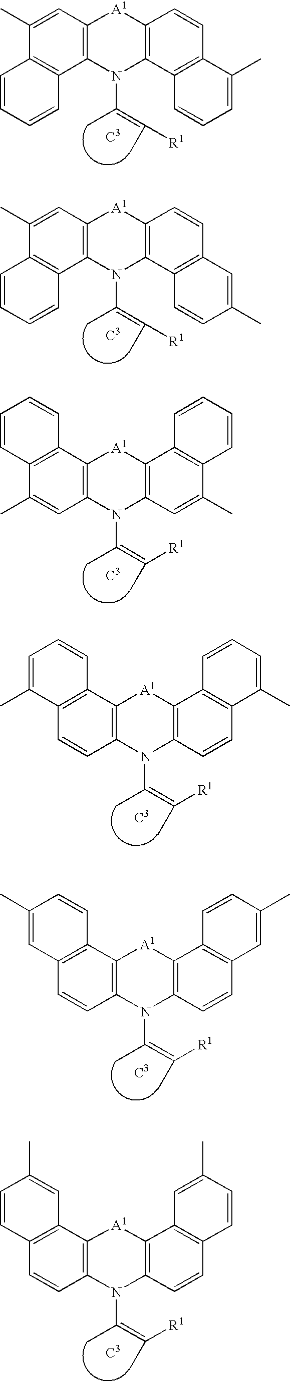

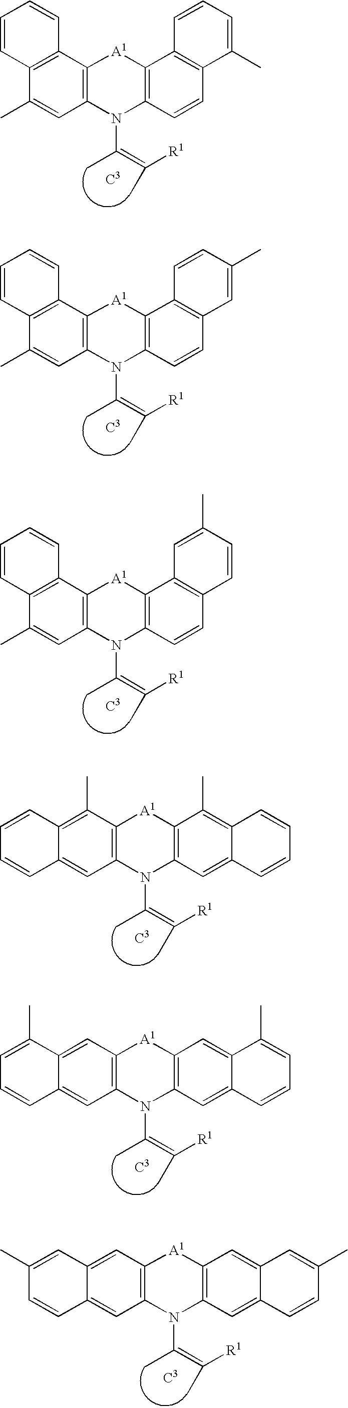

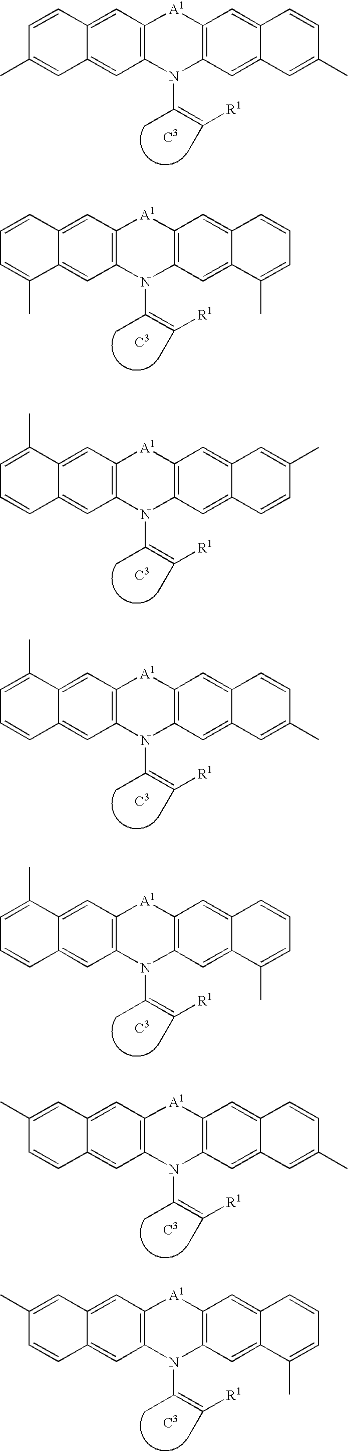

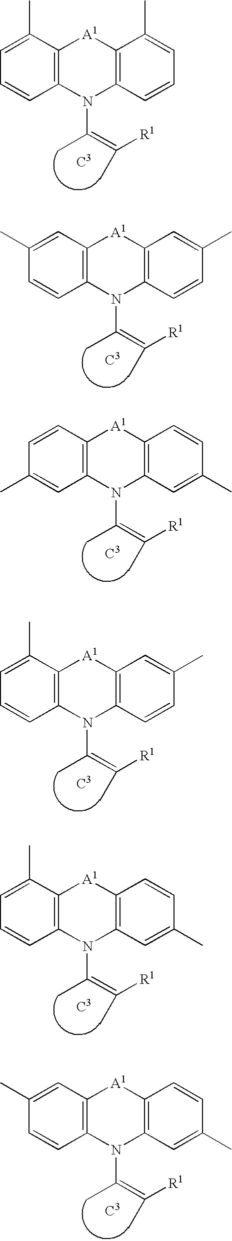

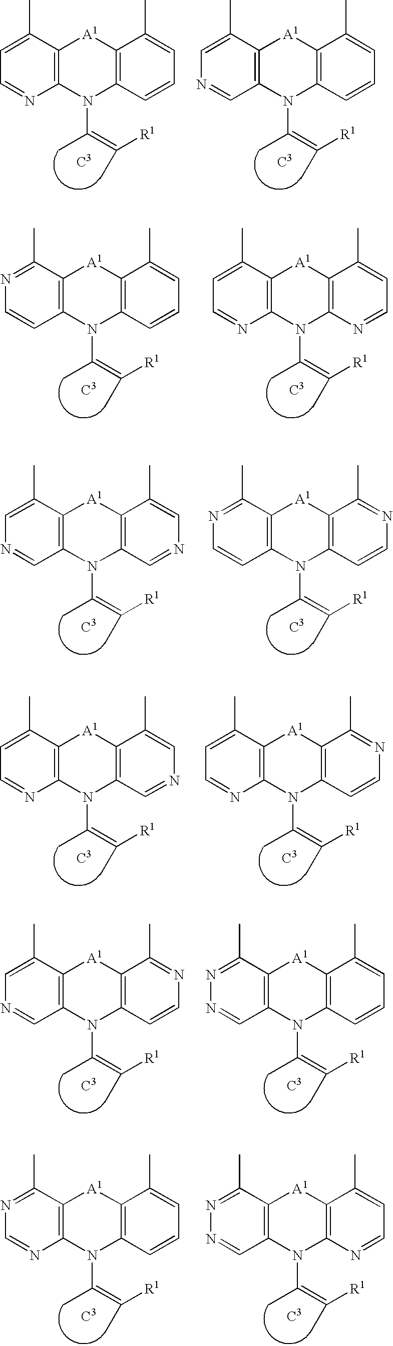

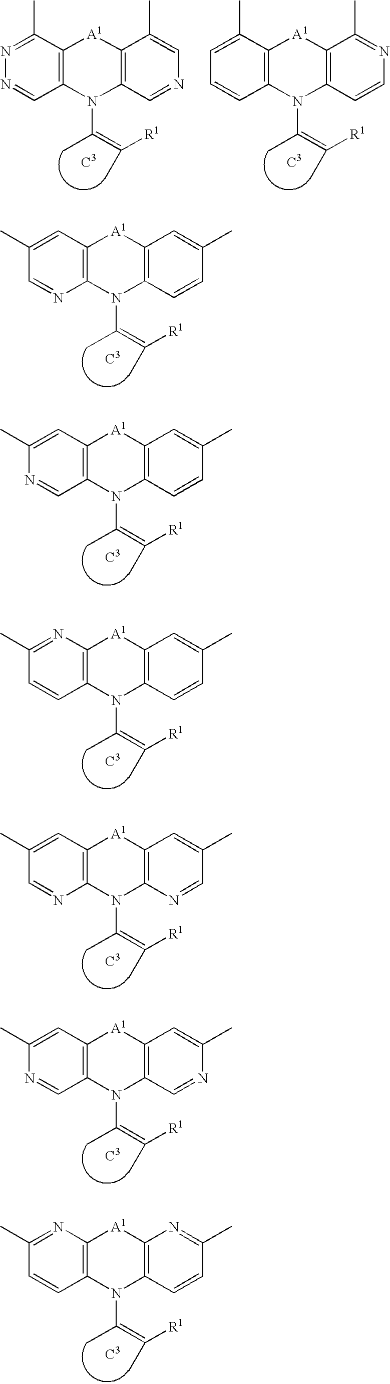

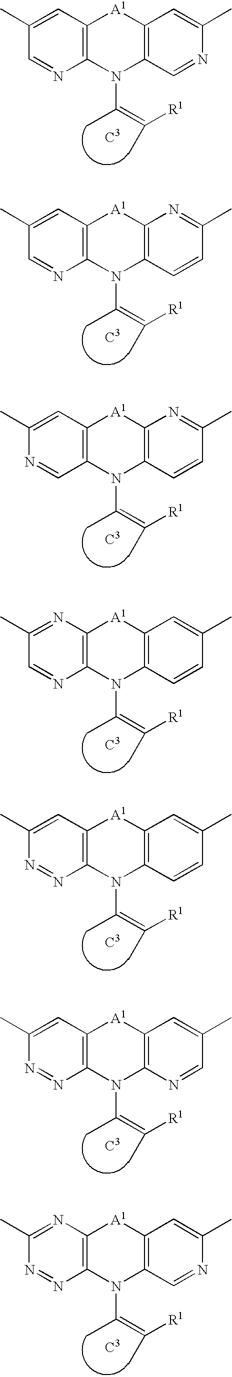

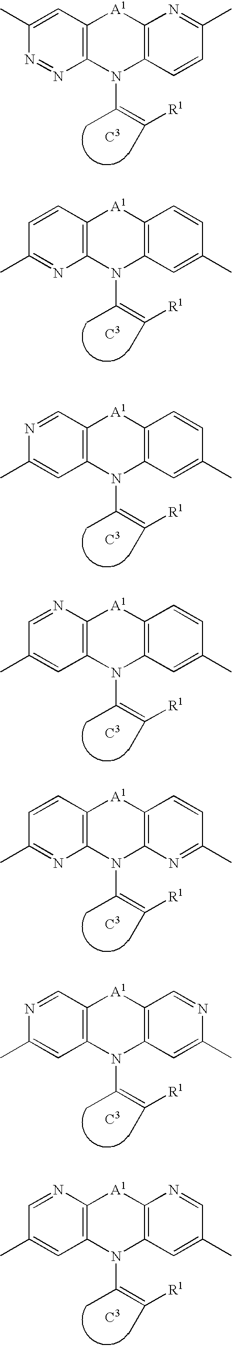

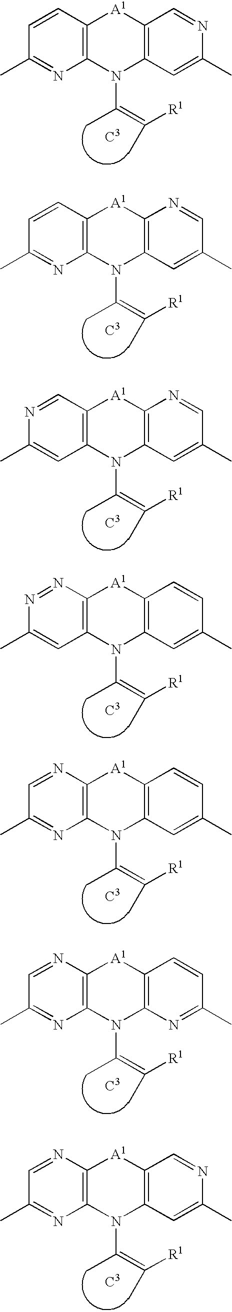

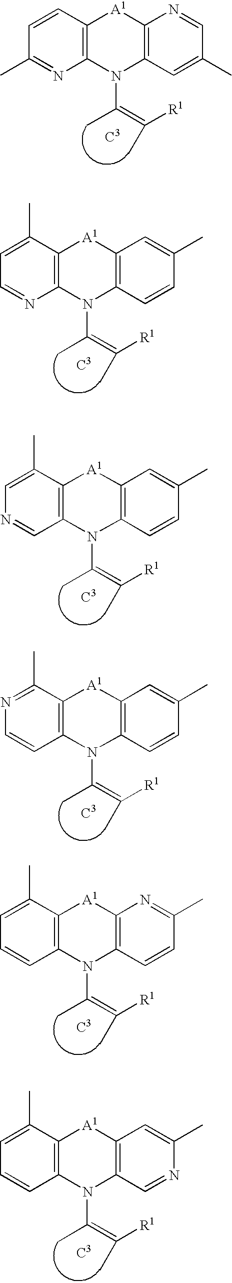

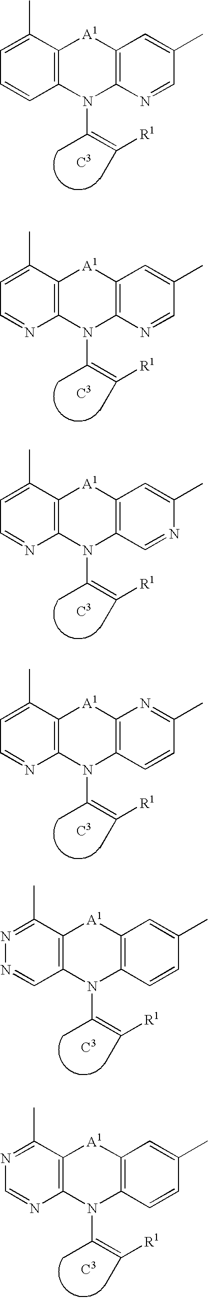

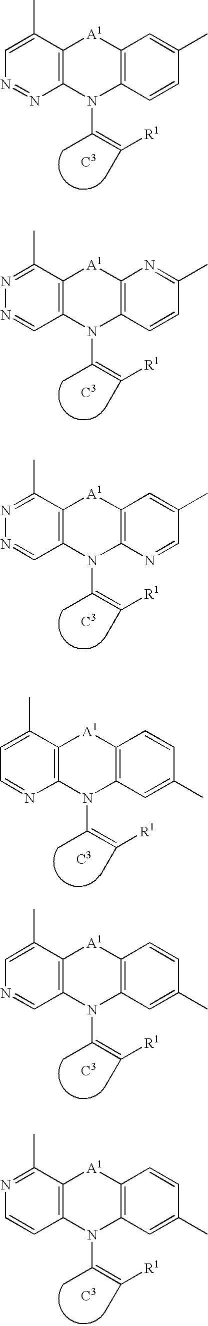

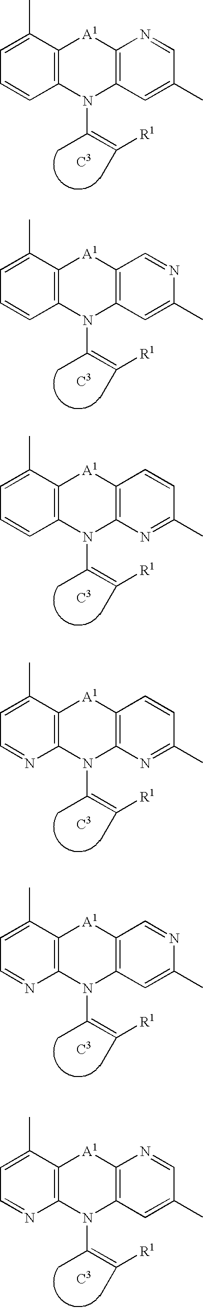

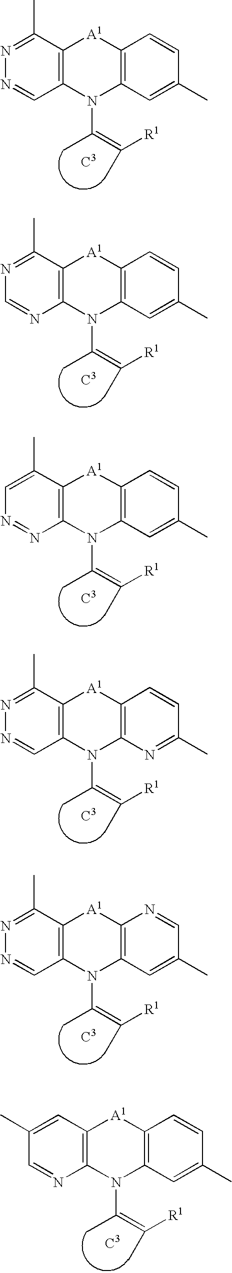

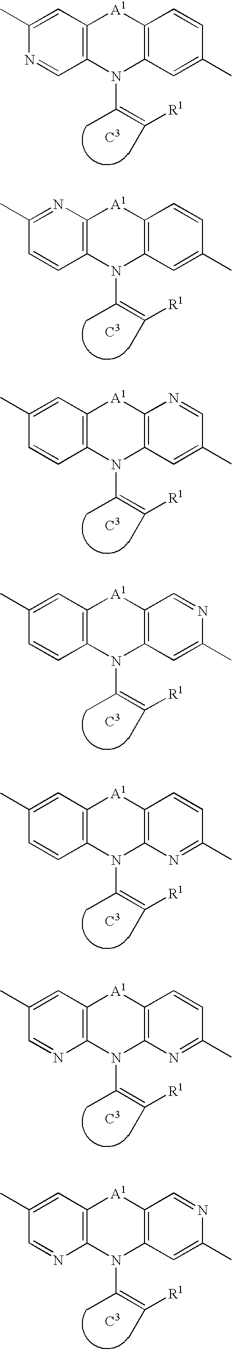

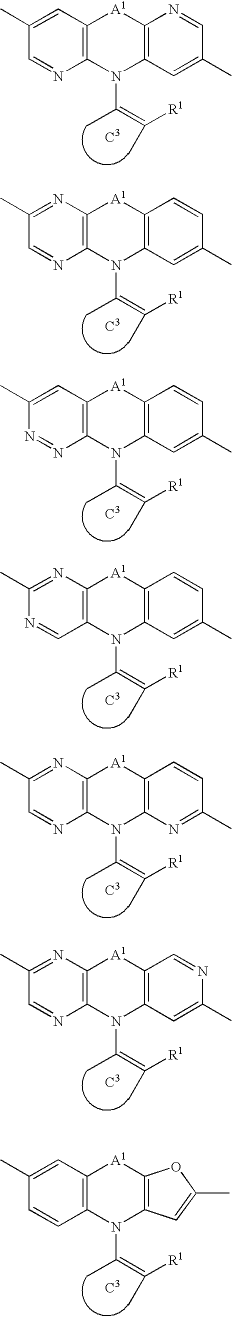

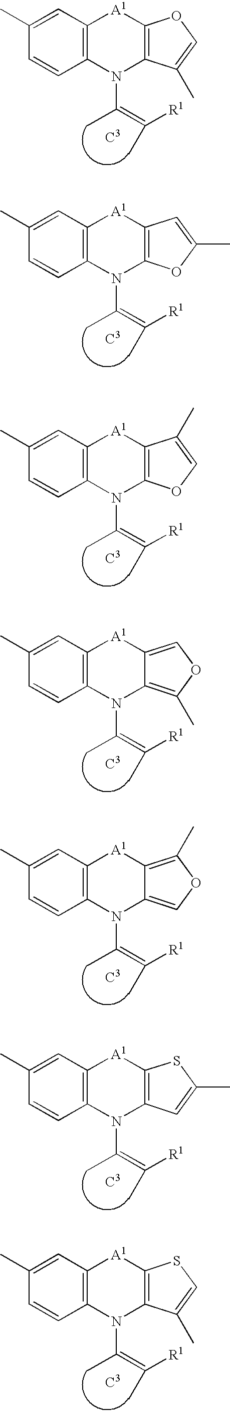

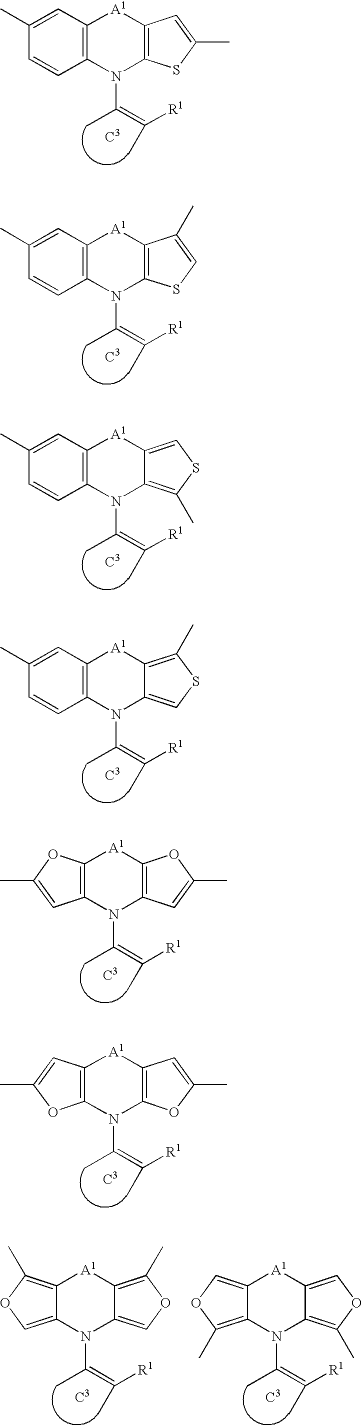

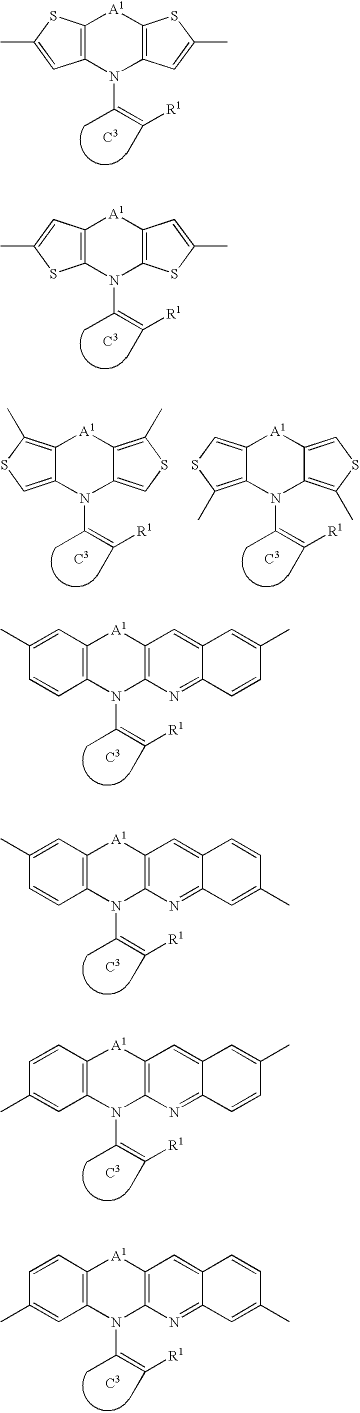

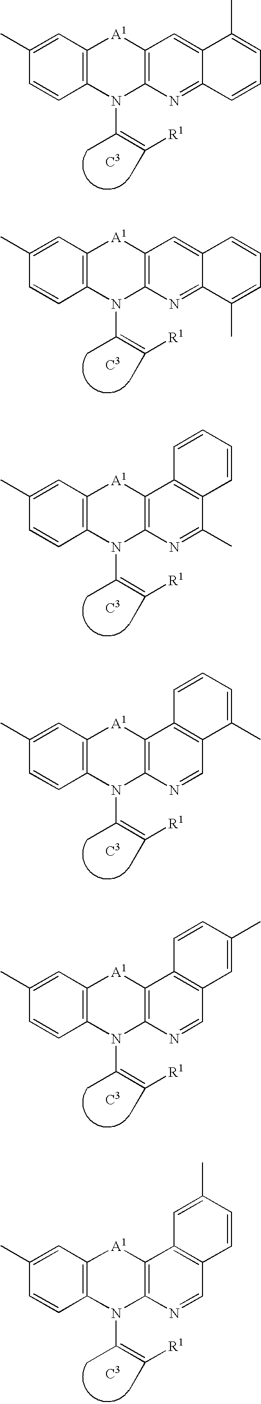

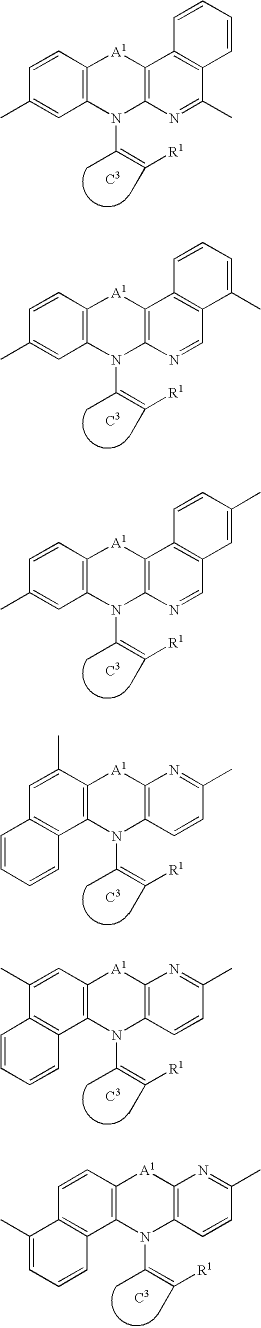

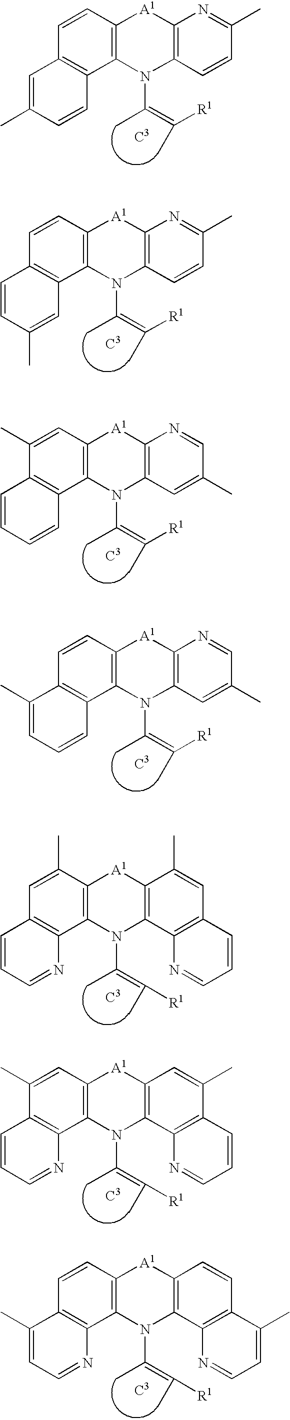

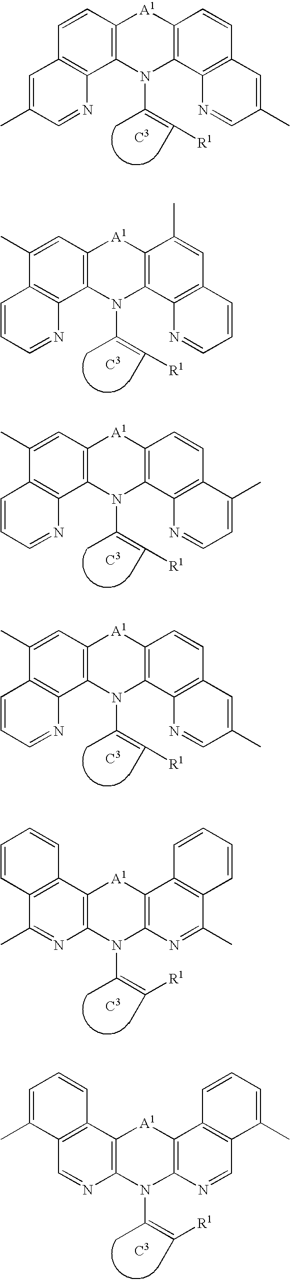

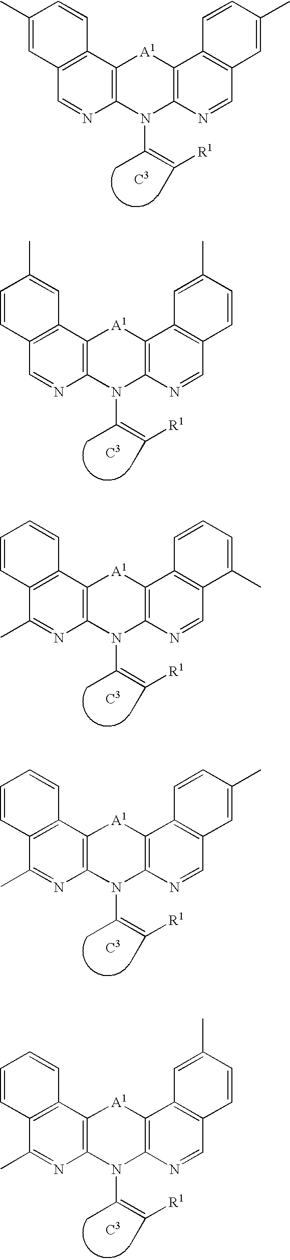

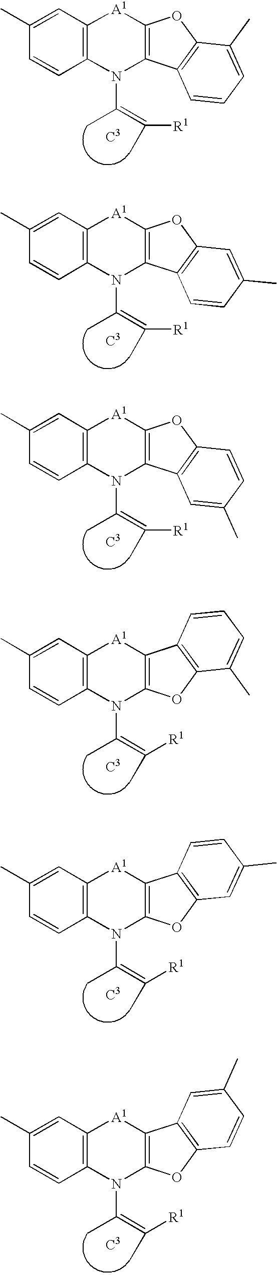

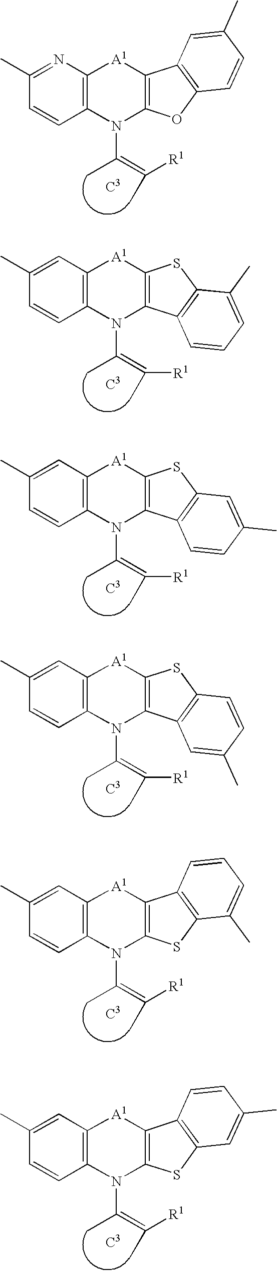

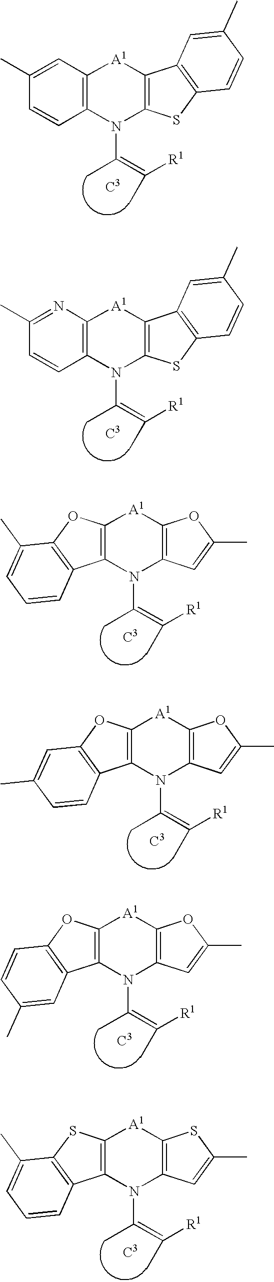

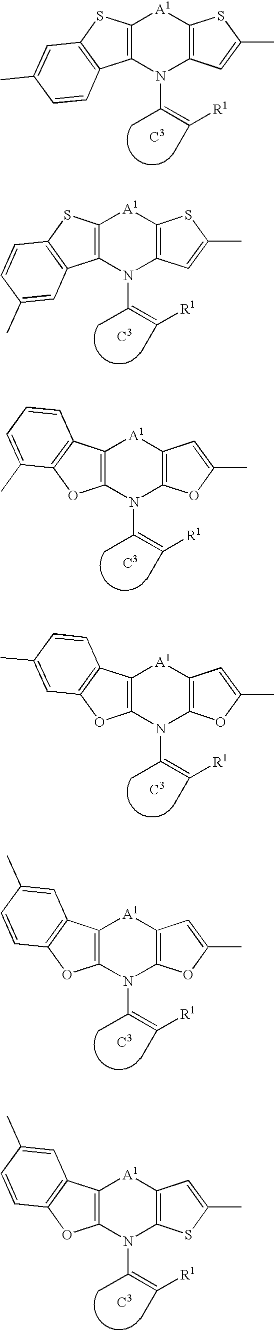

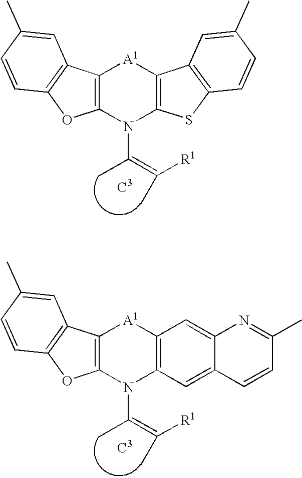

The ring C¹ and the C² are preferably a benzene ring or monocyclic hetero ring, more preferably a 6-membered ring, further preferably a benzene ring, and most preferably a moiety of the following formula (2-1).

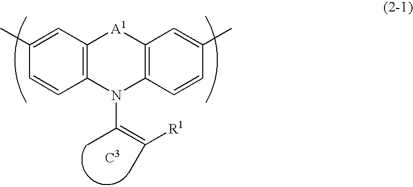

In the formula, A¹, C³ and R¹ have the same meanings as described above. A benzene ring optionally carries thereon a substituent.

As the repeating unit of the above-described formula (2), the following structures are exemplified. A benzene ring or hetero ring optionally carries thereon a substituent.

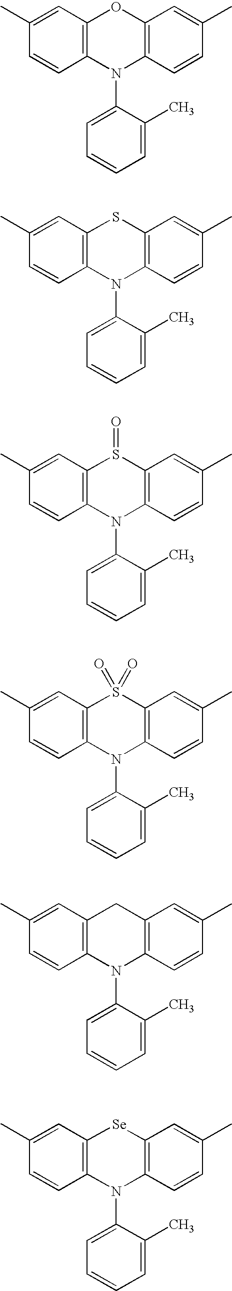

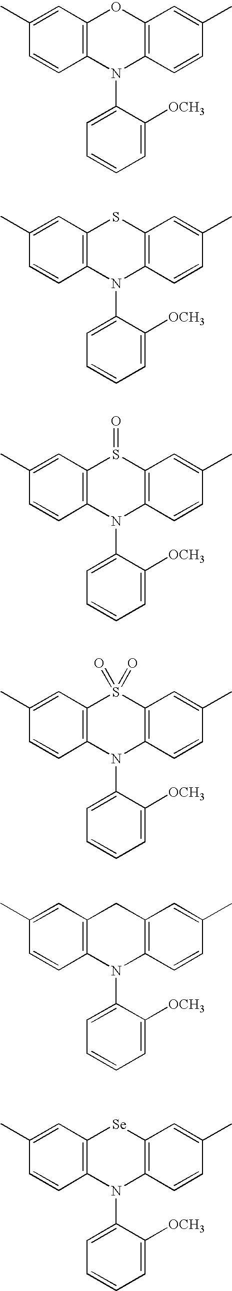

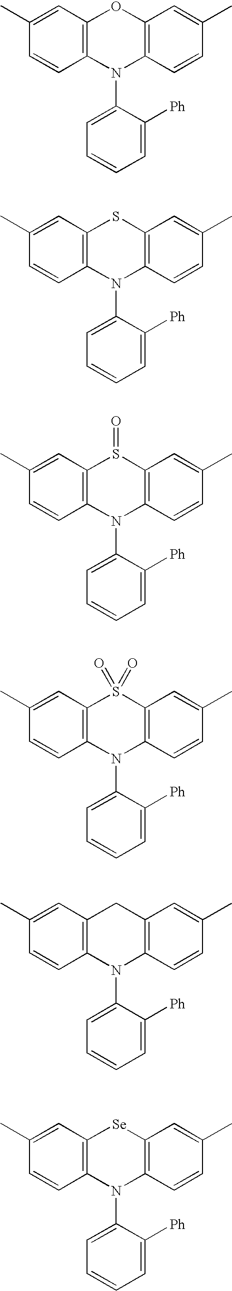

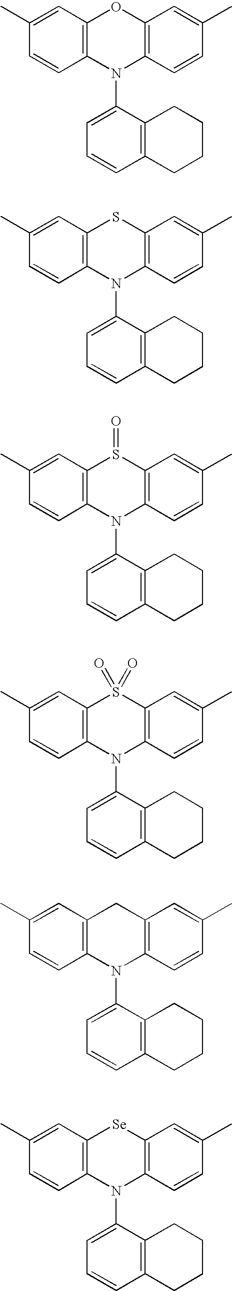

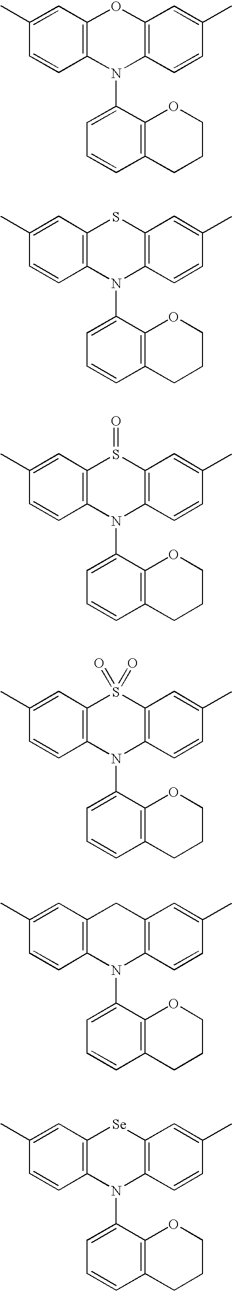

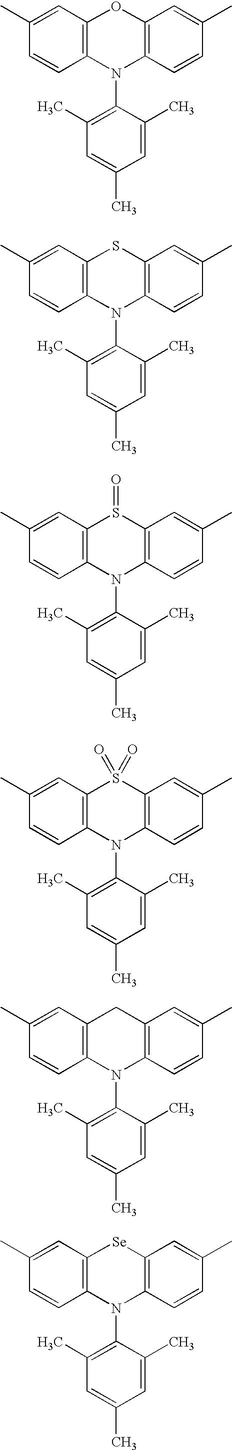

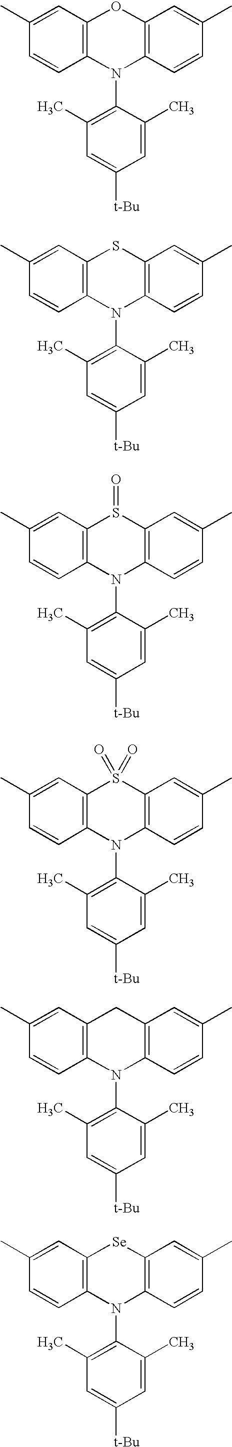

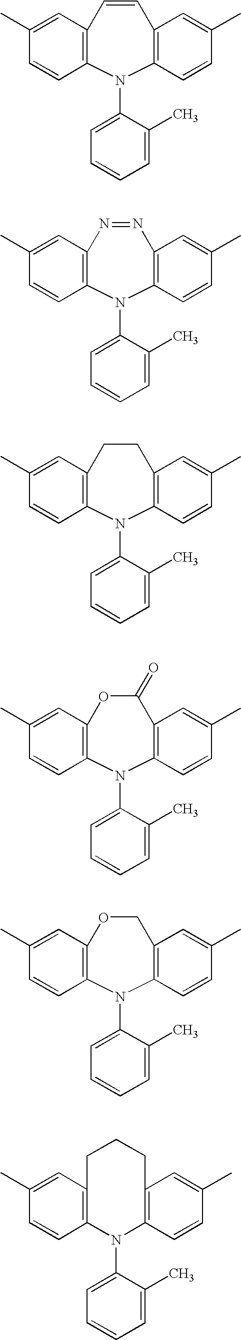

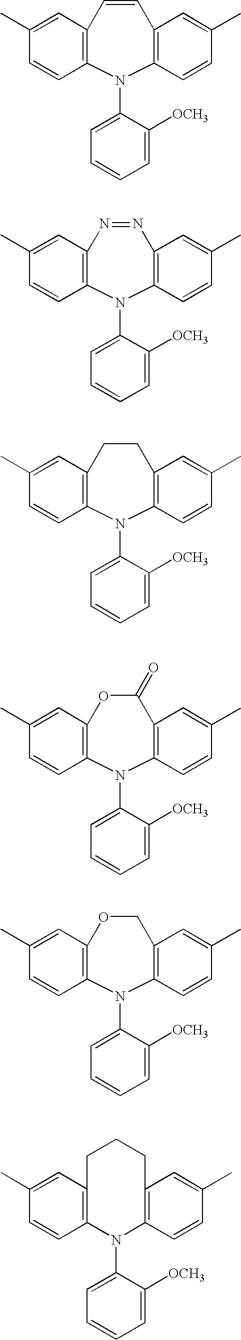

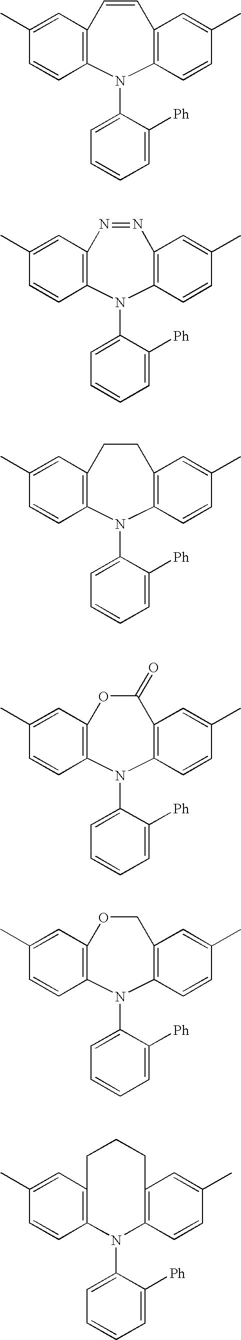

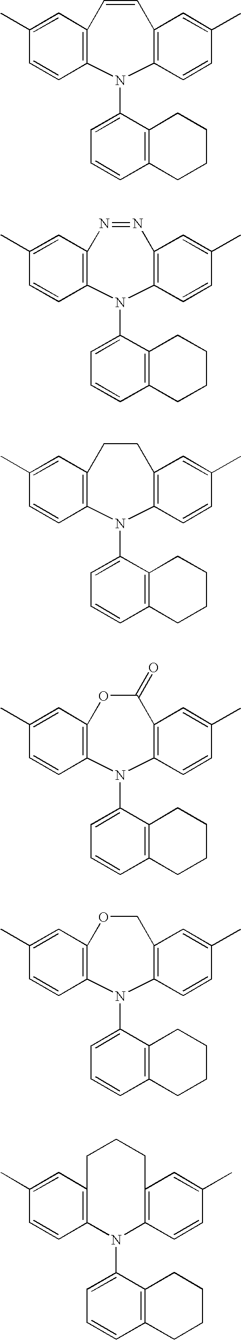

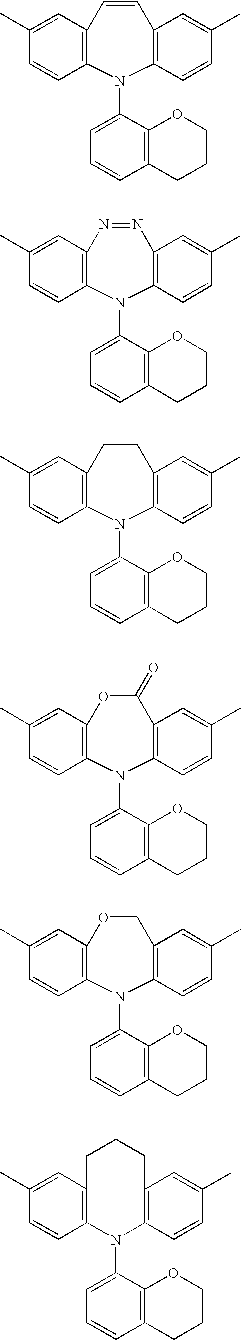

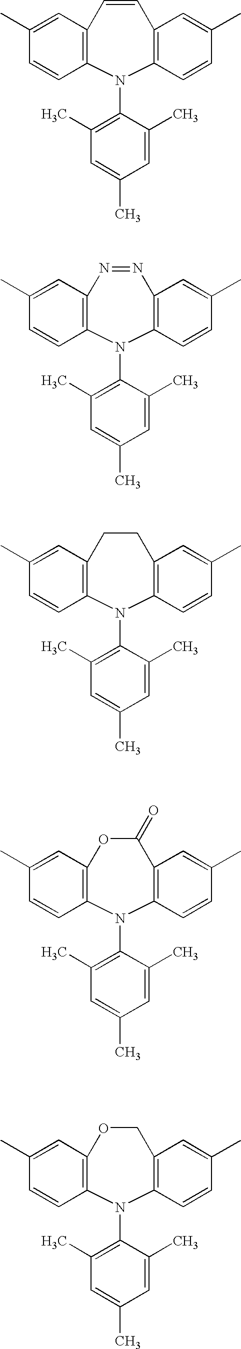

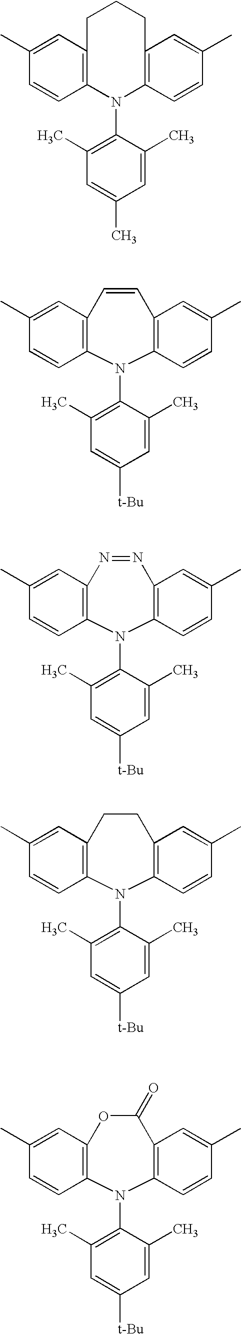

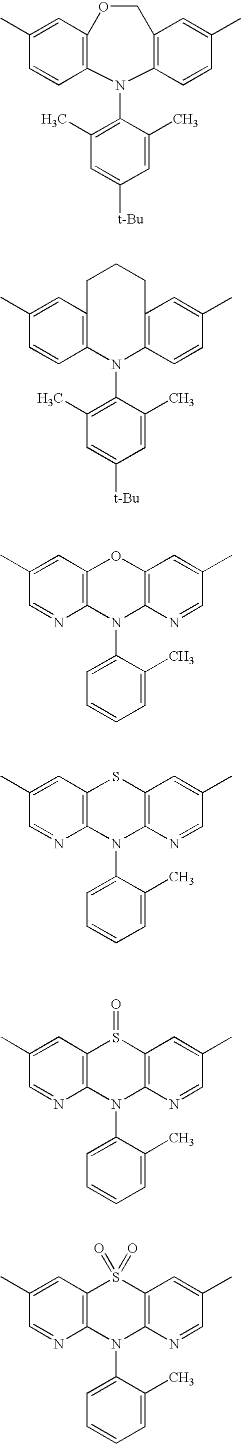

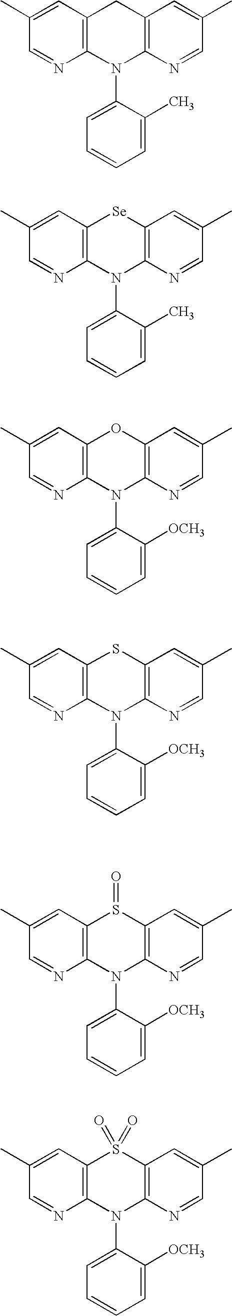

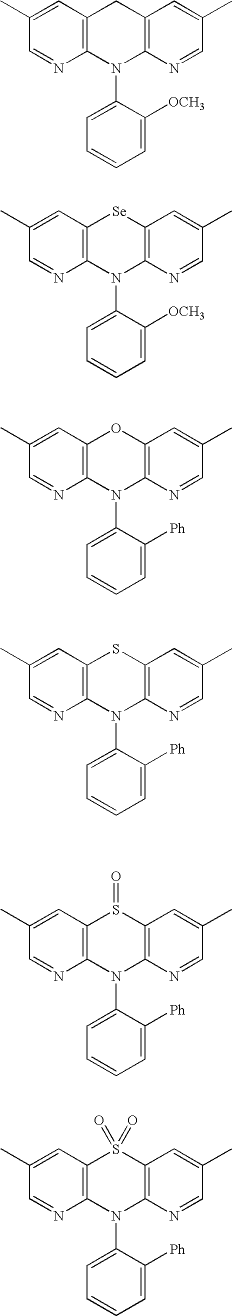

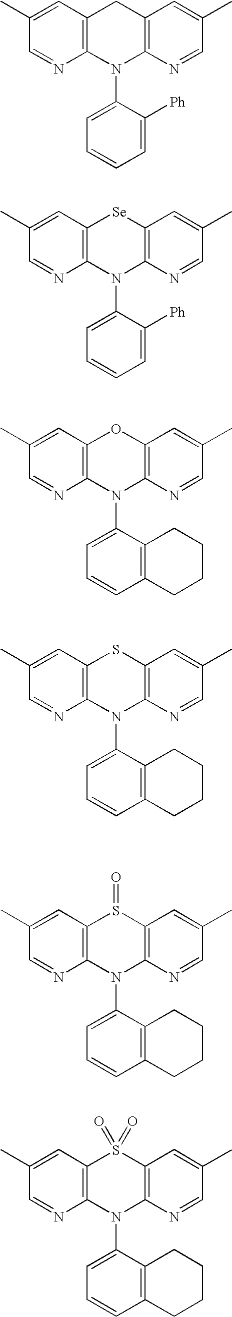

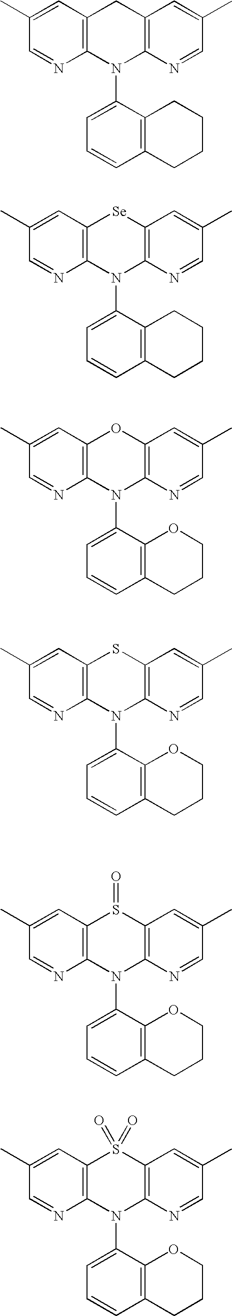

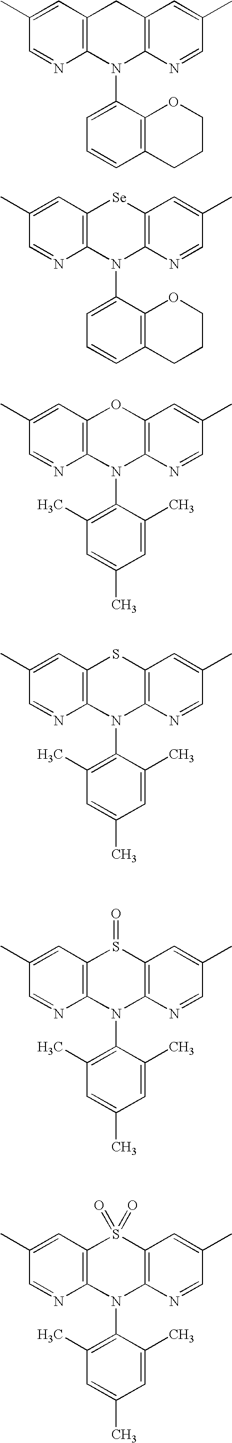

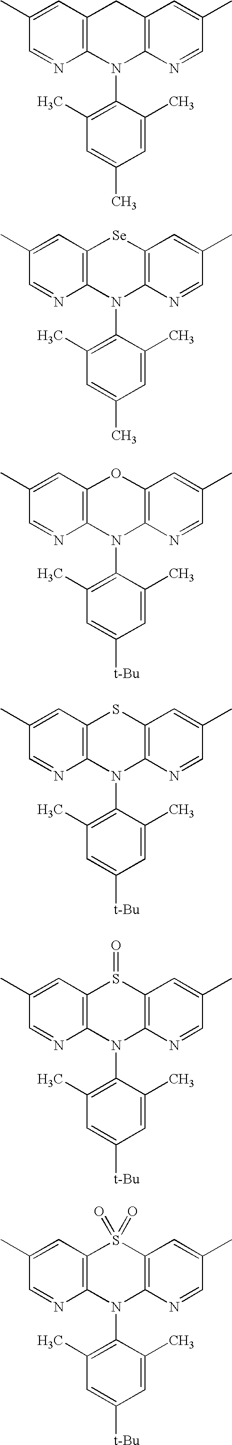

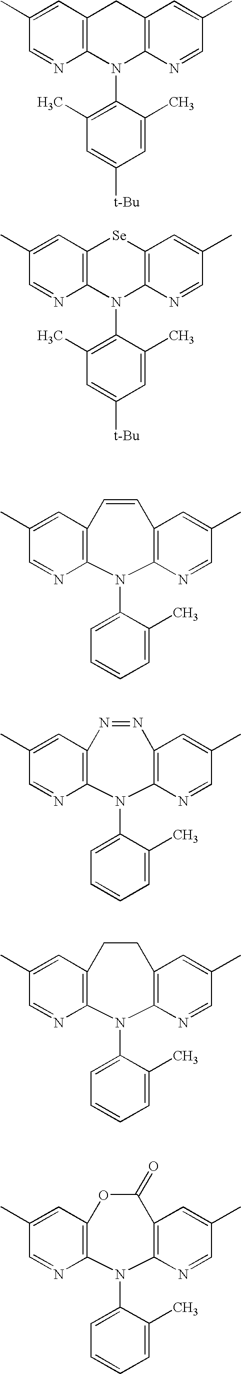

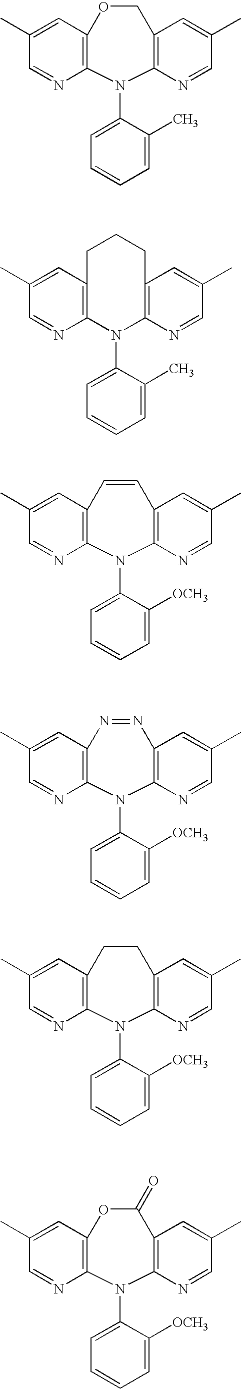

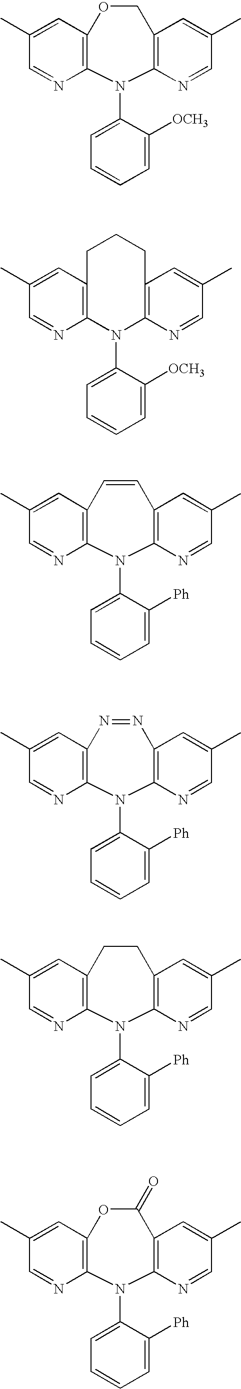

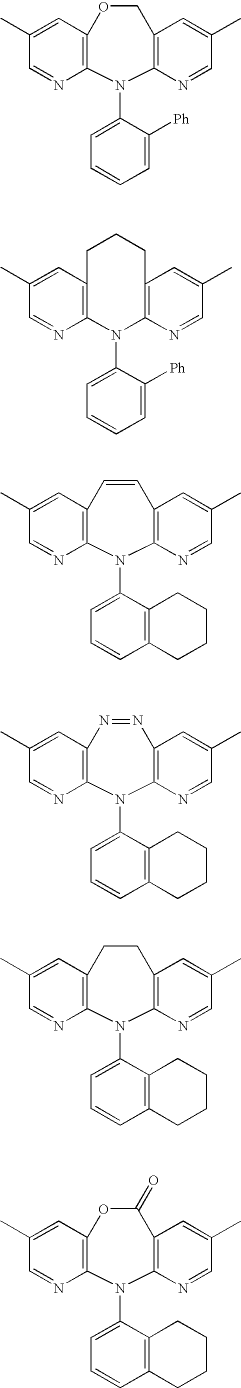

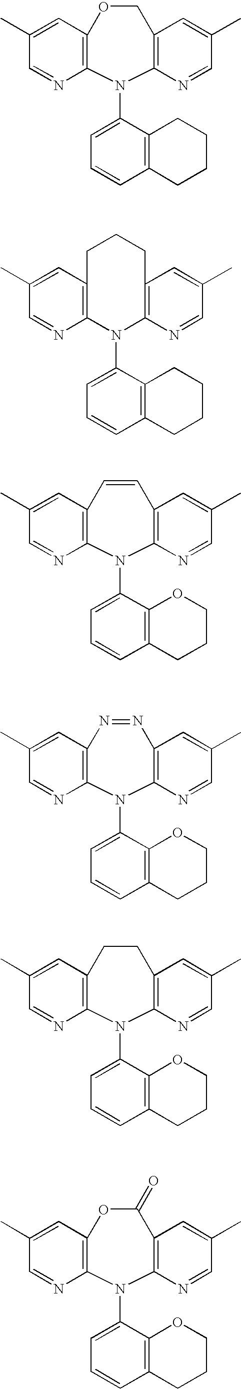

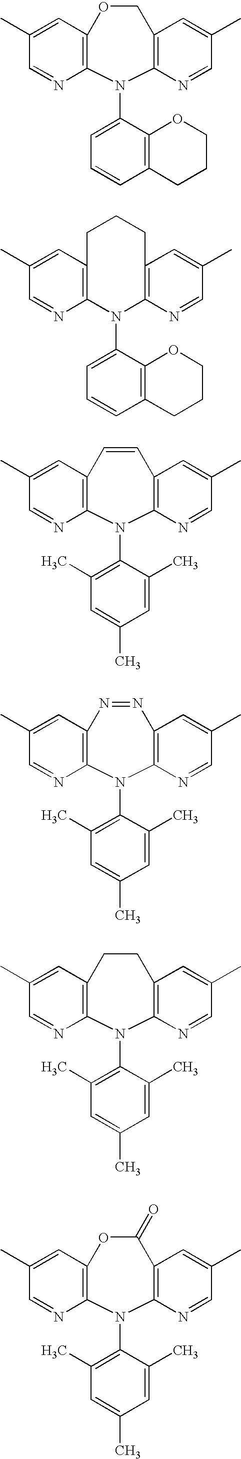

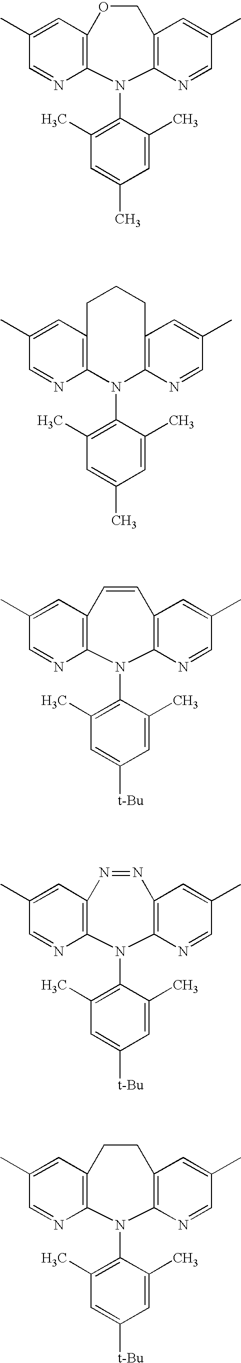

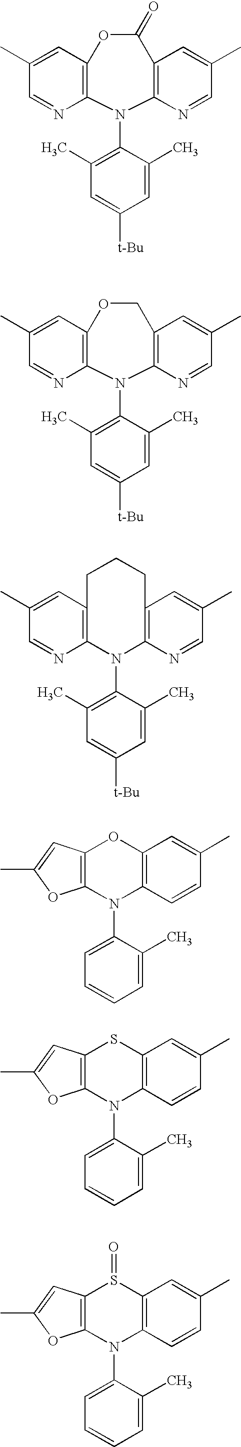

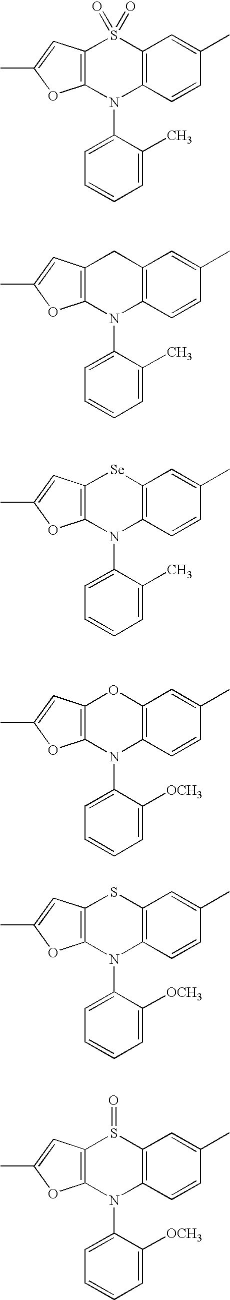

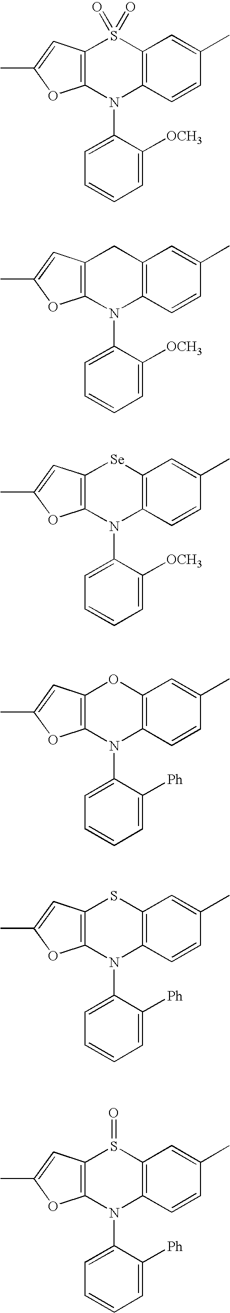

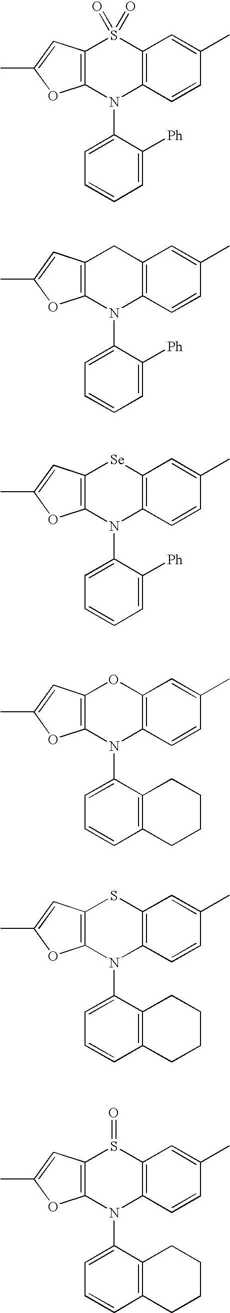

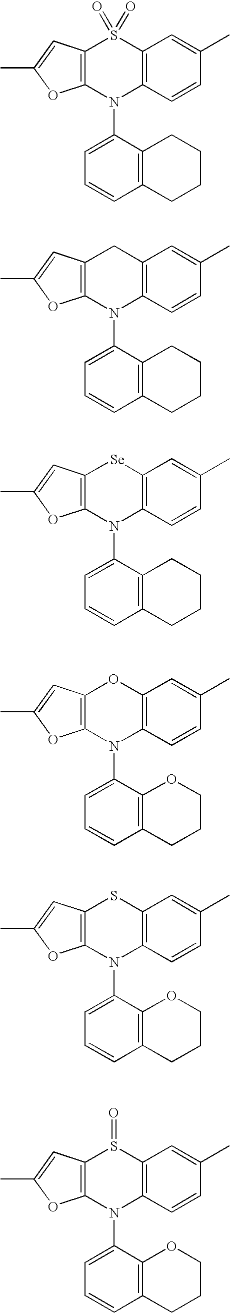

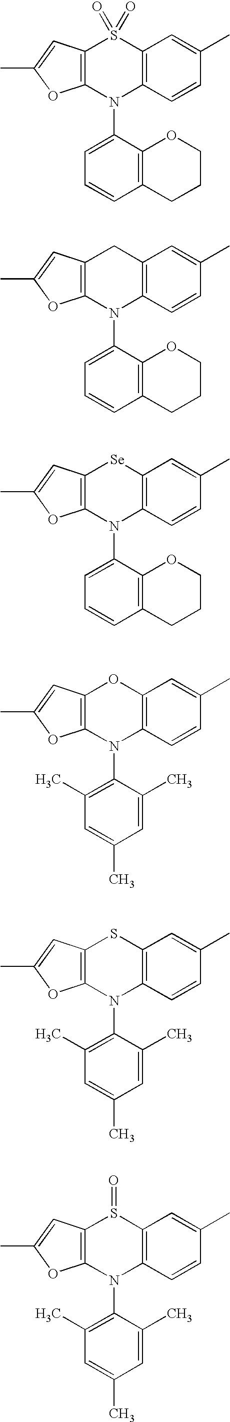

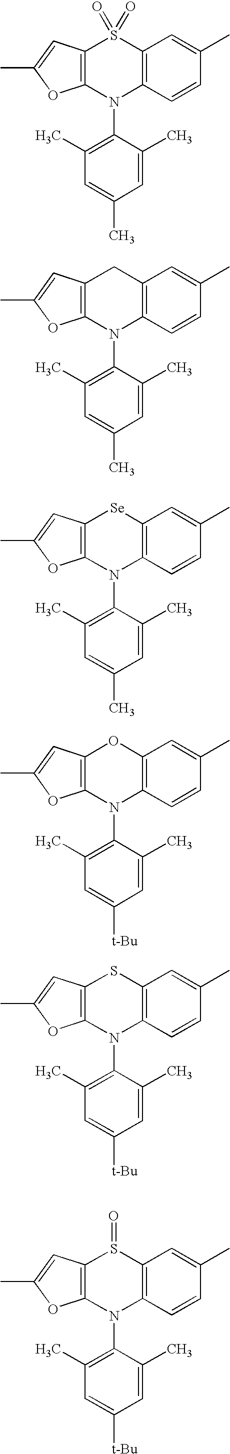

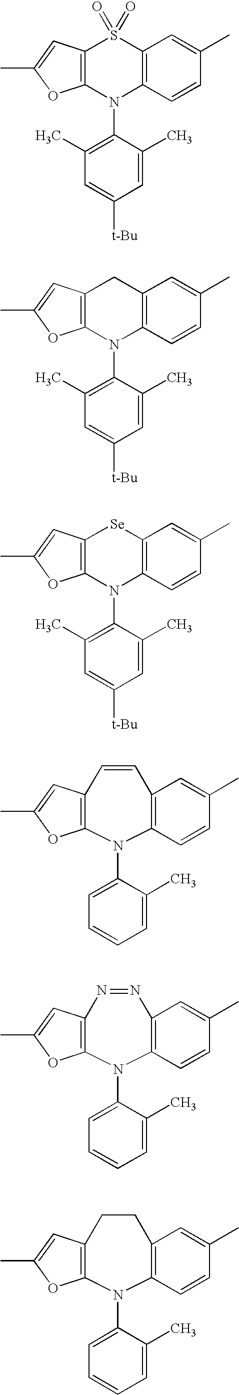

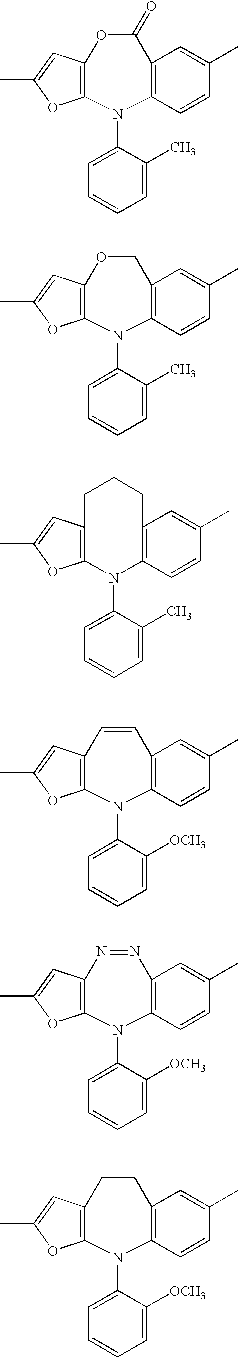

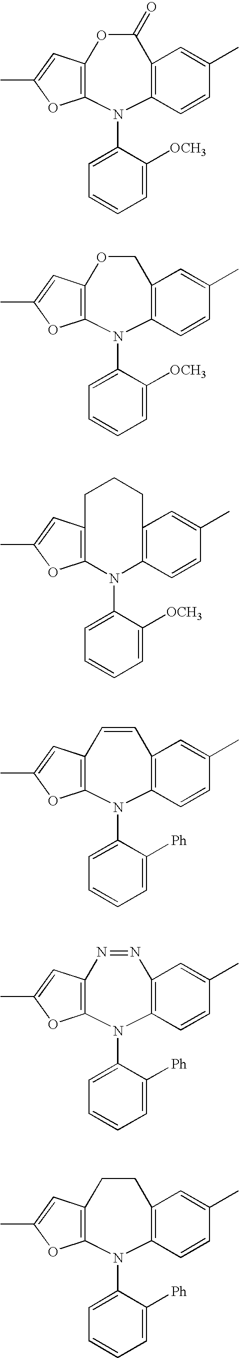

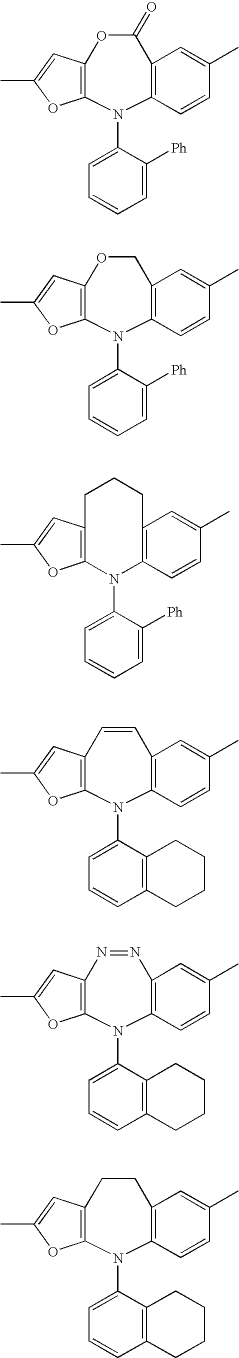

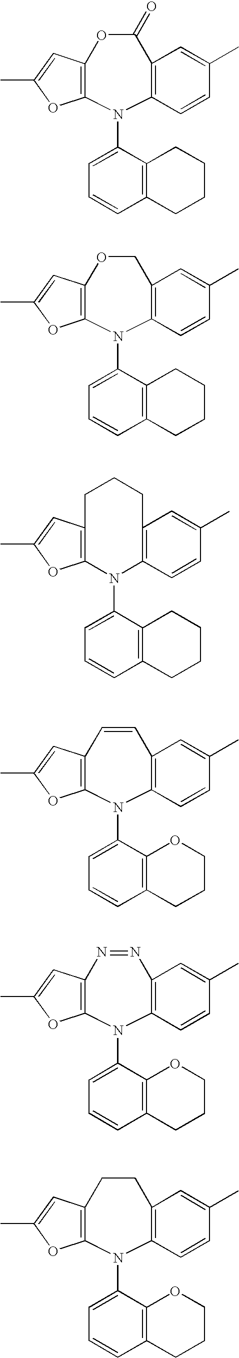

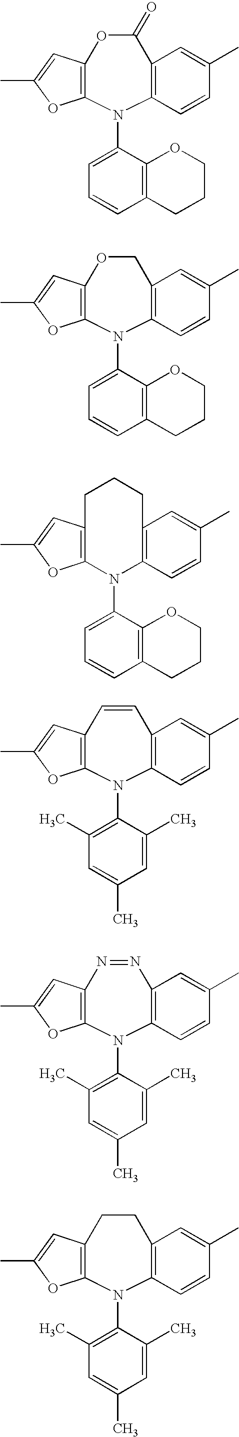

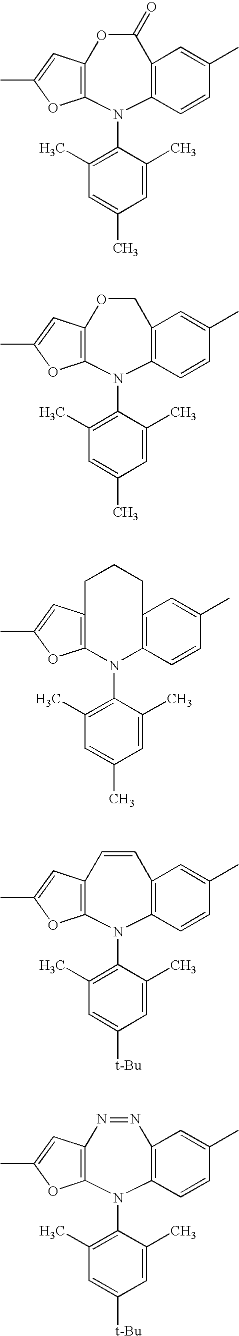

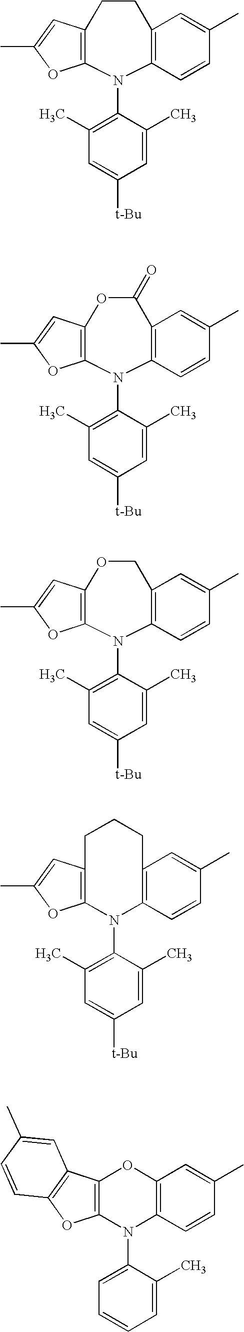

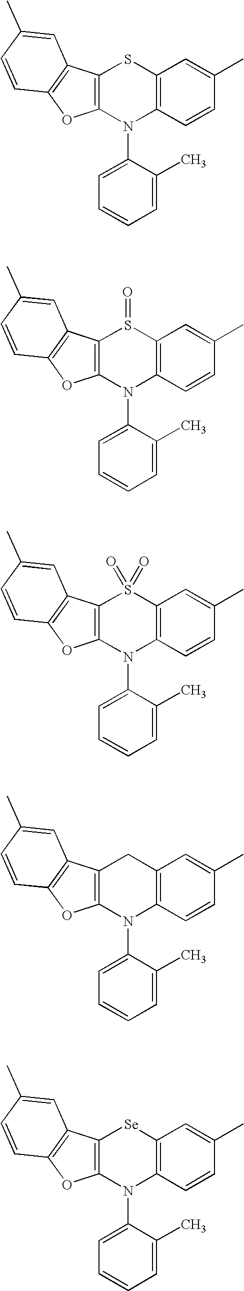

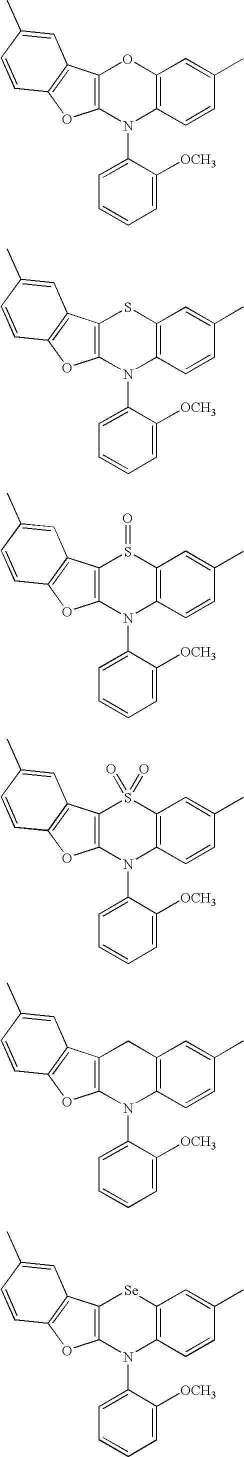

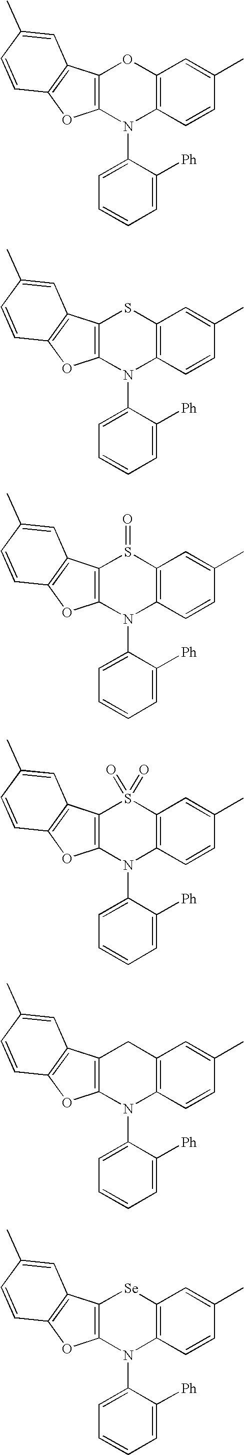

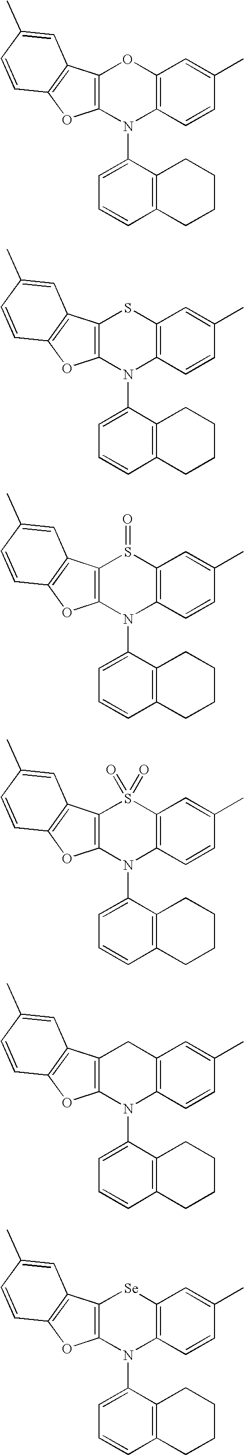

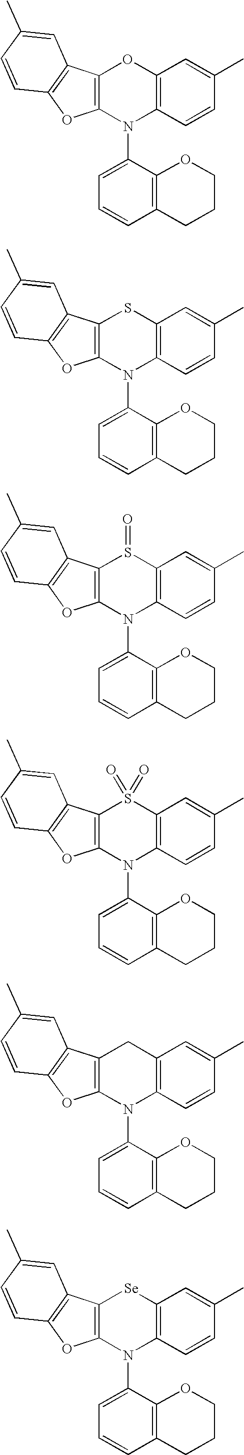

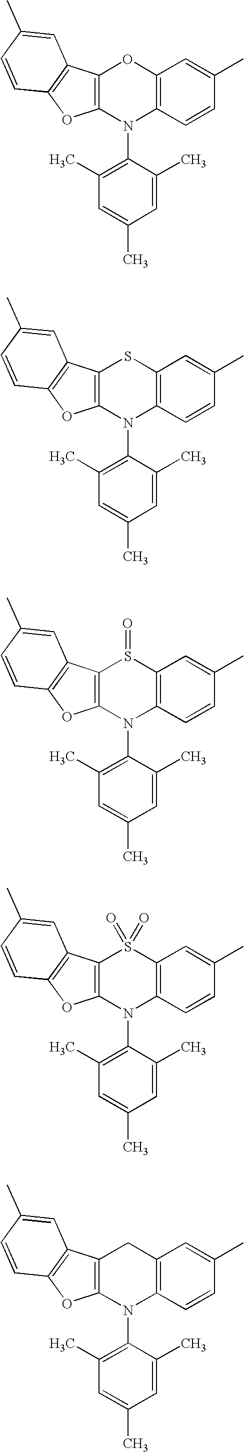

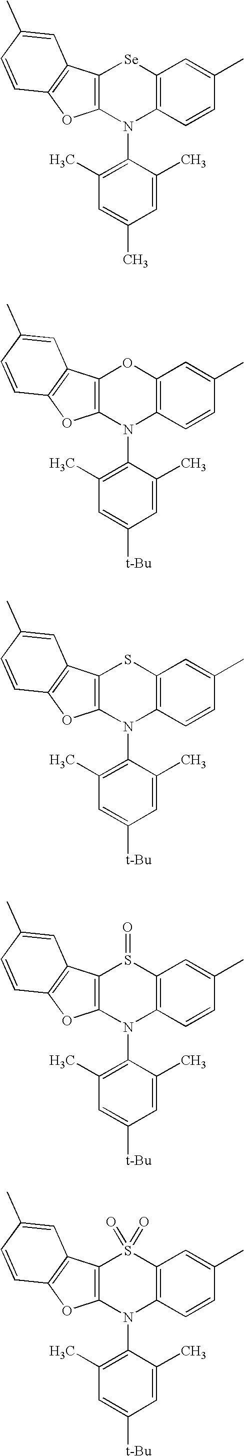

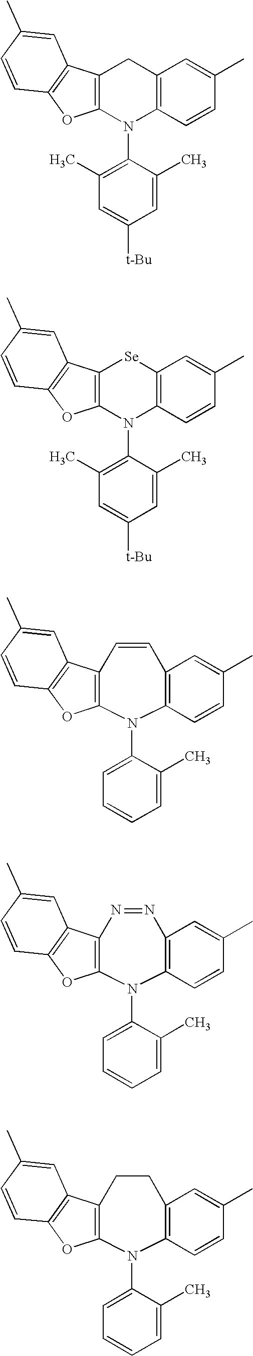

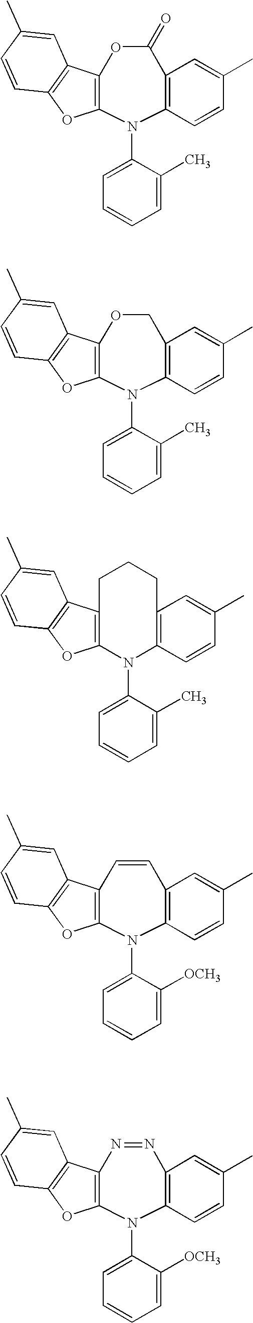

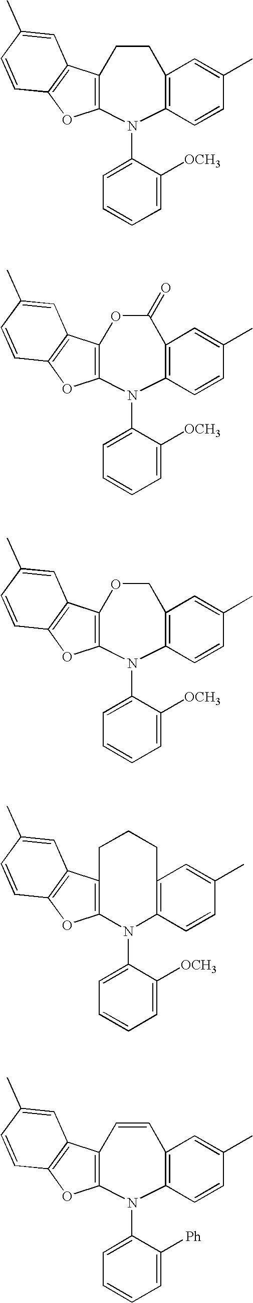

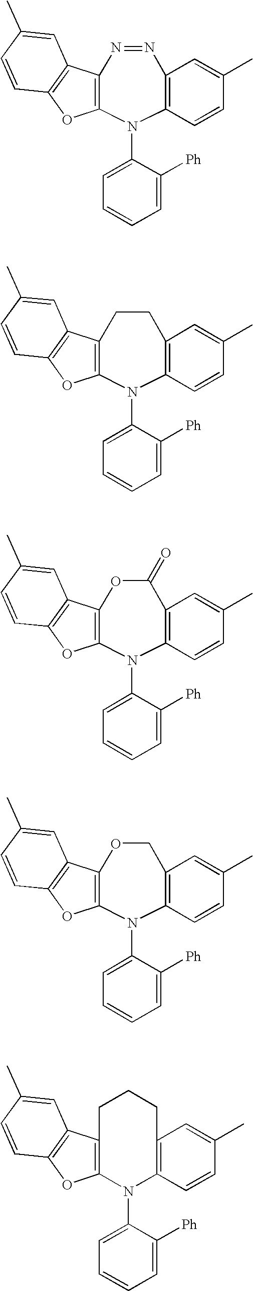

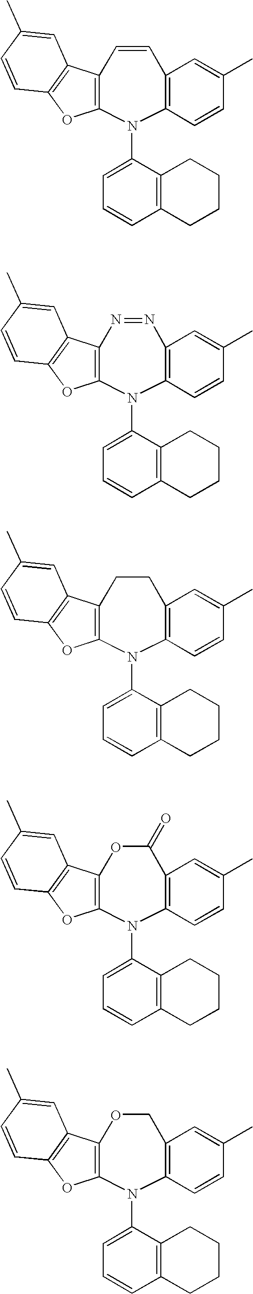

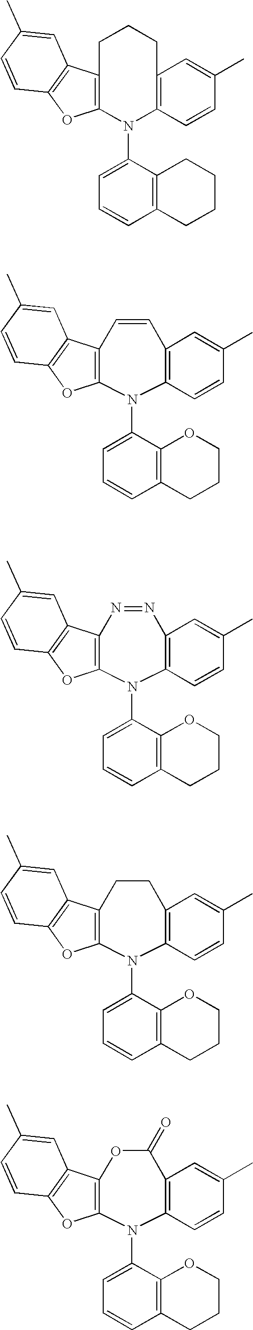

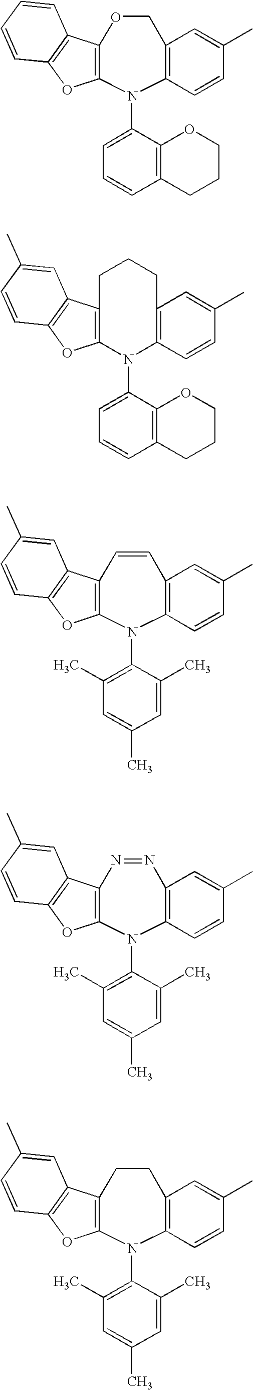

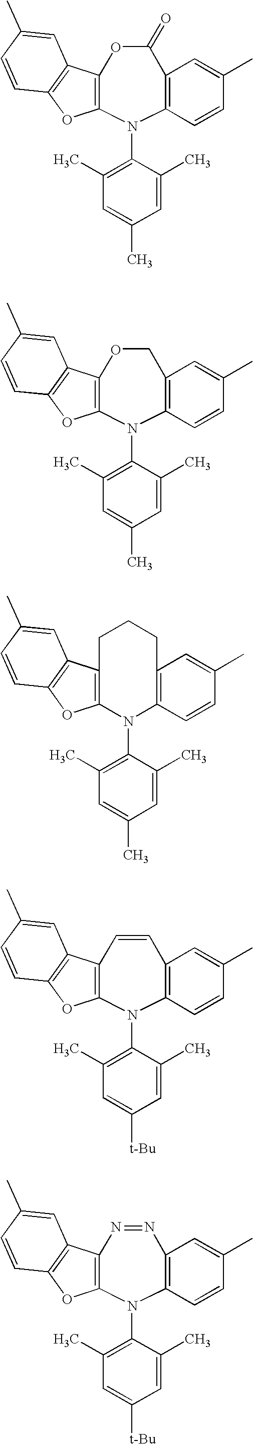

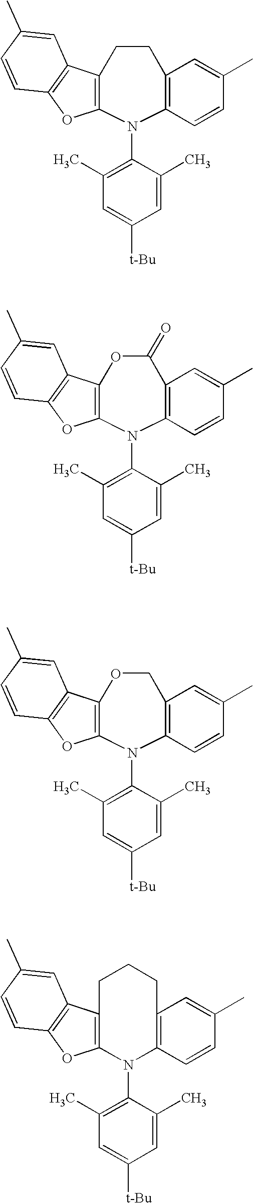

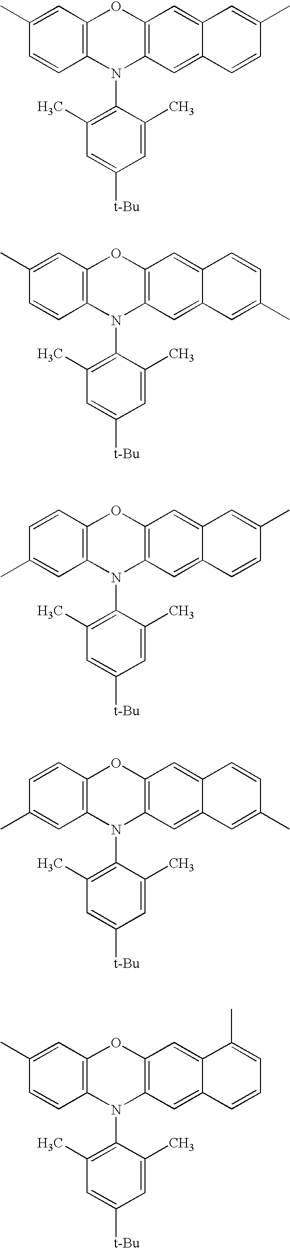

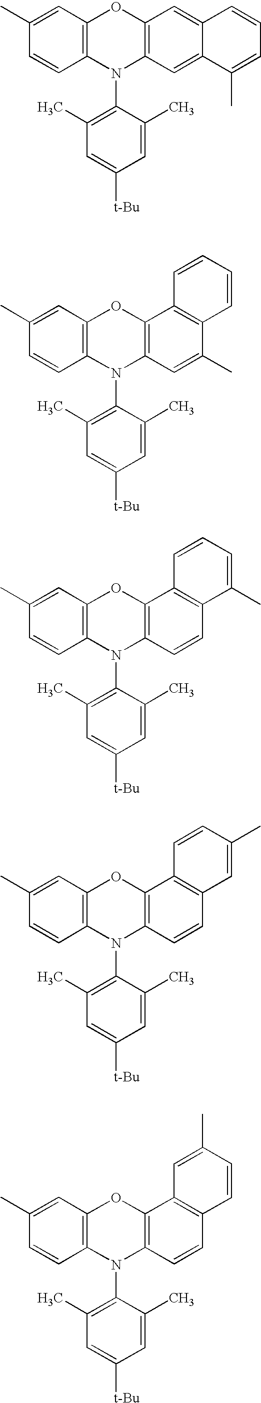

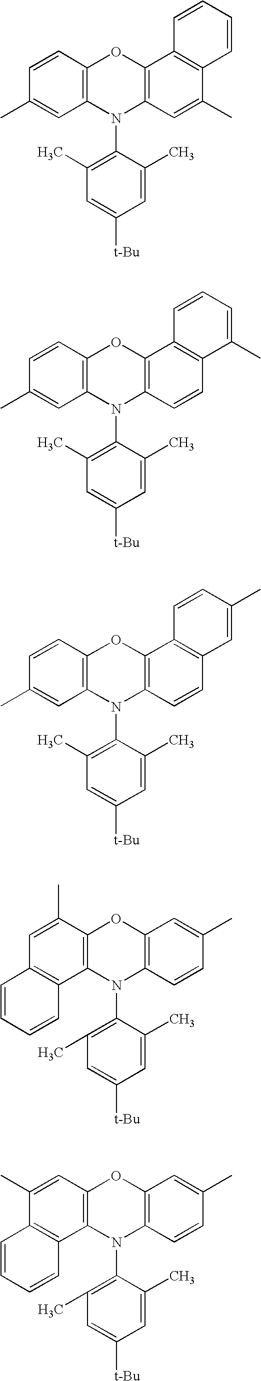

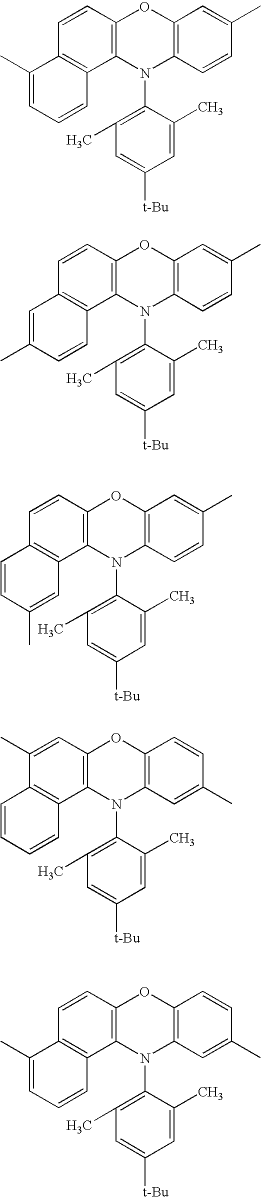

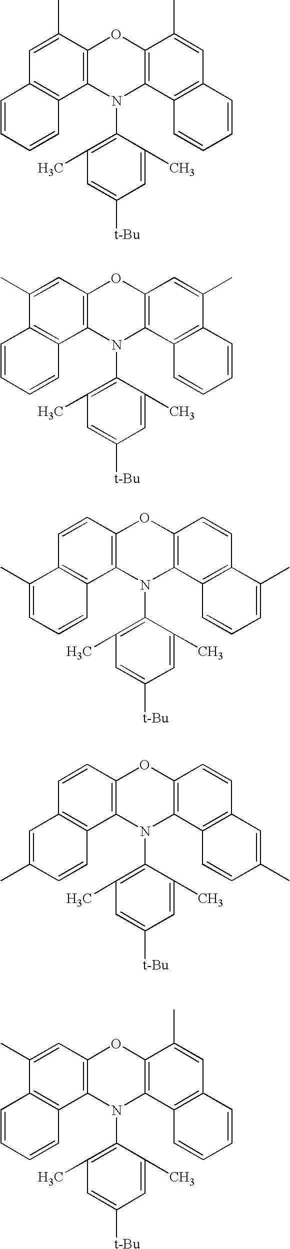

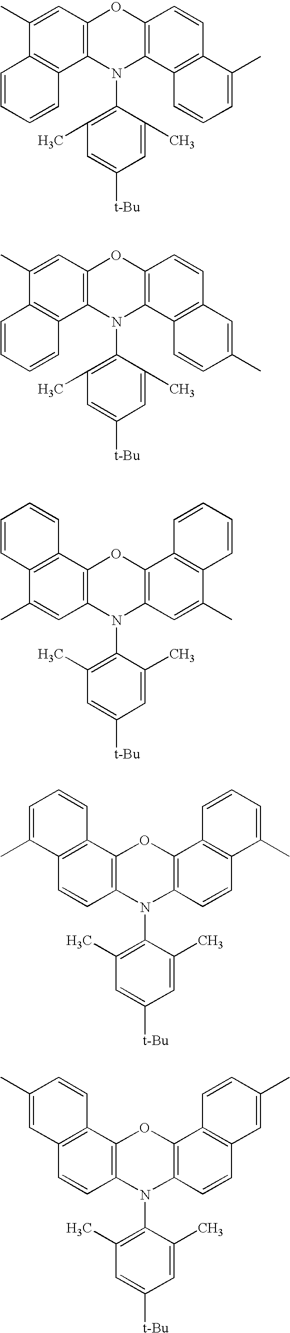

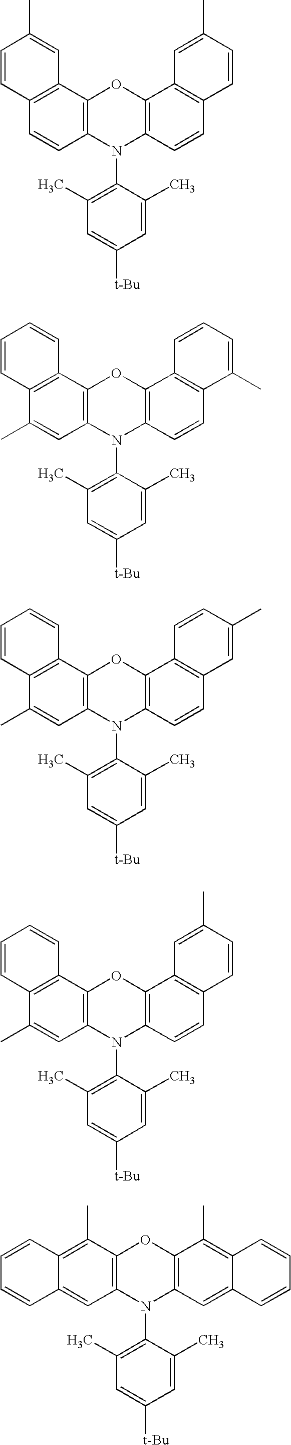

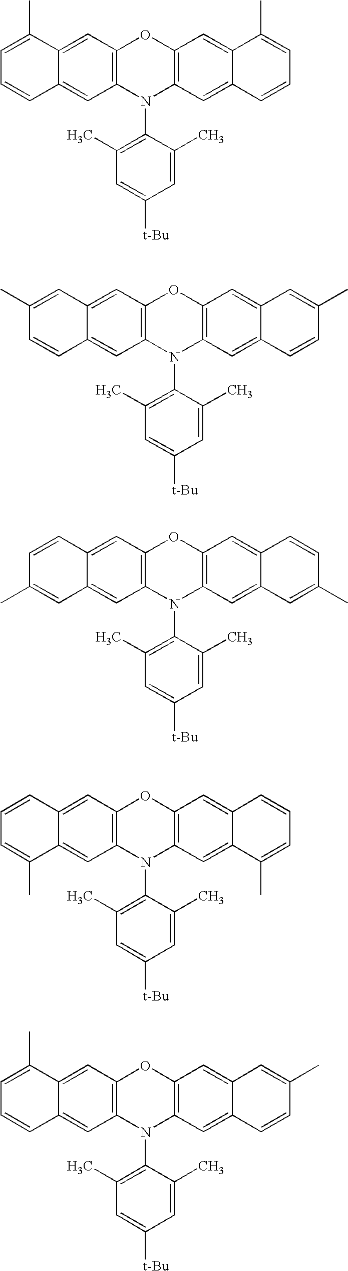

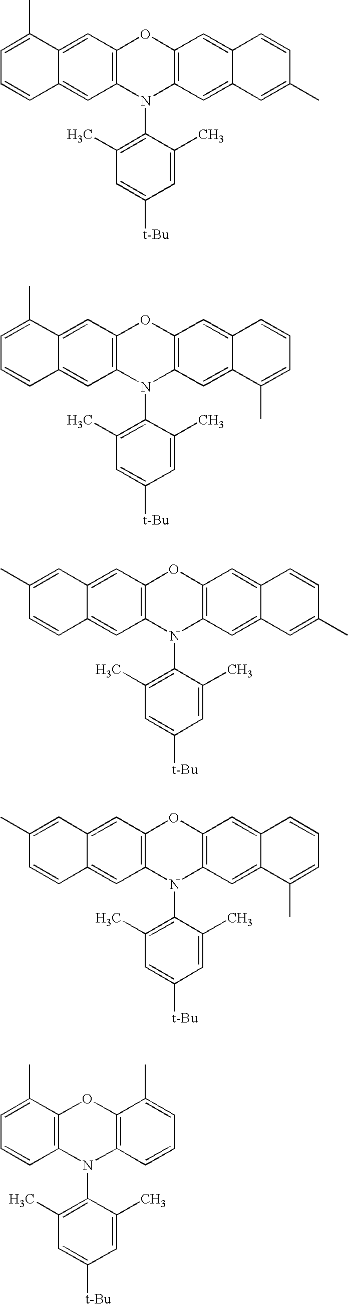

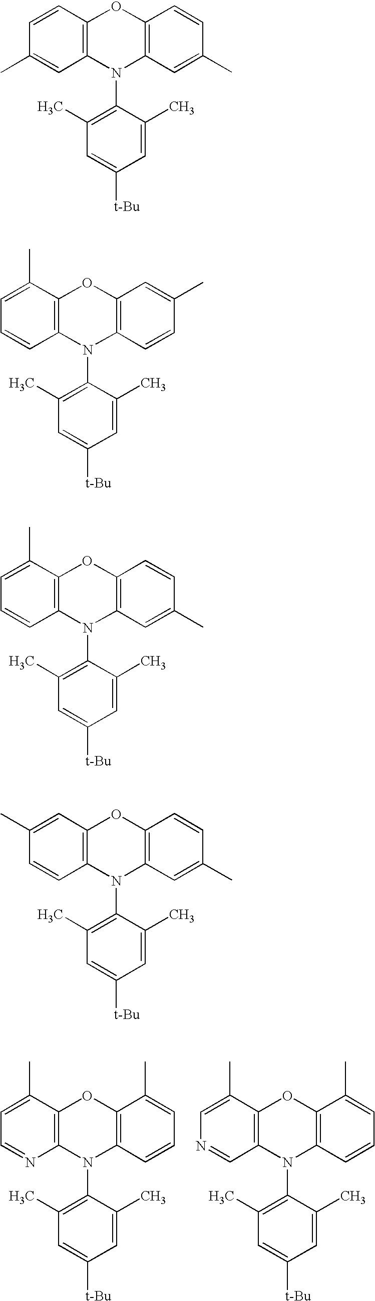

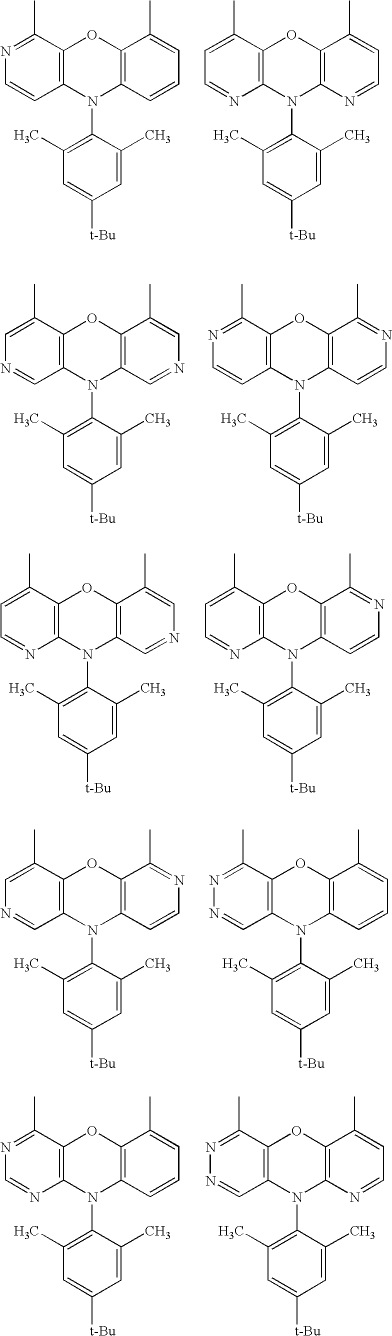

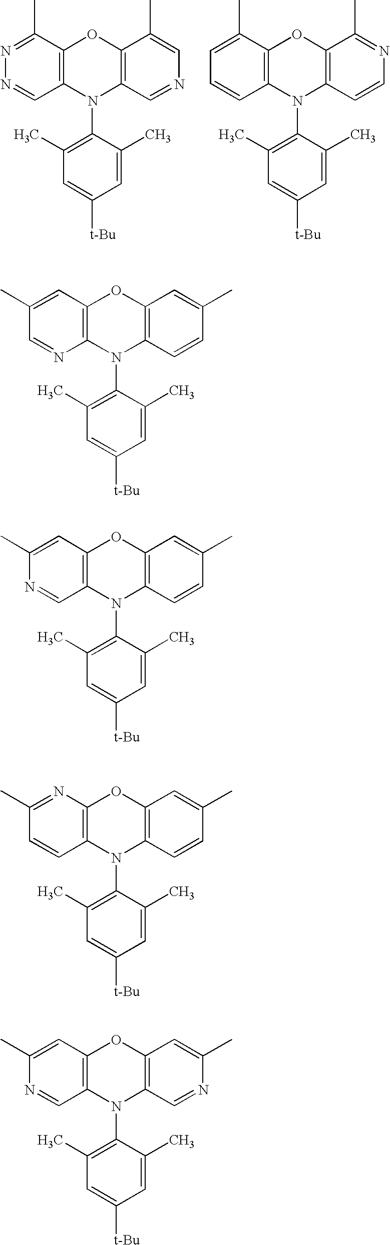

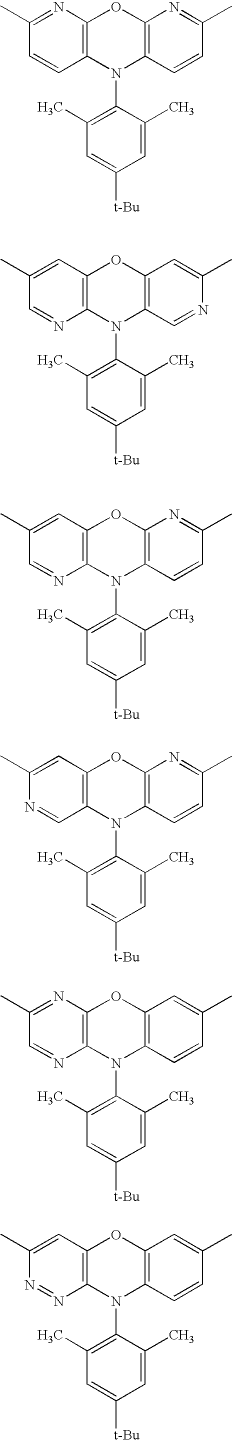

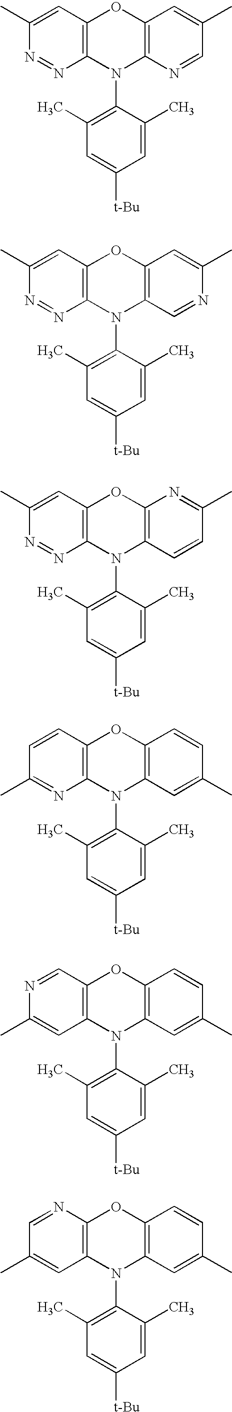

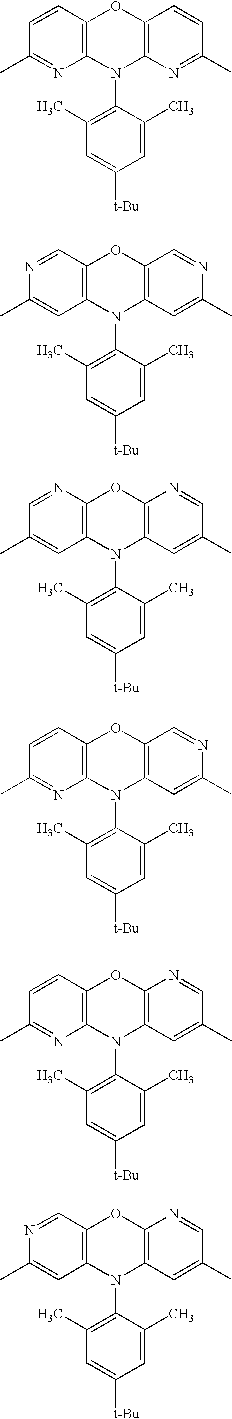

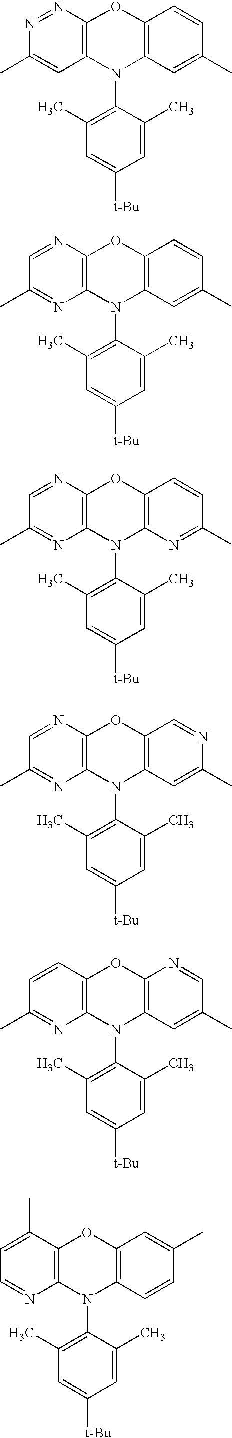

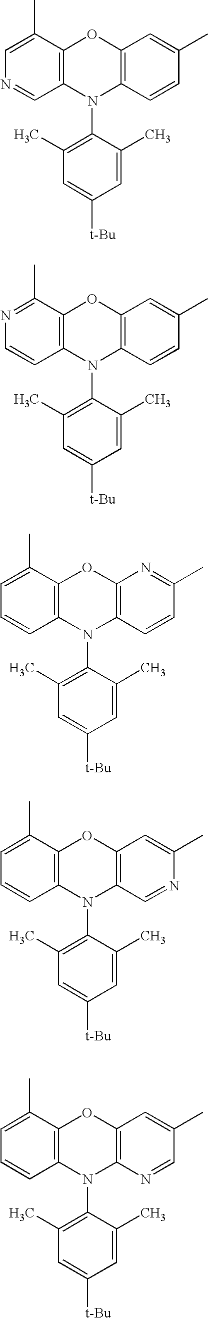

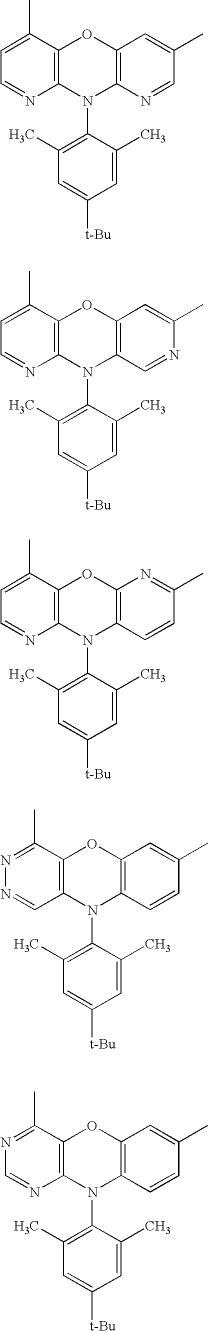

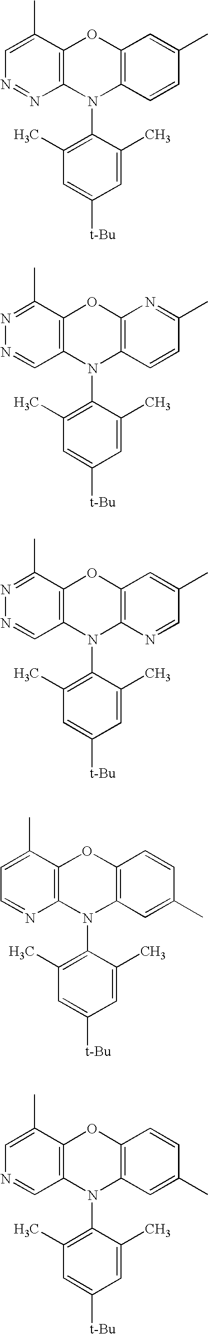

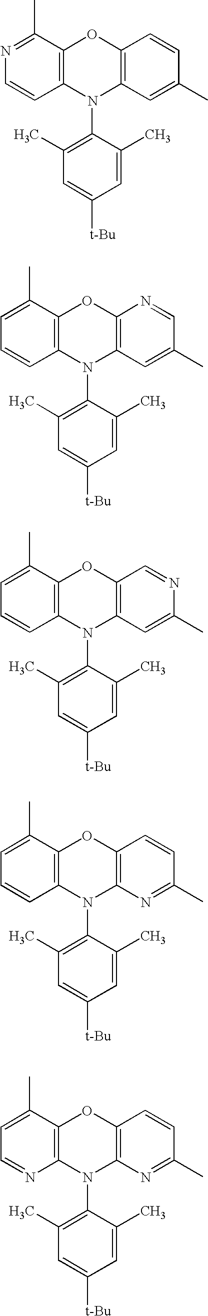

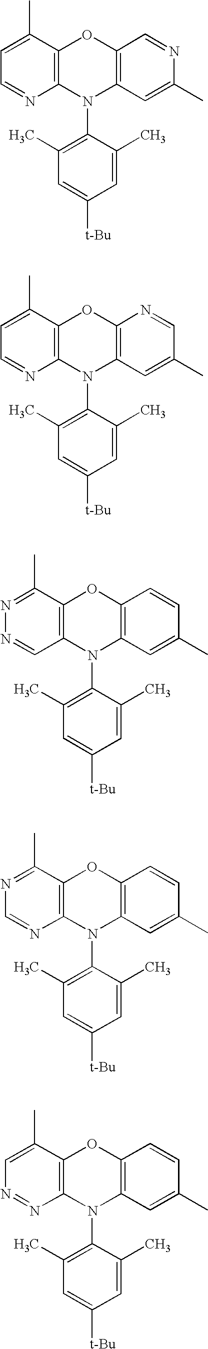

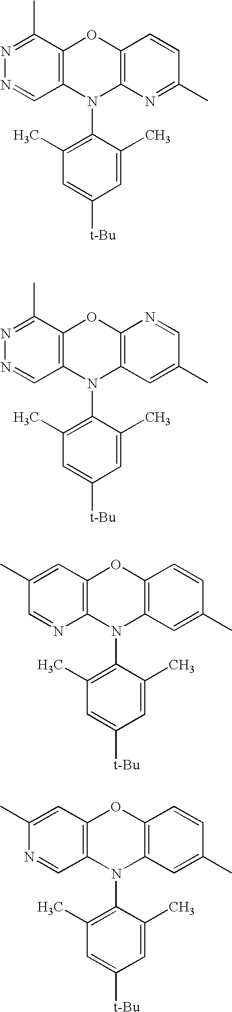

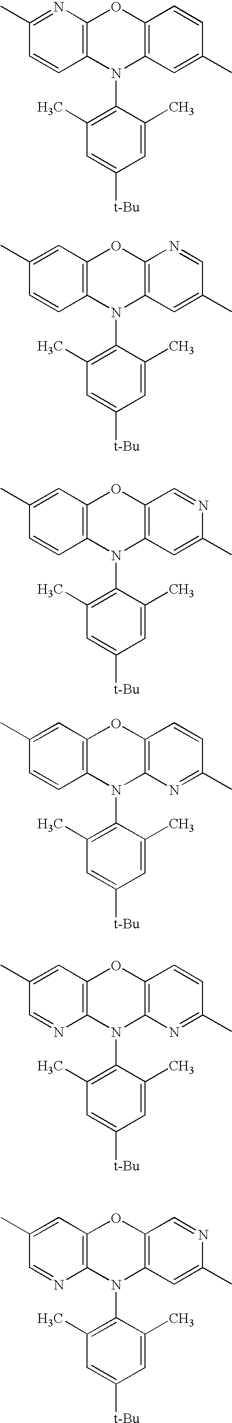

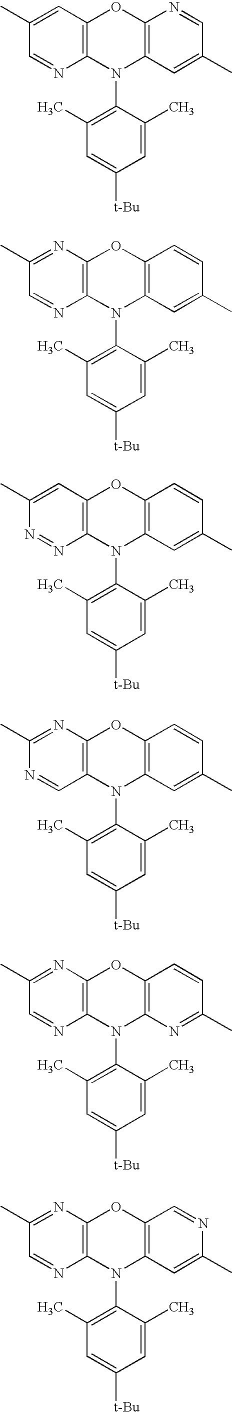

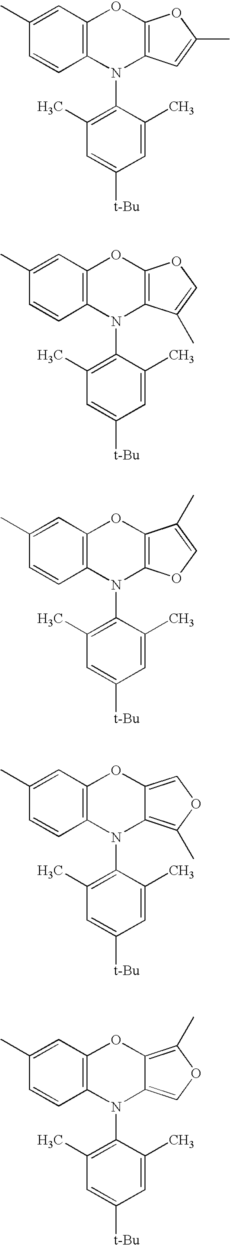

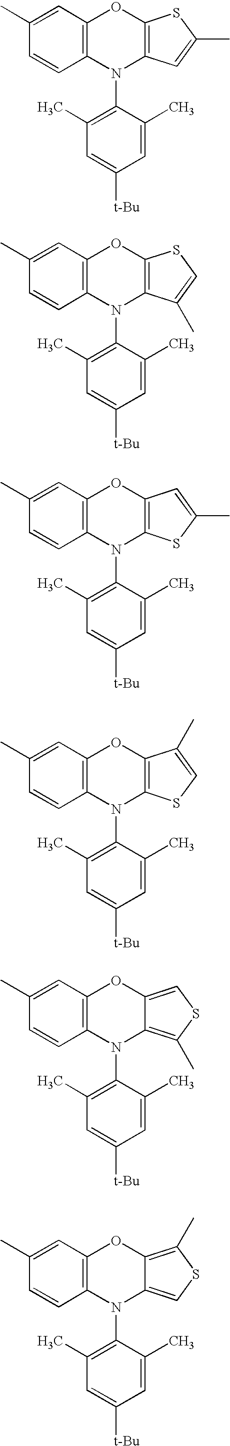

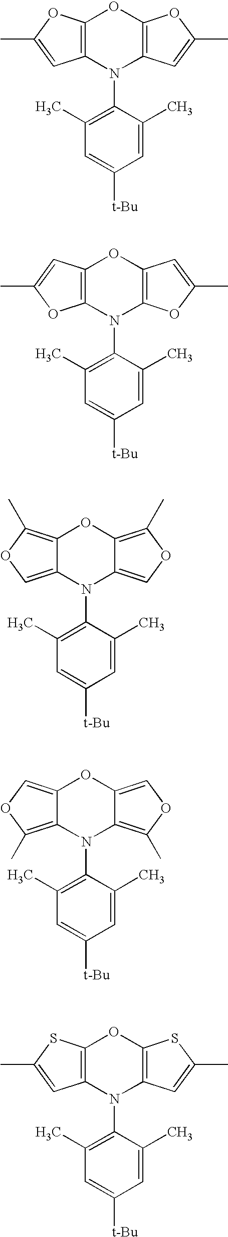

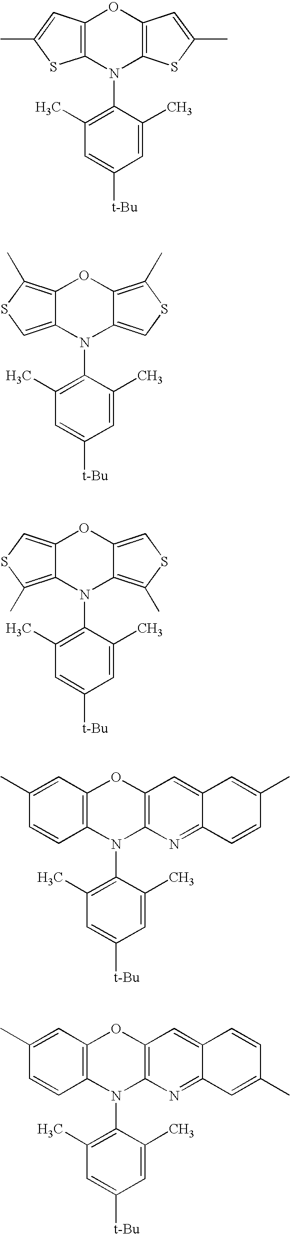

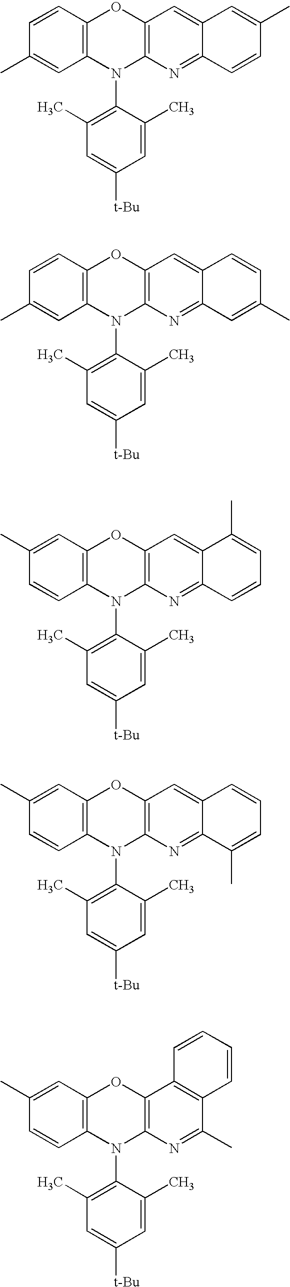

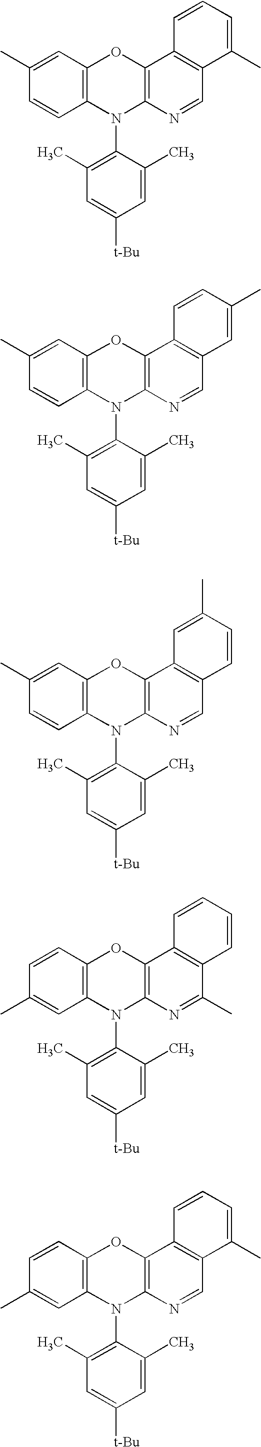

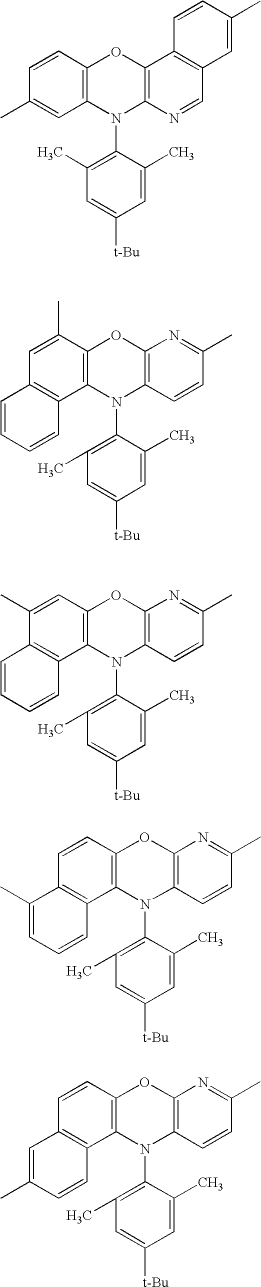

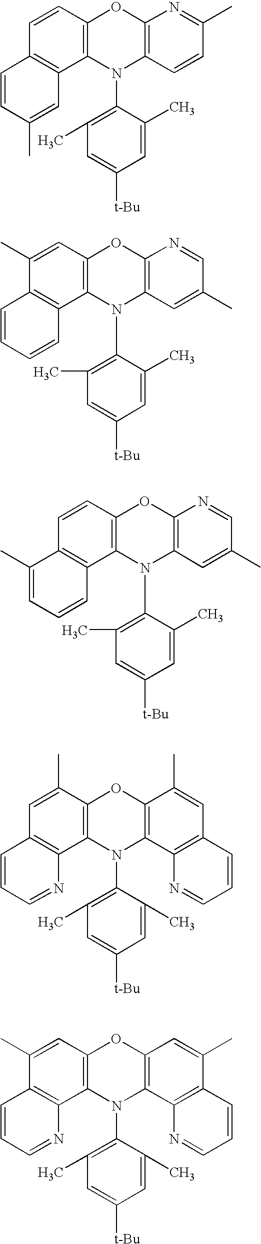

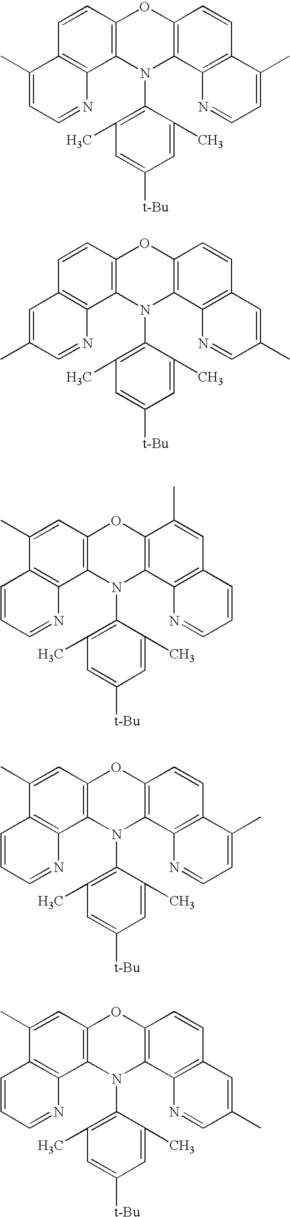

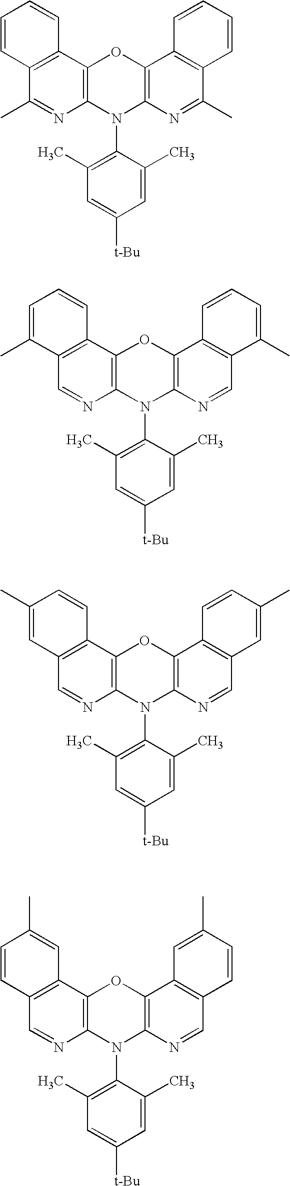

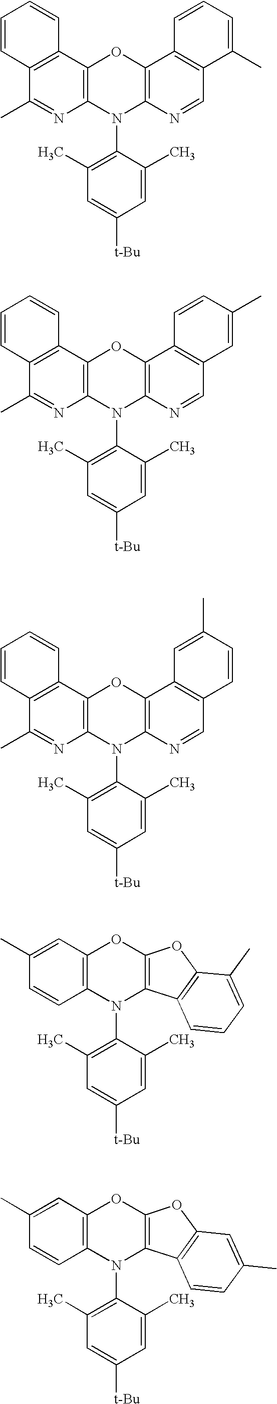

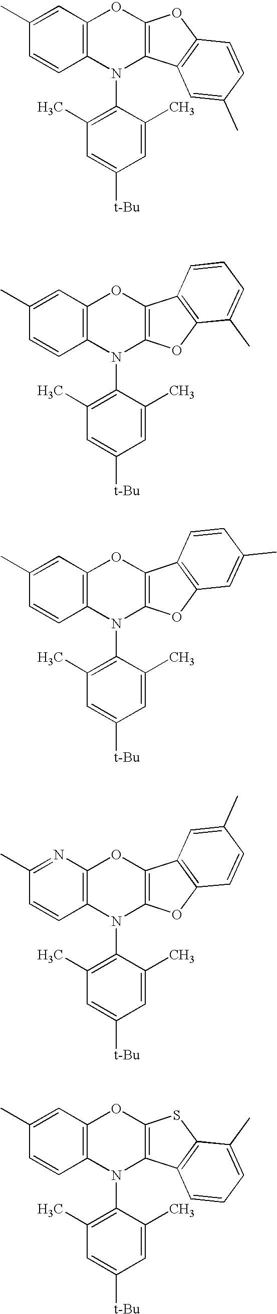

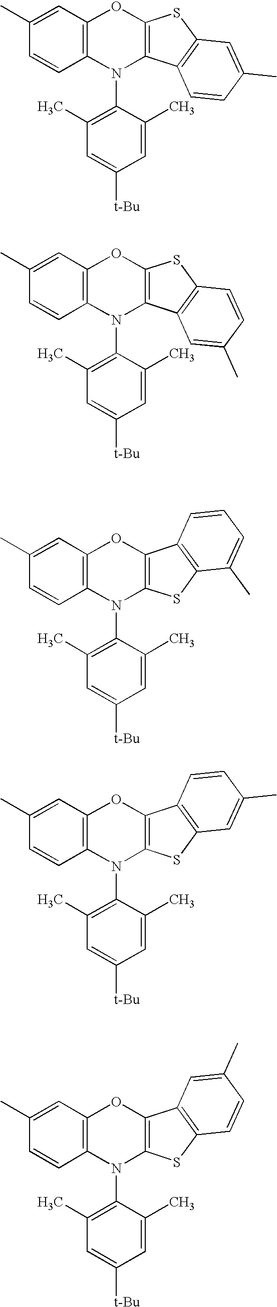

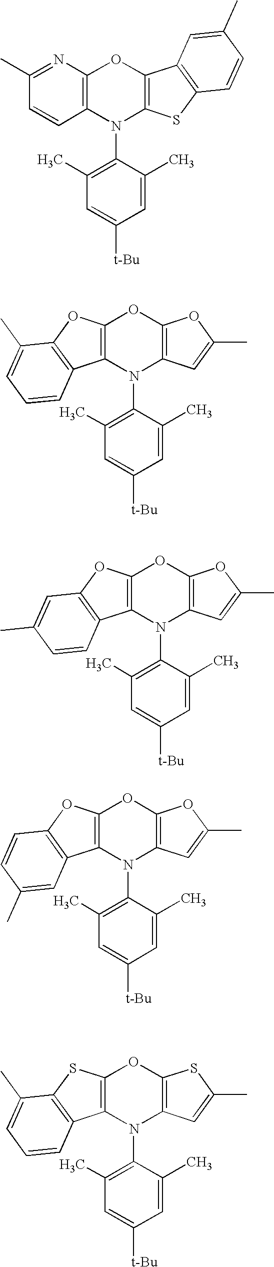

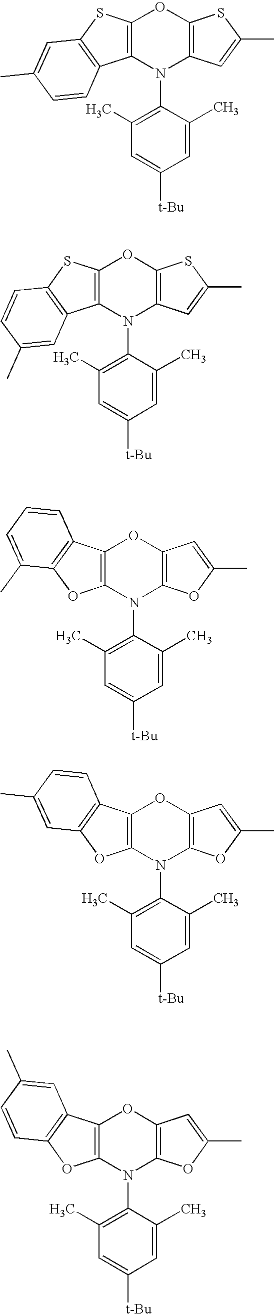

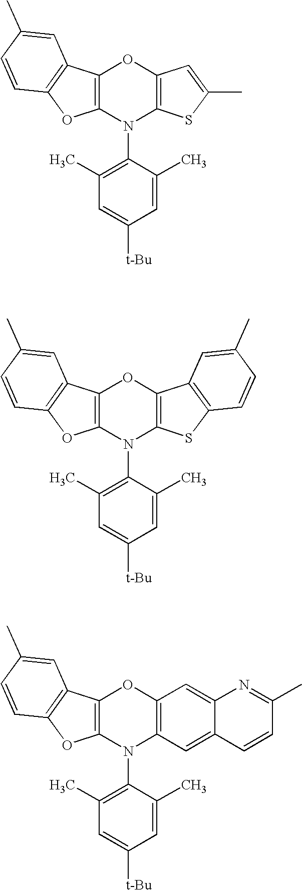

Structural units containing residues of compounds of the above-described formula (1) are, when containing a residue of the compound on the end of the main chain or containing a residue of the compound on the side chain, represented by the following formula (1-1) or (1-2).

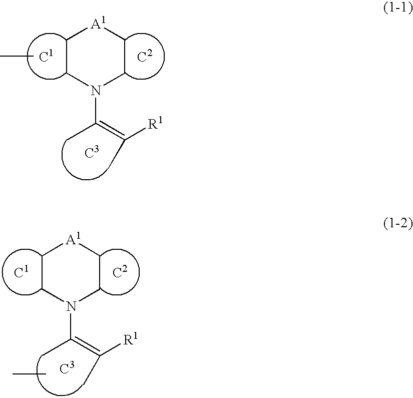

In the case of containing a structure of the above-described formula (1-1) or (1-2) on the side chain, the main chain and the structure may be connected via a single bond, oxygen atom, sulfur atom, alkylene group, arylene group, alkenylene group, alkenylene group or di-valent heterocyclic group. Further, the main chain and the structure may be connected via a di-valent group combining two or more of them.

Here, the alkylene group has a carbon number of about 1 to 30, preferably about 1 to 15. Specifically, a methylene group, ethylene group, propylene group, trimethylene group, tetramethylene group, pentamethylene group, 1,3-cyclopentylene group, 1,4-cyclohexylene group and the like are exemplified.

The alkenylene group has a carbon number of about 2 to 30, preferably about 2 to 15. Specifically, a vinylene group, propylene group and the like are mentioned. The alkenylene group includes also alkadienylene groups such as a 1,3-butadienylene group and the like.

The alkynylene group has a carbon number of about 2 to 30, preferably about 2 to 15. Specifically, an ethynylene group and the like are mentioned. The alkynylene group includes also groups having two triple bonds, and for example, a 1,3-butanediynylene group is mentioned.

The arylene group means a group obtained by removing two hydrogen atoms from an aromatic hydrocarbon ring, and the number of carbons constituting an aromatic ring is usually about 6 to 30, preferably about 6 to 15. Specific examples thereof include a phenylene group, biphenylene group, terphenylene group, naphthalenediyl group, anthracenediyl group, phenanthrenediyl group, pentalenedilyl group, indenediyl group, heptalenediyl group, indacenedilyl group, triphenylenediyl group, binaphthyldiyl group, phenylnaphthylenediyl group, stilbenediyl group, fluorenediyl group and the like.

The di-valent heterocyclic group means a group obtained by removing two hydrogen atoms from a heterocyclic compound; and the number of carbons constituting a ring of a heterocyclic group is usually about 2 to 30, preferably about 2 to 15. Specific examples thereof include a pyridinediyl group, diazaphenylene group, quinolinediyl group, quinoxalinediyl group, acridinediyl group, bipyridyldiyl group, phenanthrolinediyl group and the like.

As the structure of the above-described formula (1-1), the following groups are exemplified.

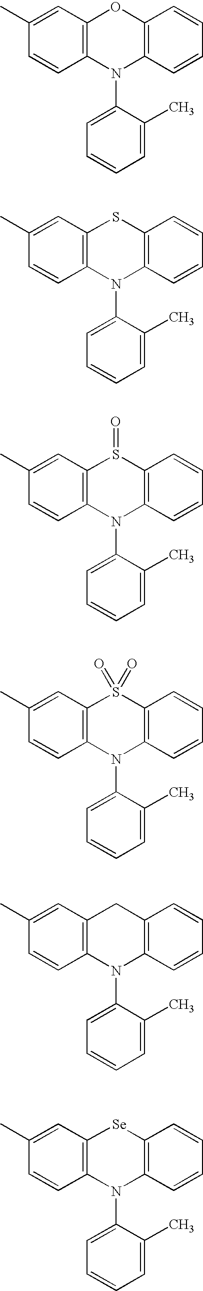

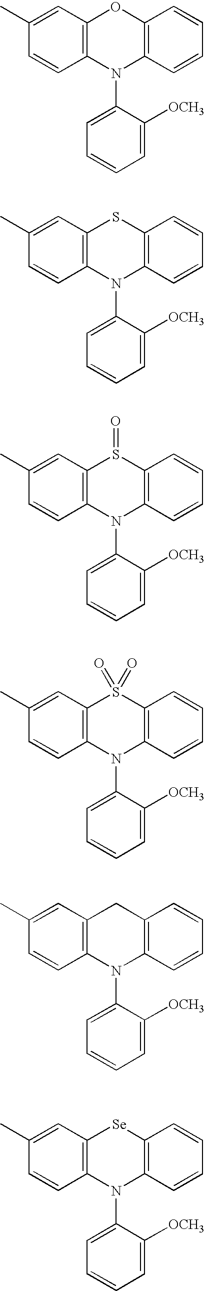

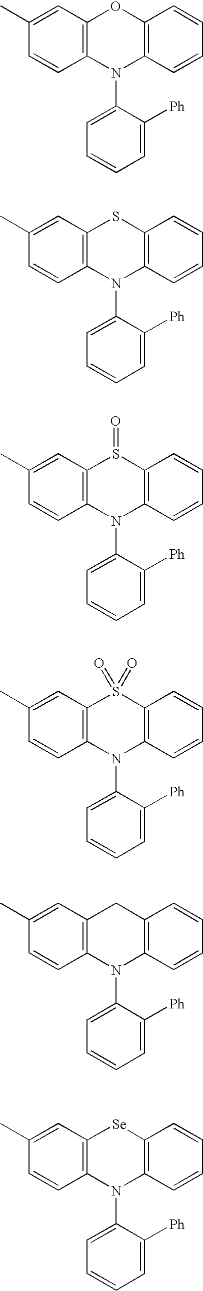

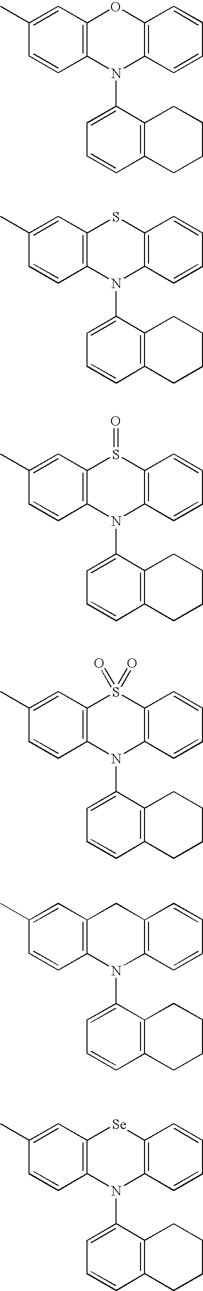

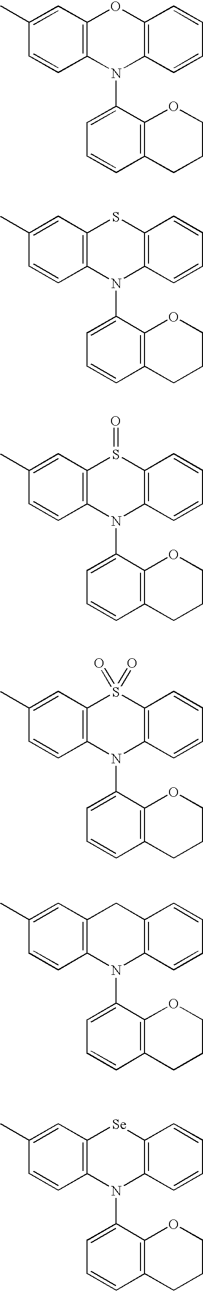

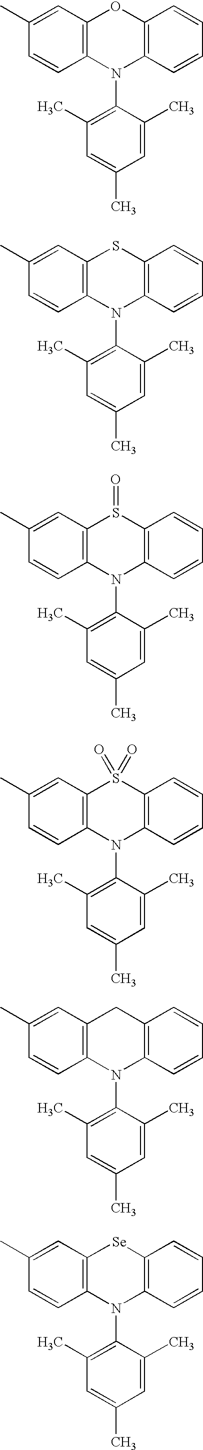

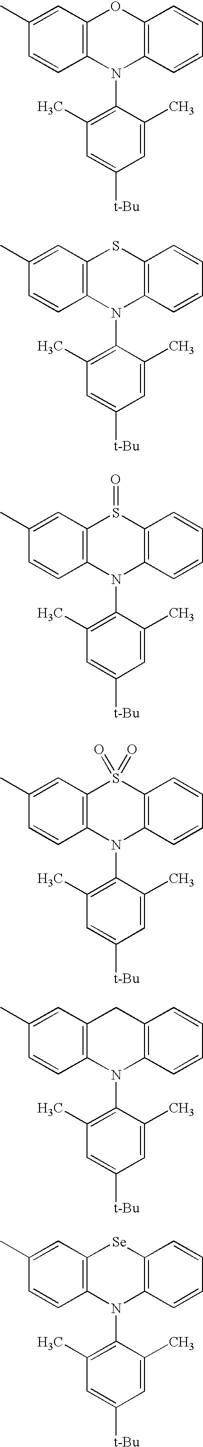

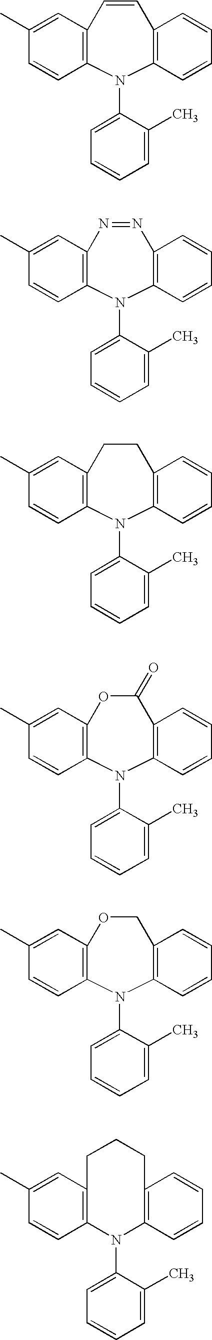

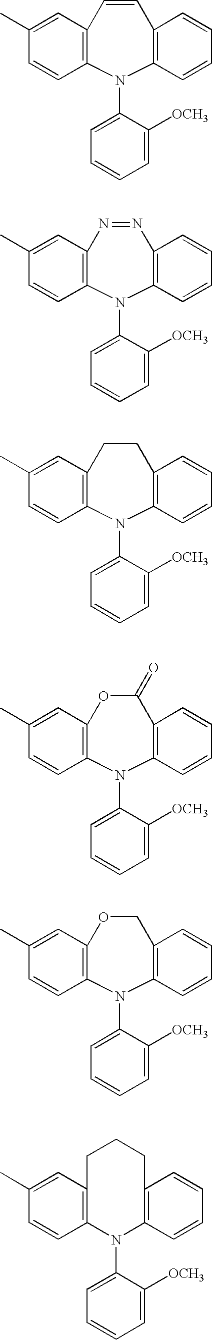

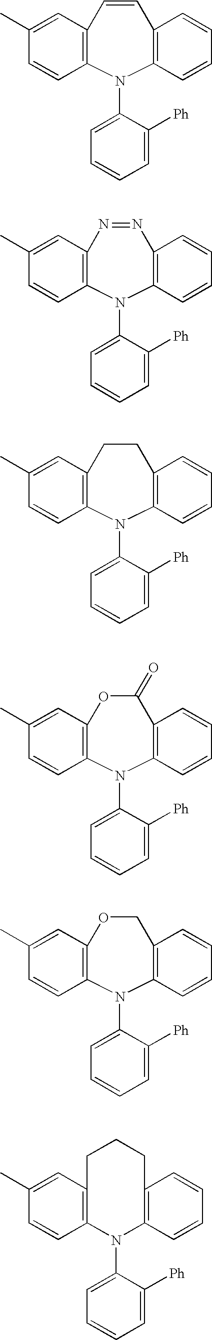

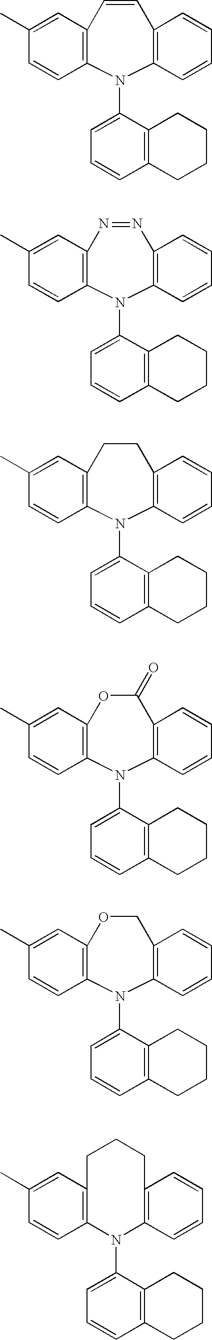

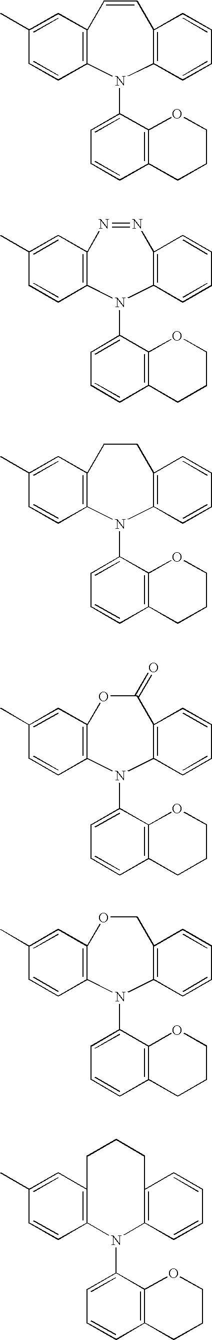

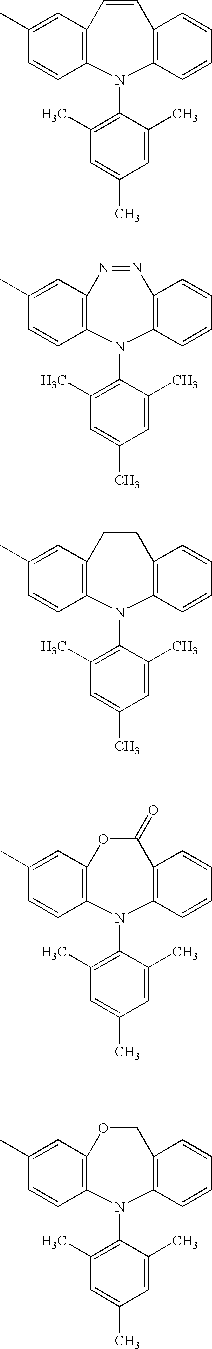

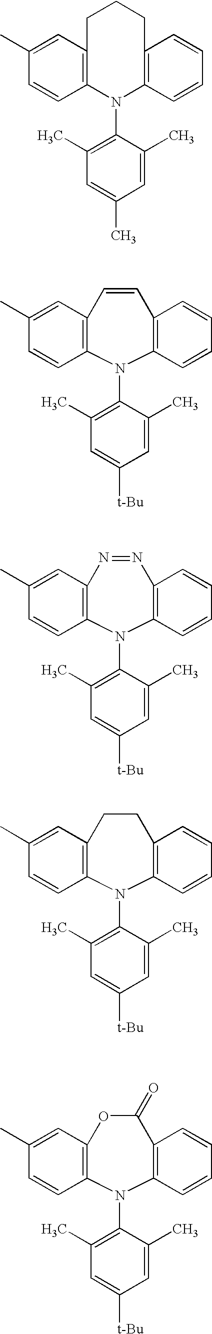

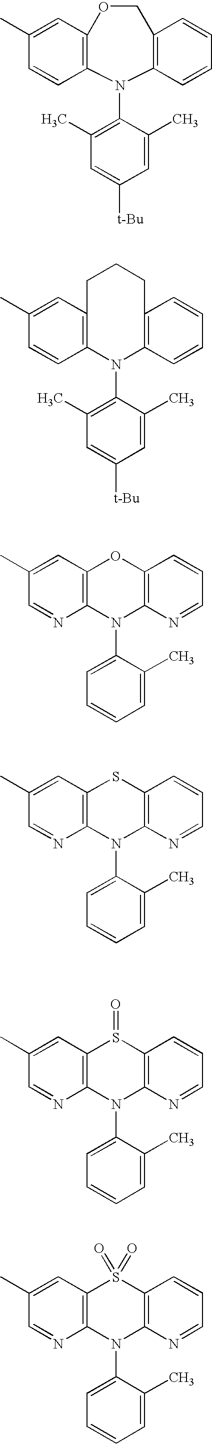

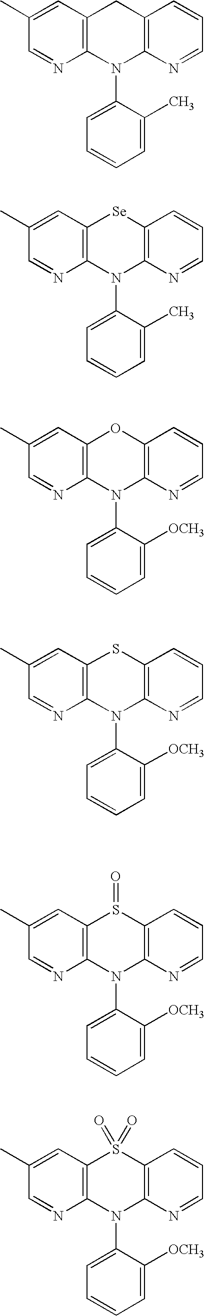

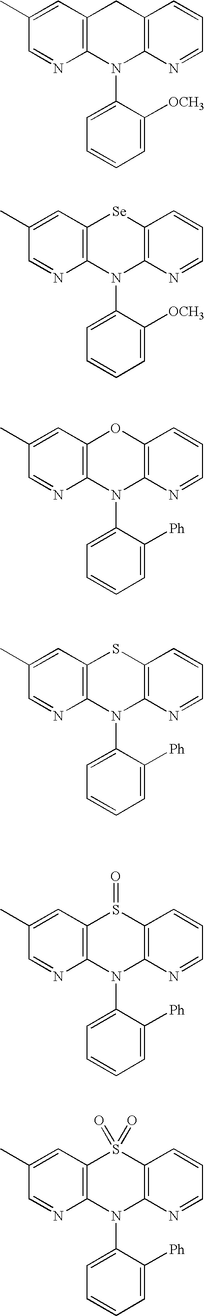

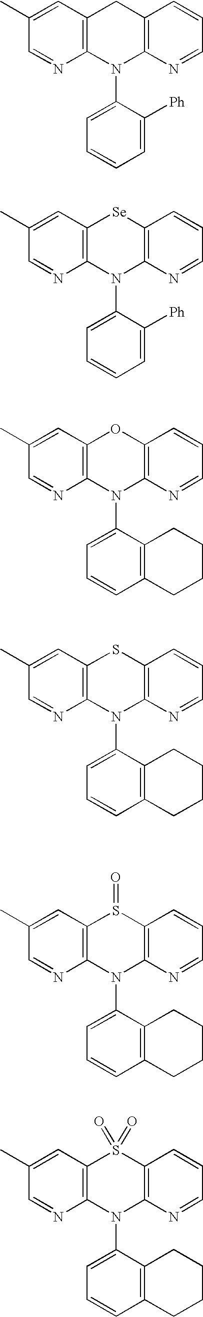

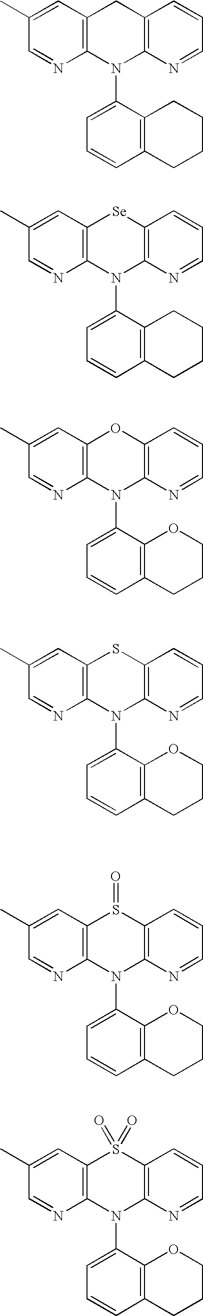

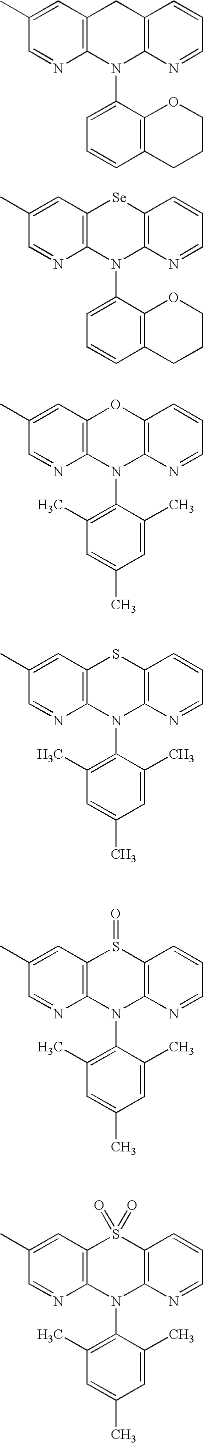

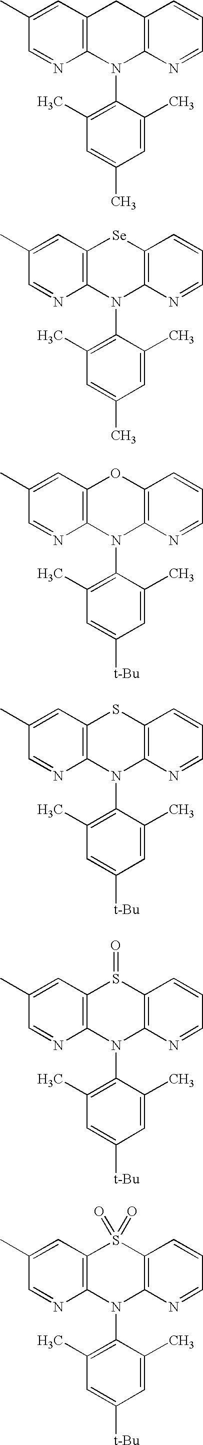

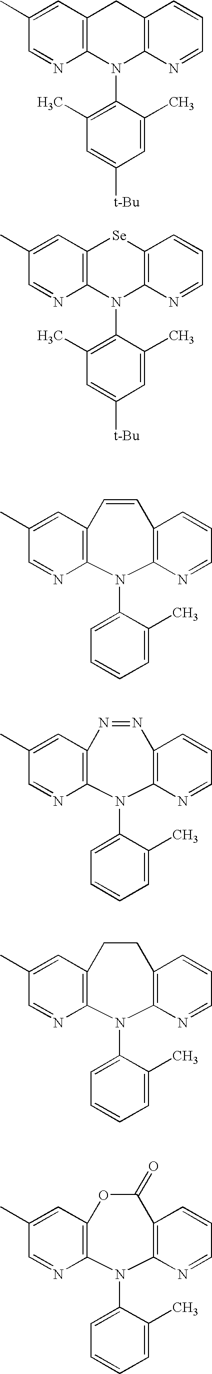

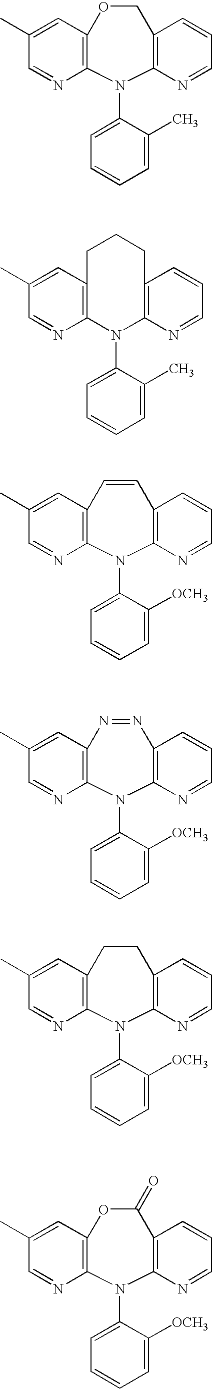

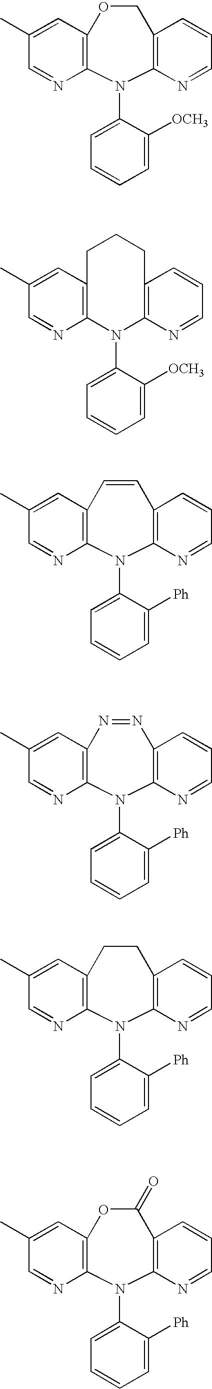

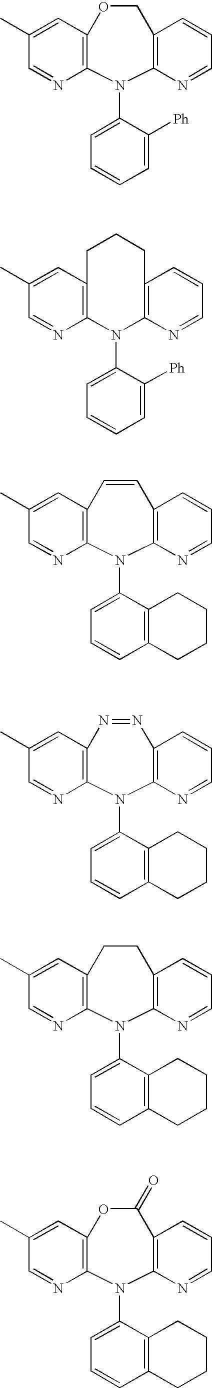

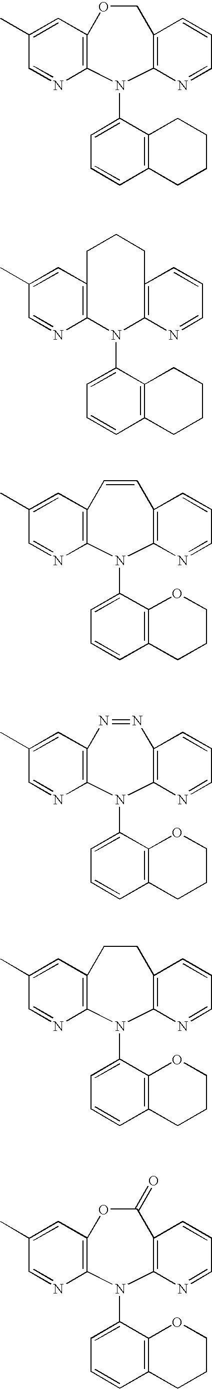

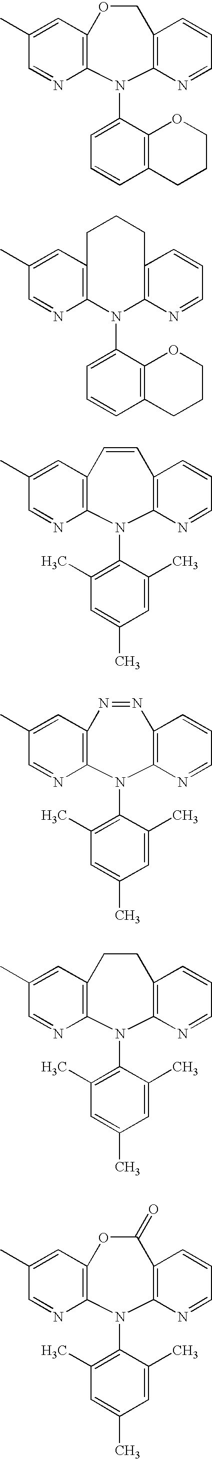

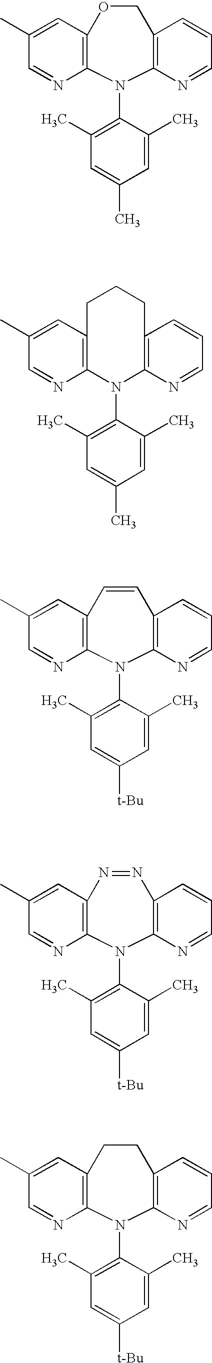

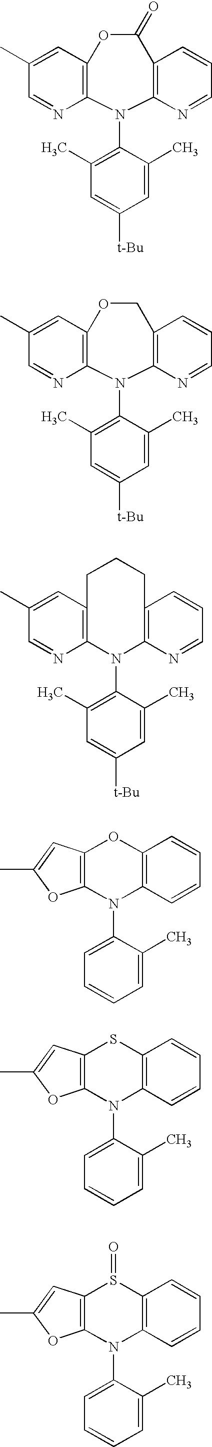

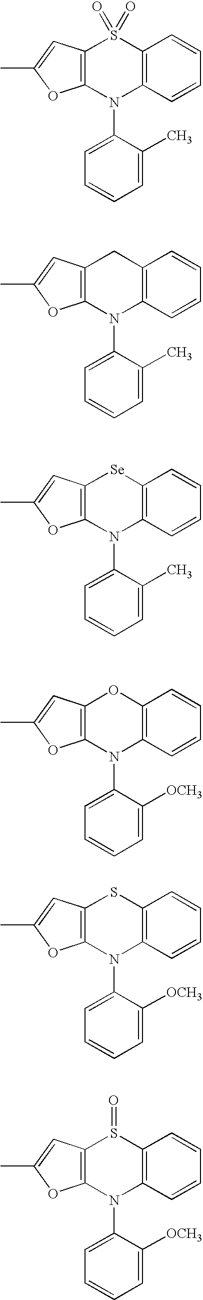

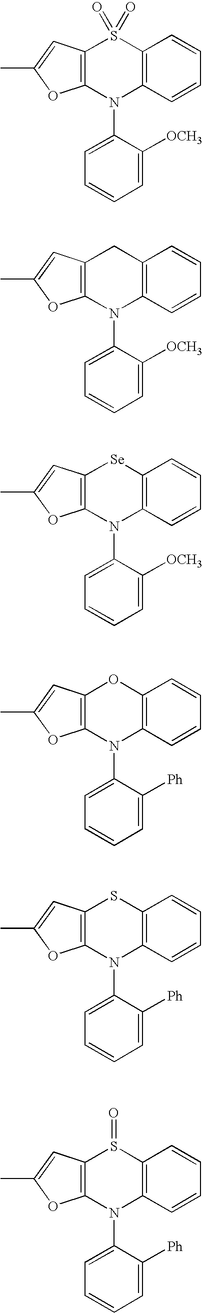

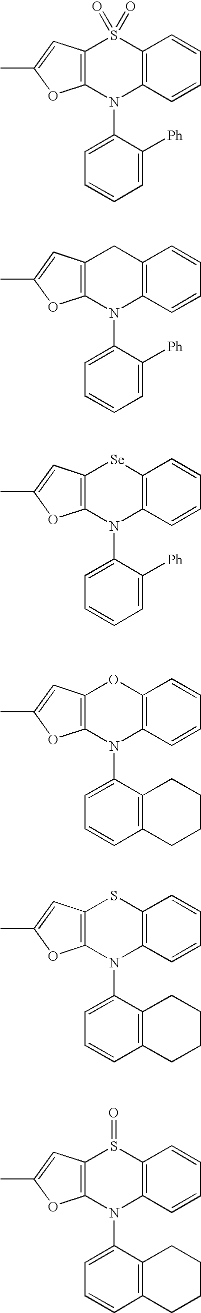

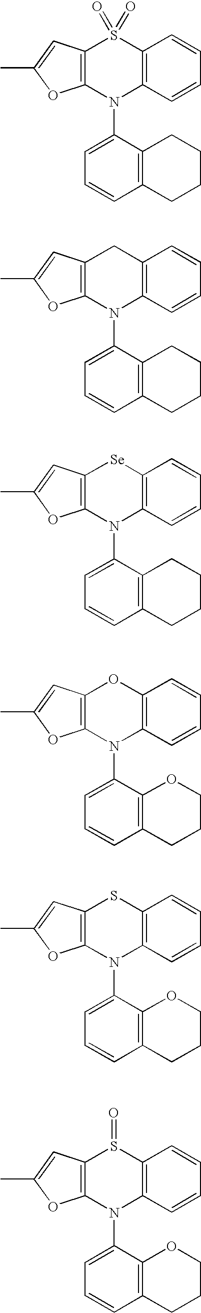

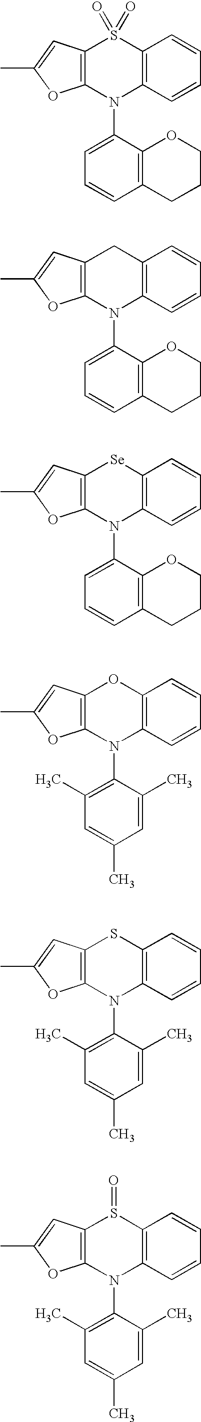

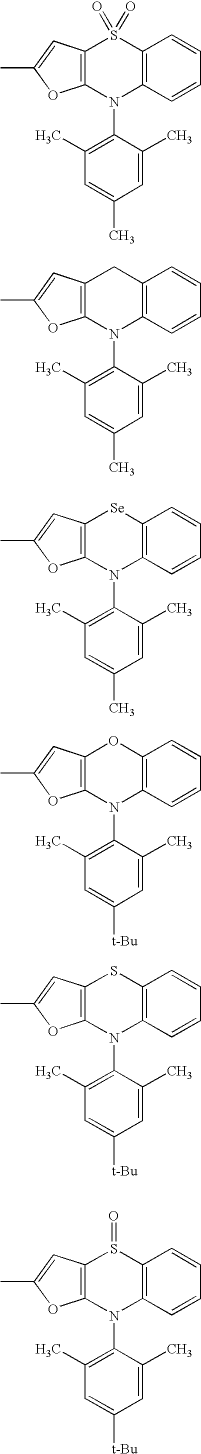

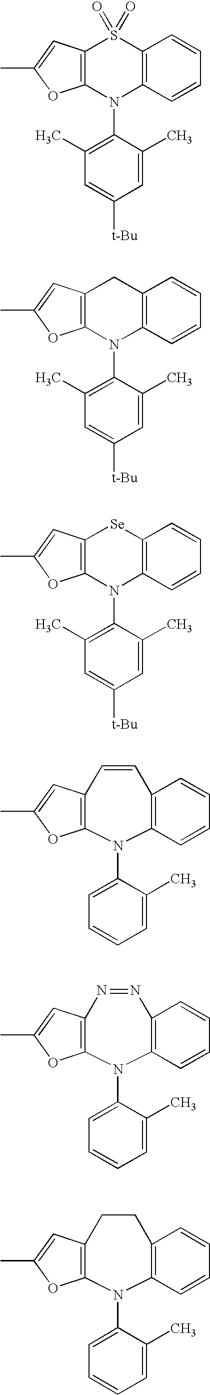

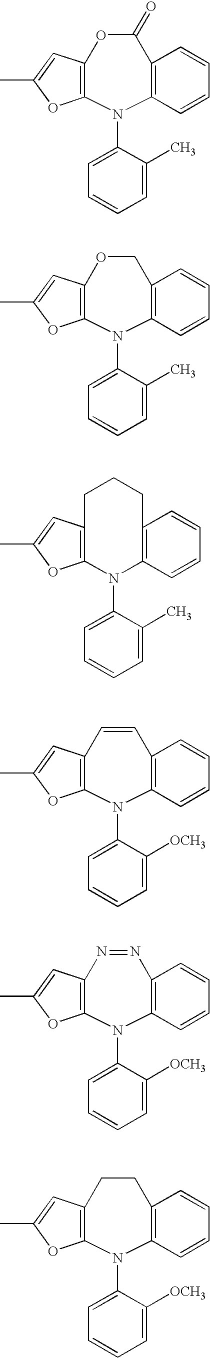

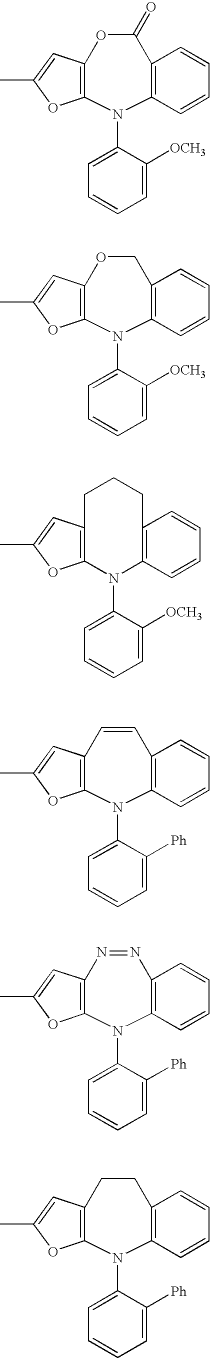

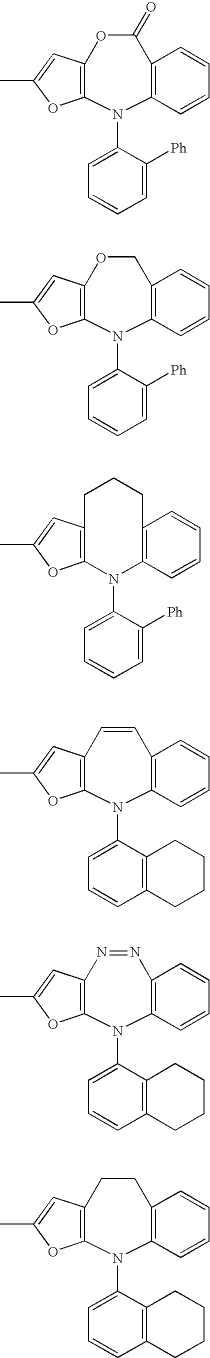

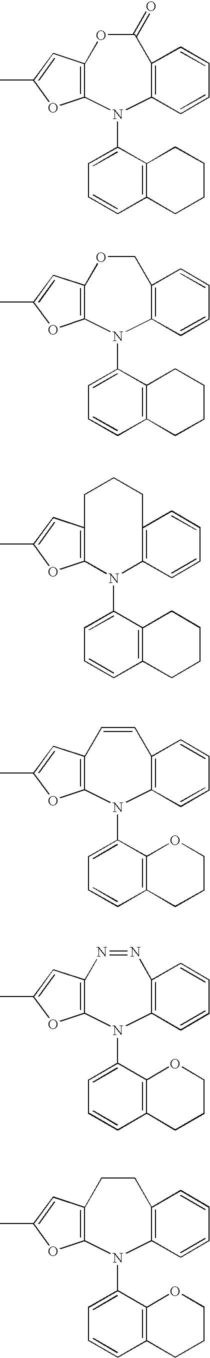

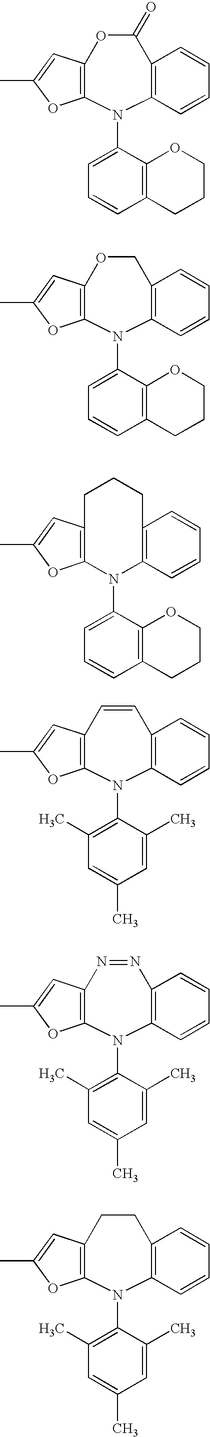

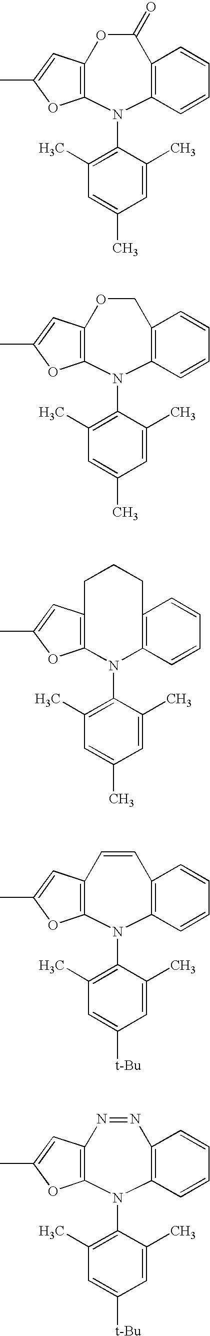

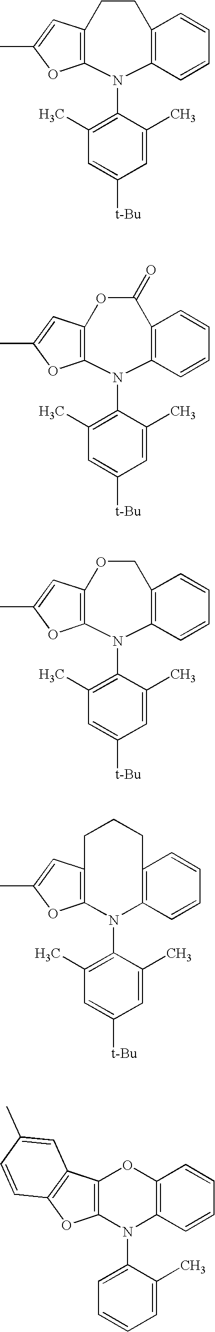

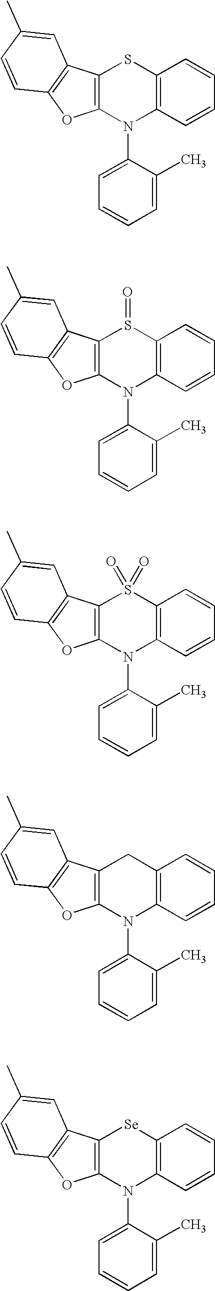

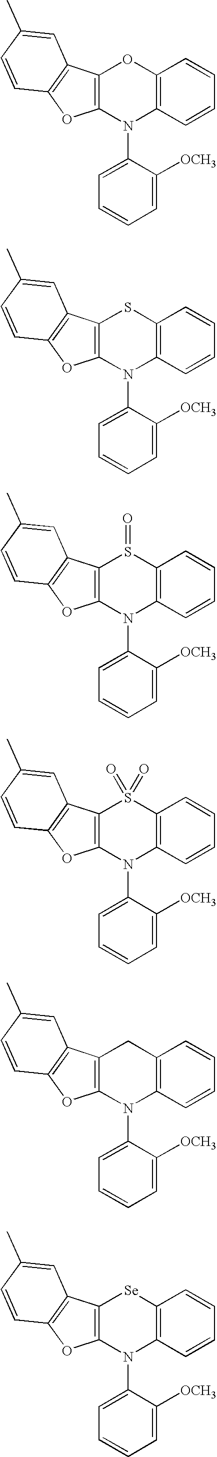

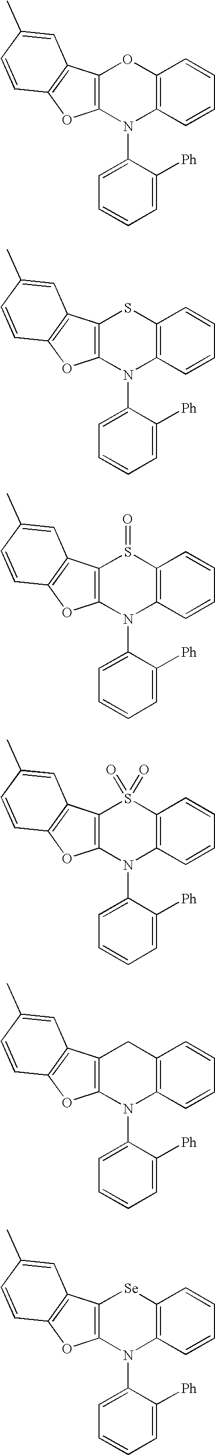

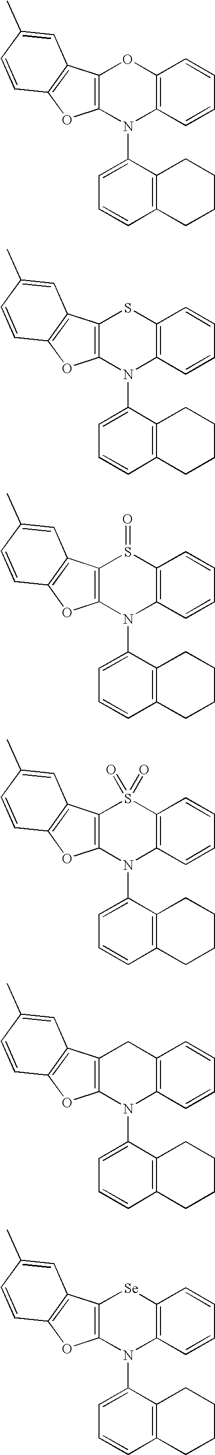

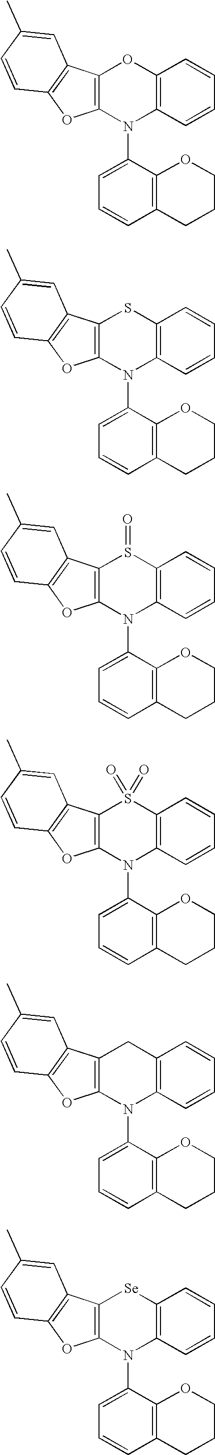

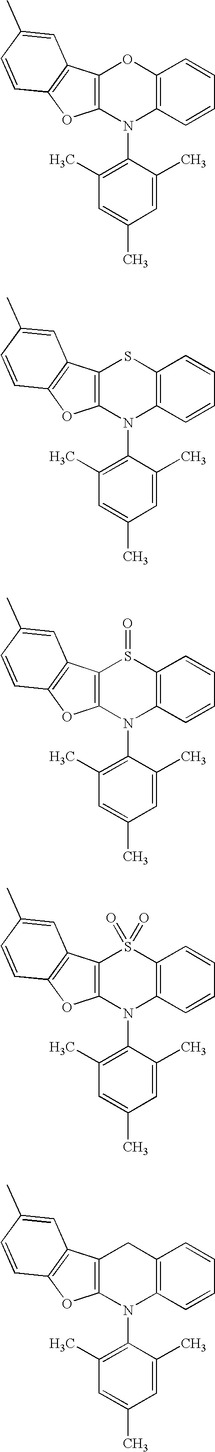

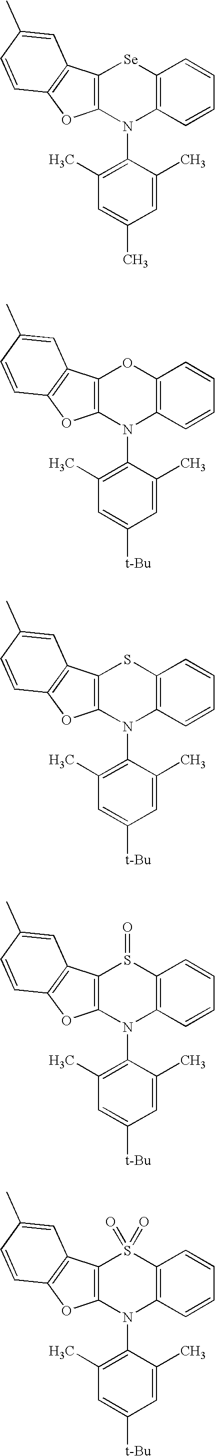

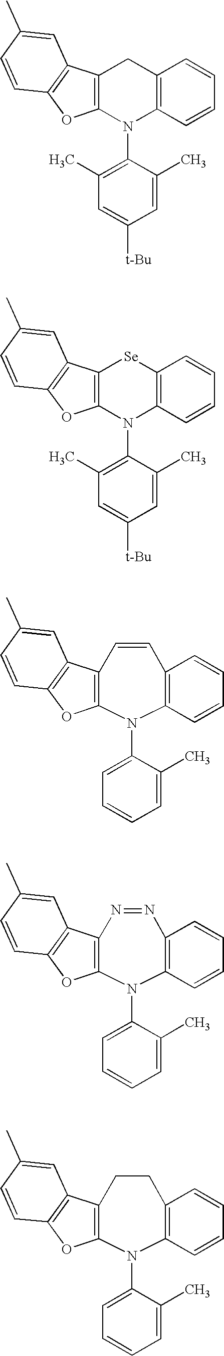

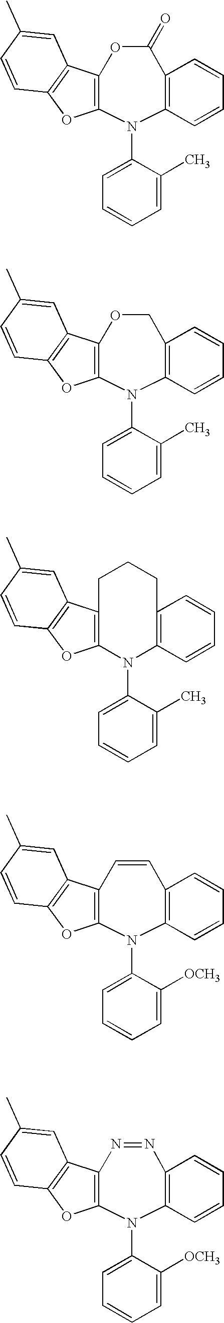

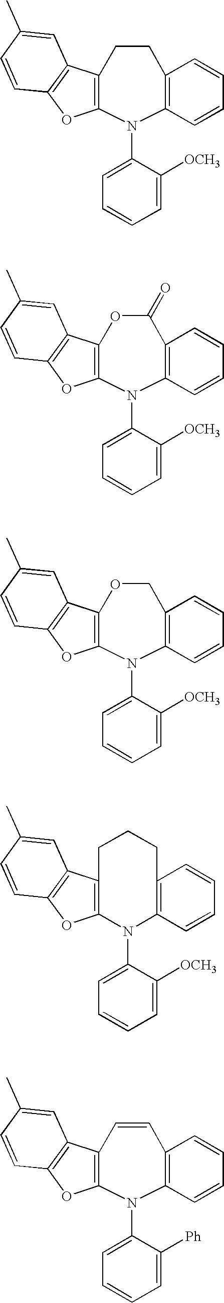

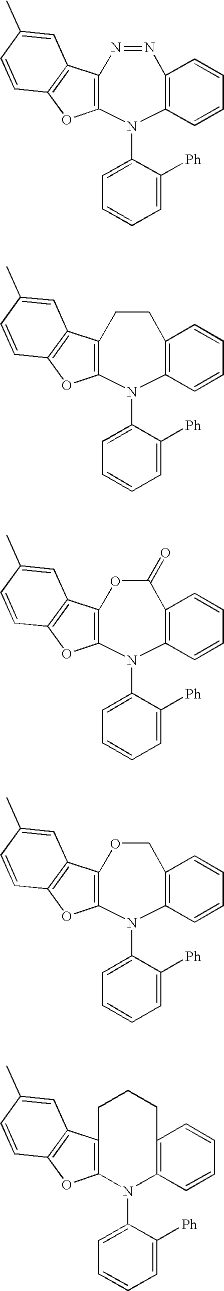

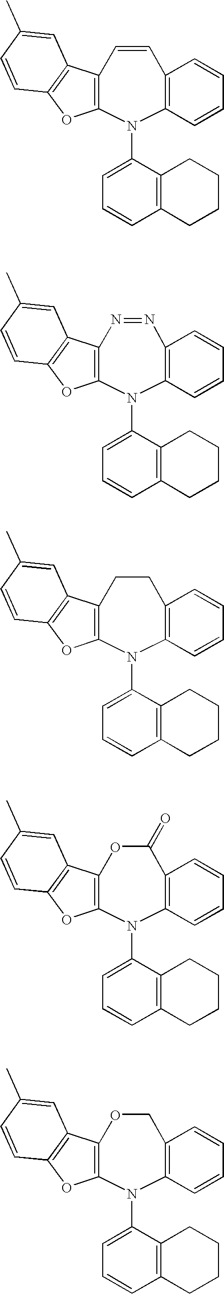

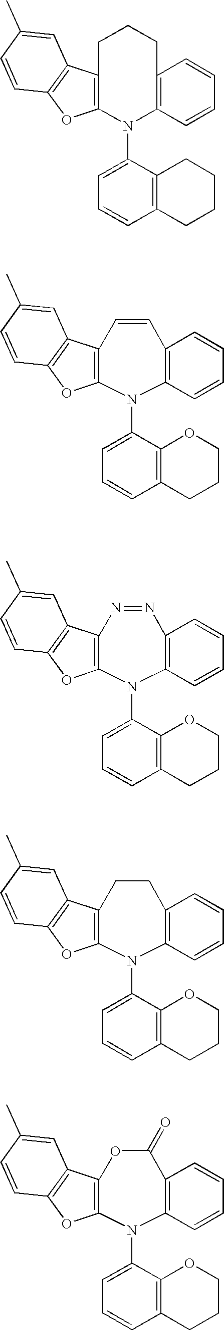

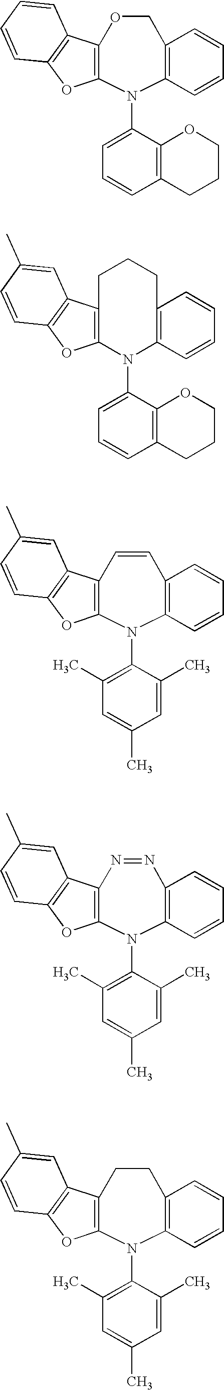

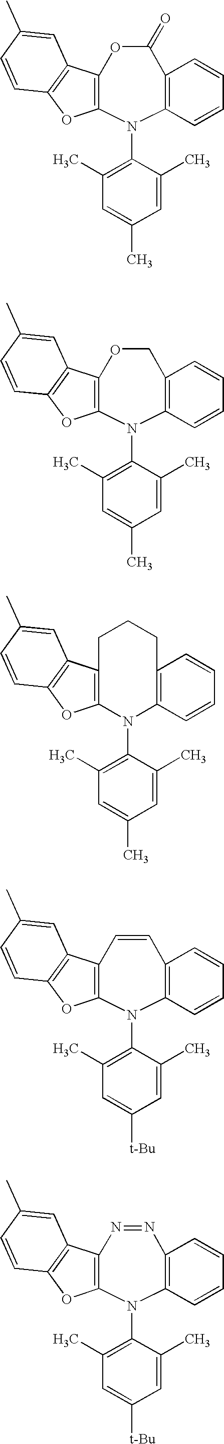

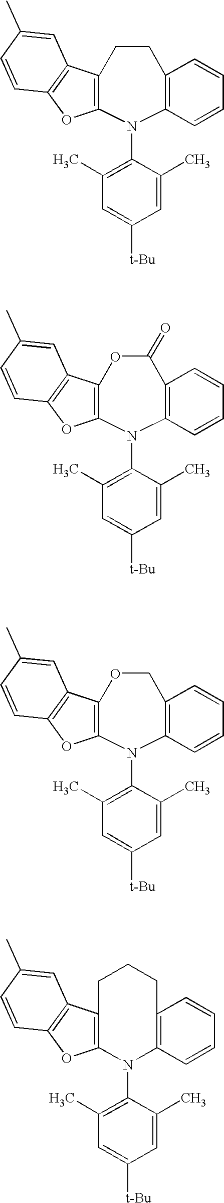

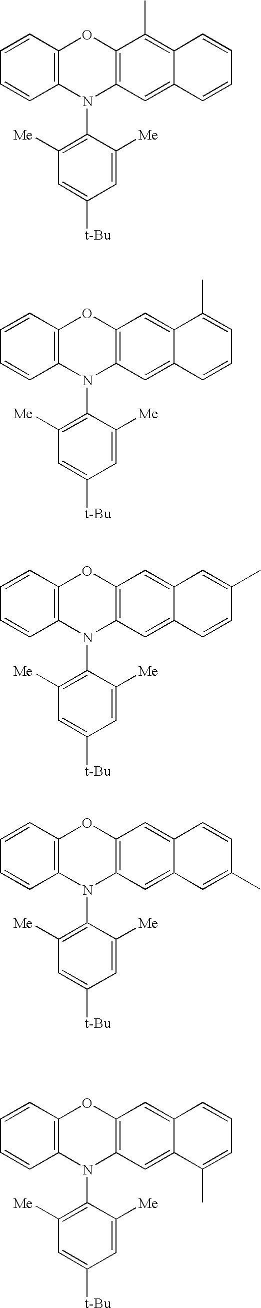

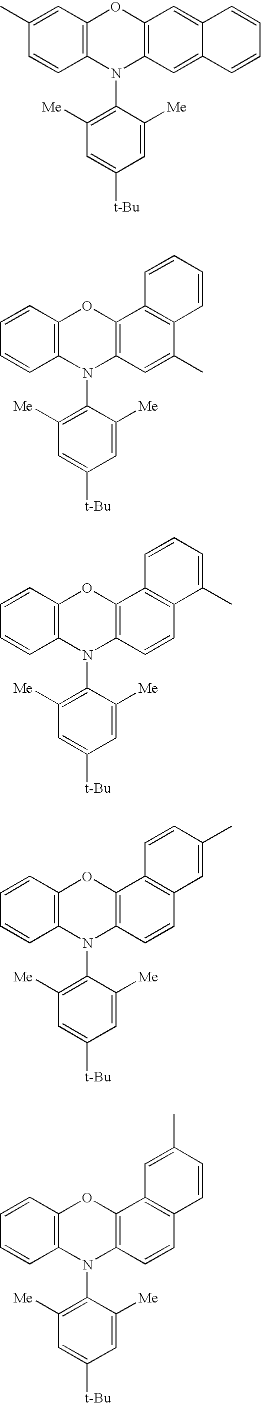

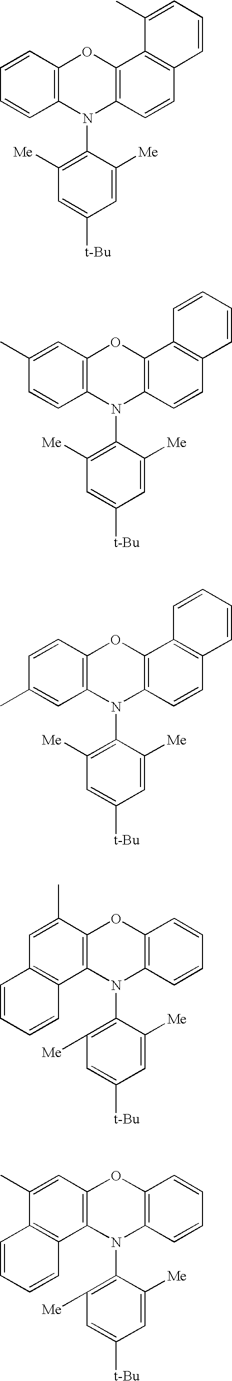

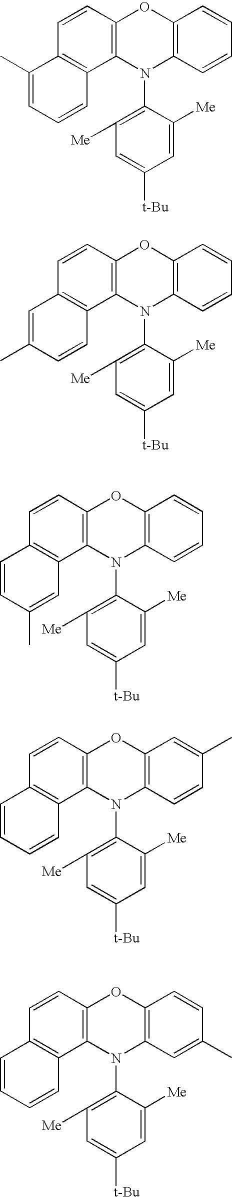

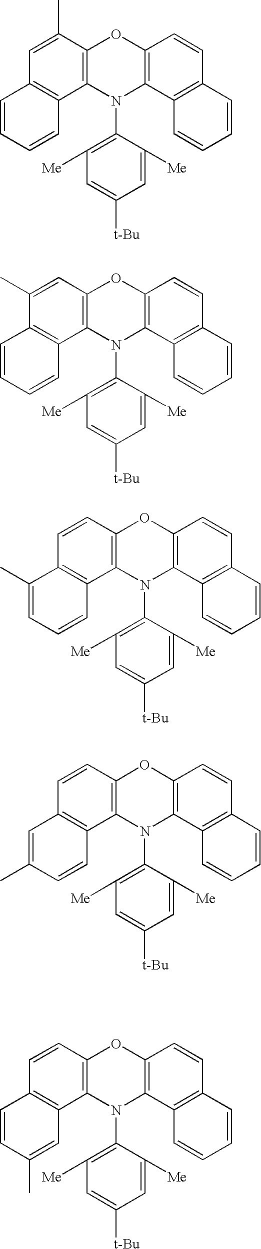

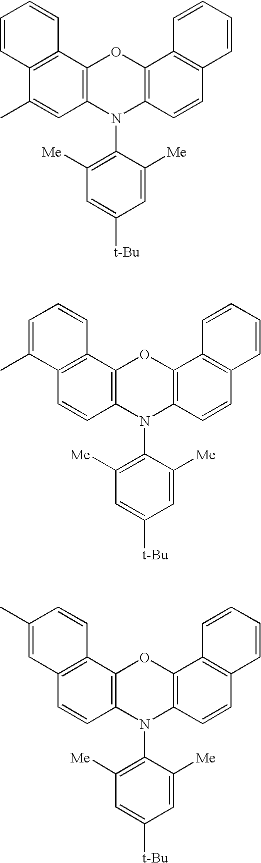

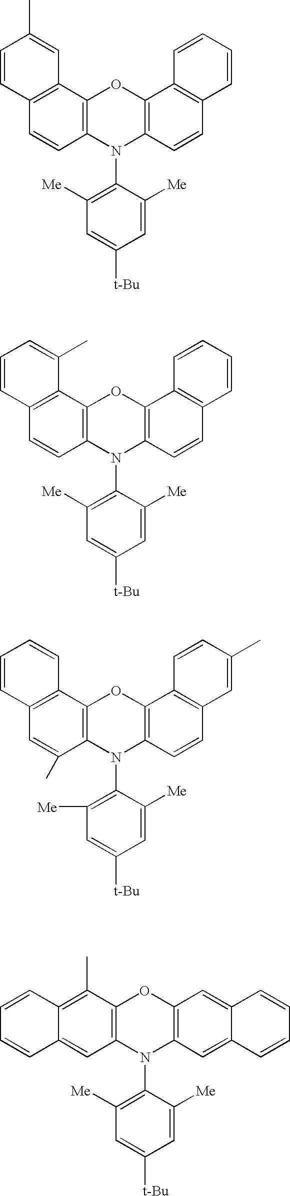

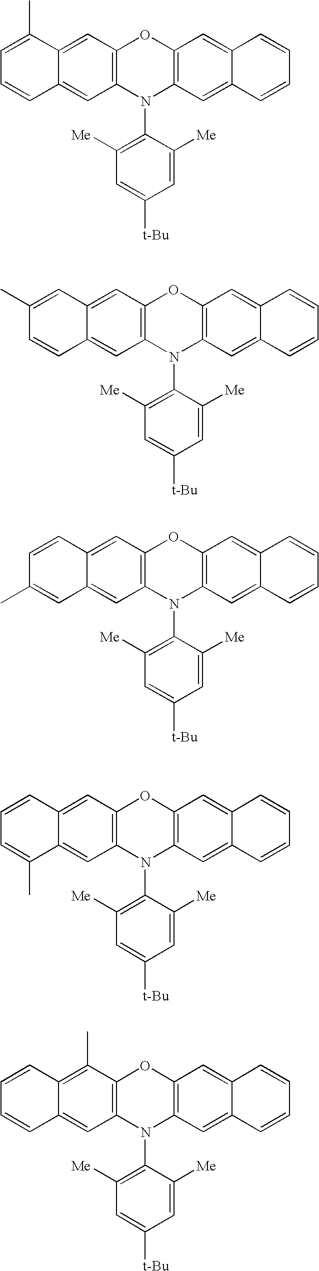

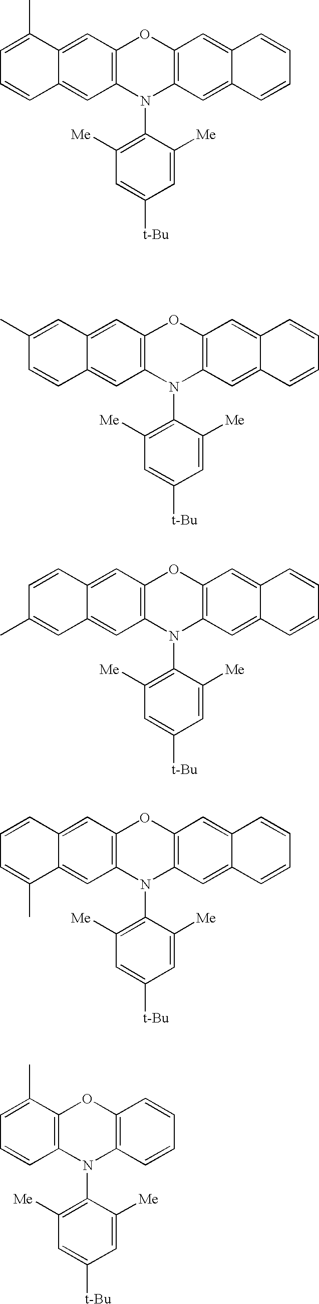

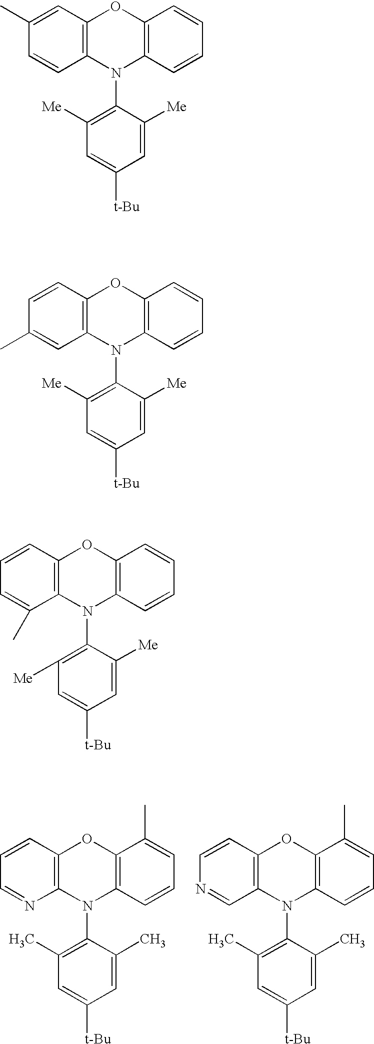

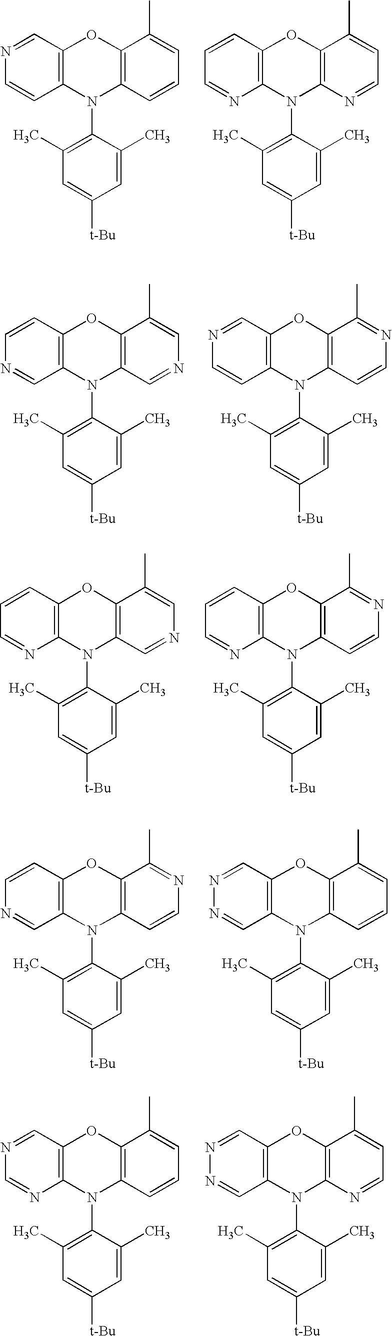

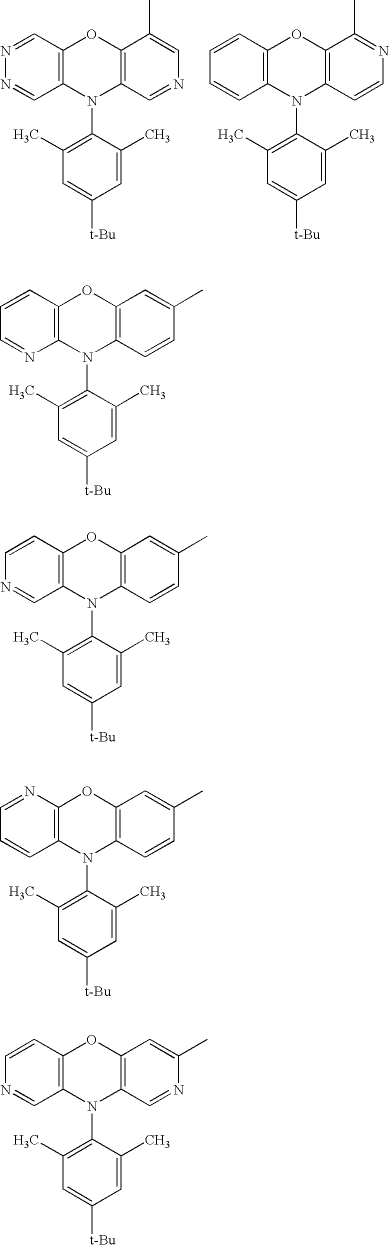

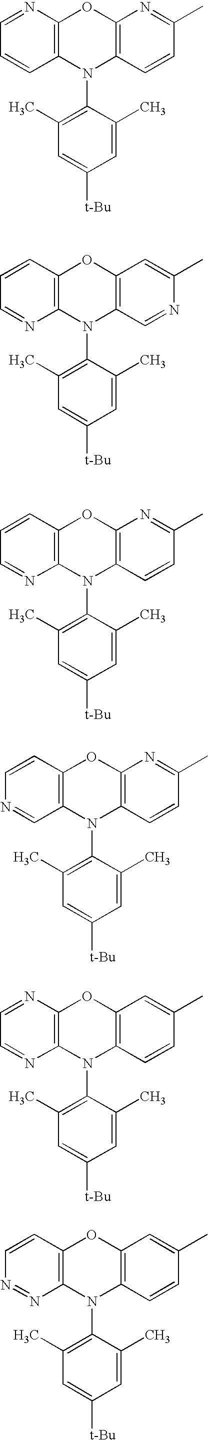

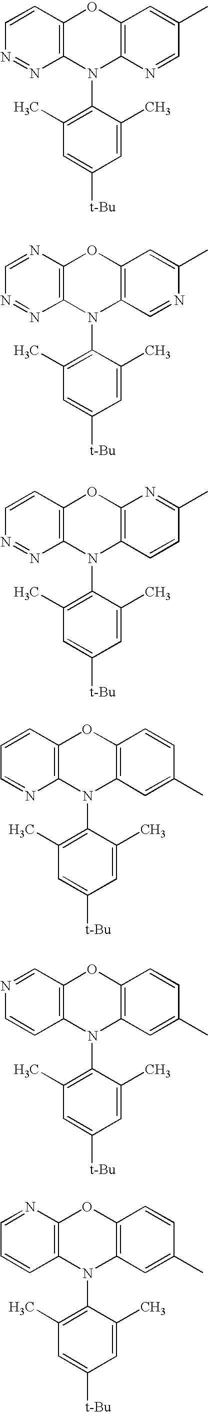

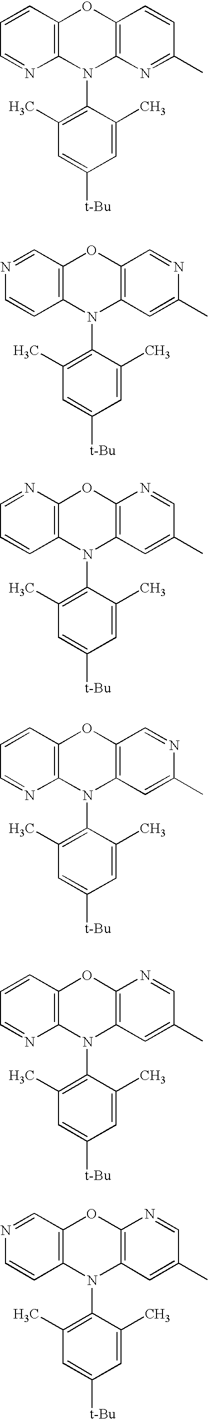

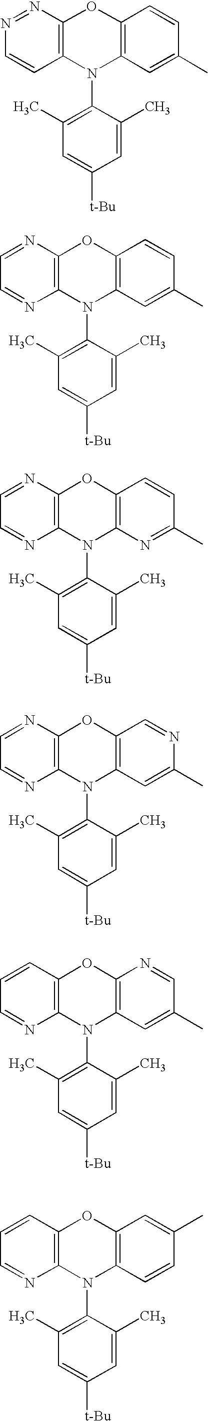

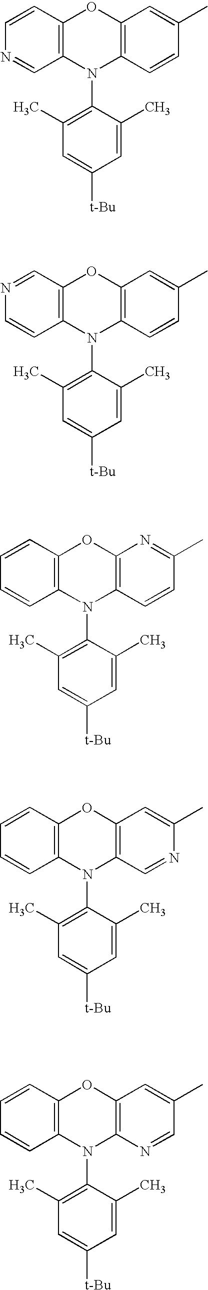

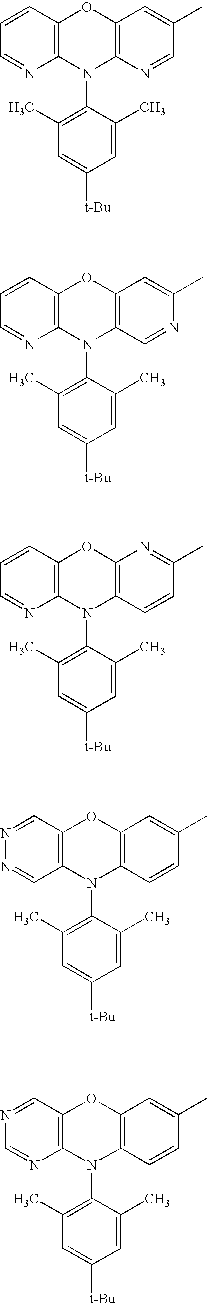

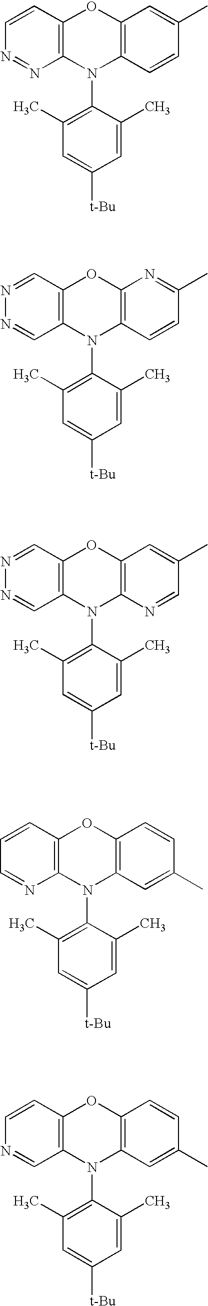

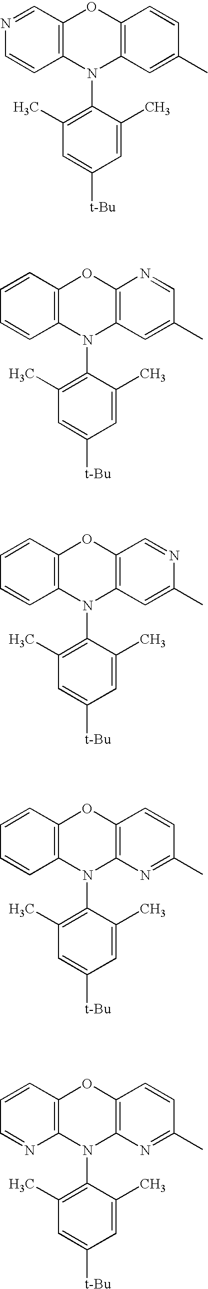

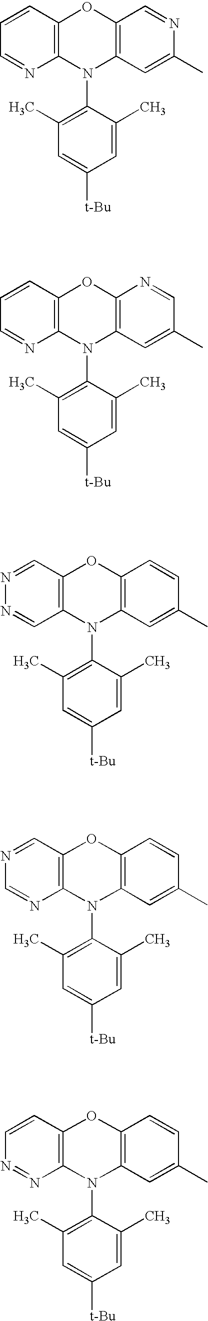

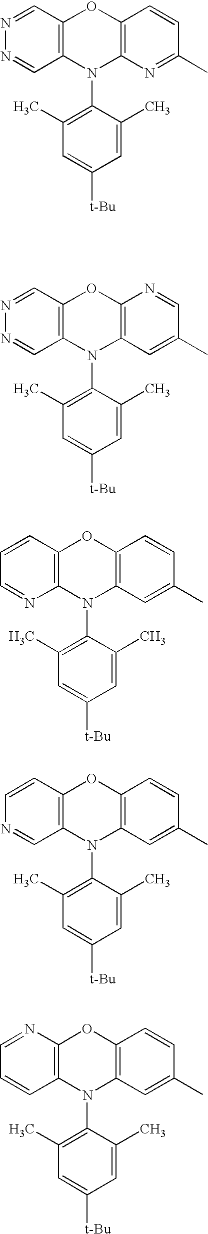

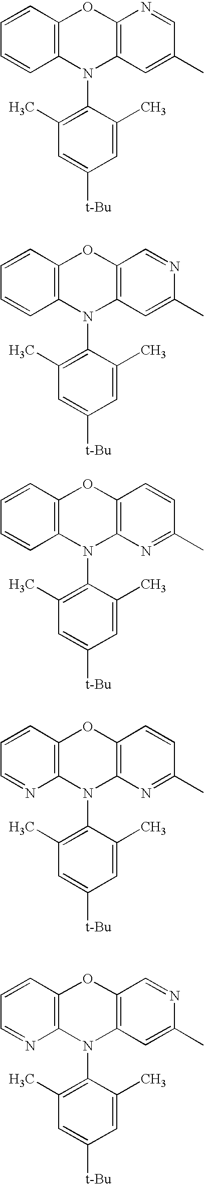

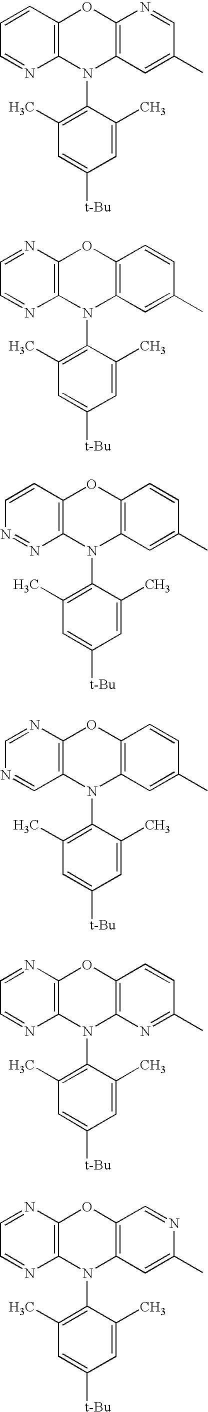

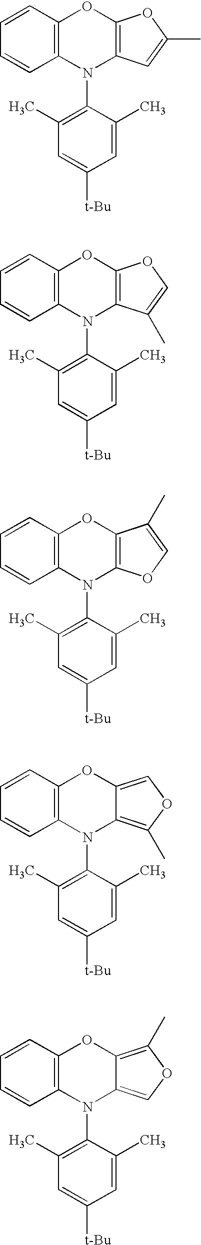

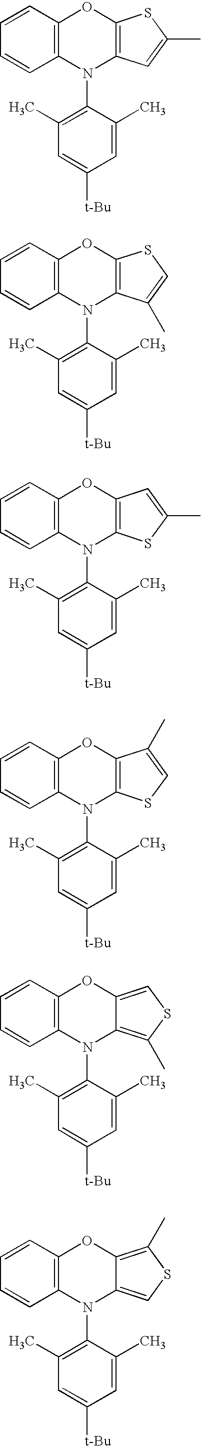

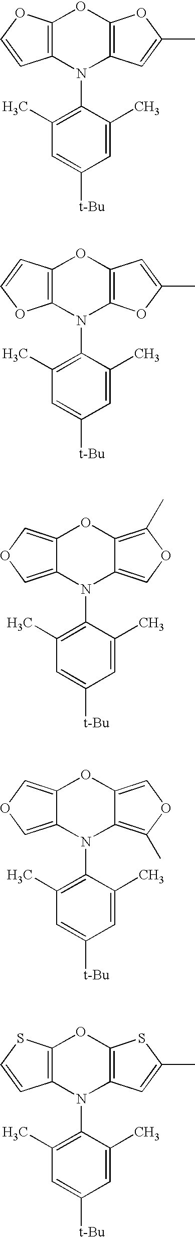

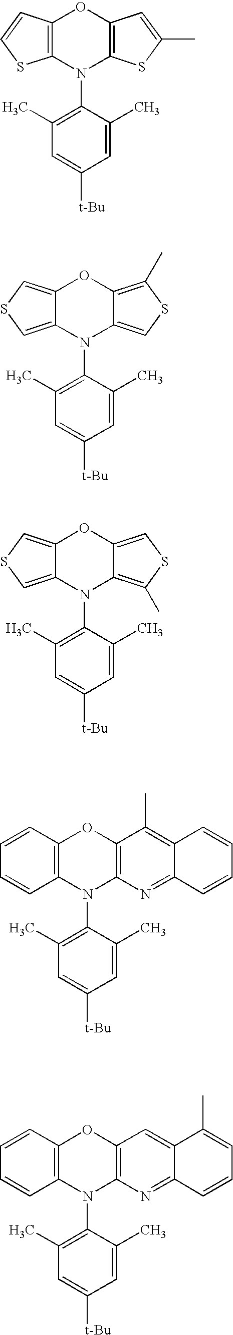

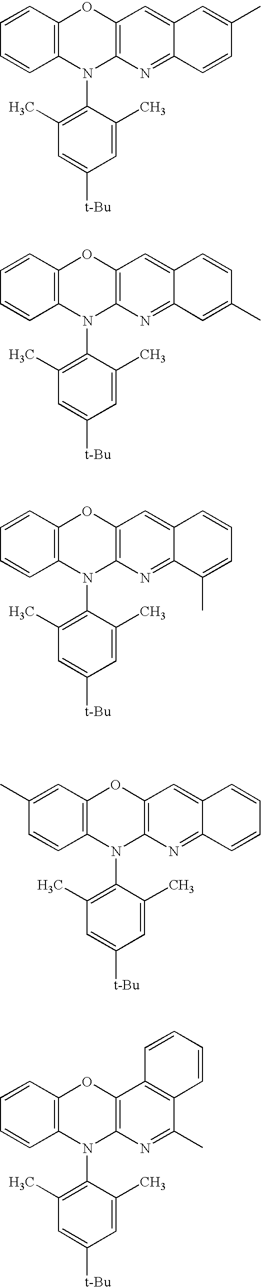

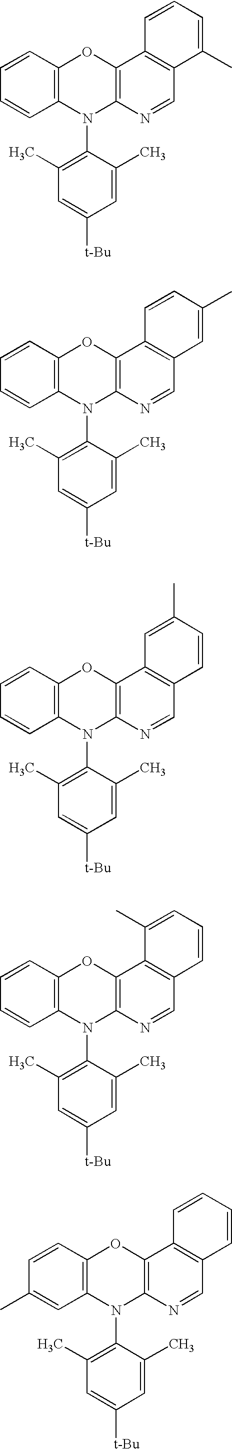

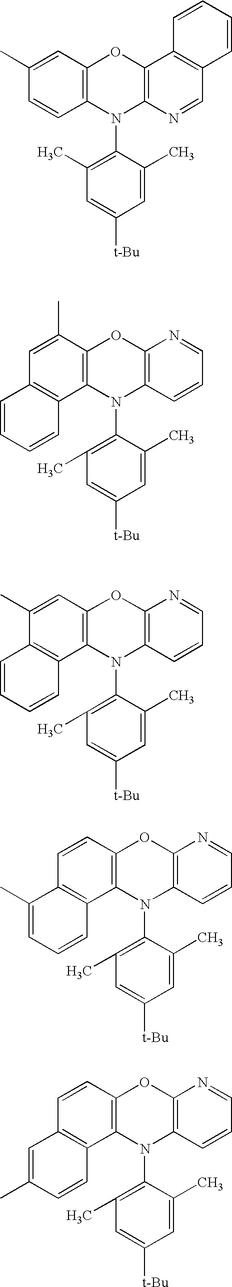

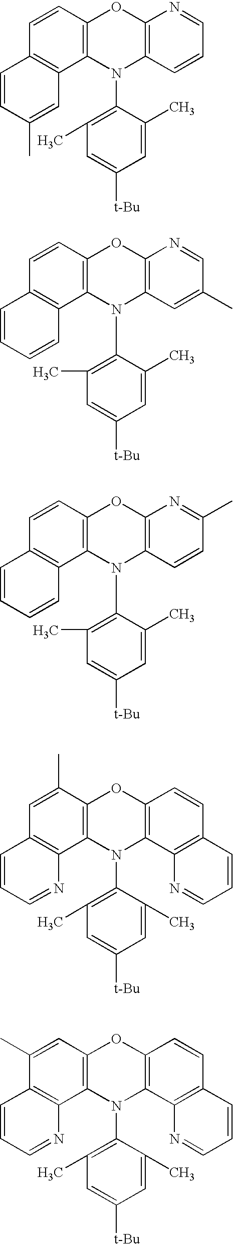

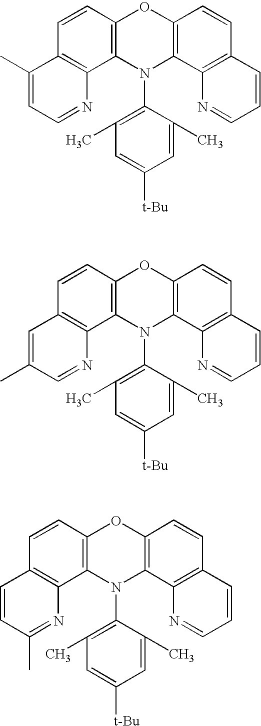

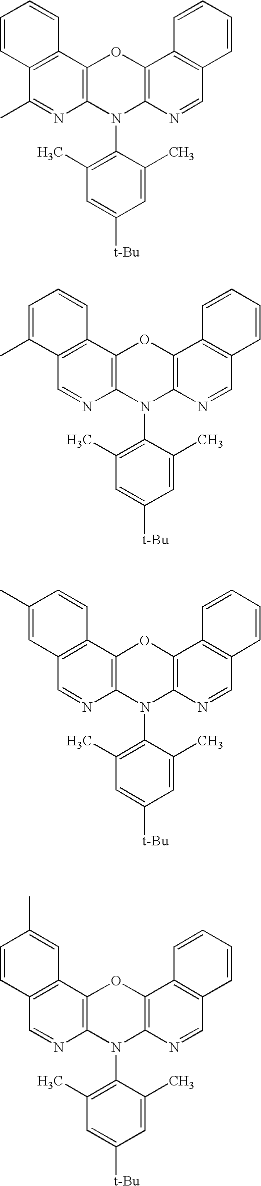

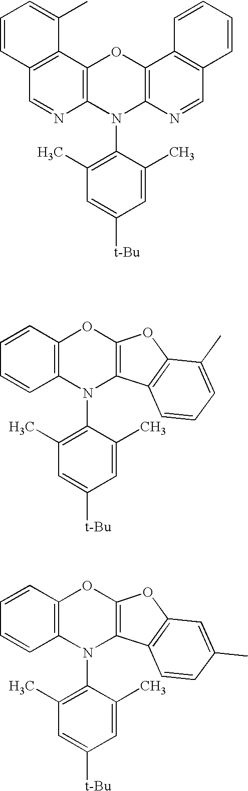

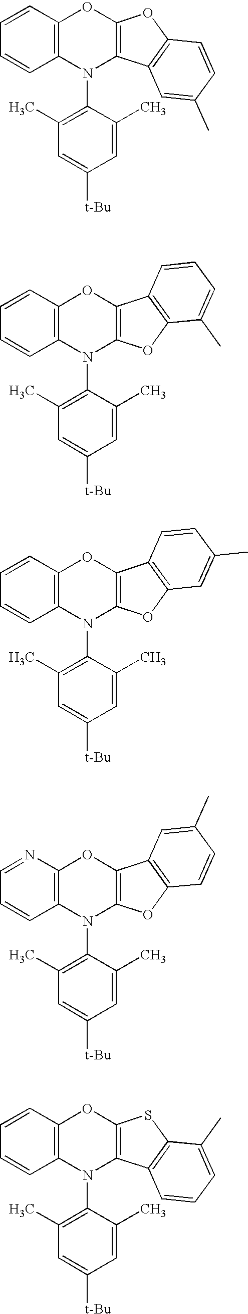

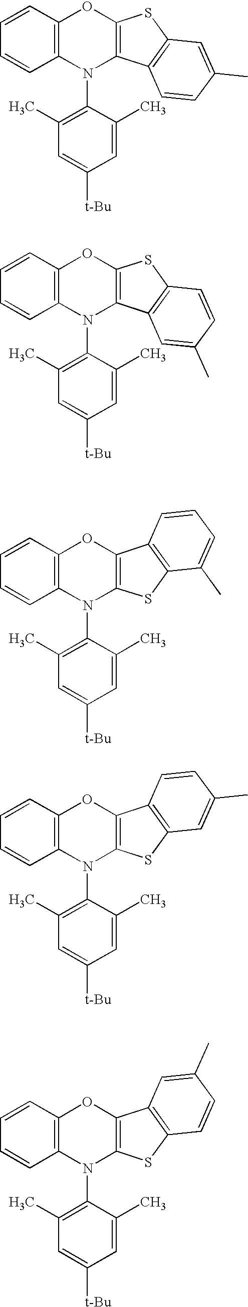

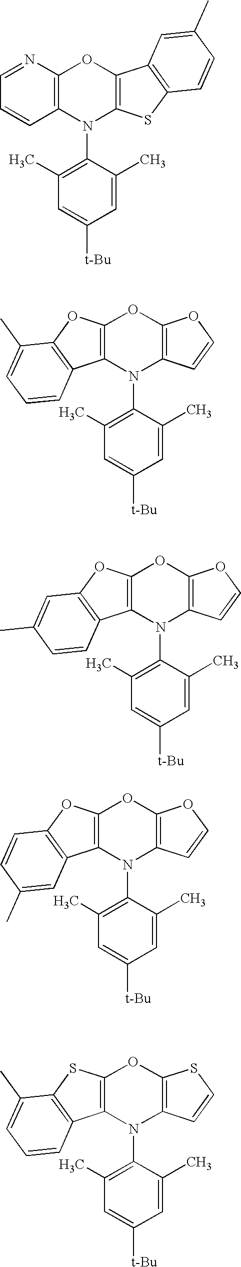

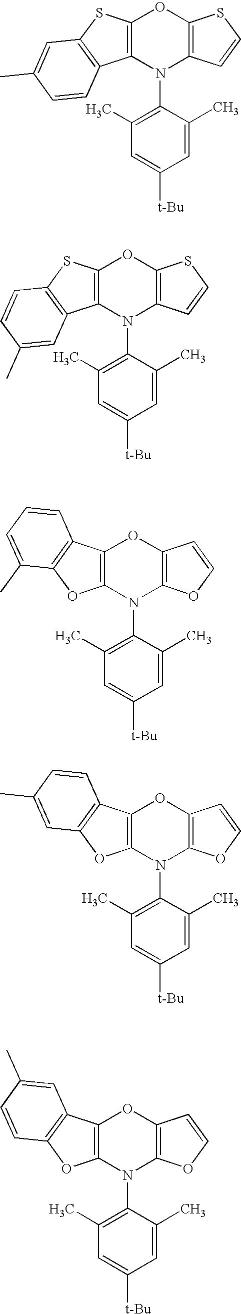

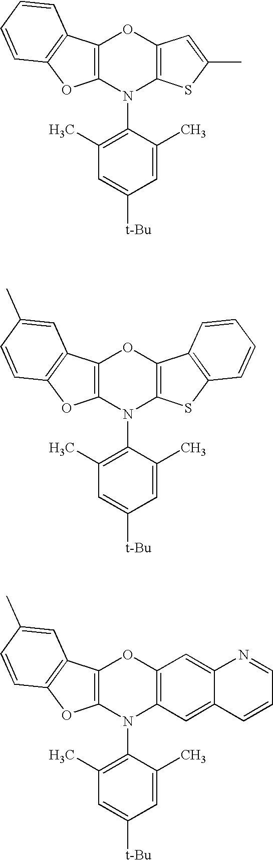

As the structure of the above-described formula (1-2), the following groups are exemplified.

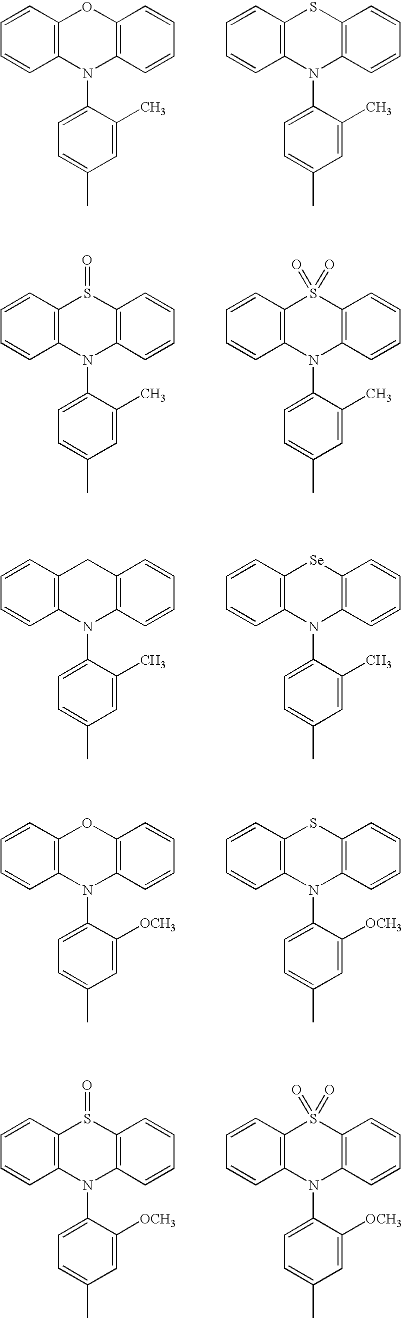

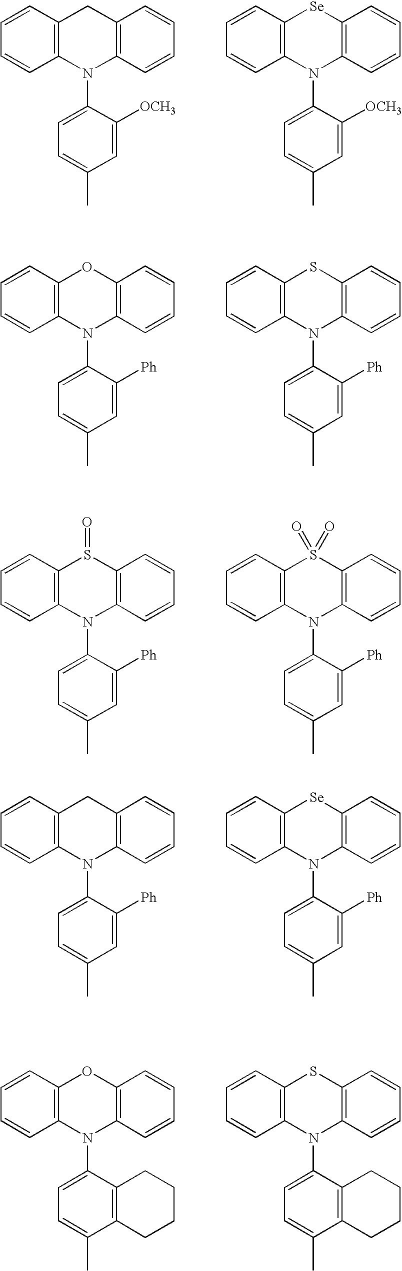

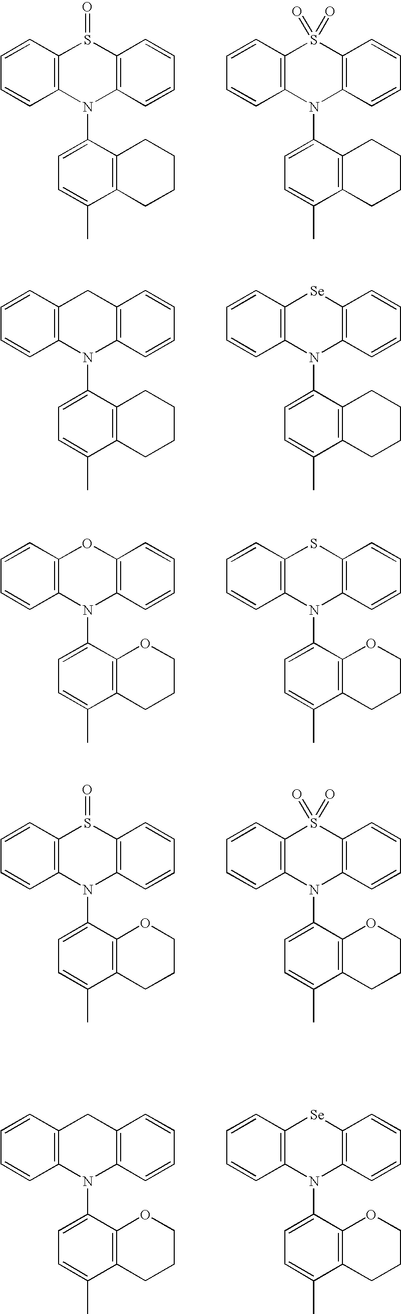

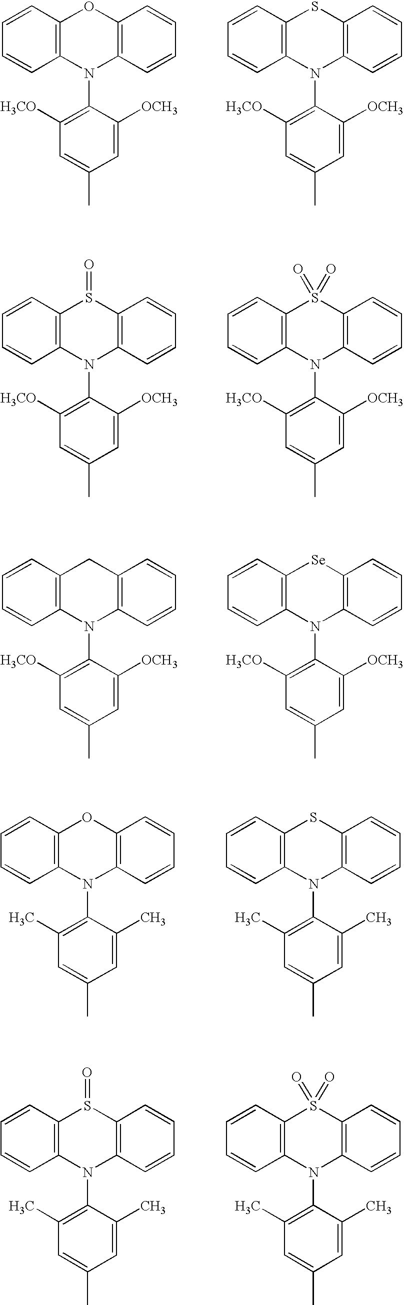

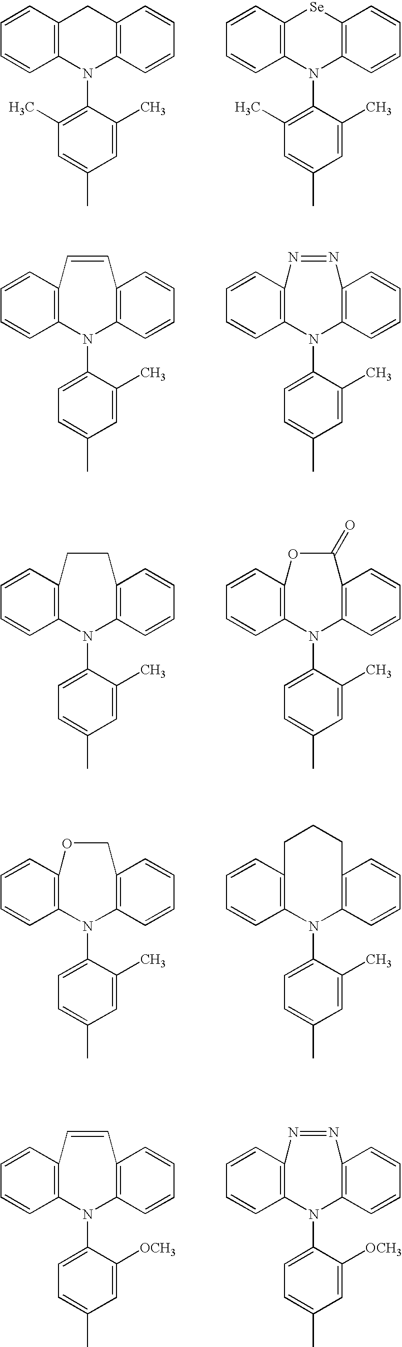

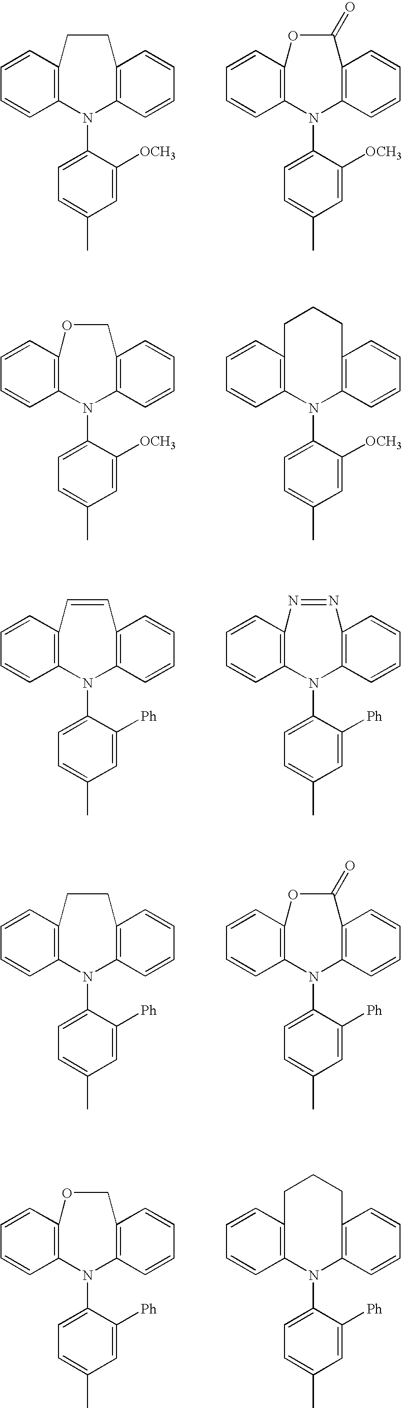

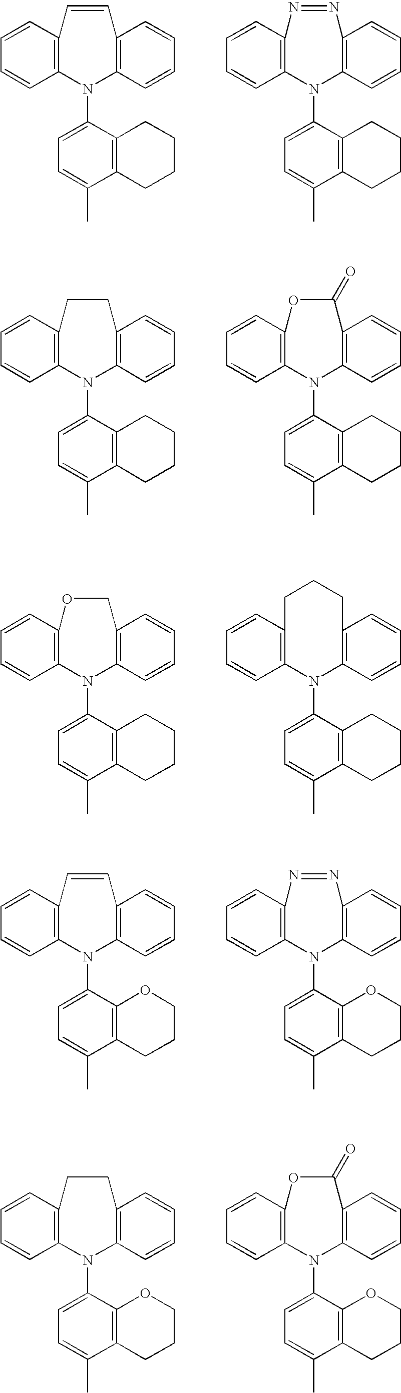

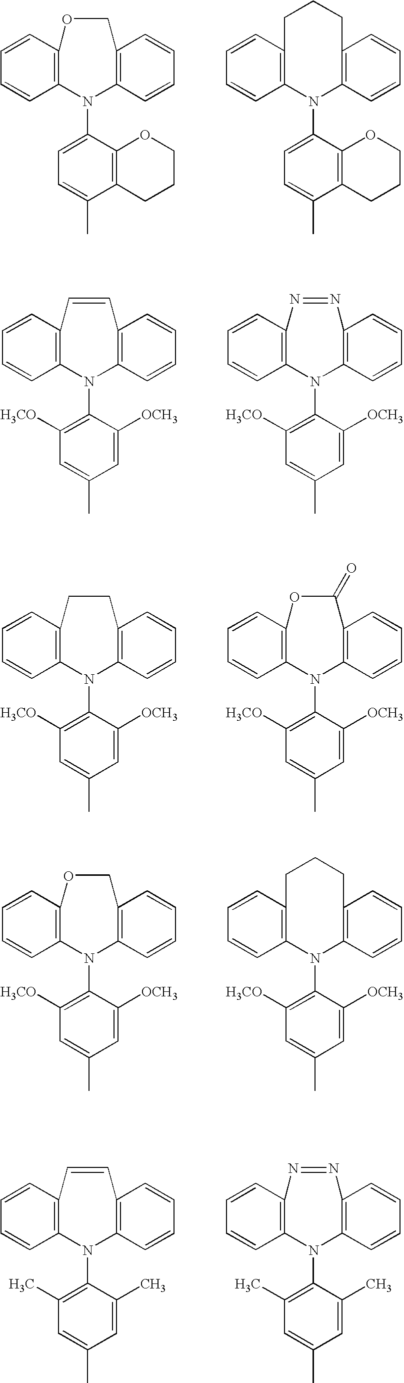

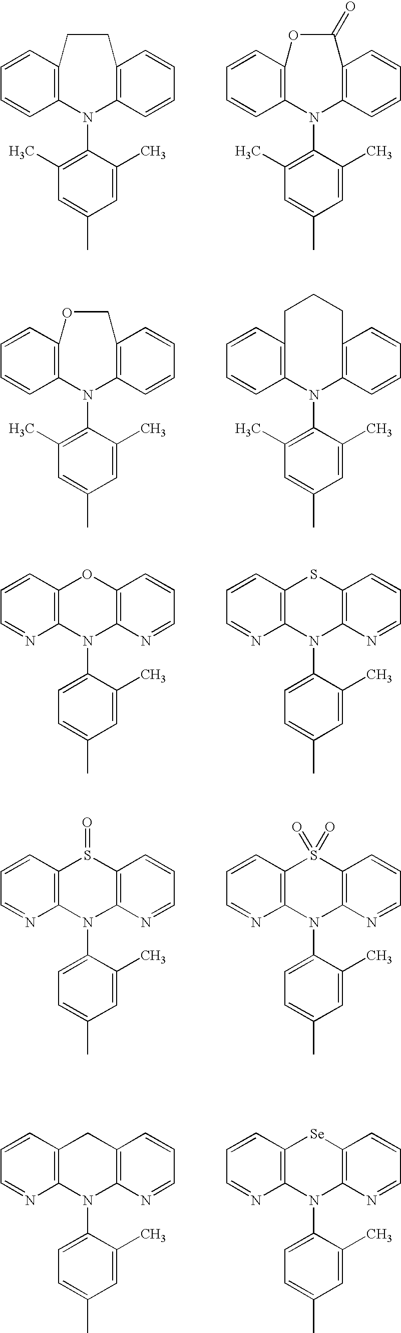

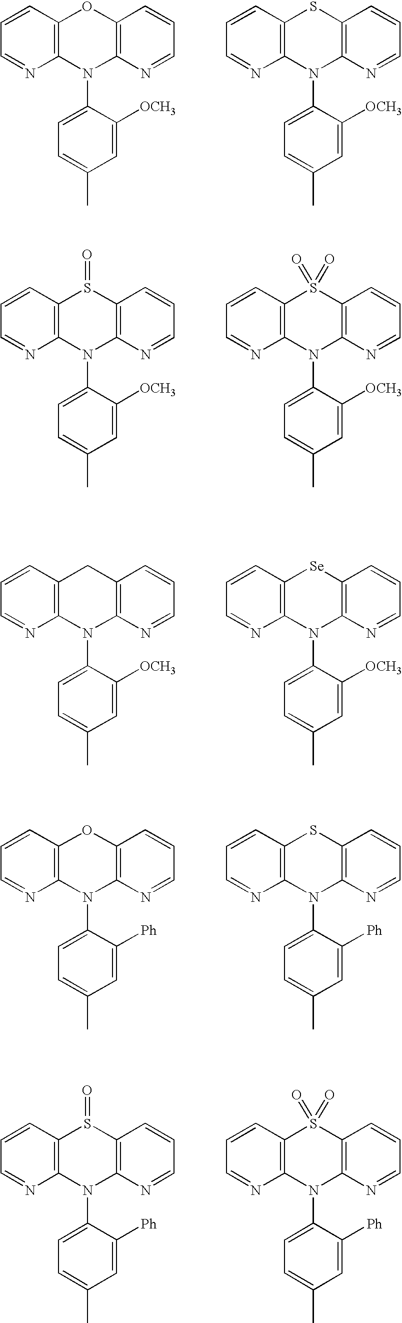

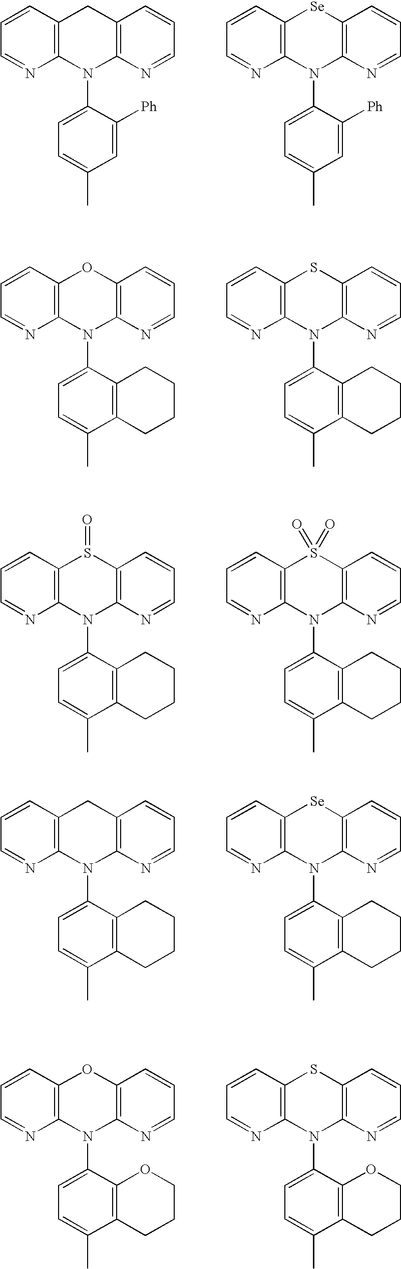

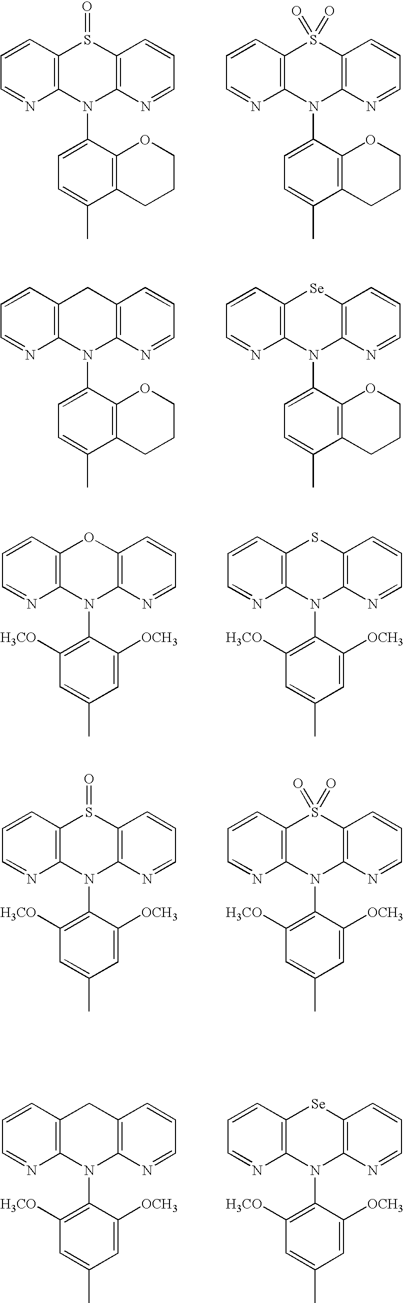

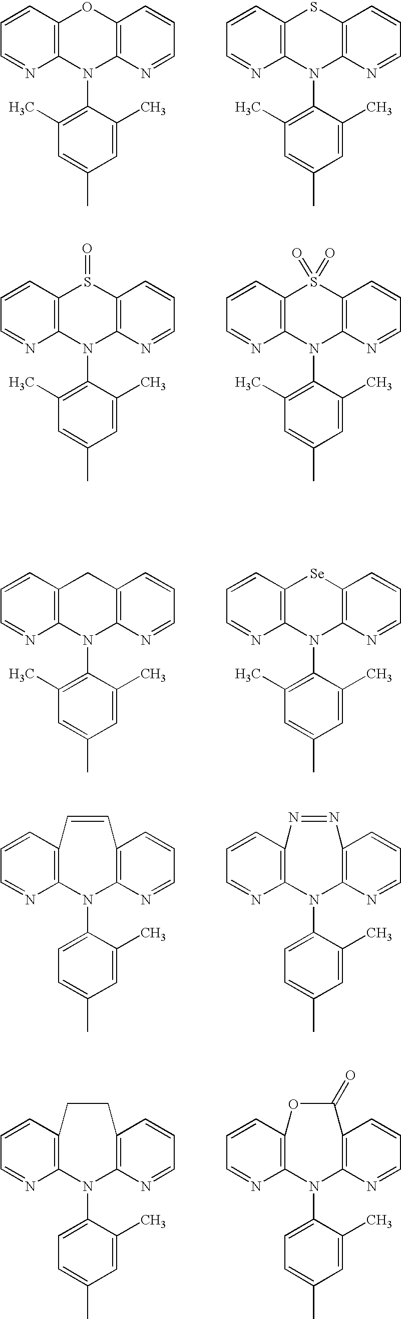

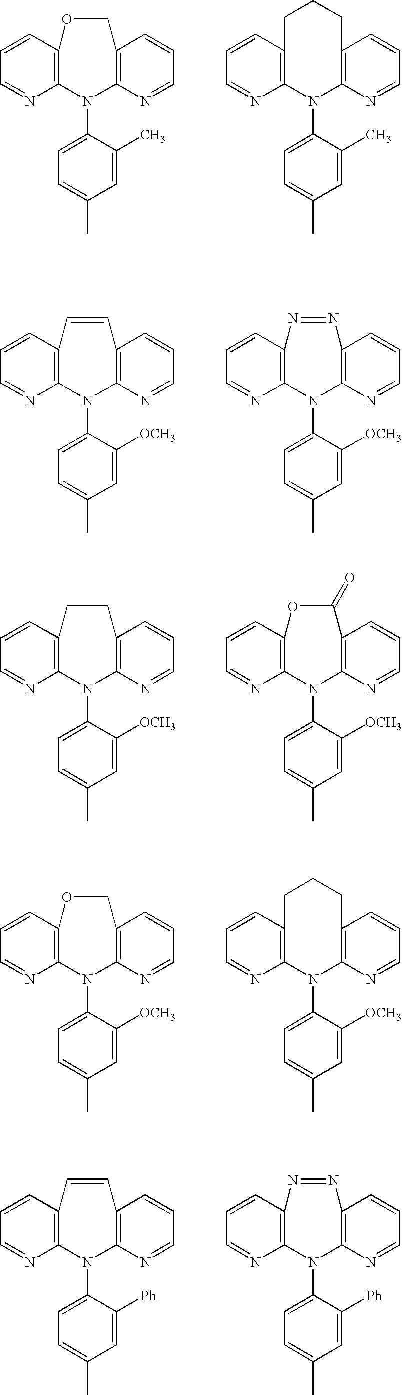

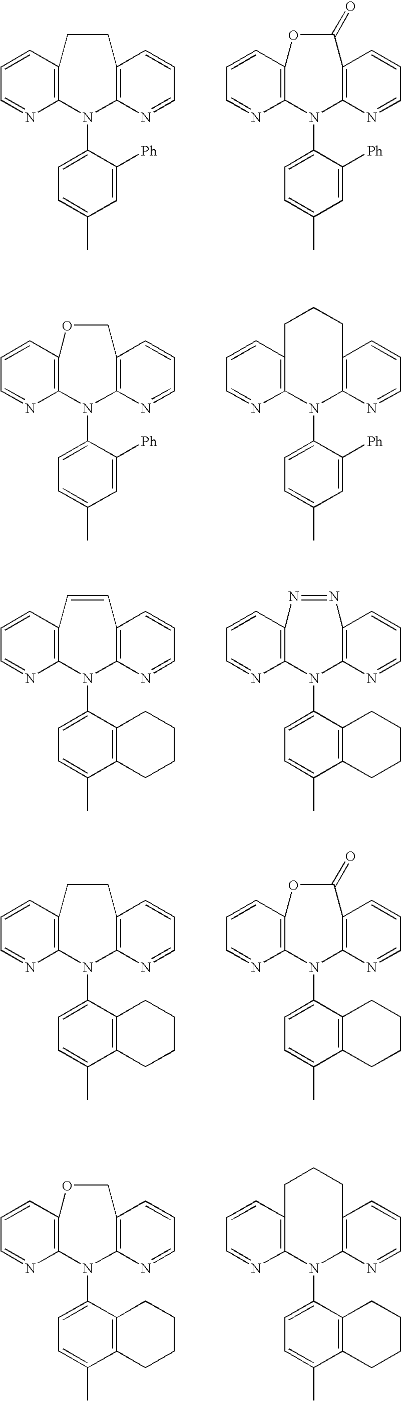

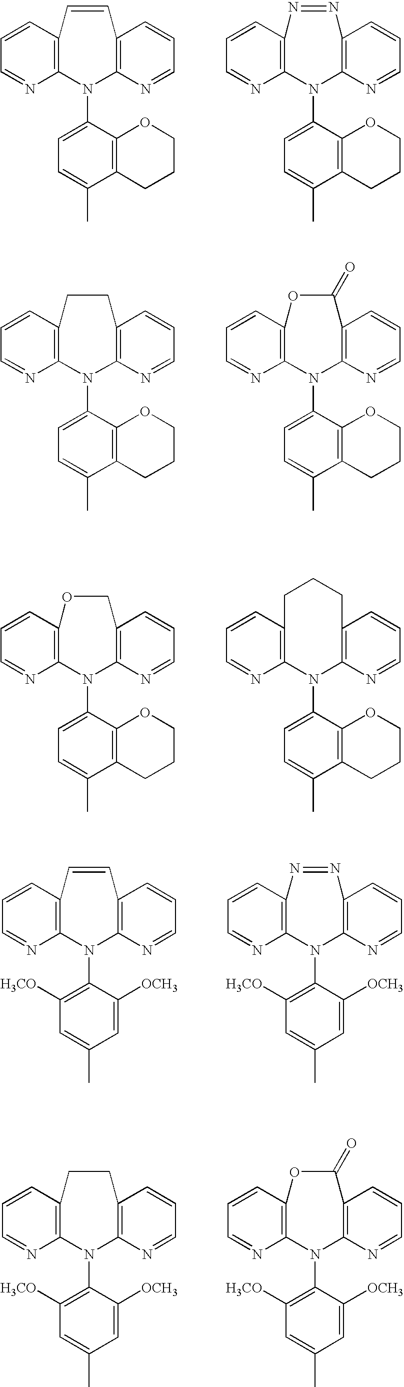

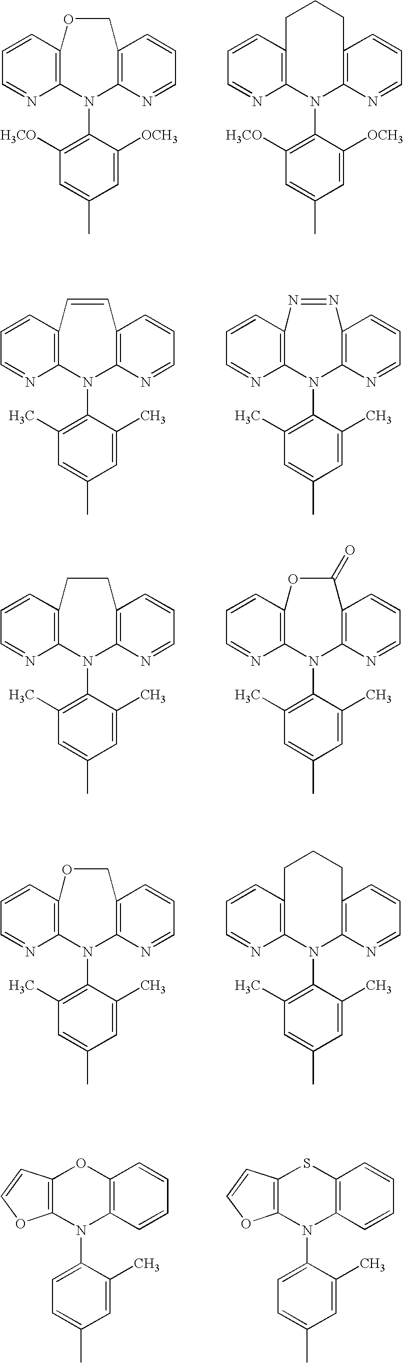

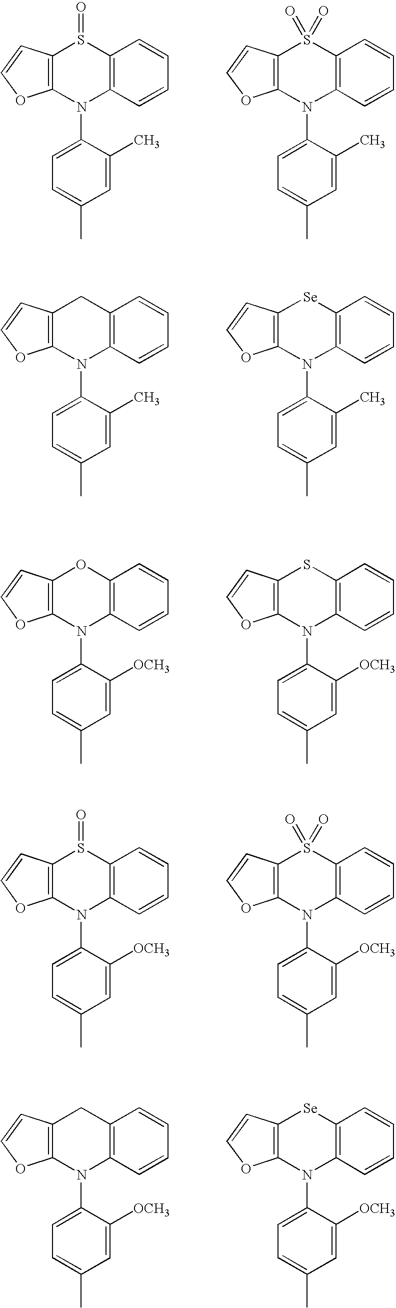

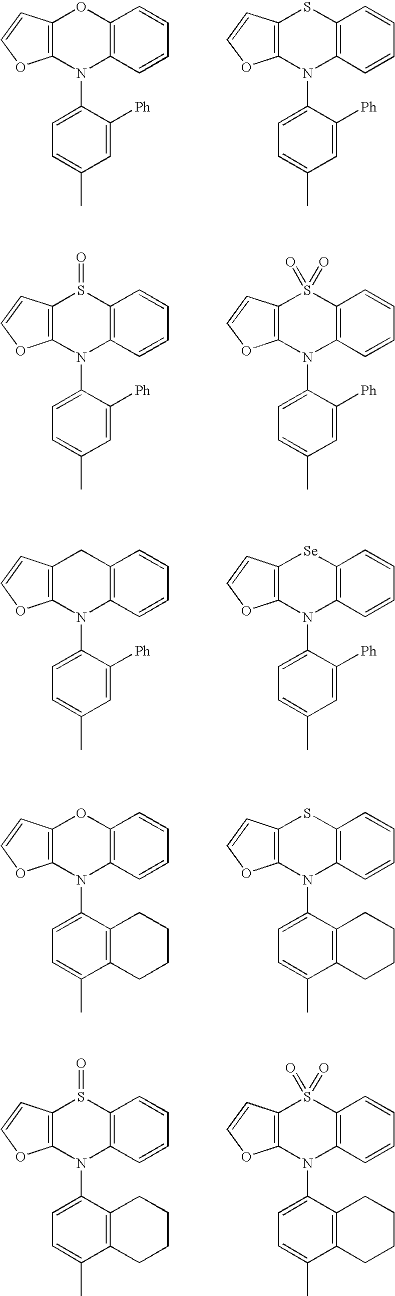

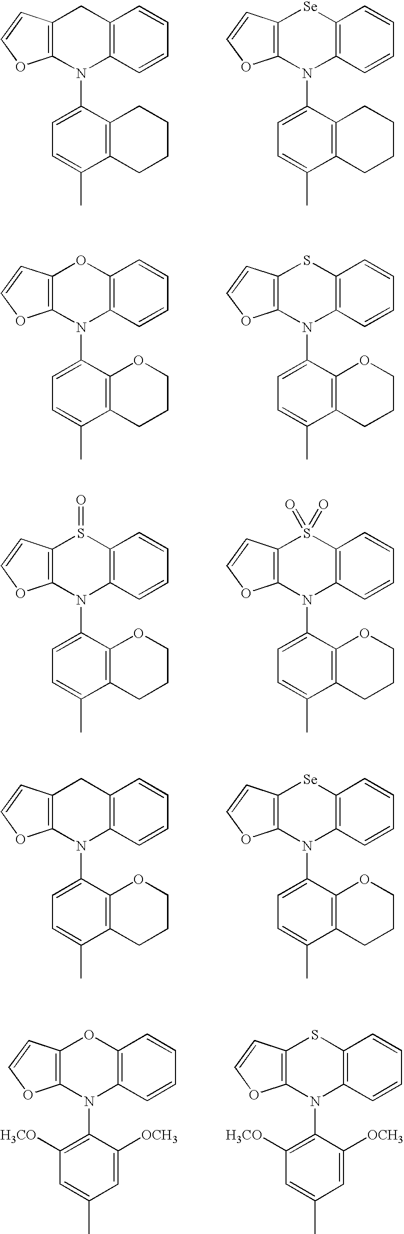

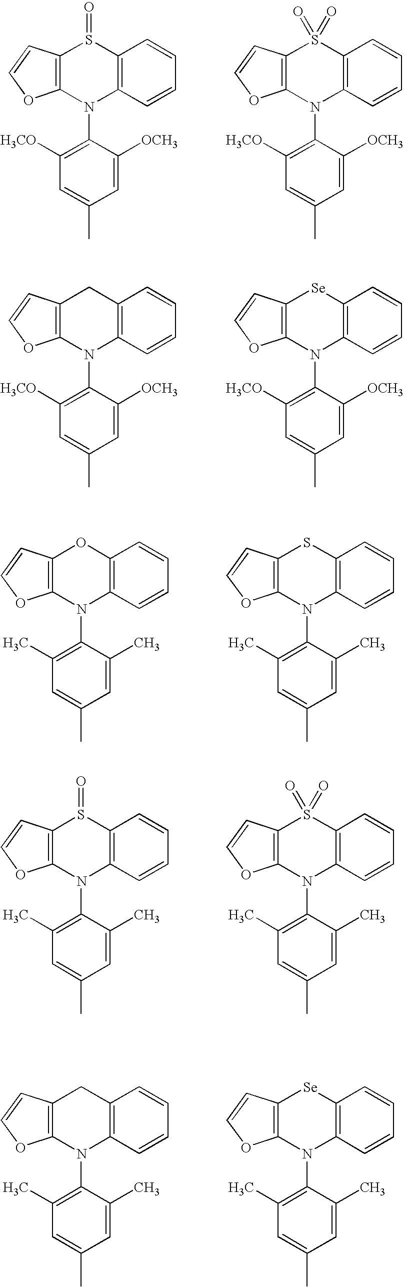

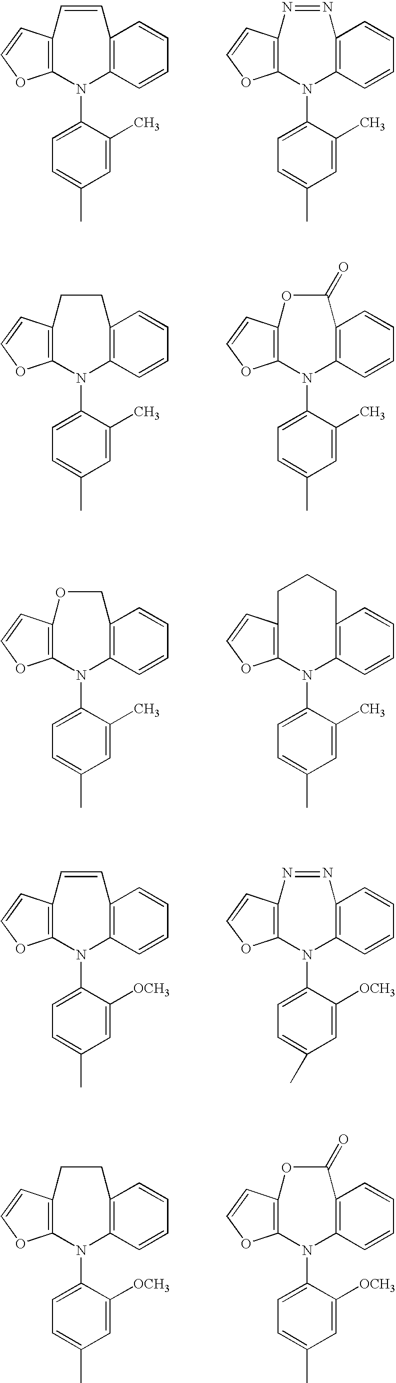

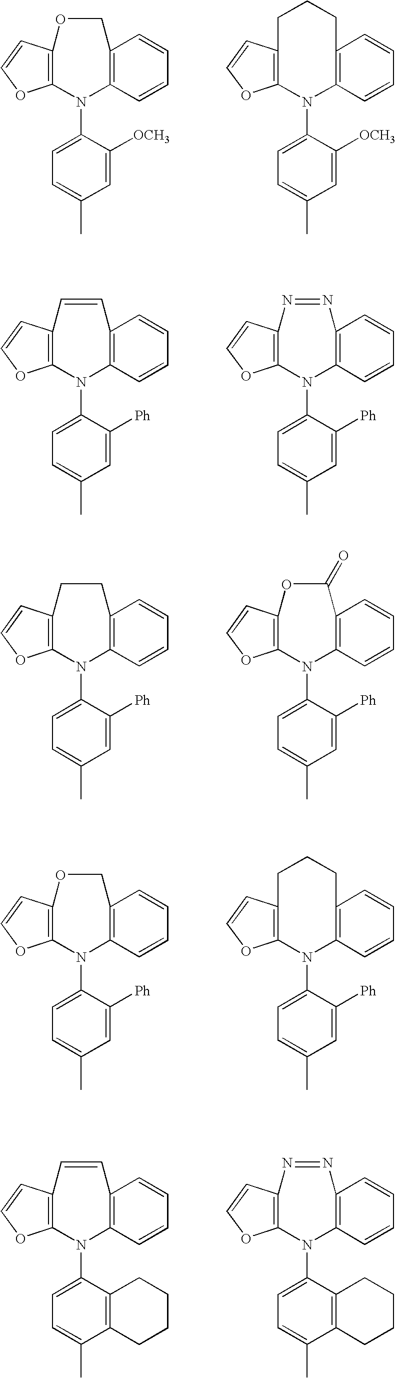

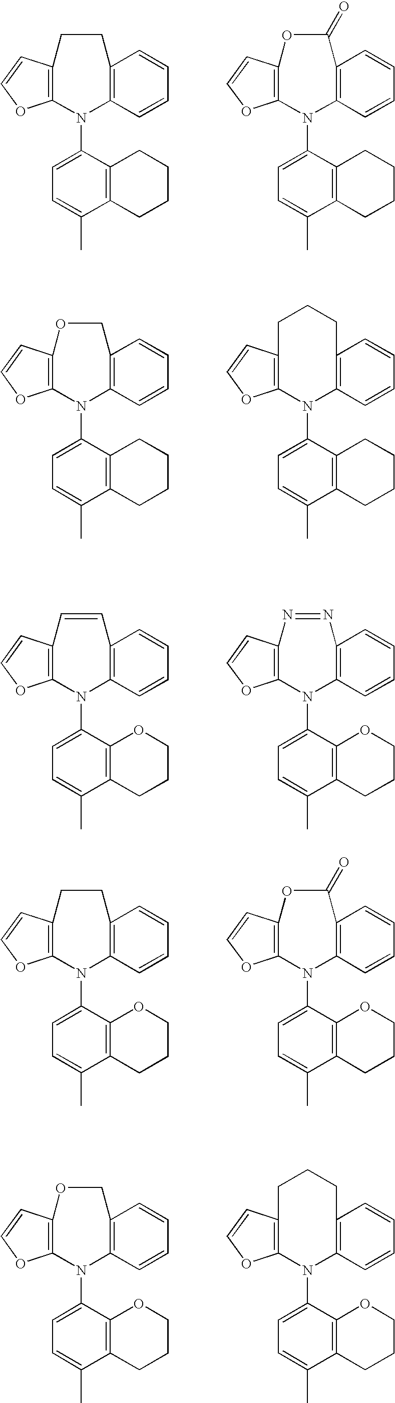

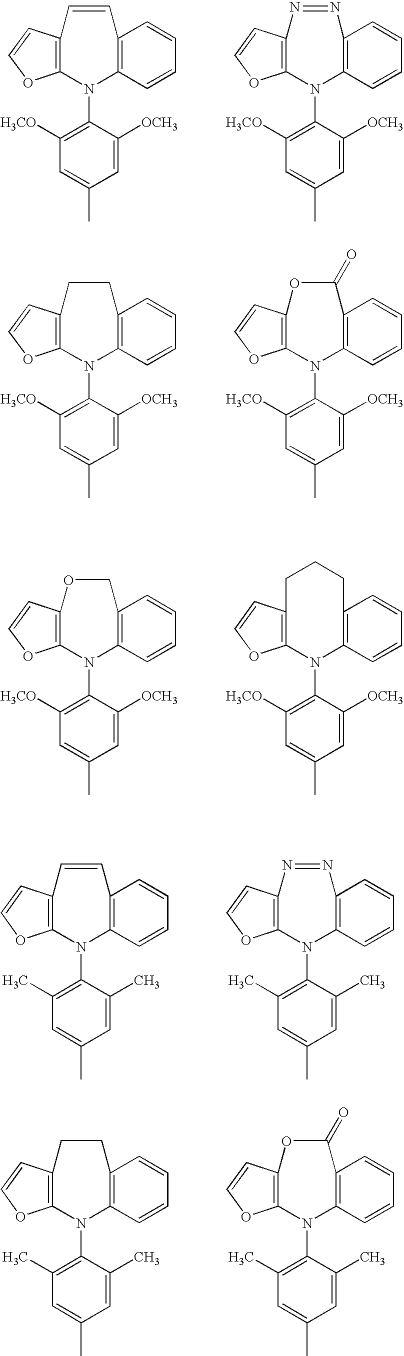

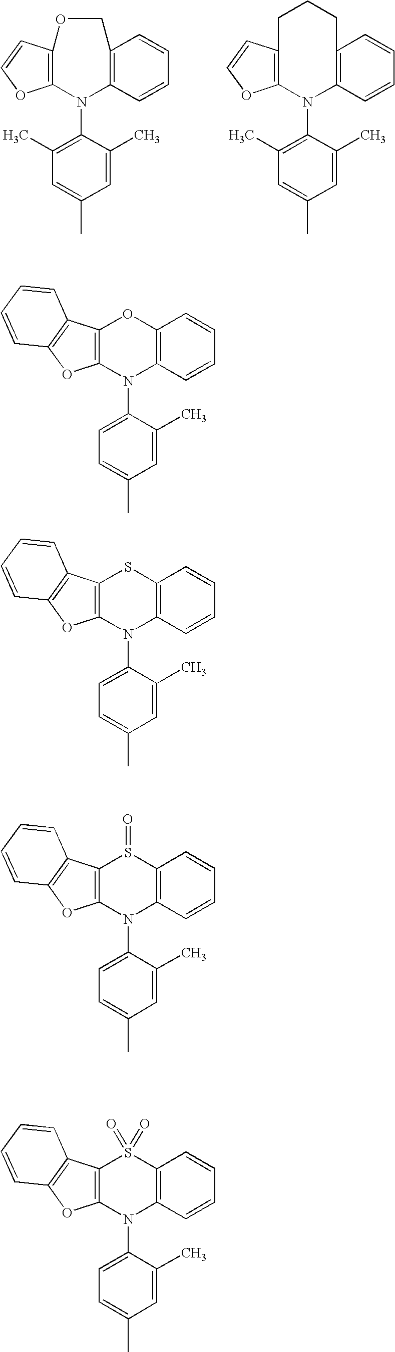

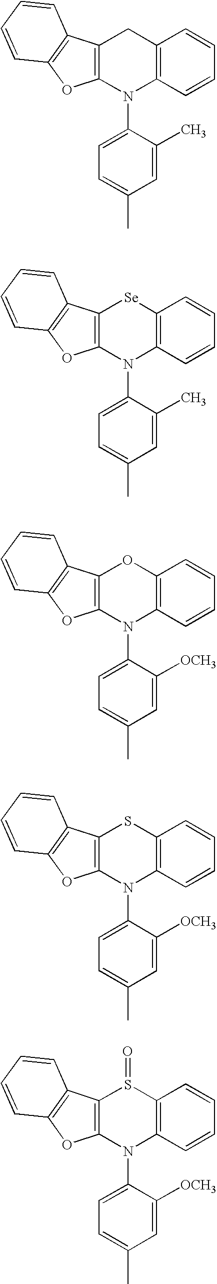

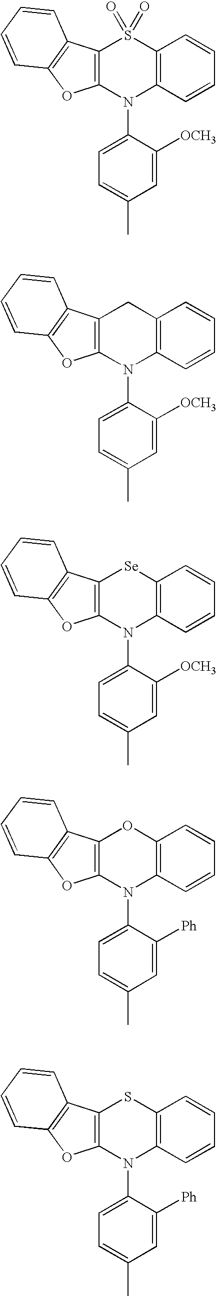

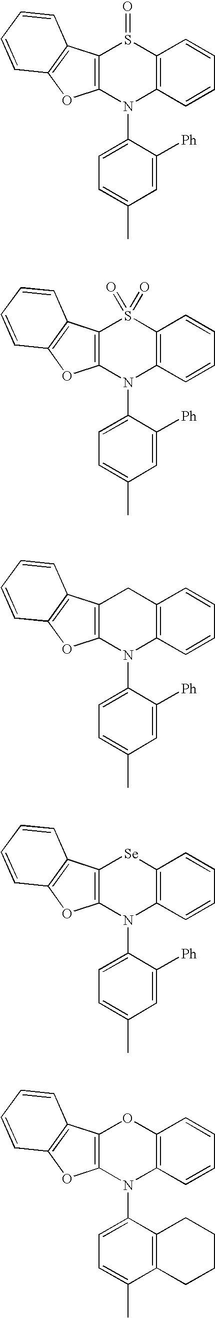

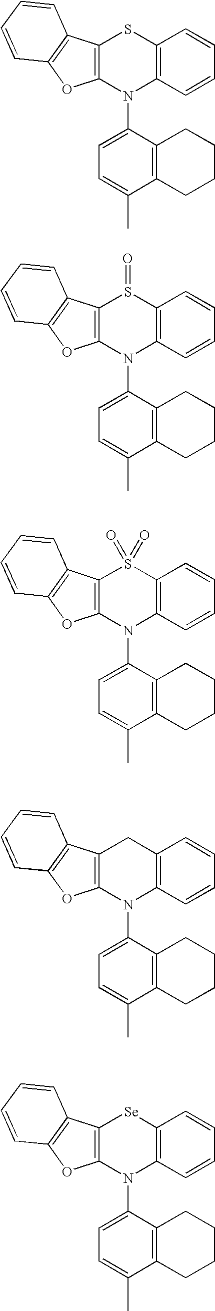

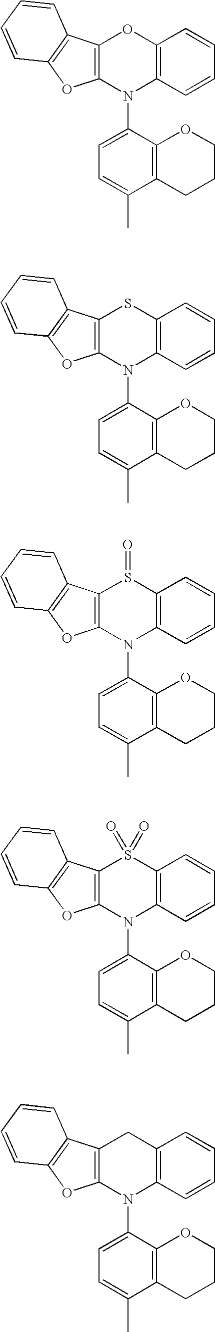

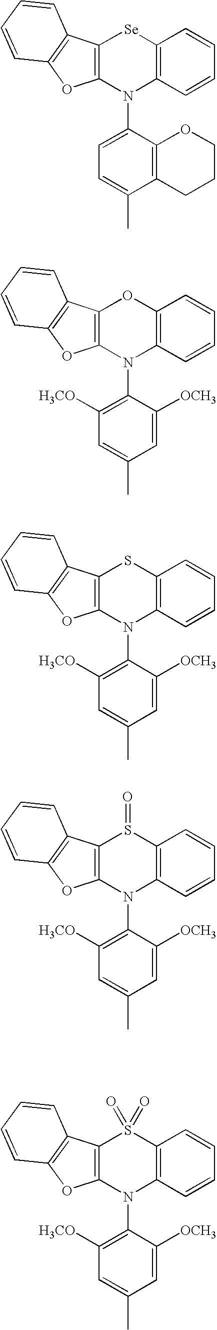

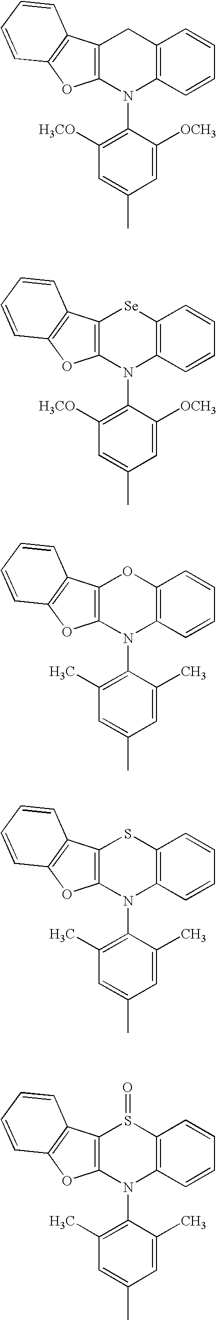

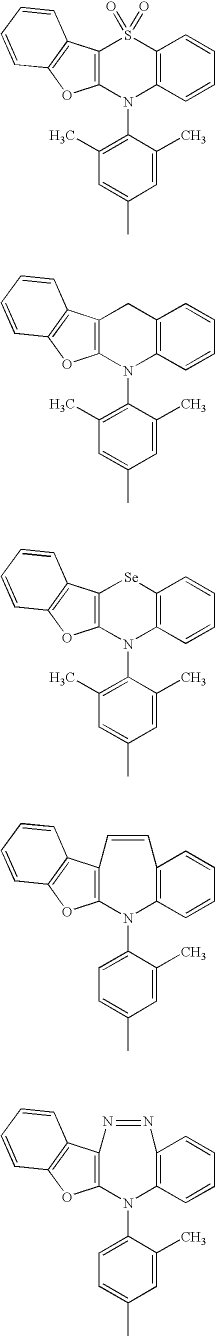

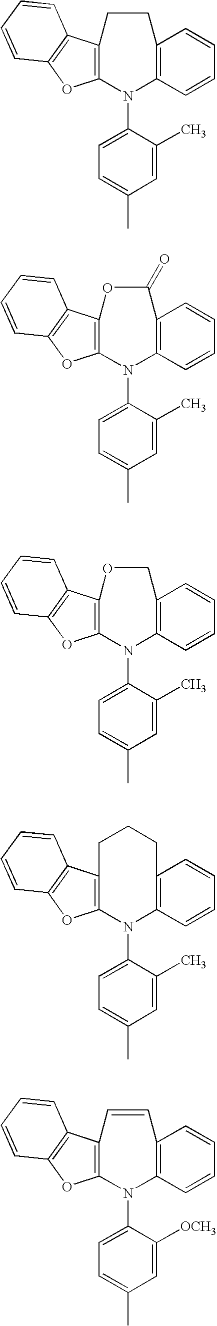

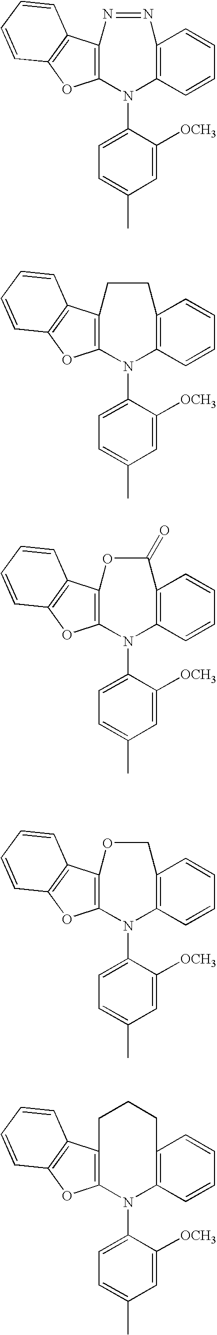

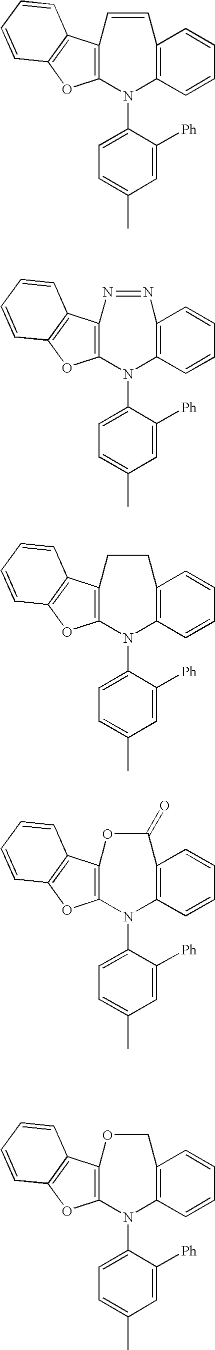

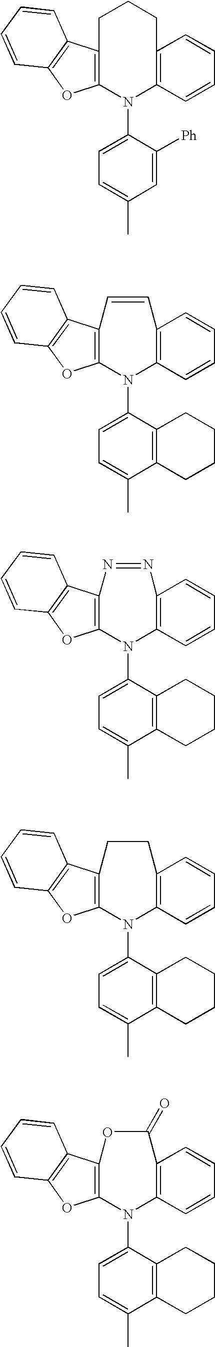

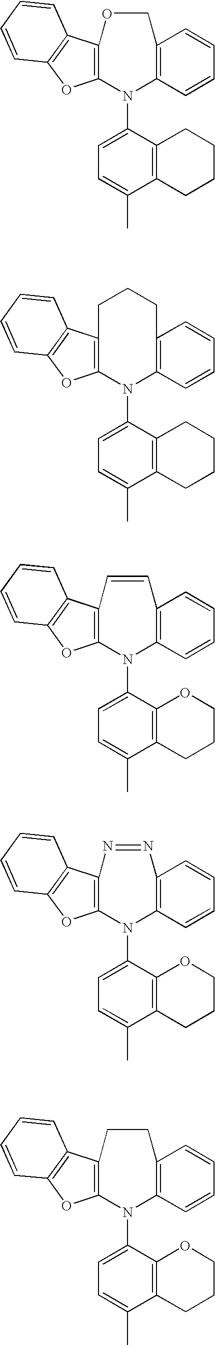

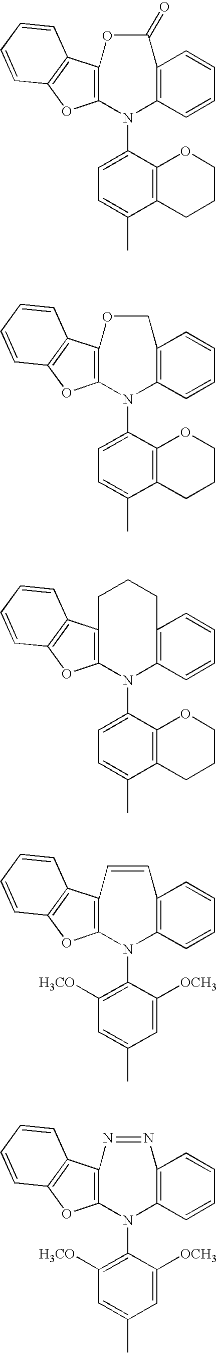

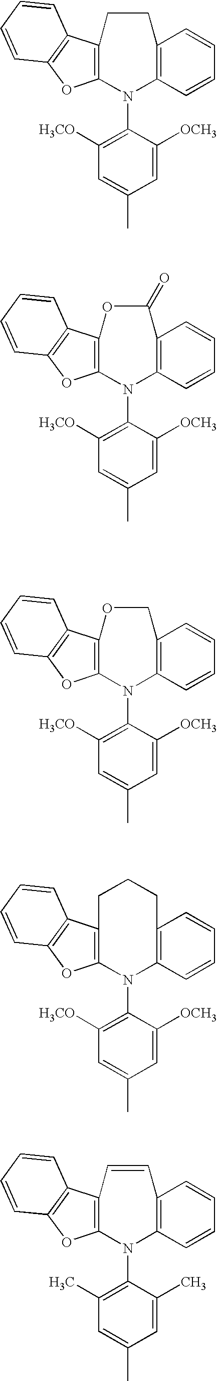

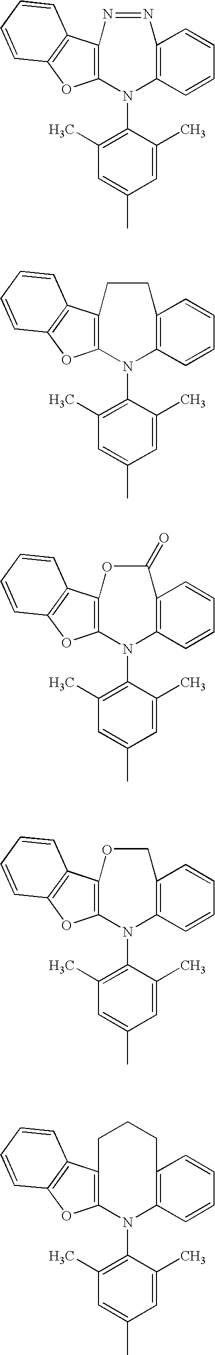

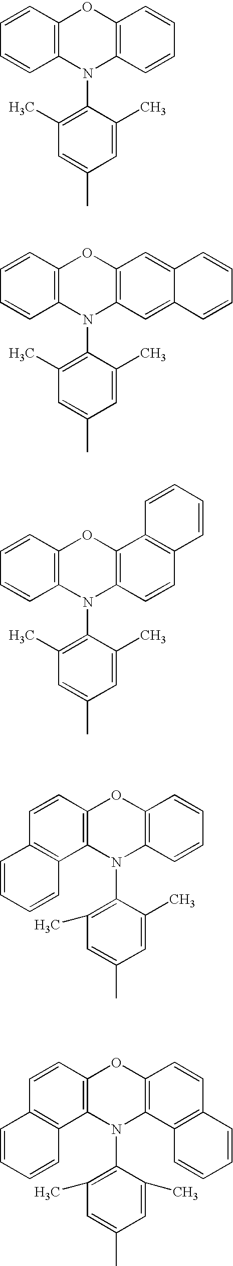

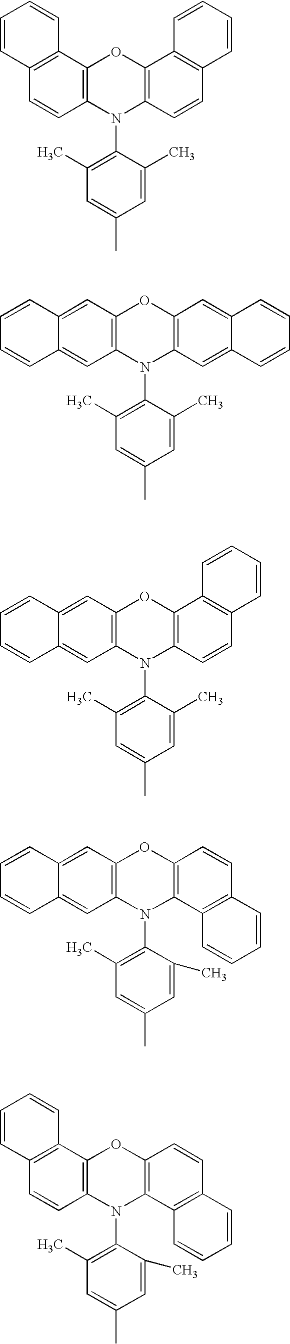

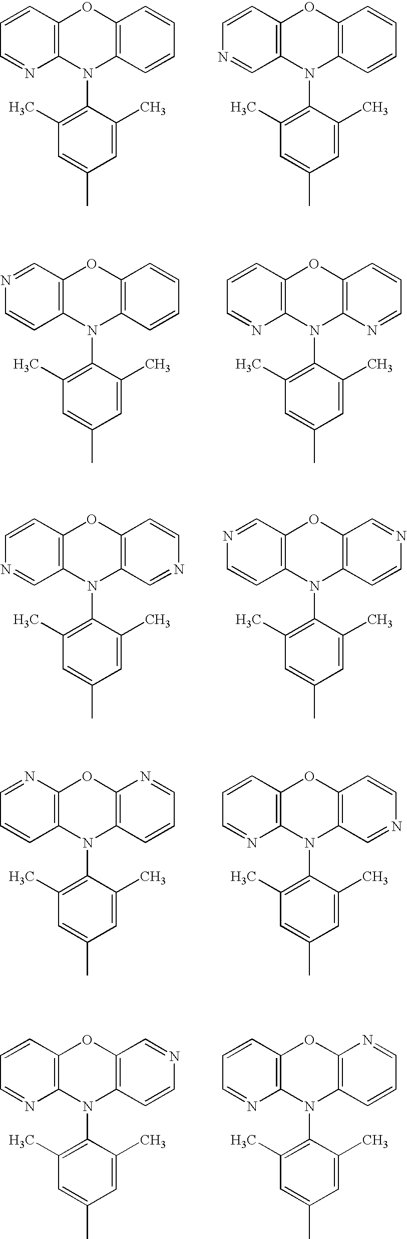

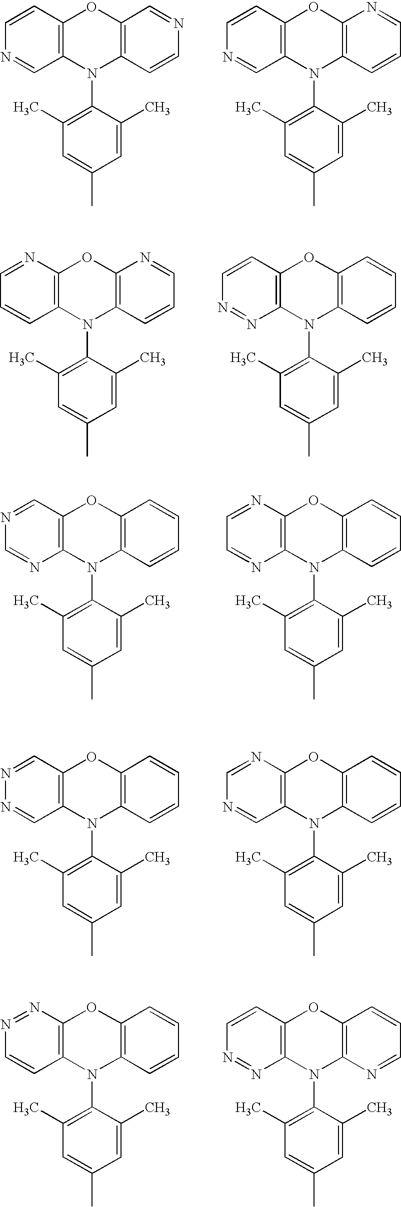

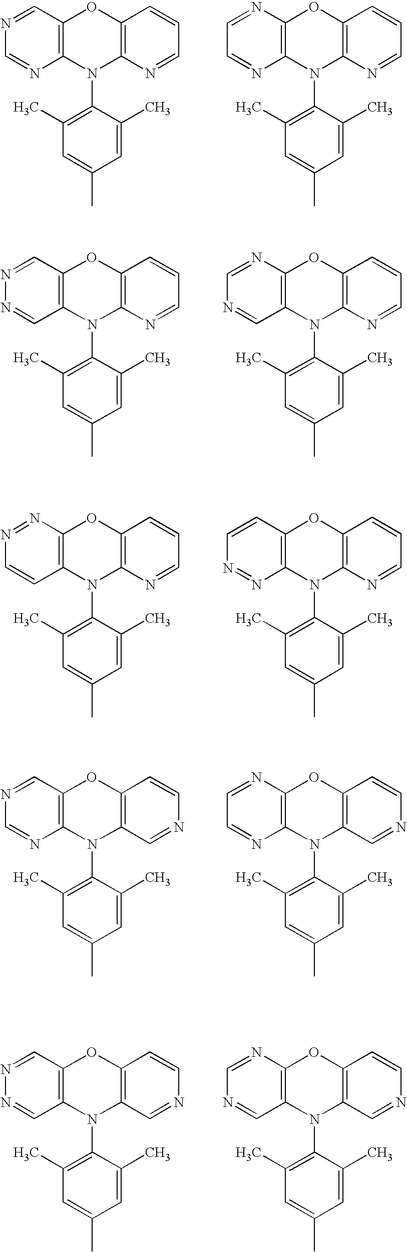

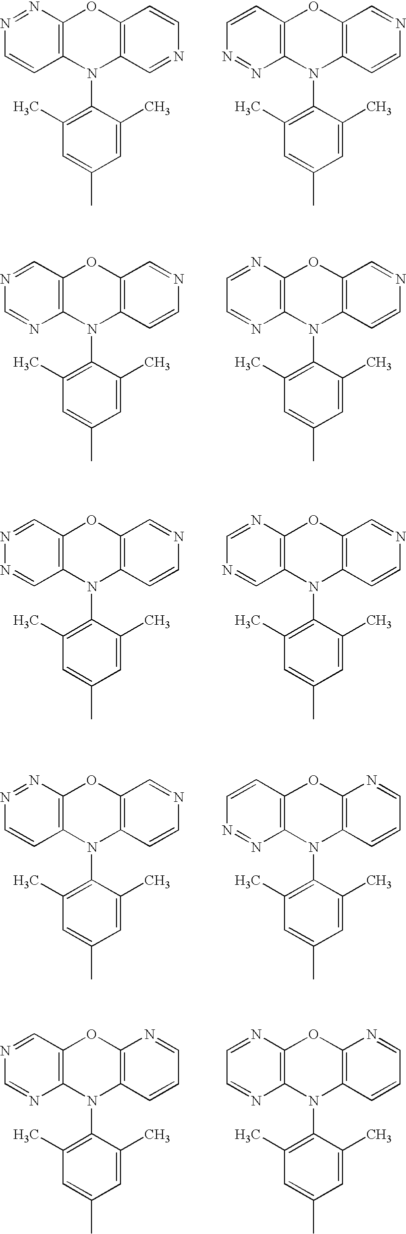

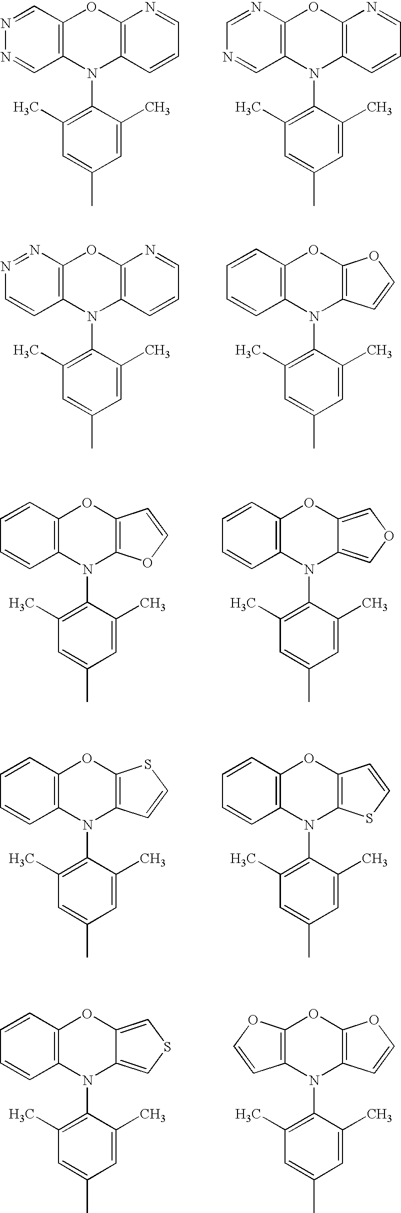

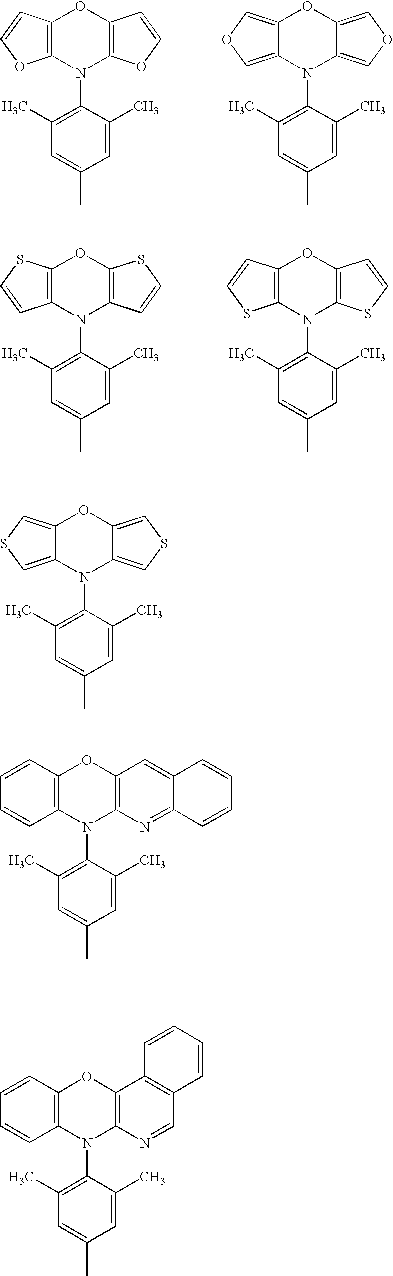

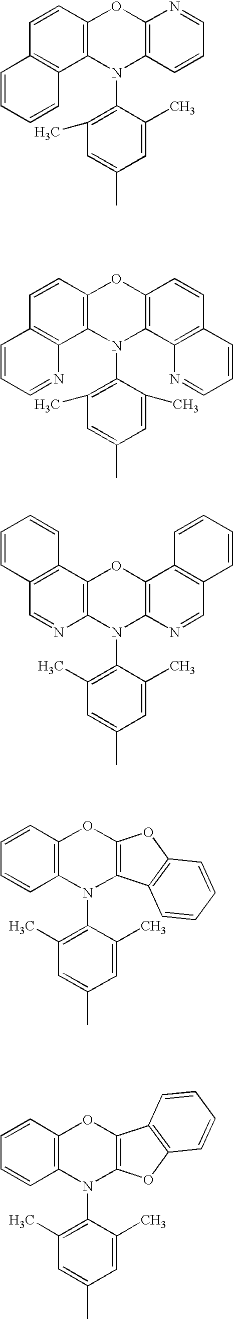

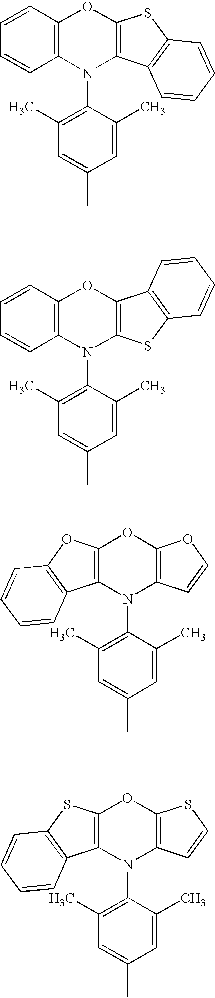

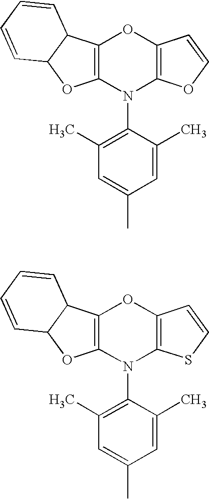

When a residue of a compound of the formula (1) is contained in a polymer compound, it may be contained only on the main chain, only on the end of the main chain, only on the side chain, or on two or more positions thereof, and preferably, contained at least on the main chain.

Regarding a residue of a compound of the formula (1), a preferable range can be confirmed also by calculation. That is, it is believed that by suppressing cleavage of a carbon-nitrogen bond showing smaller bond energy as compared with a carbon-carbon bond in a residue of a compound of the above-described formula (1), the life when manufactured into a polymer LED can be improved. It has been found that when the following formula (11) is satisfied using the shielding ratio and electron density of a N atom as a parameter correlated with cleavage of a carbon-nitrogen bond, the life is longer. It is believed that larger the shielding ratio, proximity of molecules giving a cause for a side reaction on a N atom can be suppressed more effectively, and smaller the electron density on a N atom, nucleophilic reactivity of a N atom becomes lower, thereby suppressing a side reaction.

(1−A)×√\vec{}B≦0.070 (11)

In the formula, A represents the shielding ratio of a nitrogen atom connected to the ring C³, and B represents the electron density of a nitrogen atom connected to the ring C³.

Here, the shielding ratio A is defined by the following formula.

A=1−(φf/4π)

φf is the sum of solid angles of portions where a light from a point light source illuminates inner parts of sphere having a radius (L+a) from the center original point without being shielded by an atom in the compound other than the nitrogen atom, if the distance from the original point which is the center of the nitrogen atom to the center of an atom in the compound furthest from the original point is represented by L, and the Van der Waals radius of each atom in the compound is represented by a, hypothesizing the point light source being placed at the original point, in the most stable conformation of the compound.

The expression light of a point light source referred herein is only as a matter of convenience, and there is no need to consider mutual interference, diffraction and the like of the light. It is construed that in regions within the Van der Waals radius of each atom from the center of the atom in the compound other than the nitrogen atom, the above-described light is shielded.

For determining φf, solid angles are measured in the case of no existence of other atoms than the nitrogen atom in space regions connecting the original point and fine regions on the inner surface of sphere having a radius of L+a, and the sum of the solid angles are calculated, thus, φf can be determined.

The case of no existence of other atoms than the nitrogen atom means a case in which regions within the Van der Waals radius of the other atom from the center of the atom dot not exist in the above-described space regions.

B is a value of square of the atom orbital coefficient corresponding to the nitrogen atom of the highest occupied molecular orbital which is any one selected from highest occupied molecular orbitals (HOMO) measured by the molecular orbital method, in the most stable conformation of the compound, and calculated according to the following formula.

B=(C^{HOMO})²

Here, C^{HOMO} represents the atom orbital coefficient of HOMO of the nitrogen atom.

Calculation of a value of square of the atom orbital coefficient is performed with 3 significant digits.

The atom orbital coefficient of the highest occupied molecular orbital and the most stable conformation of a compound for calculation of the sum φf solid angles and the molecular orbital can be obtained by effecting structure optimization by the AM1 method (Dewar, M. J. S. et al., J. Am. Chem. Soc., 107, 3902 (1985)) which is a semi-empirical molecular orbital method.

The calculation means are specifically explained. That is, for a monomer of a compound, calculation was performed while optimizing the structure by the AM1 method using a molecular orbital calculation program, WinMOPAC 3.0 Professional (MOPAC2000 V1.3) (keyword: AM1 PRECISE EF VECTORS).

Compounds containing a residue of a compound of the above-described formula (1) (preferably, compounds containing a repeating unit of the formula (2)) are, when further containing a repeating unit of the following formula (4), preferable from the standpoint of excellent device properties such as light emission efficiency, life and the like when used in a polymer light emitting device.

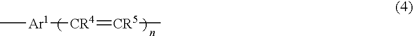

In the formula, Ar¹ represents an arylene group, di-valent heterocyclic group or di-valent group having a metal complex structure. R⁴ and R⁵ represent each independently a hydrogen atom, alkyl group, aryl group, mono-valent heterocyclic group or cyano group. n represents 0 or 1.

The arylene group Ar¹ has a carbon number of usually 6 to 60, preferably 6 to 20, and exemplified are phenylene groups (for example, formulae 1 to 3 in the following figure), naphthalenediyl groups (formulae 4 to 13 in the following figure), anthracenylene groups (formulae 14 to 19 in the following figure), biphenylene groups (formulae 20 to 25 in the following figure), triphenylene groups (formulae 26 to 28 in the following figure), condensed ring compound groups (formulae 29 to 38 in the following figure), and the like. In the formulae, Rs represent each independently a hydrogen atom, halogen atom, alkyl group, alkoxy group, alkylthio group, aryl group, aryloxy group, arylthio group, arylalkyl group, arylalkyloxy group, arylalkylthio group, alkenyl group, alkynyl group, heteroaryloxy group or heteroarylthio group. The carbon number of the arylene group does not include the carbon number of the substituent R.

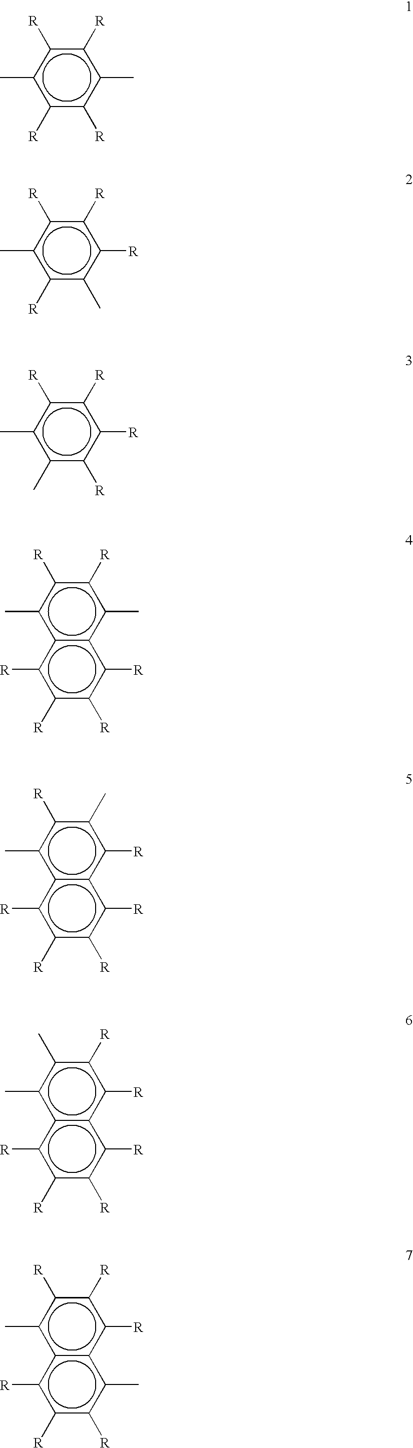

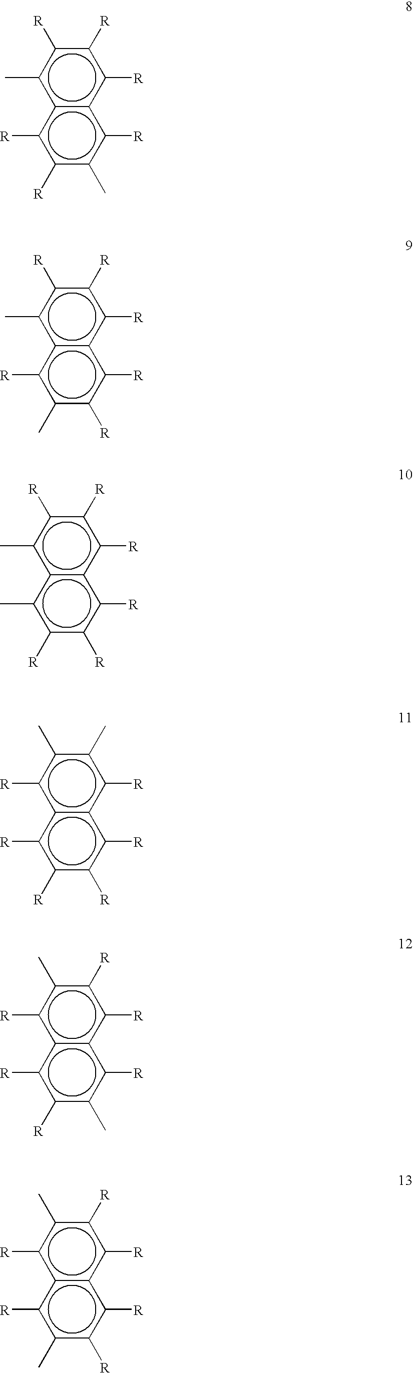

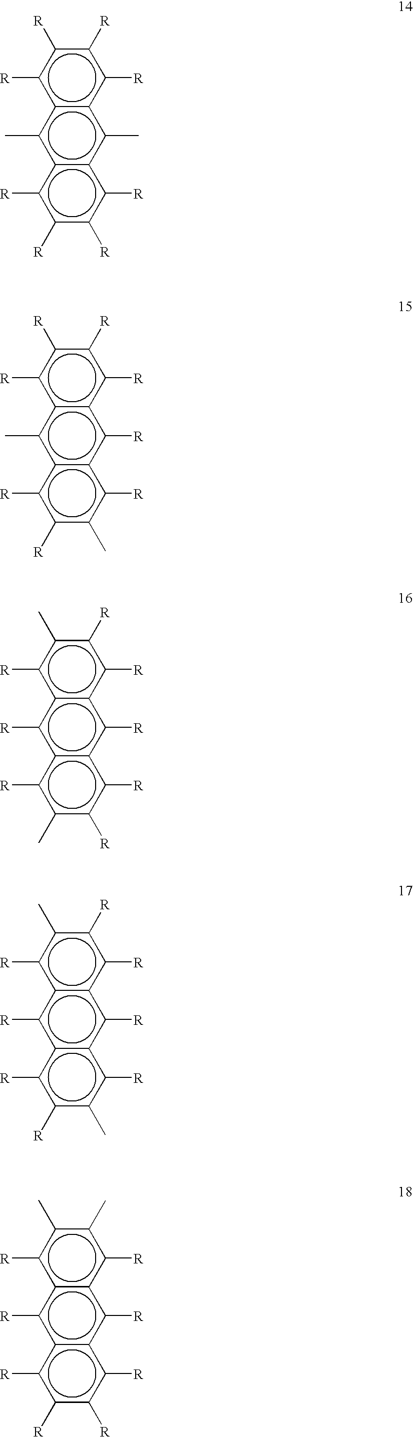

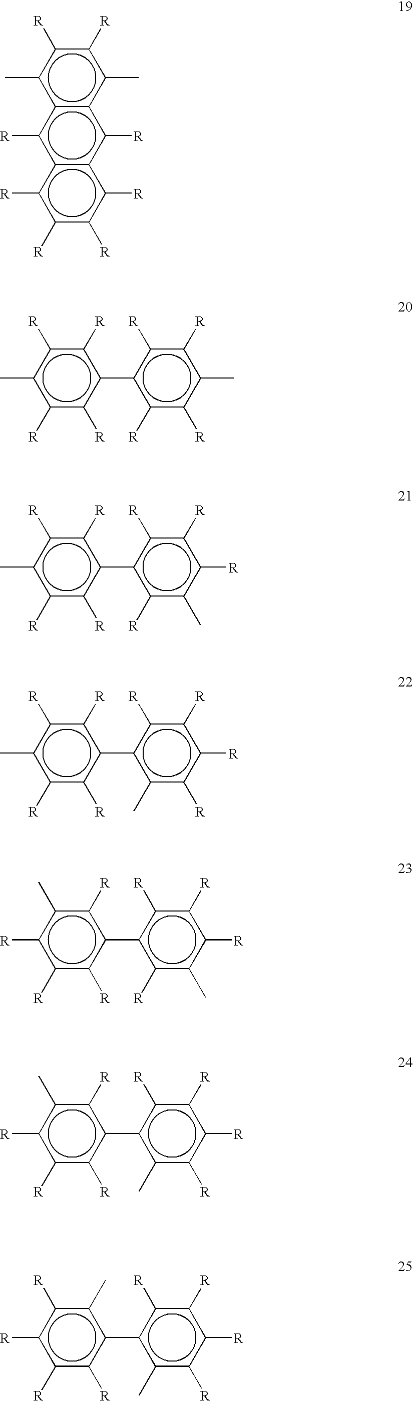

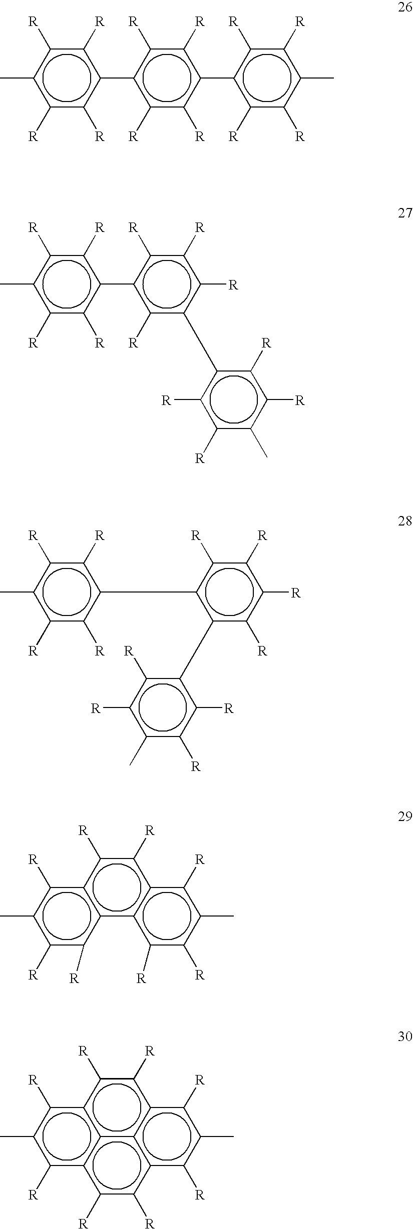

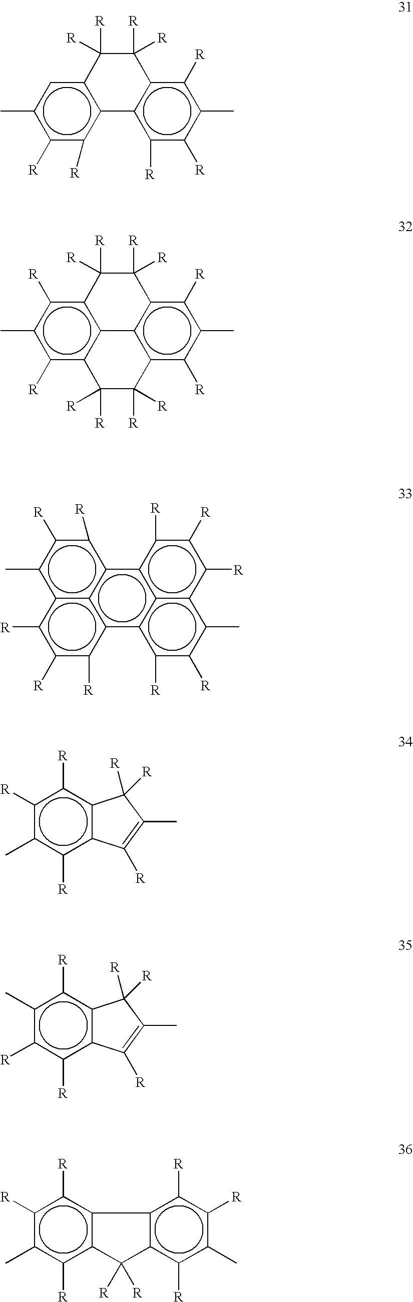

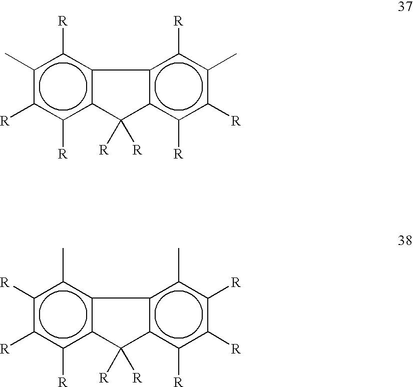

In the present invention, the divalent heterocyclic group means an atomic group remaining after removing two hydrogen atoms from a heterocyclic compound, and has a carbon number of usually 4 to 60, preferably 4 to 20. In the formulae, Rs represent each independently a hydrogen atom, halogen atom, alkyl group, alkoxy group, alkylthio group, aryl group, aryloxy group, arylthio group, arylalkyl group, arylalkyloxy group, arylalkylthio group, alkenyl group, alkynyl group, heteroaryloxy group or heteroarylthio group. The carbon number of the di-valent heterocyclic group does not include the carbon number of the substituent.

In the present invention, the divalent heterocyclic group means an atomic group remaining after removing two hydrogen atoms from a heterocyclic compound, and has a carbon number of usually 4 to 60, preferably 4 to 20. In the formulae, Rs represent each independently a hydrogen atom, halogen atom, alkyl group, alkoxy group, alkylthio group, aryl group, aryloxy group, arylthio group, arylalkyl group, arylalkyloxy group, arylalkylthio group, alkenyl group, alkynyl group, heteroaryloxy group or heteroarylthio group. The carbon number of the di-valent heterocyclic group does not include the carbon number of the substituent.

Here, the heterocyclic compound refers to organic compounds having a cyclic structure in which elements constituting the ring include not only a carbon atom, but also a hetero atom such as oxygen, sulfur, nitrogen, phosphorus, boron and the like contained in the ring.

As the di-valent heterocyclic group, for example, the following groups are mentioned.

Divalent heterocyclic groups containing nitrogen as a hetero atom; pyridine-diyl groups (formulae 39 to 44 in the following figure), diazaphenylene groups (formulae 45 to 48 in the following figure), quinolinediyl groups (formulae 49 to 63 in the following figure), quinoxalinediyl groups (formulae 64 to 68 in the following figure), acridinediyl groups (formulae 69 to 72 in the following figure), bipyridyldiyl groups (formulae 73 to 75 in the following figure), phenanthroline-diyl groups (formulae 76 to 78 in the following figure), and the like.

Groups containing silicon, nitrogen, sulfur, selenium and the like as a hetero atom, and having a fluorene structure (formulae 79 to 93 in the following figure). It is desirable to have an aromatic amine monomer such as carbazole, triphenylaminediyl group and the like of the formulae 82 to 84 containing a nitrogen atom, from the standpoint of light emission efficiency.

5-membered ring heterocyclic groups containing silicon, nitrogen, sulfur, selenium and the like as a hetero atom: (formulae 94 to 98 in the following figure) are mentioned.

5-membered ring condensed heterocyclic groups containing silicon, nitrogen, sulfur, selenium and the like as a hetero atom: (formulae 99 to 109 in the following figure), benzothiazole-4,7-diyl group, benzooxadiazole-4,7-diyl group and the like are mentioned.

5-membered ring heterocyclic groups containing silicon, nitrogen, sulfur, selenium and the like as a hetero atom, containing bonding at α-position of its hetero atom to form a dimer or oligomer: (formulae 111 to 112 in the following figure) are mentioned.

5-membered ring heterocyclic groups containing silicon, nitrogen, sulfur, selenium and the like as a hetero atom, containing bonding to a phenyl group at α-position of its hetero atom: (formulae 112 to 118 in the following figure) are mentioned.

Tricyclic groups obtained by bonding of condensed heterocyclic groups containing nitrogen, oxygen, sulfur and the like as a hetero atom to a benzene ring or monocyclic heterocyclic group: (formulae 120 to 125 in the following figure) are mentioned.

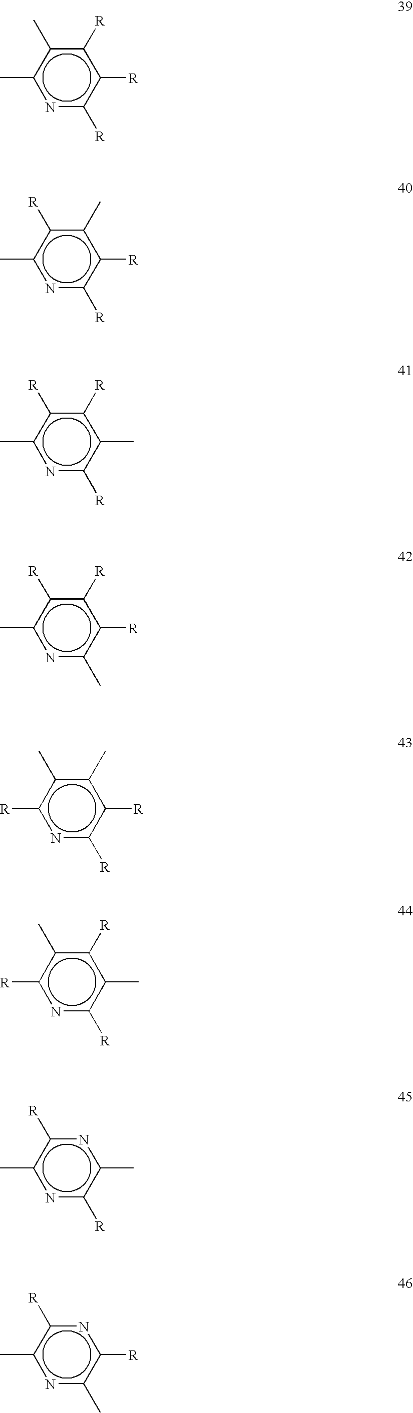

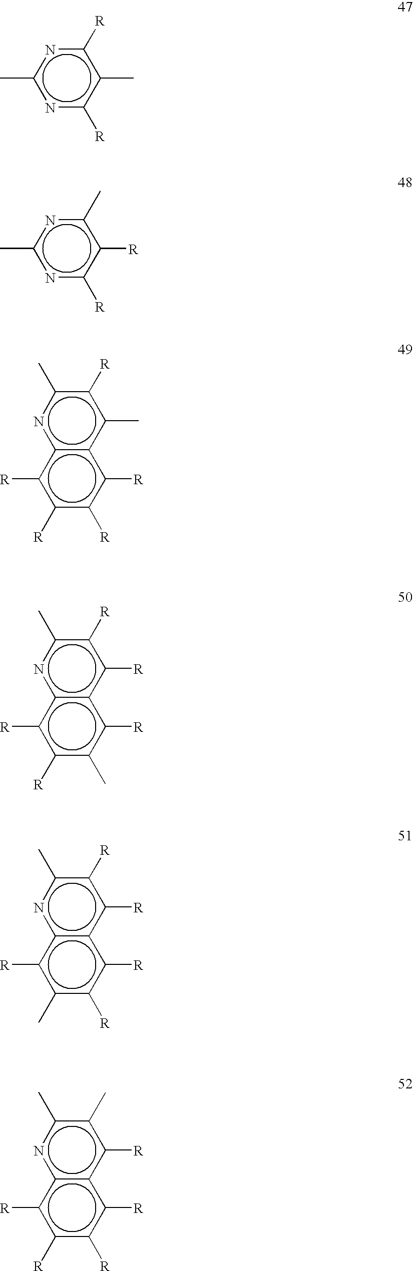

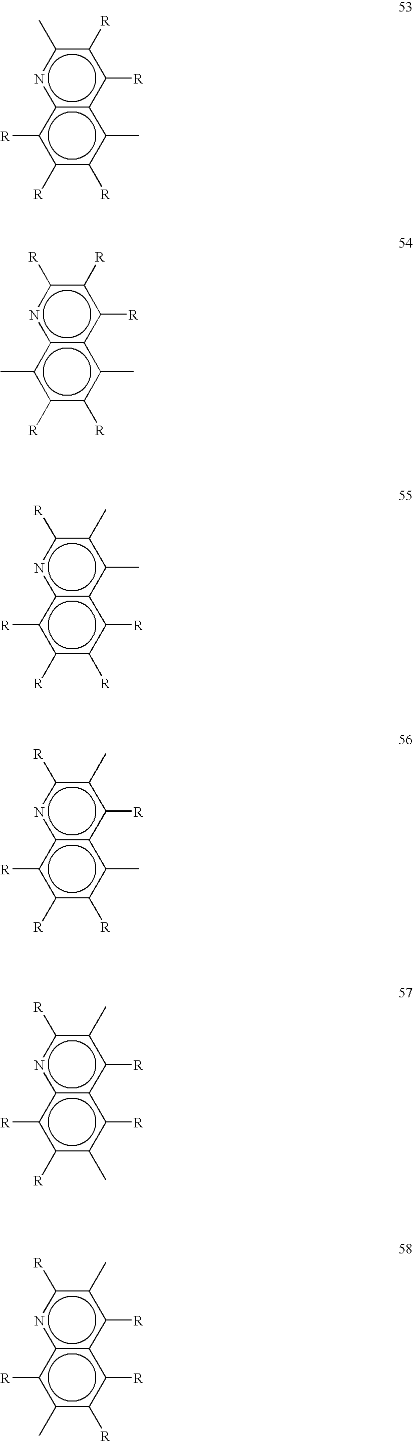

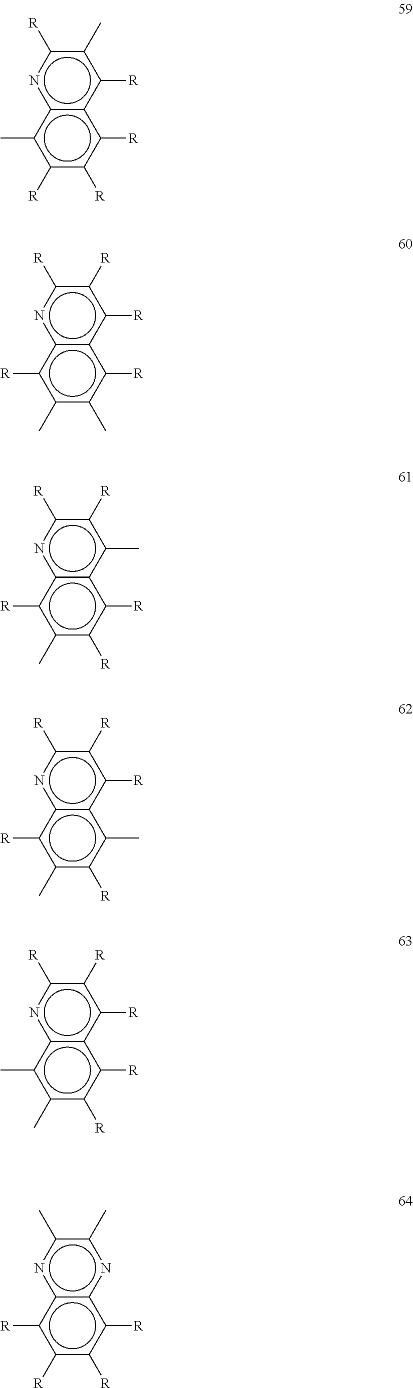

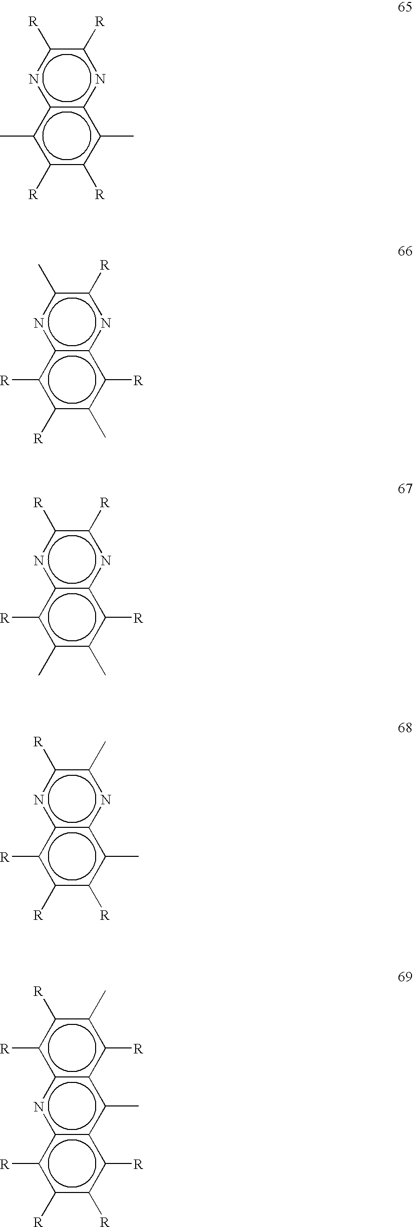

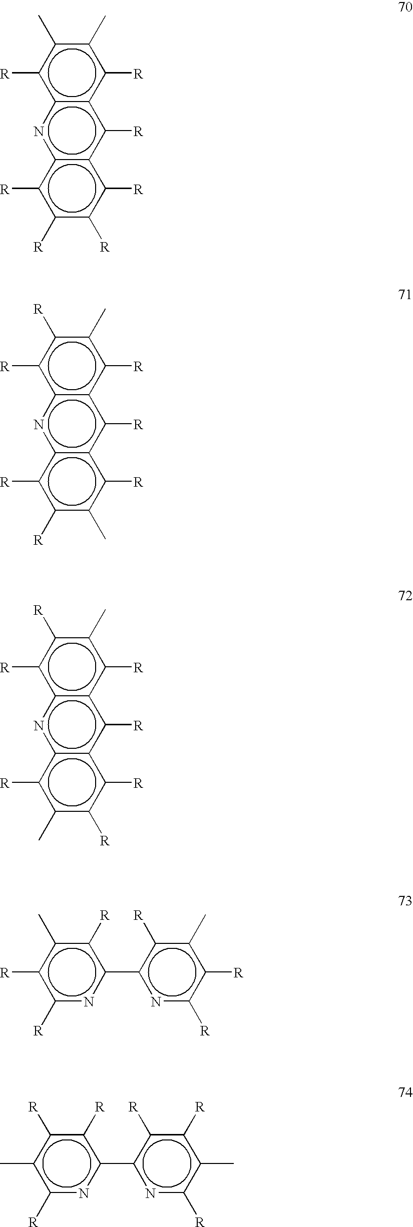

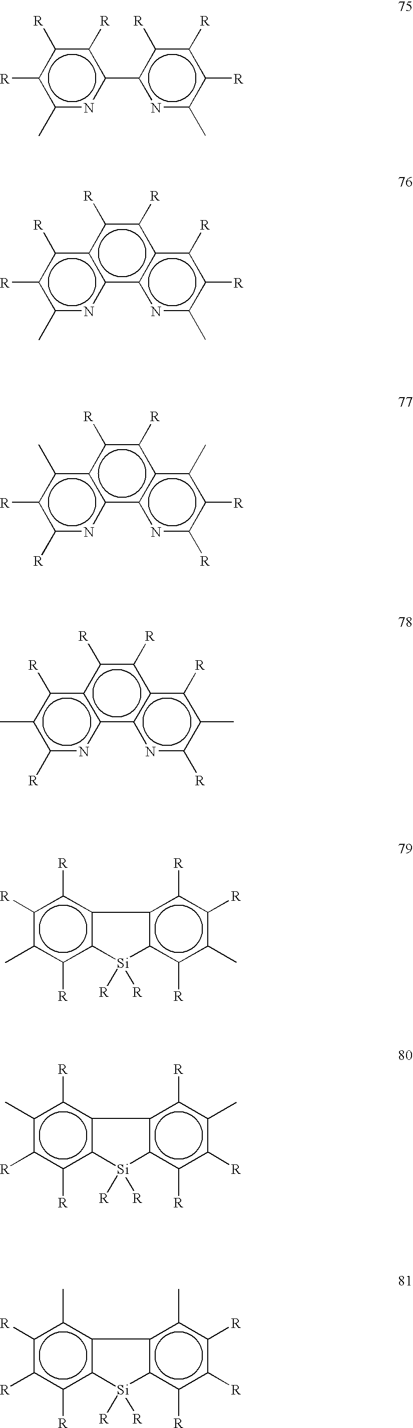

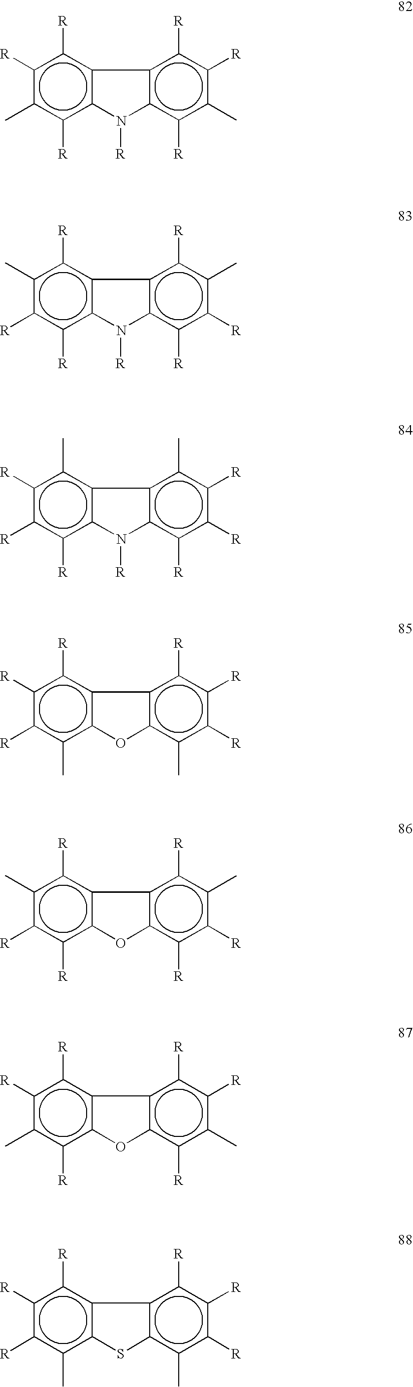

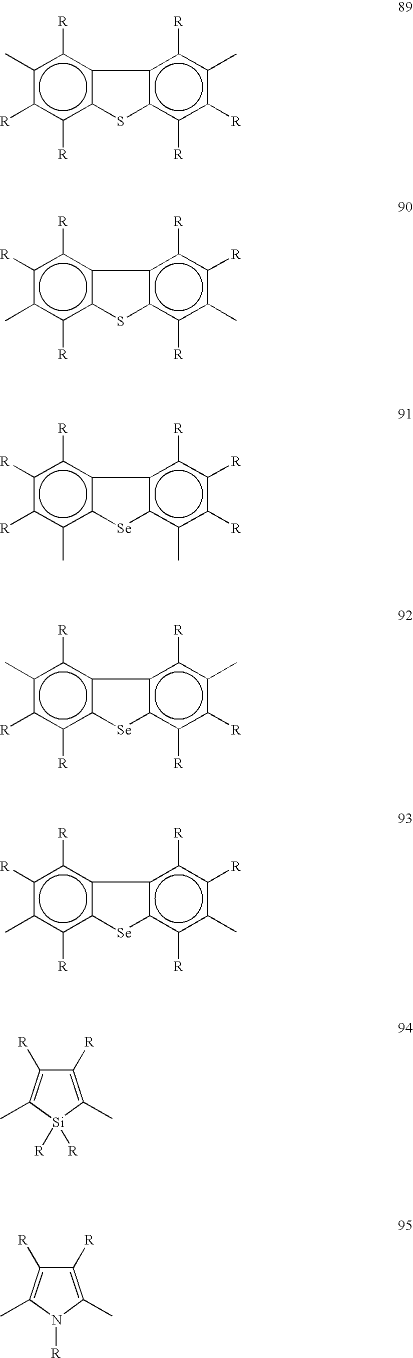

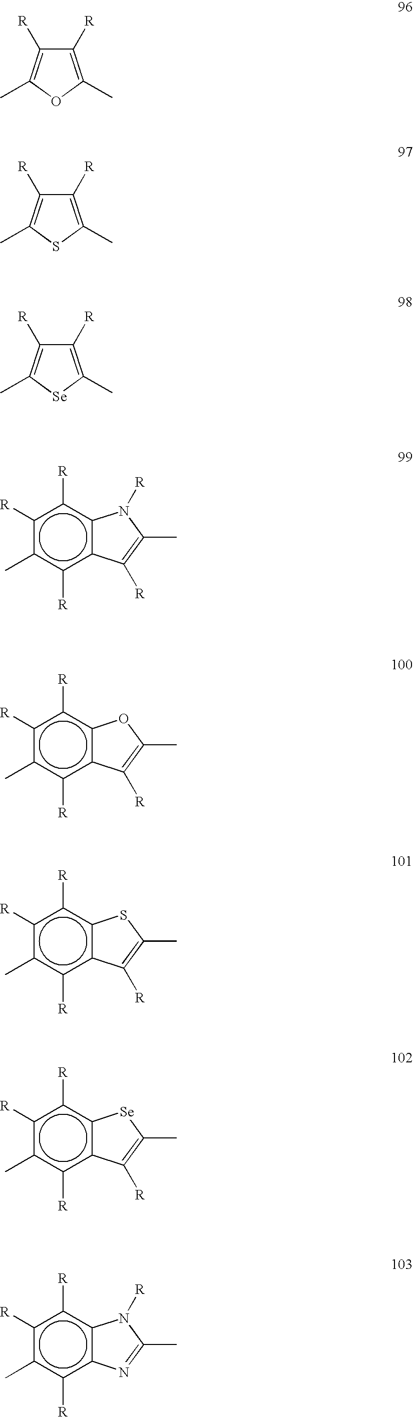

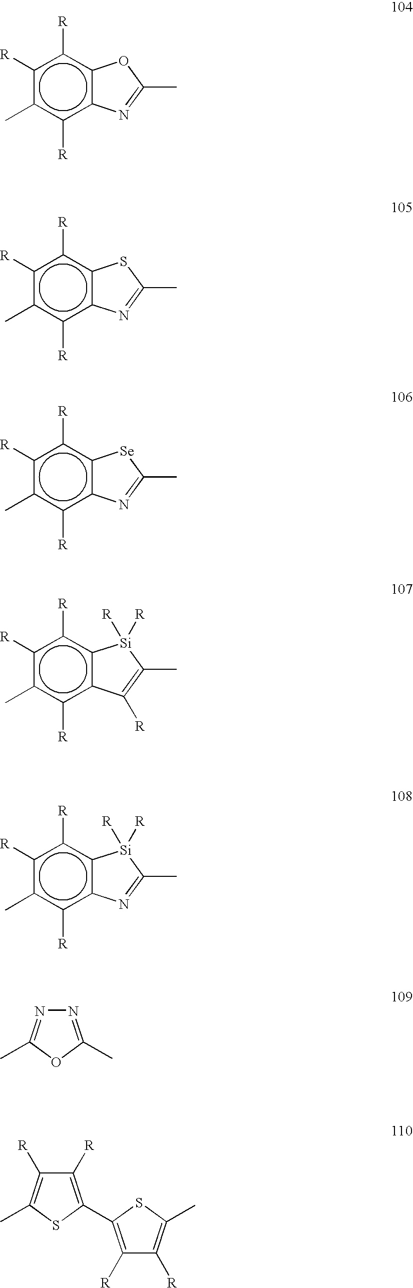

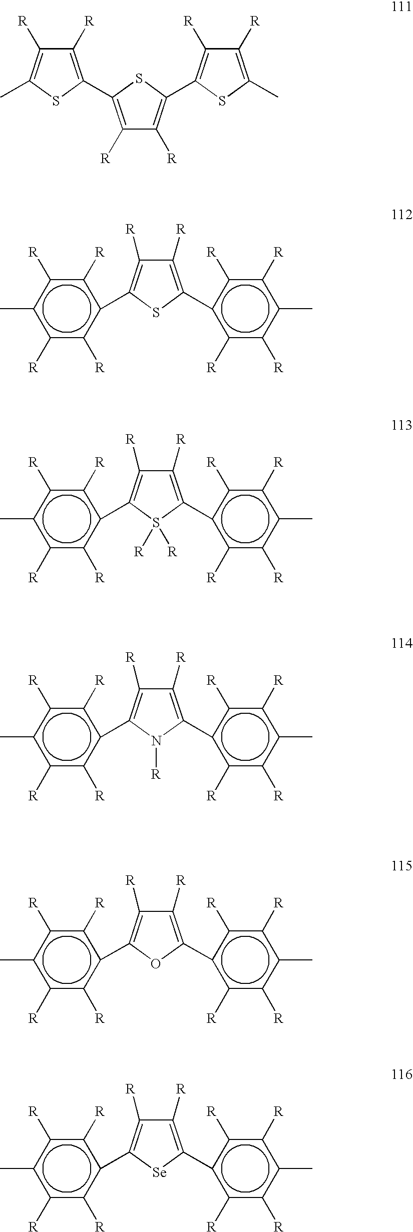

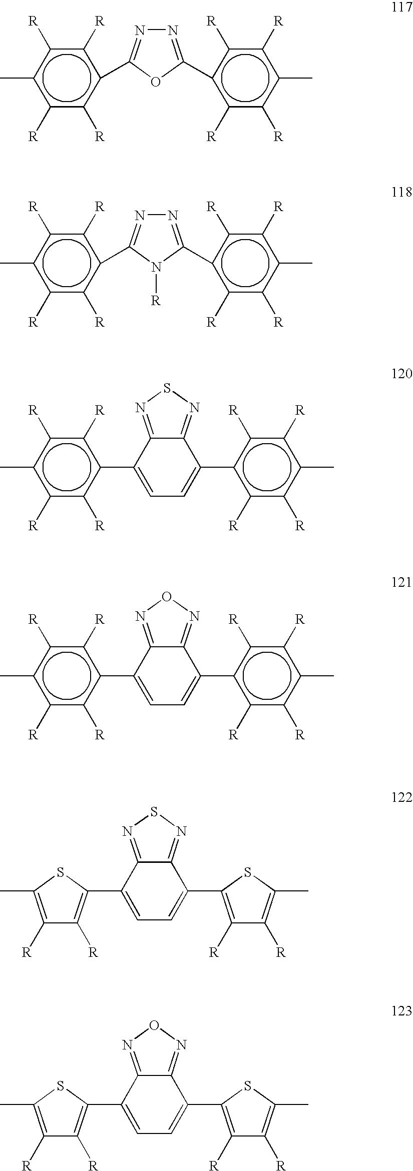

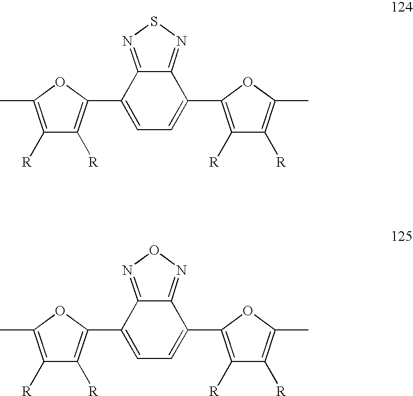

The divalent group having a metal complex structure is a divalent group remaining after removing two hydrogen atoms from an organic ligand of a metal complex having an organic ligand.

The organic ligand has a carbon number of usually about 4 to 60, and examples thereof include 8-quinolinol and derivatives thereof, benzoquinolinol and derivatives thereof, 2-phenyl-pyridine and derivatives thereof, 2-phenyl-benzothiazole and derivatives thereof, 2-phenyl-benzoxazole and derivatives thereof, porphyrin and derivatives thereof, and the like.

As the center metal of the complex, for example, aluminum, zinc, beryllium, iridium, platinum, gold, europium, terbium and the like are mentioned.

As the metal complex having an organic ligand, metal complexes, triplet emitting complexes, and the like known as fluorescent materials and phosphorescence materials of lower molecular weight are mentioned.

As the divalent group having a metal complex structure, the following (126 to 132) are specifically exemplified. In the formulae, Rs represent each independently a hydrogen atom, halogen atom, alkyl group, alkoxy group, alkylthio group, aryl group, aryloxy group, arylthio group, arylalkyl group, arylalkyloxy group, arylalkylthio group, alkenyl group, alkynyl group, heteroaryloxy group or heteroarylthio group. The carbon number of the di-valent group having a metal complex structure does not include the carbon number of the substituent.

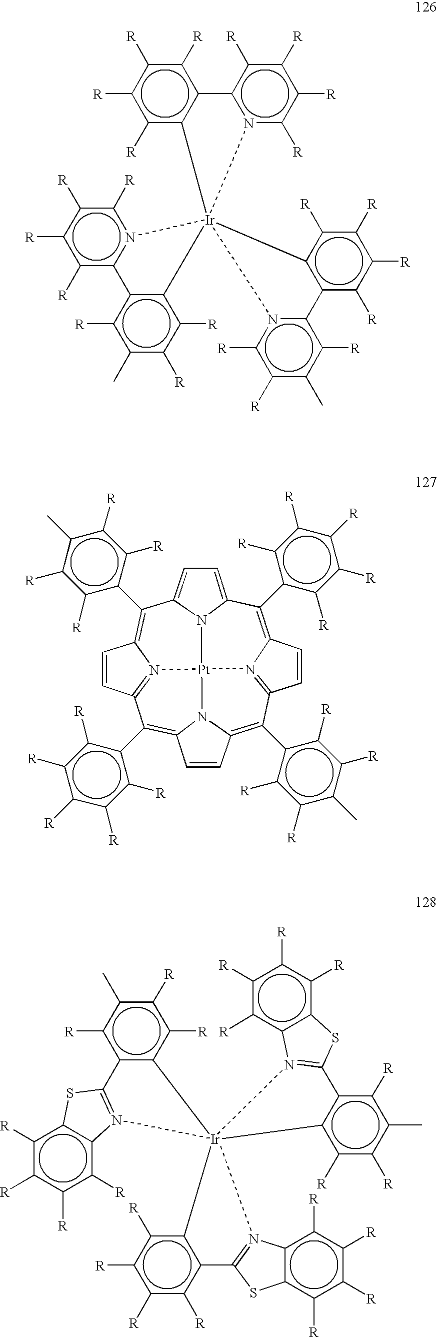

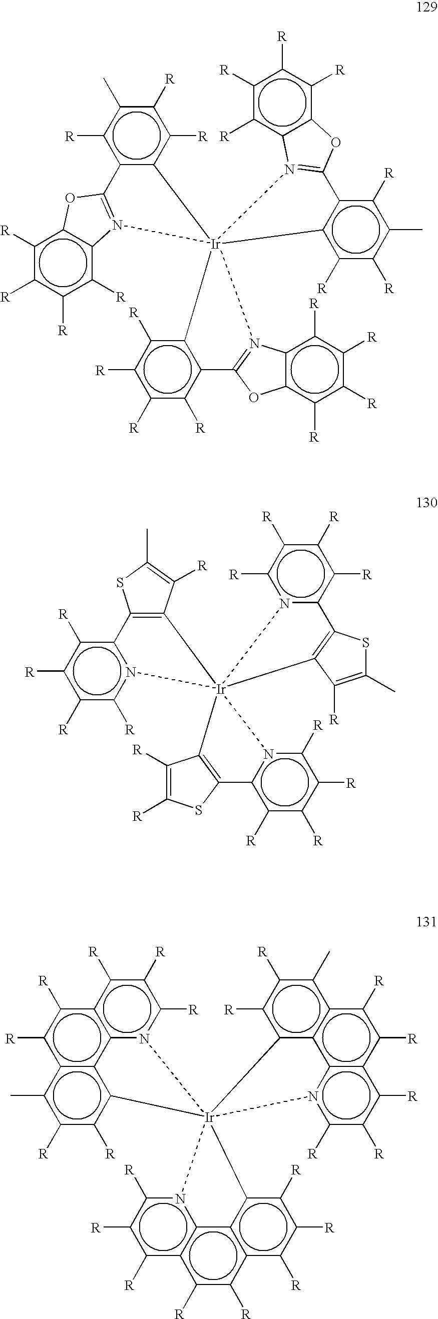

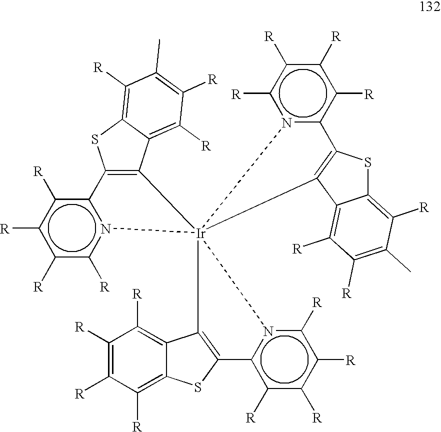

As the repeating unit of the above-described formula (4), those in which n is 0 are preferable, and those in which Ar¹ is an arylene group are more preferable.

As the repeating unit of the above-described formula (4), structures of the following formula (4-1) are further preferable.

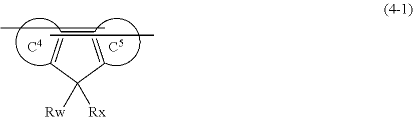

In the formulae, the ring C⁴ and ring C⁵ represent each independently an aromatic hydrocarbon ring optionally having a substituent, and two connecting bonds are present on the ring C⁴ and/or ring C⁵ respectively, and Rw and Rx represent each independently a hydrogen atom, alkyl group, alkoxy group, alkylthio group, aryl group, aryloxy group, arylthio group, arylalkyl group, arylalkyloxy group, arylalkylthio group, alkenyl group, alkynyl group, di-substituted amino group, tri-substituted silyl group, acyl group, acyloxy group, imine residue, amide group, acid imide group, mono-valent heterocyclic group, substituted carboxyl group, heteroaryloxy group or heteroarylthio group, and Rw and Rx may be mutually connected to form a ring.

The aromatic hydrocarbon ring has a carbon number of about 6 to 30, preferably about 6 to 15, and represents a benzene ring or condensed aromatic hydrocarbon ring. The carbon number of the aromatic hydrocarbon ring does not include the carbon number of a substituent. Specifically, a benzene ring, naphthalene ring, anthracene ring, phenanthrene ring, phenalene ring, naphthacene ring, triphenylene ring, pyrene ring, chrysene ring, pentacene ring, perylene ring, pentalene ring, indene ring, azulene ring, biphenylene ring, fluorene ring, acenaphthylene ring and the like are exemplified.

The alkyl group, alkoxy group, alkylthio group, aryl group, aryloxy group, arylthio group, arylalkyl group, arylalkyloxy group, arylalkylthio group, alkenyl group, alkynyl group, di-substituted amino group, tri-substituted silyl group, acyl group, acyloxy group, imine residue, amide group, acid imide group, mono-valent heterocyclic group, substituted carboxyl group, heteroaryloxy group or heteroarylthio group represented by Rw and Rx are the same as the descriptions for the above-described substituents on C¹, C² and C³.

As the repeating unit of the above-described formula (4-1), specifically mentioned are the following groups, and the following groups having a substituent selected from alkyl groups, alkoxy groups, alkylthio groups, aryl groups, aryloxy groups, arylthio groups, arylalkyl groups, arylalkoxy groups, arylalkylthio groups, alkenyl groups, alkynyl groups, di-substituted amino groups, tri-substituted silyl groups, acyl groups, acyloxy groups, imine residues, amide groups, acid imide groups, monovalent heterocyclic groups, substituted carboxyl groups, heteroaryloxy groups, heteroarylthio groups and halogen atoms. In the following descriptions, connecting bonds of aromatic hydrocarbons are capable of existing at any positions.

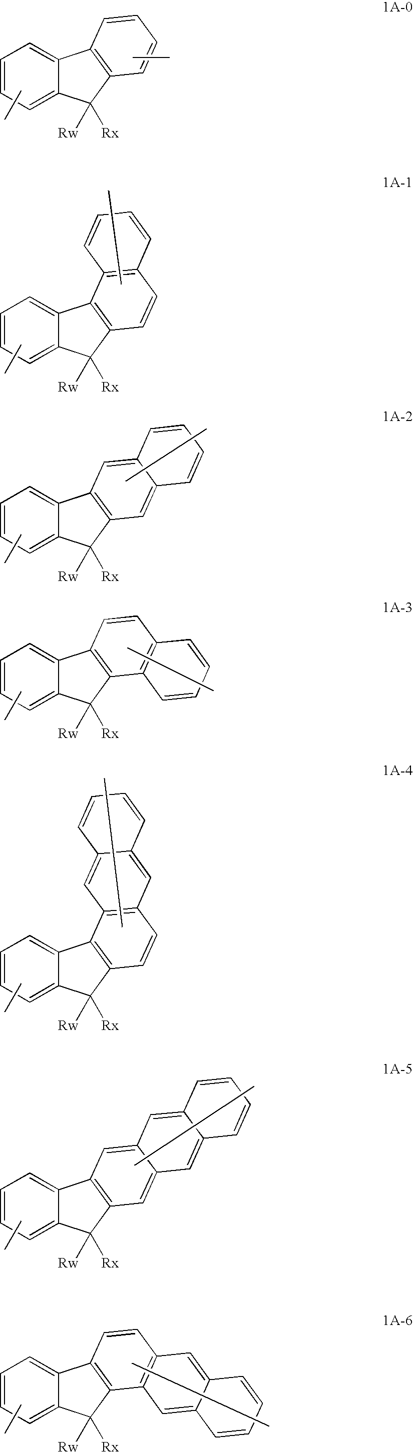

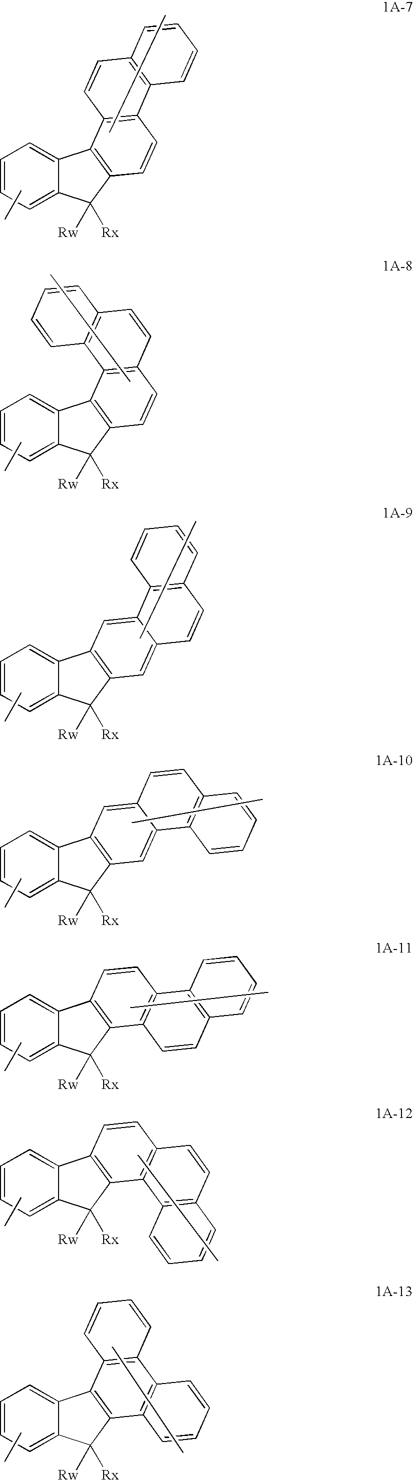

Of them, repeating units represented by 1A-0, 1A-1, 1A-2 and 1A-3 are preferable, and 1A-0 is most preferable.

The polymer compound of the present invention preferably contains one or more repeating units, more preferably contains one or two repeating units of the following formula (5), from the standpoint of improvements of device properties such as enhancement of heat resistance, improvement of charge transportability, enhancement of light emission efficiency and the like.

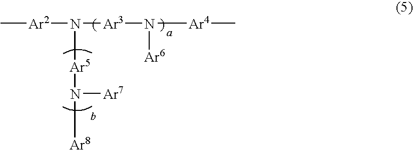

In the formula, Ar², Ar³, Ar⁴ and Ar⁵ represent each independently an arylene group or divalent heterocyclic group. Ar⁶, Ar⁷ and Ar⁸ represent each independently an aryl or monovalent heterocyclic group. a and b represent each independently 0 or a positive integer. Ar², Ar³, Ar⁴, Ar⁵, Ar⁶, Ar⁷ and Ar⁸ may have a substituent.

In the present invention, the repeating unit of the above-described formula (5) is contained in an amount of preferably 2 mol % or more and 40 mol % or less, more preferably 5 mol % or more and 30 mol % or less based on all repeating units, from the standpoint of device properties such as light emission intensity, device life property and the like.

As specific examples of the repeating unit of the above-described formula (5), those of the following (formulae 133 to 140) are mentioned.

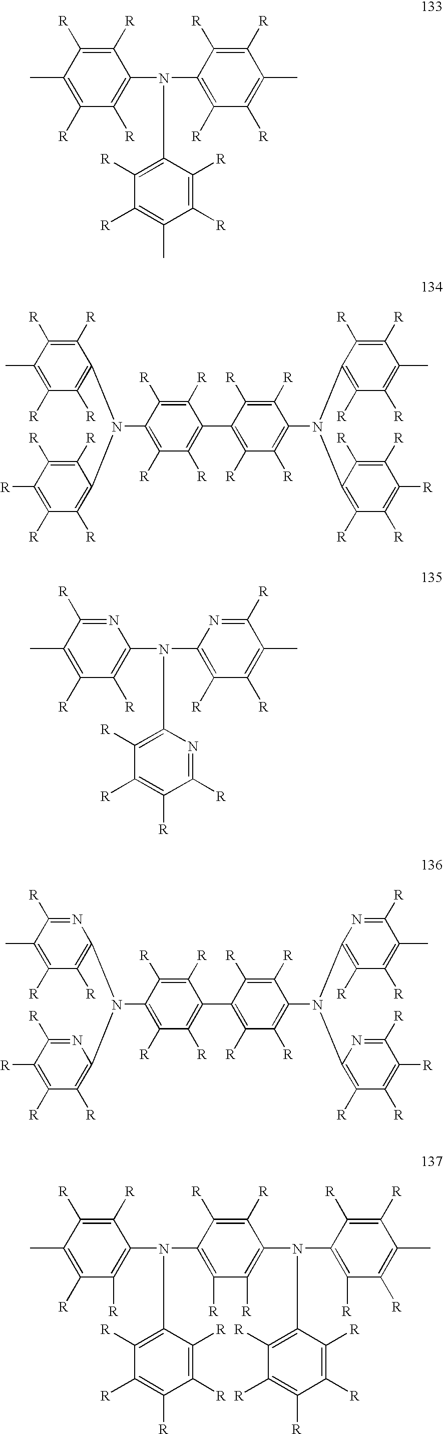

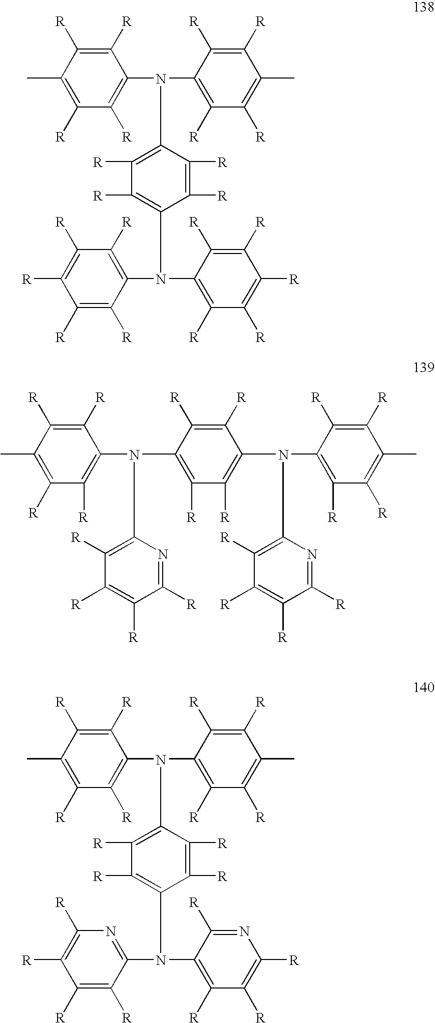

In the above-described formulae, Rs represent each independently a hydrogen atom, alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, mono-valent heterocyclic group, carboxyl group, substituted carboxyl group or cyano group.

In substituents R containing an alkyl in the above-described formulae, it is preferable that a cyclic or branched alkyl is contained in one or more of the substituents, for enhancing solubility of a polymer compound in an organic solvent.

Further, when R contains partially an aryl group or heterocyclic group in the above-described formulae, these groups optionally further have one or more substituents.

Among structures of the above-described formulae 133 to 140, structures of the above-described formulae 134 and 137 are preferable from the standpoint of regulation of light emission wavelength.

Repeating units of the above-described formula (5) in which Ar², Ar³, Ar⁴ and Ar⁵ represent each independently an arylene group and Ar⁶, Ar⁷ and Ar⁸ represent each independently an aryl group are preferable from the standpoint of device properties such as light emission wavelength regulation, device life and the like.

Ar², Ar³ and Ar⁴ represent each independently preferably a non-substituted phenylene group, non-substituted biphenyl group, non-substituted naphthylene group or non-substituted anthracenediyl group.

Ar⁶, Ar⁷ and Ar⁸ represent each independently preferably an aryl group having one or more substituents, more preferably an aryl group having three or more substituents, from the standpoint of solubility in an organic solvent, device properties and the like. Ar⁶, Ar⁷ and Ar⁸ are more preferably a phenyl group having three or more substituents, a naphthyl group having three or more substituents or an anthranyl group having three or more substituents, and Ar⁶, Ar⁷ and Ar⁸ are further preferably a phenyl group having three or more substituents.

Of them, those in which Ar⁶, Ar⁷ and Ar⁸ represent each independently a group of the following formula (5-1) and a+b≦3 are preferable, and those in which a+b=1 are more preferable, and those in which a=1 and b=0 are further preferable.

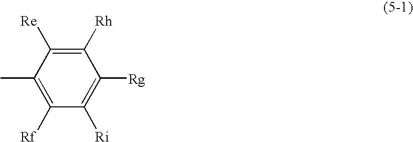

(wherein, Re, Rf and Rg represent each independently an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, silyloxy group, substituted silyloxy group, mono-valent heterocyclic group or halogen atom. A hydrogen atom contained in Re, Rf and Rg may be substituted by a fluorine atom. Rh and Ri represent each independently a hydrogen atom, alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, silyloxy group, substituted silyloxy group, mono-valent heterocyclic group or halogen atom. A hydrogen atom contained in Re, Rf and Rg may be substituted by a fluorine atom. Two adjacent substituents on a benzene ring may be mutually connected to form a ring.).

More preferably mentioned are those in which Re and Rf represent each independently an alkyl group having a carbon number of 3 or less, an alkoxy group having a carbon number of 3 or less or an alkylthio group having a carbon number of 3 or less and Rg represents an alkyl group having a carbon number of 1 to 30, an alkoxy group having a carbon number of 1 to 30 or an alkylthio group having a carbon number of 1 to 30, in the above-described formula (5-1).

In the repeating unit of the above-described formula (5), Ar³ is preferably the following formula (5-2) or (5-3).

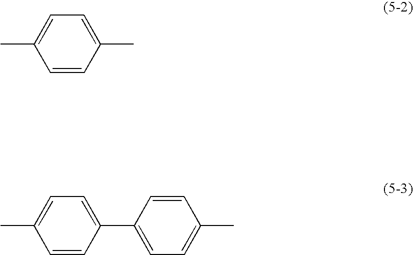

(wherein, the benzene rings contained in the structures (5-2), (5-3) are preferably non-substituted and optionally have each independently one or more and four or less substituents. These substituents may be mutually the same or different. A plurality of substituents may be connected to form a ring. To the benzene ring, other aromatic hydrocarbon rings or hetero rings may be condensed.).

As the repeating unit of the above-described formula (5), those of the following (formulae 141 to 143) are mentioned as particularly preferable specific examples.

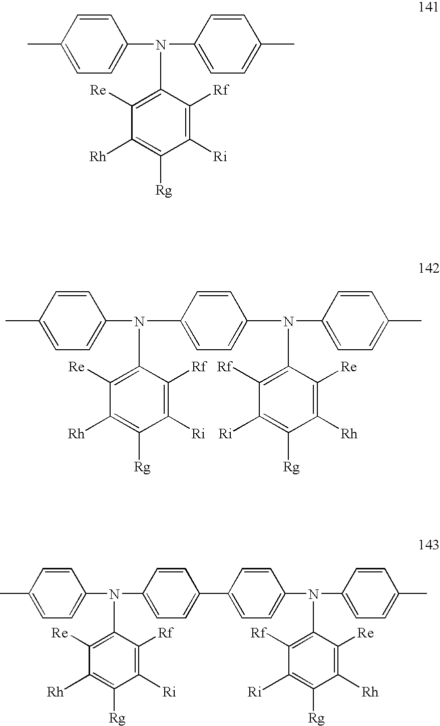

In the formulae, Re, Rf, Rg, Rh and Ri are the same as described above.

As specific examples of the above-described formula (5), repeating units of the following formulae (22), (23) and (24) are preferable from the standpoint of device properties such as fluorescence intensity, light emission wavelength regulation, heat resistance and the like.

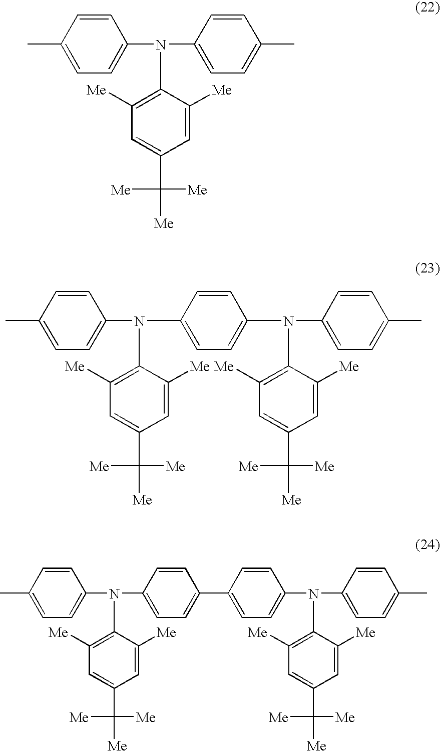

Among the polymer compounds of the present invention, conjugated polymers are preferable from the standpoint of charge transportability when manufactured into a thin film, and device properties such as light emission efficiency, life and the like when used in a polymer light emitting device. Here, the conjugated polymers means a polymer in which a nonlocalized at electron pair is present along the main skeleton of a polymer. This nonlocalized electron includes also a case in which an unpaired electron or lone electron pair takes part in resonance instead of a double bond.

Repeating units may be connected via a nonconjugated unit or a nonconjugated portion thereof may be contained in repeating units, within the range not deteriorating a light emission property or charge transportation property. As the nonconjugated bond structure, exemplified are those shown below, and combinations of two or more of those shown below. Here, Rs represent each independently a hydrogen atom, halogen atom, alkyl group, alkoxy group, alkylthio group, aryl group, aryloxy group, arylthio group, arylalkyl group, arylalkyloxy group, arylalkylthio group, alkenyl group, alkynyl group, heteroaryloxy group or heteroarylthio group. Ar represents an aromatic hydrocarbon ring or hetero ring.

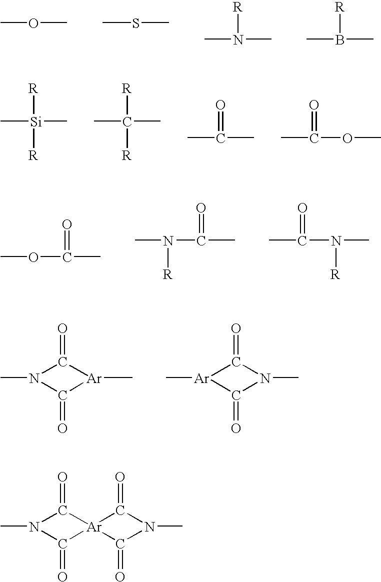

The polymer compound of the present invention may be a random, block or graft copolymer, or a polymer having an intermediate structure, for example, a random copolymer having a block property. From the standpoint of obtaining a polymer light emitting body having high quantum yield of fluorescence or phosphorescence, a random copolymer having a block property and a block or graft copolymer are more preferable than a complete random copolymer. Those having branching in the main chain and thus having 3 or more end parts, and dendrimers are also included.

The polymer compounds of the present invention contain a structural unit containing a structure of the above-described formula (1) in an amount of preferably 0.1 mol % or more and 40 mol % or less, more preferably 1 mol % or more and 30 mol % or less based on all structural units, from the standpoint of device properties such as light emission efficiency, life and the like when used in a polymer light emitting device.

The polymer compounds of the present invention have preferably a polystyrene-reduced number average molecular weight of 10³ to 10⁸, more preferably a polystyrene-reduced weight average molecular weight of 5×10⁴ to 10⁷, from the standpoint of device properties such as light emission efficiency, life and the like when used in a polymer light emitting device.

The polymer compound of the present invention can be produced by a method for polymerization using a compound of the above-described formula (6) as a raw material.

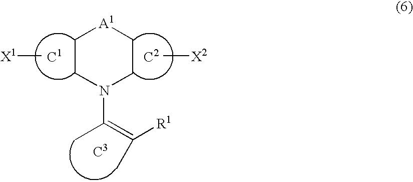

In the formula, a ring C¹, ring C², ring C³, A¹ and R¹ have the same meanings as described above. X¹ and X² represent each independently a substituent correlatable with polymerization.

As the substituent correlatable with polymerization, mentioned are halogen atoms, alkyl sulfonate groups, aryl sulfonate groups, aryl alkyl sulfonate groups, borate groups, sulfoniummethyl groups, phosphoniummethyl groups, phosphonatemethyl groups, methyl monohalide groups, magnesium halide groups, stannyl group, —B(OH)₂, formyl group, cyano group, vinyl group and the like.

Of them, preferable are —B(OH)₂, borate groups, magnesium halide groups, stannyl group, halogen atoms, alkyl sulfonate groups, aryl sulfonate groups or aryl alkyl sulfonate groups.

Here, mentioned as the halogen atom are a fluorine atom, chlorine atom, bromine atom and iodine atom, preferably a chlorine atom, bromine atom or iodine atom, more preferably a bromine atom.

As the alkyl sulfonate group, a methane sulfonate group, ethane sulfonate group, trifluoromethane sulfonate group and the like are exemplified, as the aryl sulfonate group, a benzene sulfonate group, p-toluene sulfonate group and the like are exemplified, and as the aryl alkyl sulfonate group, a benzyl sulfonate group and the like are exemplified.

As the borate group, a dialkyl ester, diaryl ester and diaryl alkyl ester are mentioned, and groups of the following formulae are exemplified.

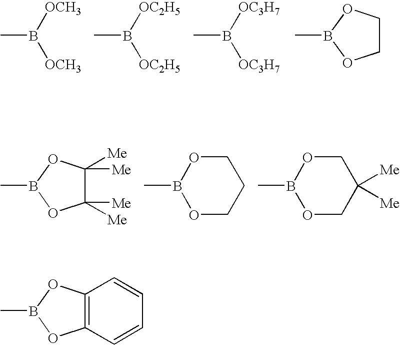

As the sulfoniummethyl group, groups of the following formulae are exemplified.

—CH₂S⁺Me₂X⁻,—CH₂S⁺Ph₂X⁻

(wherein, X represents a halogen atom and Ph represents a phenyl group.)

As the phosphoniummethyl group, groups of the following formula are exemplified.

—CH₂P⁺Ph₃X⁻ (X represents a halogen atom.)

As the phosphonatemethyl group, groups of the following formula are exemplified.

—CH₂PO(OR′)₂ (X represents a halogen atom, R′ represents an alkyl group, aryl group or arylalkyl group.)

As the methyl monohalide group, a methyl fluoride group, methyl chloride group, methyl bromide group and methyl iodide group are exemplified.

As the magnesium halide group, a magnesium chloride group, magnesium bromide group and magnesium iodide group are exemplified.

The stannyl group means a stannyl group having three substituents selected from a hydrogen atom, halogen atoms, alkyl groups, aryl groups and arylalkyl groups, and exemplified are a stannyl group, trichlorostannyl group, trimethylstannyl group, triethylstannyl group, tri-n-butylstannyl group, triphenylstannyl group and tribenzylstannyl group.

A preferable substituent as the substituent correlatable with polymerization differs depending on the kind of the polymerization reaction, and in the case of use of a 0-valent nickel complex such as, for example, Yamamoto coupling reaction and the like, mentioned are halogen atoms, alkyl sulfonate groups, aryl sulfonate group or aryl alkyl sulfonate groups. In the case of use of a nickel catalyst or palladium catalyst such as Suzuki coupling reaction and the like, mentioned are alkyl sulfonate groups, halogen atoms, borate groups, —B(OH)₂ and the like.

The production method of the present invention can be carried out, specifically, by dissolving a compound having a plurality of substituents correlated with polymerization, as a monomer, in an organic solvent if necessary, and using, for example, an alkali and a suitable catalyst, at temperatures of not lower than the melting point and not higher than the boiling point of the organic solvent. For example, known methods can be used described in “Organic Reactions”, vol. 14, p. 270 to 490, John Wiley & Sons, Inc., 1965, “Organic Syntheses”, Collective Volume VI, p. 407 to 411, John Wiley & Sons, Inc., 1988, Chem. Rev., vol. 95, p. 2457 (1995), J. Organomet. Chem., vol. 576, p. 147 (1999), Makromol. Chem., Macromol. Symp., vol. 12, p. 229 (1987), and the like.

In the method for producing a polymer compound of the present invention, a known condensation reaction can be used depending on the substituent correlatable with polymerization of a compound of the above-described formula (5).

A copolymer can be produced by performing polymerization in the co-existence of a compound having two or more substituents correlatable with polymerization. A polymer compound having a branched structure can be produced by copolymerizing a compound having three or more substituents correlatable with polymerization.

When the polymer compound of the present invention generates a double bond in polymerization, for example, a method described in JP-A No. 5-202355 is mentioned. Namely, polymerization by the Wittig reaction of a compound having a formyl group and a compound having a phosphoniummethyl group, or of a compound having a formyl group and a phosphoniummethyl group, polymerization by the Heck reaction of a compound having a vinyl group and a compound having a halogen atom, polycondensation by a dehydrohalogenation method of a compound having two or more methyl monohalide groups, polycondensation by a sulfonium salt decomposition method of a compound having two or more sulfoniummethyl groups, polymerization by the Knoevenagel reaction of a compound having a formyl group and a compound having a cycno group, polymerization by the McMurry reaction of compound having two or more formyl groups, and the like, are exemplified.

When the polymer compound of the present invention generates a triple bond in the main chain by condensation polymerization, for example, the Heck reaction and Sonogashira reaction can be utilized.

In the case of generating no double bond or triple bond, for example, a method of polymerization by the Suzuki coupling reaction from the corresponding monomer, a method of polymerization by the Grignard method, a method of polymerization by a Ni(0) complex, a method of polymerization by an oxidizer such as FeCl₃ and the like, a method of electrochemical oxidation polymerization, a method by decomposition of an intermediate polymer having a suitable leaving group, and the like, are exemplified.

Of them, polymerization by the Wittig reaction, polymerization by the Heck reaction, polymerization by the Knoevenagel reaction, method of polymerization by the Suzuki coupling reaction, method of polymerization by the Grignard reaction and method of polymerization by a nickel O-valent complex are preferable from the standpoint of easiness of control of molecular weight and the standpoint of easiness of control of formulation ratio in the case of copolymerization.

Of them, the method of polymerization by the Suzuki coupling reaction and the method of polymerization by a nickel 0-valent complex are more preferable, and the method of polymerization by the Suzuki coupling reaction is most preferable.

Of the production methods of the present invention, preferable is a production method in which substituents correlatable with polymerization are selected each independently from halogen atoms, alkyl sulfonate groups, aryl sulfonate groups or aryl alkyl sulfonate groups, and condensation polymerization is carried out in the present of a nickel O-valent complex or palladium catalyst.

The raw material compounds include dihalogenated compounds, bis(alkyl sulfonate) compounds, bis(aryl sulfonate) compounds, bis(aryl alkyl sulfonate) compounds or halogen-alkyl sulfonate compounds, halogen-aryl sulfonate compounds, halogen-aryl alkyl sulfonate compounds, alkyl sulfonate-aryl sulfonate compounds, alkyl sulfonate-aryl alkyl sulfonate compounds, and aryl sulfonate-aryl alkyl sulfonate compounds.

In this case, there is mentioned a method for producing a polymer compound in which the direction and sequence of repeating units are controlled, by using, for example, a halogen-alkyl sulfonate compound, halogen-aryl sulfonate compound, halogen-aryl alkyl sulfonate compound, alkyl sulfonate-aryl sulfonate compound, alkyl sulfonate-aryl alkyl sulfonate compound, and aryl sulfonate-aryl alkyl sulfonate compound as a raw material compound.

Among the production methods of the present invention, preferable is a production method in which substituents correlatable with polymerization are selected each independently from halogen atoms, alkyl sulfonate groups, aryl sulfonate groups, aryl alkyl sulfonate groups, —B(OH)₂, or borate groups, the ratio of the sum (J) of mol numbers of halogen atoms, alkyl sulfonate groups, aryl sulfonate groups and aryl alkyl sulfonate groups to the sum (K) of mol numbers of —B(OH)₂ and borate groups, in all raw material compounds, is substantially 1 (usually, K/J is in a range of 0.7 to 1.2), and condensation polymerization is carried out using a nickel catalyst or palladium catalyst.

As specific combinations of raw material compounds, there are mentioned combinations of a dihalogenated compound, bis(alkyl sulfonate) compound, bis(aryl sulfonate) compound or bis(aryl alkyl sulfonate) compound with a diboric acid compound or diborate compound.

Further mentioned are a halogen-boric acid compound, halogen-borate compound, alkyl sulfonate-boric acid compound, alkyl sulfonate-borate compound, aryl sulfonate-boric acid compound, aryl sulfonate-borate compound, aryl alkyl sulfonate-boric acid compound, aryl alkyl sulfonate-boric acid compound and aryl alkyl sulfonate-borate compound.

In this case, there is mentioned a method for producing a polymer compound in which the direction and sequence of repeating units are controlled, by using, for example, a halogen-boric acid compound, halogen-borate compound, alkyl sulfonate-boric acid compound, alkyl sulfonate-borate compound, aryl sulfonate-boric acid compound, aryl sulfonate-borate compound, aryl alkyl sulfonate-boric acid compound, aryl alkyl sulfonate-boric acid compound or aryl alkyl sulfonate-borate compound as a raw material compound.

The organic solvent differs depending on the compound and reaction to be used, and for suppressing a side reaction, in general, it is preferable that a solvent to be used is subjected to a sufficient deoxidation treatment and the reaction is progressed in an inert atmosphere. Further, it is preferable to perform a dehydration treatment likewise. However, this is not the case when a reaction in a two-phase system with water such as the Suzuki coupling reaction is conducted.

The solvent varies depending on the compound and reaction to be used, and exemplified are saturated hydrocarbons such as pentane, hexane, heptane, octane, cyclohexane and the like, unsaturated hydrocarbons such as benzene, toluene, ethylbenzene, xylene and the like, halogenated saturated hydrocarbons such as carbon tetrachloride, chloroform, dichloromethane, chlorobutane, bromobutane, chloropentane, bromopentane, chlorohexane, bromohexane, chlorocyclohexane, bromocyclohexane and the like, halogenated unsaturated hydrocarbons such as chlorobenzene, dichlorobenzene, trichlorobenzene and the like, alcohols such as methanol, ethanol, propanol, isopropanol, butanol, t-butyl alcohol and the like, carboxylic acids such as formic acid, acetic acid, propionic acid and the like, ethers such as dimethyl ether, diethyl ether, methyl-t-butyl ether, tetrahydrofuran, tetrahydropyran, dioxane and the like, amines such as trimethylamine, triethylamine, N,N,N′,N′-tetramethylethylenediamine, pyridine and the like, amides such as N,N-dimethylformamide, N,N-dimethylacetamide, N,N-diethylacetamide, N-methylmorpholine oxide, and the like, and single solvents or mixed solvents thereof may also be used.

For reacting, an alkali or suitable catalyst is appropriately added. These may be advantageously selected depending on the reaction to be used. As the alkali or catalyst, those sufficiently dissolved in the solvent used in the reaction are preferable. As the method of mixing an alkali or catalyst, there is exemplified a method in which a solution of an alkali or catalyst is added slowly while stirring the reaction liquid under an inert atmosphere such as argon and nitrogen and the like, or reversely, the reaction liquid is slowly added to a solution of an alkali or catalyst.

When the polymer compound of the present invention is used in a polymer LED and the like, its purity exerts an influence on the performance of a device such as a light emitting property and the like, therefore, it is preferable that a monomer before polymerization is purified by a method such as distillation, sublimation purification, re-crystallization and the like before polymerization. Further, it is preferable that, after polymerization, a purification treatment such as re-precipitation purification, fractionation by chromatography, and the like is carried out.

For producing the polymer compound of the present invention, it is preferable to carry out polymerization using a compound of the following formula (7), (8) or (9).

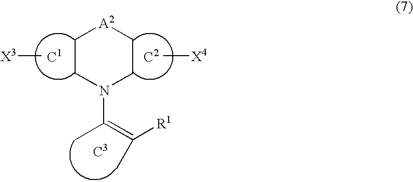

In the formula, a ring C¹, ring C², ring C³ and R¹ have the same meanings as described above. A² represents a group represented by —BR′—, —C(R′)₂—, —NR′—, —O—, —PR′—, —P(═O)R′—, —Se—, —Se(═O)— or —Se(═O)₂—. R′s represent each independently an alkyl group, alkoxy group, alkylthio group, aryl group, aryloxy group, arylthio group, arylalkyl group, arylalkyloxy group, arylalkylthio group, alkenyl group, alkynyl group, di-substituted amino group, tri-substituted silyl group, acyl group, acyloxy group, mono-valent heterocyclic group, substituted carboxyl group, heteroaryloxy group or heteroarylthio group. X³ and X⁴ represent each independently a halogen atom, alkyl sulfonate group, aryl sulfonate group, aryl alkyl sulfonate group, borate group, sulfoniummethyl group, phosphoniummethyl group, phosphonatemethyl group, methyl monohalide group, magnesium halide group, substituted silyl group, stannyl group, —B(OH)₂, formyl group, cyano group or vinyl group.

It is preferable that the ring C¹ and ring C² are a benzene ring or monocyclic hetero ring in the above-described formula (7) from the standpoint of stability of the compound. More preferably, the ring C¹ and ring C² are a 6-membered ring, and further preferably, a benzene ring.

The ring C³ is preferably an aromatic hydrocarbon ring, more preferably a benzene ring, most preferably a ring of the above-described formula (3).

A² is preferably an oxygen atom, selenium atom, —Se(═O)— or —Se(═O)₂—, more preferably an oxygen atom or selenium atom, most preferably an oxygen atom.

X³ and X⁴ represent each independently preferably —B(OH)₂, borate group, halogen atom, alkyl sulfonate group, aryl sulfonate group or aryl alkyl sulfonate group, more preferably —B(OH)₂, borate group or halogen atom, further preferably a halogen atom, and among others, a chlorine atom, bromine atom and iodine atom are preferable, and a bromine atom is most preferable.

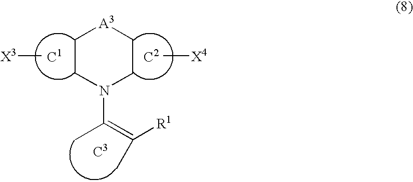

In the formula, a ring C¹, ring C², ring C³, R¹, X³ and X⁴ have the same meanings as described above. A³ represents a di-valent group containing a boron atom, carbon atom, nitrogen atom, oxygen atom, phosphorus atom, sulfur atom or selenium atom and forming a 7-membered ring or 8-membered ring together with the ring C¹, N atom and ring C².

It is preferable that the ring C¹ and ring C² are a benzene ring or monocyclic hetero ring in the above-described formula (8) from the standpoint of stability of the compound. More preferably, the ring C¹ and ring C² are a 6-membered ring, and further preferably, a benzene ring.

The ring C³ is preferably an aromatic hydrocarbon ring, more preferably a benzene ring, most preferably a ring of the above-described formula (3).

A³ preferably forms a 7-membered ring together with the ring C¹, N atom and ring C², and it is more preferable from the standpoint of stability of the compound that crosslinking is attained with two carbon atoms.

X³ and X⁴ represent each independently preferably —B(OH)₂, borate group, halogen atom, alkyl sulfonate group, aryl sulfonate group or aryl alkyl sulfonate group, more preferably —B(OH)₂, borate group or halogen atom, further preferably a halogen atom, and among others, a chlorine atom, bromine atom and iodine atom are preferable, and a bromine atom is most preferable.

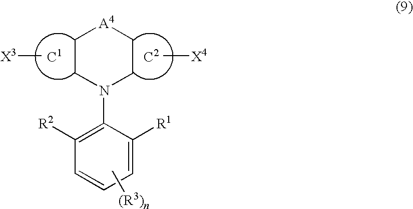

In the formula, a ring C¹, ring C², R¹, R², R³, n, X³ and X⁴ have the same meanings as described above. A⁴ represents a group represented by —C(═O)—, —C(═CR′₂)—, —S—, —S(═O)— or —S(═O)₂—.

It is preferable that the ring C¹ and ring C² are a benzene ring or monocyclic hetero ring in the above-described formula (9) from the standpoint of stability of the compound. More preferably, the ring C¹ and ring C² are a 6-membered ring, and further preferably, a benzene ring.

R¹ and R² represent each independently preferably an alkyl group, alkoxy group, alkylthio group, aryl group, aryloxy group, arylthio group, arylalkyl group, arylalkyloxy group, arylalkylthio group, di-substituted amino group, mono-valent heterocyclic group, heteroaryloxy group or heteroarylthio group, more preferably an alkyl group, aryl group, arylalkyl group or mono-valent heterocyclic group, further preferably an alkyl group.

A⁴ is preferably a sulfur atom, —S(═O)— or —S(═O)₂—, more preferably a sulfur atom.

X³ and X⁴ represent each independently preferably —B(OH)₂, borate group, halogen atom, alkyl sulfonate group, aryl sulfonate group or aryl alkyl sulfonate group, more preferably —B(OH)₂, borate group or halogen atom, further preferably a halogen atom, and among others, a chlorine atom, bromine atom and iodine atom are preferable, and a bromine atom is most preferable.

As the compound of the above-described formula (7), structures of the following formulae are exemplified.

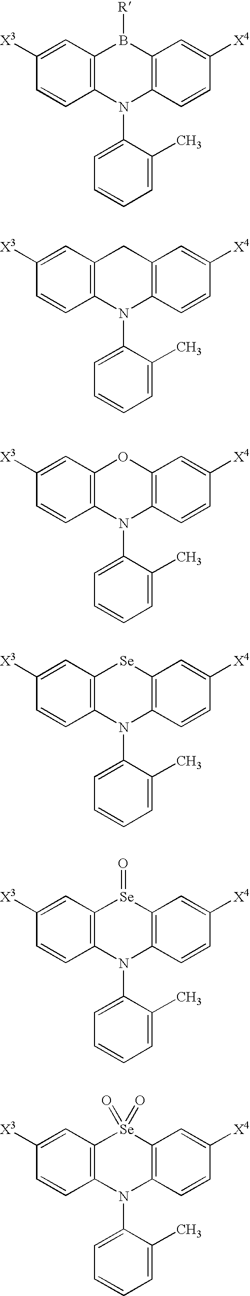

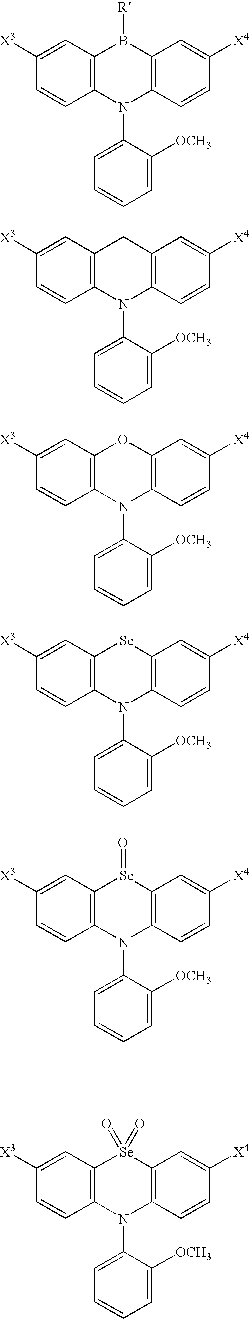

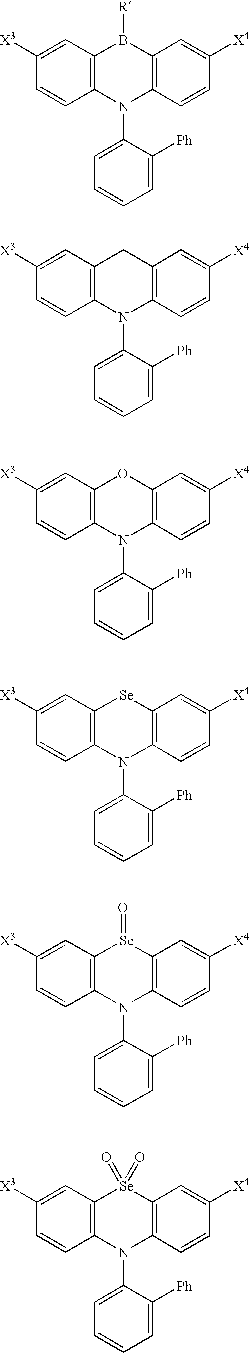

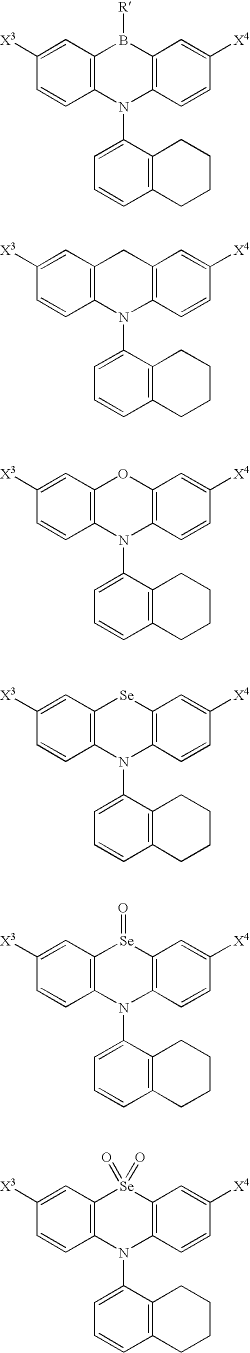

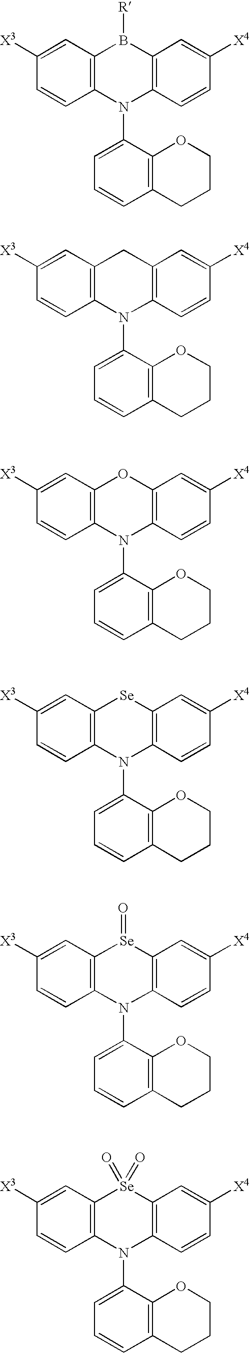

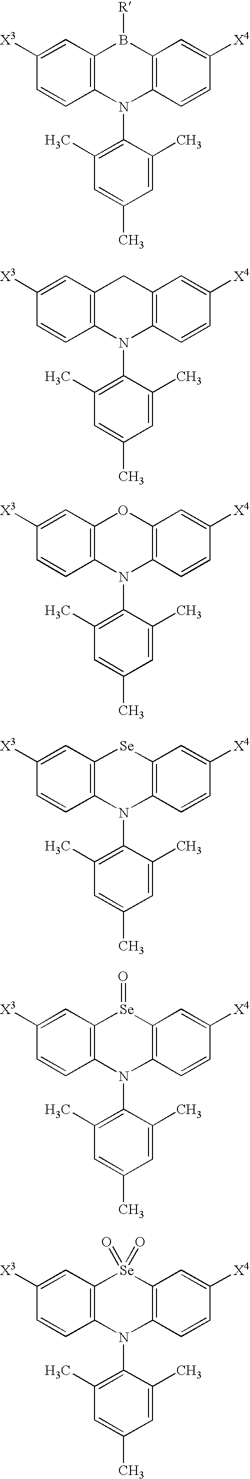

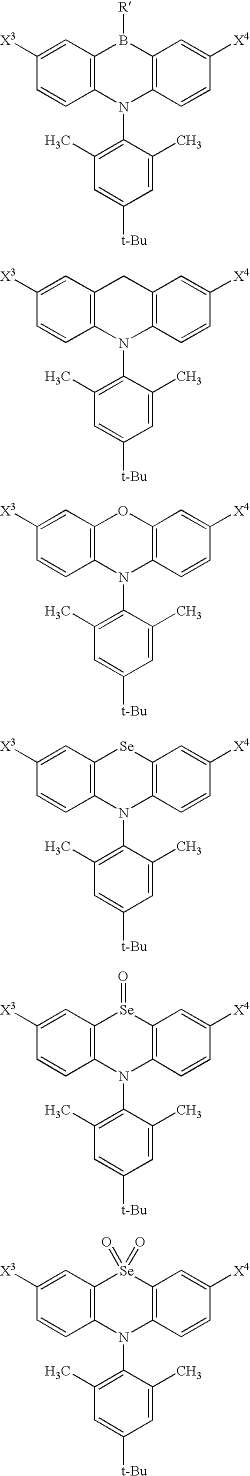

In the formulae, R′, X³ and X⁴ have the same meanings as described above. The structures of the above-described formulae optionally have a substituent.

X³ and X⁴ preferably represent each independently a halogen atom, alkyl sulfonate group, aryl sulfonate group, aryl alkyl sulfonate group, —B(OH)₂, or borate group. Particularly, X³ and X⁴ are preferably a halogen atom since conversion into an alkyl sulfonate group, aryl sulfonate group, aryl alkyl sulfonate group, —B(OH)₂, or borate group is easy when X³ and X⁴ are a halogen. A bromine atom is most preferable.

As the compound of the above-described formula (8), structures of the following formulae are exemplified.

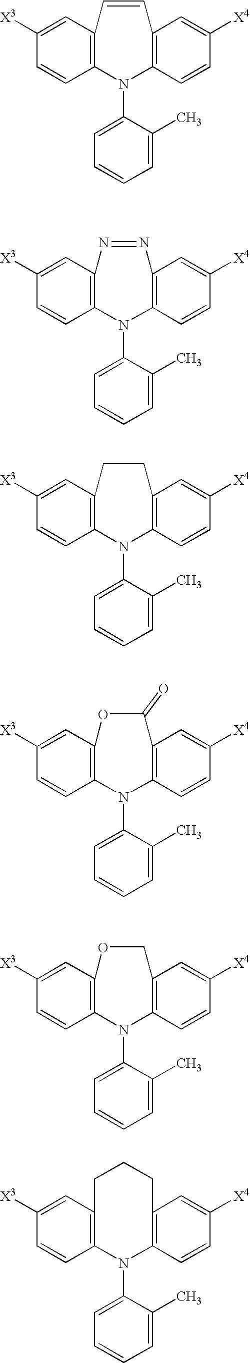

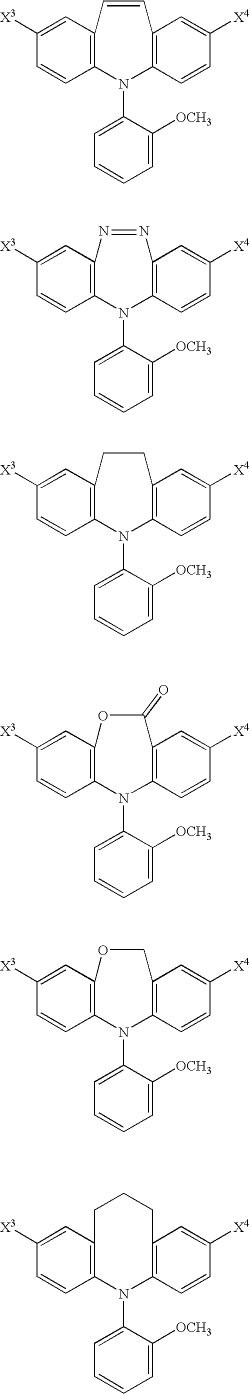

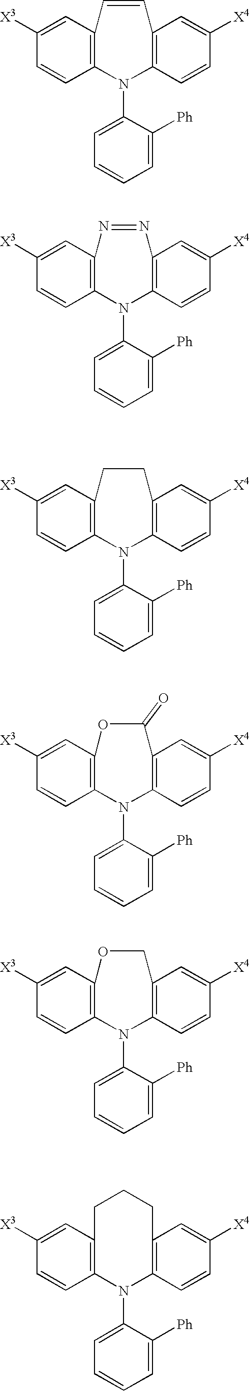

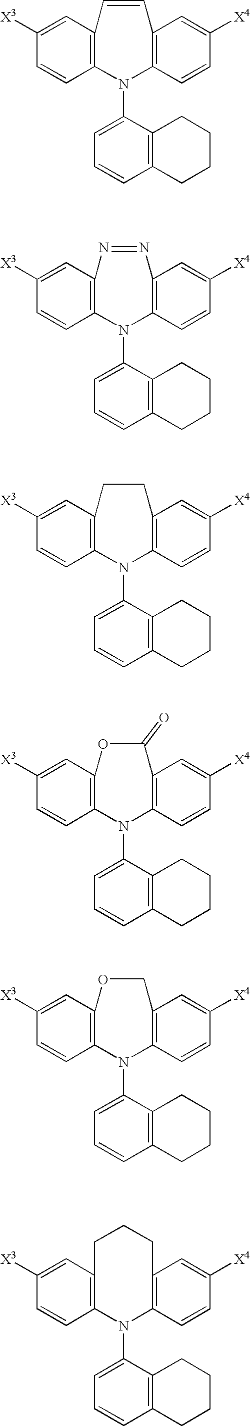

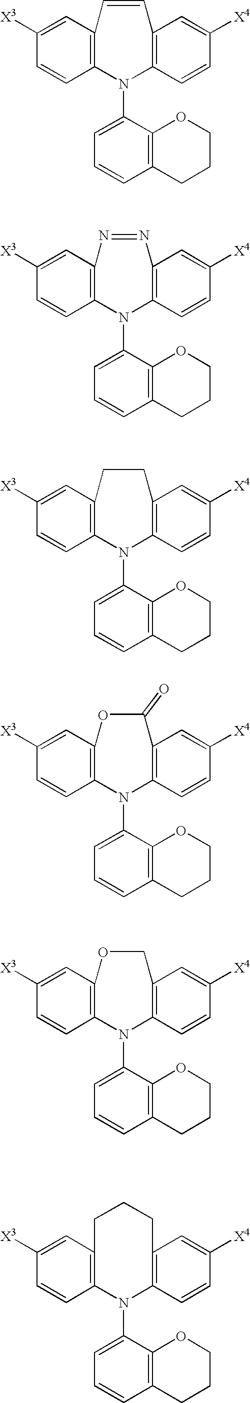

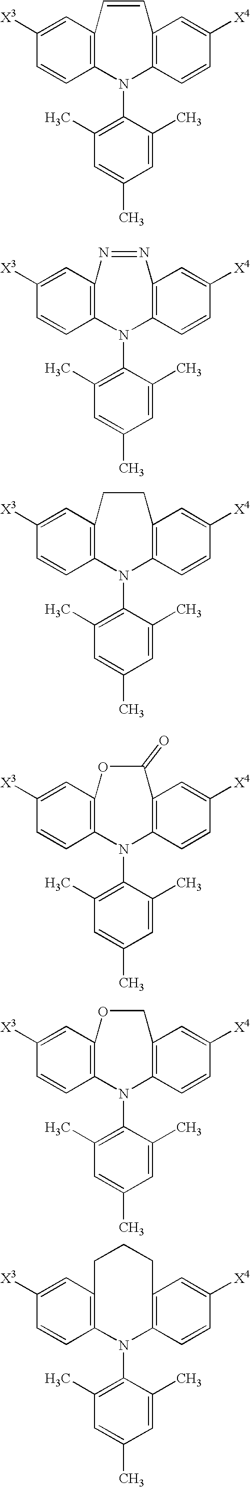

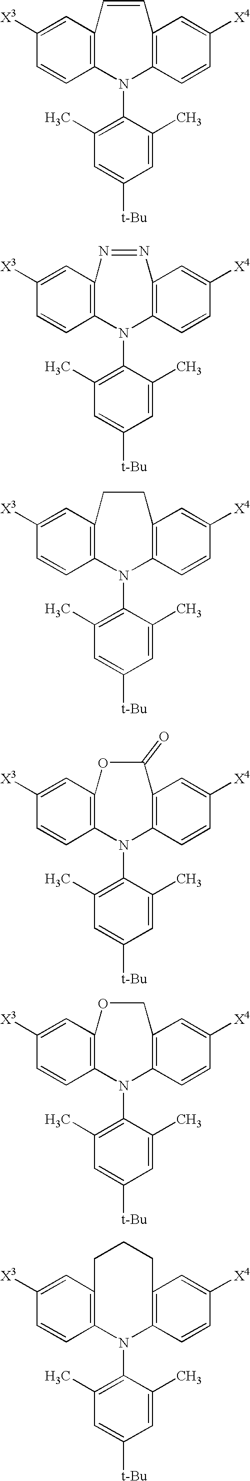

In the formulae, X³ and X⁴ have the same meanings as described above. The structures of the above-described formulae optionally have a substituent.

X³ and X⁴ preferably represent each independently a halogen atom, alkyl sulfonate group, aryl sulfonate group, aryl alkyl sulfonate group, —B(OH)₂, or borate group. Particularly, X³ and X⁴ are preferably a halogen atom since conversion into an alkyl sulfonate group, aryl sulfonate group, aryl alkyl sulfonate group, —B(OH)₂, or borate group is easy when X³ and X⁴ are a halogen. A bromine atom is most preferable.

As the compound of the above-described formula (9), structures of the following formulae are exemplified.

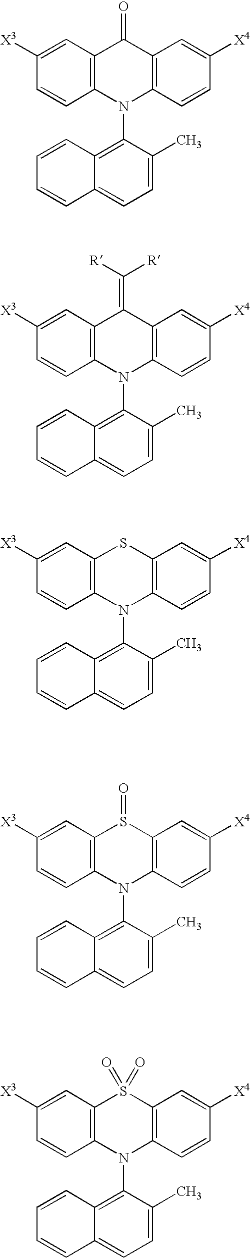

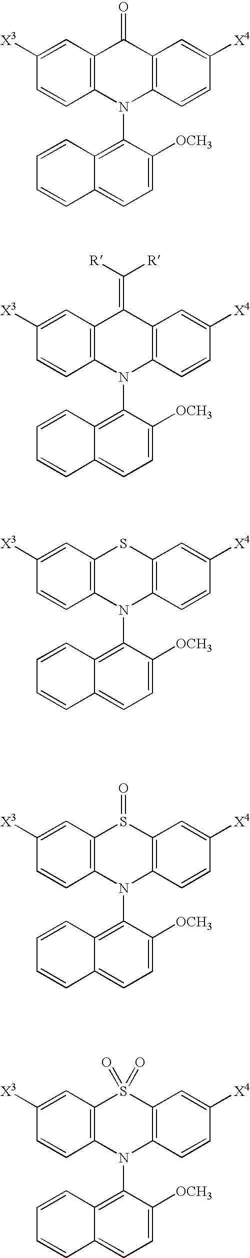

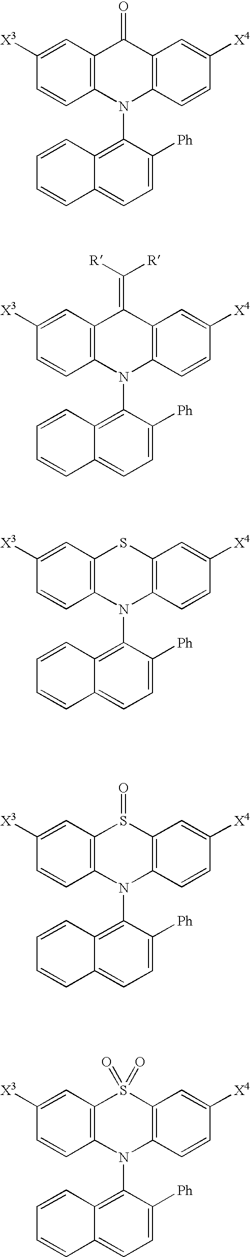

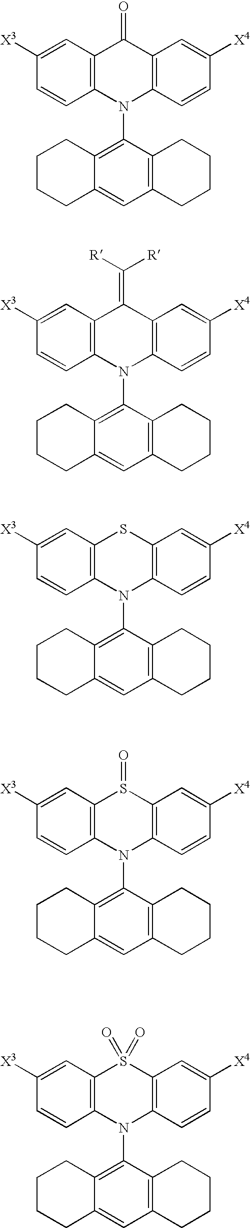

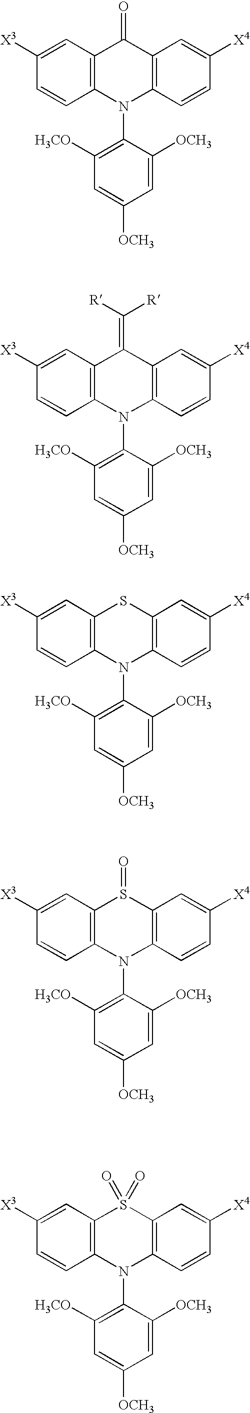

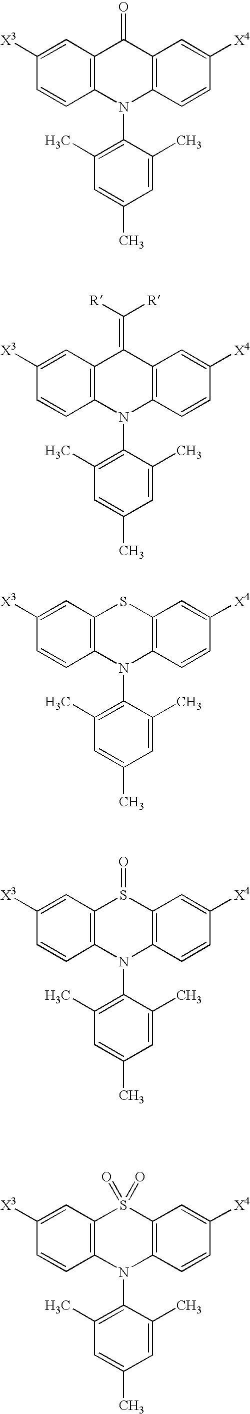

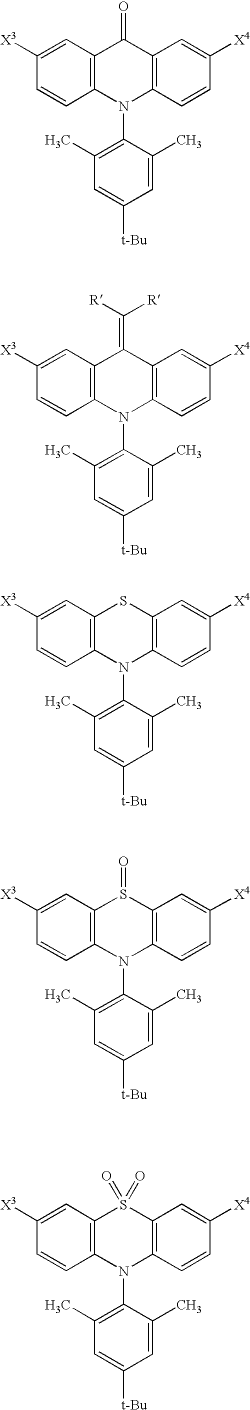

In the formulae, R′, X³ and X⁴ have the same meanings as described above. The structures of the above-described formulae optionally have a substituent.

X³ and X⁴ preferably represent each independently a halogen atom, alkyl sulfonate group, aryl sulfonate group, aryl alkyl sulfonate group, —B(OH)₂, or borate group. Particularly, X³ and X⁴ are preferably a halogen atom since conversion into an alkyl sulfonate group, aryl sulfonate group, aryl alkyl sulfonate group, —B(OH)₂, or borate group is easy when X³ and X⁴ are a halogen. A bromine atom is most preferable.

Next, applications of the polymer compound of the present invention will be illustrated.

The polymer compound of the present invention usually emits fluorescence or phosphorescence in solid state and can be used as a polymer light emitting body (light emitting material of high molecular weight).

The polymer compound has an excellent charge transporting ability, and can be suitably used as a polymer LED material or charge transporting material. The polymer LED using this polymer light emitting body is a high performance polymer LED which can be driven at low voltage with high efficiency. Therefore, the polymer LED can be preferably used for a back light of a liquid crystal display, curved of plane light source for illumination, segment type display, flat panel display of dot matrix, and the like.

The polymer compound of the present invention can also be used as a coloring matter for laser, organic solar battery material, and conductive thin film material such as an organic semiconductor for organic transistor, conductive thin film, organic semiconductor thin film and the like.

Further, it can be used also as a light emitting thin film material which emits fluorescence or phosphorescence.

Next, the polymer LED of the present invention will be illustrated.

The polymer LED of the present invention is characterized in that an organic layer is present between electrodes composed of an anode and a cathode and the organic layer contains a polymer compound of the present invention.

The organic layer may be any of a light emitting layer, hole transporting layer, hole injection layer, electron transporting layer, electron injection layer, interlayer layer and the like, and the organic layer is preferably a light emitting layer.

Here, the light emitting layer means a layer having a function of light emission, the hole transporting layer means a layer having a function of transporting holes, and the electron transporting layer means a layer having a function of transporting electrons. The interlayer layer means a layer which is present adjacent to a light emitting layer between the light emitting layer and an anode, and has a function of insulating a light emitting layer and an anode, or a light emitting layer and a hole injection layer or hole transporting layer. The electron transporting layer and the hole transporting layer are generically called a charge transporting layer. The electro injection layer and the hole injection layer are generically called a charge injection layer. Two or more light emitting layers, two or more hole transporting layers, two or more hole injection layers, two or more electron transporting layers and two or more electron injection layers may be used each independently.

When the organic layer is a light emitting layer, the light emitting layer as an organic layer may further contain a hole transporting material, electron transporting material or light emitting material. Here, the light emitting material means a material showing fluorescence or phosphorescence.

When the polymer compound and the hole transporting material of the present invention are mixed, the mixing ratio of the hole transporting material based on the whole mixture is 1 wt % to 80 wt %, preferably 5 wt % to 60 wt %. When the polymer material and electron transporting material of the present invention are mixed, the mixing ratio of the electron transporting material based on the whole mixture is 1 wt % to 80 wt %, preferably 5 wt % to 60 wt %. Further, when the polymer compound and light emitting material of the present invention are mixed, the mixing ratio of the light emitting material based on the whole mixture is 1 wt % to 80 wt %, preferably 5 wt % to 60 wt %. When the polymer compound, light emitting material, hole transporting material and/or electron transporting material of the present invention are mixed, the mixing ratio of the light emitting material based on the whole mixture is 1 wt % to 50 wt %, preferably 5 wt % to 40 wt %, the ratio of the sum the hole transporting material and electron transporting material is 1 wt % to 50 wt %, preferably 5 wt % to 40 wt %. Thus, the content of the polymer compound of the present invention is 98 wt % to 1 wt %, preferably 90 wt % to 20 wt %.

As the hole transporting material, electron transporting material and light emitting material to be mixed, known low molecular weight compounds, triplet light emitting complexes or polymer compounds can be used, and polymer compounds are preferably used.

Exemplified as the hole transporting material, electron transporting material and light emitting material as polymer compounds are polyfluorene, its derivatives and copolymers, polyarylene, its derivatives and copolymers, polyarylenevinylene, its derivatives and copolymers, and aromatic amine, its derivatives and copolymers disclosed in WO99/13692, WO99/48160, GB2340304A, WO00/53656, WO01/19834, WO00/55927, GB2348316, WO00/46321, WO00/06665, WO99/54943, WO99/54385, U.S. Pat. No. 5,777,070, WO98/06773, WO97/05184, WO00/35987, WO00/53655, WO01/34722, WO99/24526, WO00/22027, WO00/22026, WO98/27136, US573636, WO98/21262, U.S. Pat. No. 5,741,921, WO97/09394, WO96/29356, WO96/10617, EP0707020, WO95/07955, JP-A Nos. 2001-181618, 2001-123156, 2001-3045, 2000-351967, 2000-303066, 2000-299189, 2000-252065, 2000-136379, 2000-104057, 2000-80167, 10-324870, 10-114891, 9-111233, 9-45478 and the like.

As the fluorescent material of lower molecular weight, there can be used, for example, naphthalene derivatives, anthracene or its derivatives, perylene or its derivatives, and polymethine, xanthene, coumarin and cyanine coloring matters, metal complexes of 8-hydrozyquinoline or its derivatives, aromatic amine, tetraphenylcyclopentadiene or its derivatives, or tetraphenylbutadiene or its derivatives, and the like.

Specifically, known compounds such as those described in, for example, JP-A Nos. 57-51781, 59-194393, and the like can be used.

As the triplet light emitting complex, for example, Ir(ppy)₃, Btp₂Ir(acac) containing iridium as a center metal, PtOEP containing platinum as a center metal, Eu(TTA)3phen containing europium as a center metal, and the like are mentioned.

The triplet light emitting complex is described, for example, in Nature, (1998),

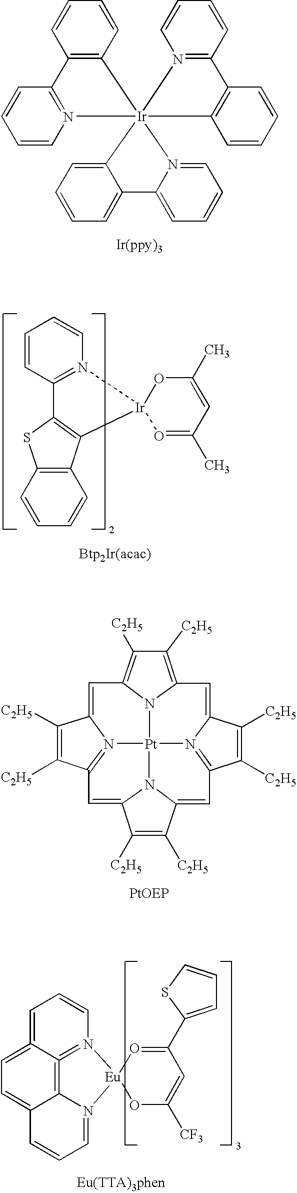

The triplet light emitting complex is described, for example, in Nature, (1998), 395, 151, Appl. Phys. Lett. (1999), 75(1), 4, Proc. SPIE-Int. Soc. Opt. Eng. (2001), 4105 (Organic Light-Emitting Materials and Devices IV), 119, J. Am. Chem. Soc., (2001), 123, 4304, Appl. Phys. Lett., (1997), 71(18), 2596, Syn. Met., (1998), 94(1), 103, Syn. Met., (1999), 99(2), 1361, Adv. Mater., (1999), 11(10), 852, Jpn. J. Appl. Phys., 34, 1883 (1995), and the like.

A composition containing at least one material selected from hole transporting materials, electron transporting materials and light emitting materials, and a polymer compound of the present invention, can be used as the light emitting material or charge transporting material.

The content ratio of at least one material selected from hole transporting materials, electron transporting materials and light emitting materials to a polymer compound of the present invention may be determined depending on use, and in the case of use of a light emitting material, the same content ratio as in the above-mentioned light emitting layer is preferable.

Two or more polymer compounds of the present invention can also be mixed and used as a composition. For enhancing the property of a polymer LED, preferable is a composition containing two or more of polymer compounds containing a hole injection and transporting group on the side chain, polymer compounds containing an electron injection and transporting group on the side chain, and polymer compounds containing a light emitting group on the side chain.

The thickness of a light emitting layer of a polymer LED of the present invention may be advantageously selected so as to give optimum driving voltage and light emission efficiency though the optimum value varies depending on the material to be used, and it is, for example, 1 nm to 1 μm, preferably 2 nm to 500 nm, further preferably 5 nm to 200 nm.

As the method for forming a light emitting layer, a method of film formation from a solution is exemplified. As the film formation method from a solution, application methods such as a spin coat method, casting method, micro gravure coat method, gravure coat method, bar coat method, roll coat method, wire bar coat method, dip coat method, spray coat method, screen printing method, flexo pringing method, offset printing method, inkjet printing method and the like can be used. Printing methods such as a screen printing method, flexo printing method, offset printing method, inkjet printing method and the like are preferable since pattern formation and multicolor separate painting are easy.

As the ink composition (solution) used in printing methods, at least one of polymer compounds of the present invention may be advantageously contained, and in addition to the polymer compound of the present invention, additives such as a hole transporting material, electron transporting material, light emitting material, solvent, stabilizer and the like may be contained.

The ratio of a polymer compound of the present invention in the ink composition is usually 20 wt % to 100 wt %, preferably 40 wt % to 100 wt % based on the total weight of the composition excepting a solvent.

The ratio of a solvent when the ink composition contains a solvent is 1 wt % to 99.9 wt %, preferably 60 wt % to 99.5 wt %, further preferably 80 wt % to 99.0 wt % based on the total weight of the composition.

Though the viscosity of an ink composition varies depending on a printing method, when an ink composition passes through a discharge apparatus such as in inkjet print method and the like, the viscosity at 25° C. is preferably in a range of 1 to 20 mPa·s, for preventing clogging and curving in flying in discharging.

The solution of the present invention may contain additives for regulating viscosity and/or surface tension in addition to the polymer compound of the present invention. As the additive, a polymer compound (thickening agent) having high molecular weight for enhancing viscosity and a poor solvent, a compound of low molecular weight for lowering viscosity, a surfactant for decreasing surface tension, and the like may be appropriately combined and used.

As the above-mentioned polymer compound having high molecular weight, a compound which is soluble in the same solvent as for the polymer compound of the present invention and which does not disturb light emission and charge transportation may be used. For example, polystyrene of high molecular weight, polymethyl methacrylate, polymer compounds of the present invention having larger molecular weights, and the like can be used. The weight-average molecular weight is preferably 500000 or more, more preferably 1000000 or more.

It is also possible to use a poor solvent as a thickening agent. Namely, by adding a small amount of poor solvent for the solid content in a solution, viscosity can be enhanced. When a poor solvent is added for this purpose, the kind and addition amount of the solvent may be advantageously selected within a range not causing deposition of solid components in a solution. When stability in preservation is taken into consideration, the amount of a poor solvent is preferably 50 wt % or less, further preferably 30 wt % or less based on the whole solution.

The solution of the present invention may contain an antioxidant in addition to the polymer compound of the present invention for improving preservation stability. As the antioxidant, a compound which is soluble in the same solvent as for the polymer compound of the present invention and which does not disturb light emission and electric charge transportation is permissible, and exemplified are phenol-type antioxidants, phosphorus-based antioxidants and the like.

When the solution of the present invention is used as an ink composition, though a solvent to be used is not particularly restricted, compounds which can dissolve or uniformly disperse materials other than the solvent constituting the ink composition are preferable. Exemplified as the solvent are chlorine-based solvents such as chloroform, methylene chloride, 1,2-dichloroethane, 1,1,2-trichloroethane, chlorobenzene, o-dichlorobenzene and the like, ether solvents such as tetrahydrofuran, dioxane, anisole and the like, aromatic hydrocarbon solvents such as toluene, xylene and the like, aliphatic hydrocarbon solvents such as cyclohexane, methylcyclohexane, n-pentane, n-hexane, n-heptane, n-octan, n-nonane, n-decane and the like, ketone solvents such as acetone, methyl ethyl ketone, cyclohexanone, benzophenone acetophenone and the like, ester solvents such as ethyl acetate, butyl acetate, ethylcellosolve acetate, methyl benzoate, phenyl acetate and the like, polyhydric alcohols such as ethylene glycol, ethylene glycol monobutyl ether, ethylene glycol monoethyl ether, ethylene glycol monomethyl ether, dimethoxyethane, propylene glycol, diethoxymethane, triethylene glycol monoethyl ether, glycerin, 1,2-hexanediol and the like and derivatives thereof, alcohol solvents such as methanol, ethanol propanol, isopropanol, cyclohexanol and the like, sulfoxide solvents such as dimethyl sulfoxide and the like, amide solvents such as N-methyl-2-pyrrolidone, N,N-dimethylformamide and the like. These organic solvents can be used singly or in combination of two or more.

Of them, preferable from the standpoint of solubility of a polymer compound and the like, uniformity in film formation, viscosity property and the like are aromatic hydrocarbon solvents, aliphatic hydrocarbon solvents, ester solvents and ketone solvents, and mentioned are toluene, xylene, ethylbenzene, diethylbenzene, trimethylbenzene, n-propylbenzene, i-propylbenzene, n-butylbenzene, butylbenzene, s-butylbenzene, anisole, ethoxybenzene, 1-methylnaphthalene, cyclohexane, cyclohexanone, cyclohexylbenzene, bicyclohexyl, cyclohexenylcyclohexanone, n-heptylcyclohexane, n-hexylcyclohexanone, 2-propylcyclohexanone, 2-heptanone, 3-heptanone, 4-heptanone, 2-octanone, 2-nonanone, 2-decanone, dicyclohexylketone, acetophenone and benzophenone.

The number of solvents in a solution is preferably 2 or more, more preferably 2 to 3, further preferably 2, from the standpoint of a film forming property and from the standpoint of device properties and the like.

The number of solvents in a solution is preferably 2 or more, more preferably 2 to 3, further preferably 2, from the standpoint of a film forming property and from the standpoint of device properties and the like.

When two solvents are contained in a solution, one of them may be solid at 25° C. From the standpoint of a film forming property, it is preferable that one solvent has a boiling point of 180° C. or higher, and a solvent having a boiling point of 200° C. or higher is more preferable. From the standpoint of viscosity, it is preferable that an aromatic polymer is dissolved in an amount of 1 wt % or more at 60° C. in both solvents, and it is preferable that one of two solvents dissolves an aromatic polymer in an amount of 1 wt % or more at 25° C.

When two or more solvents are contained in a solution, the content of a solvent having highest boiling point is preferably 40 to 90 wt %, more preferably 50 to 90 wt %, further preferably 65 to 85 wt % based on the weight of all solvents in the solution from the standpoint of viscosity and film forming property.

The polymer compounds of the present invention may be contained singly or in combination of two or more in a solution, and a polymer compound other than the polymer compound of the present invention may also be contained in a range not deteriorating device properties and the like.

The solution of the present invention may contain water, metal and its salt in an amount of 1 to 1000 ppm. As the metal, specifically, lithium, sodium, calcium, potassium, iron, copper, nickel, aluminum, zinc, chromium, manganese, cobalt, platinum, iridium and the like are mentioned. Further, silicon, phosphorus, fluorine, chlorine or bromine may be contained in an amount of 1 to 1000 ppm.

Using the solution of the present invention, a thin film can be formed by a spin coat method, casting method, micro gravure coat method, gravure coat method, bar coat method, roll coat method, wire bar coat method, dip coat method, spray coat method, screen printing method, flexo pringing method, offset printing method, inkjet printing method and the like. Particularly, the solution of the present invention is preferably used for film formation by a screen printing method, flexo printing method, offset printing method or inkjet printing method, and more preferably used for film formation by an inkjet method.

A thin film containing a polymer compound of the present invention can be produced, for example, by using the solution of the present invention. Examples thereof include a light emitting thin film, electrically conductive thin film and organic semiconductor thin film.

The electrically conductive thin film of the present invention preferably has a surface resistance of 1 KΩ/or less. By doping a thin film with a Lewis acid, ionic compound and the like, electric conductivity can be enhanced. The surface resistance is preferably 100Ω/or less, further preferably 10Ω/or less.

In the organic semiconductor thin film of the present invention, one larger parameter of electron mobility or hole mobility is preferably 10⁻⁵ cm²/V/s or more. More preferably, it is 10⁻³ cm²/V/s or more, and further preferably 10⁻¹ cm²/V/s or more.

By forming the organic semiconductor thin film on a Si substrate carrying a gate electrode and an insulation film of SiO₂ and the like formed thereon, and forming a source electrode and a drain electrode with Au and the like, an organic transistor can be obtained.

As the polymer LED of the present invention, mentioned are a polymer LED having an electron transporting layer provided between a cathode and a light emitting layer, a polymer LED having a hole transporting layer provided between an anode and a light emitting layer, a polymer LED having an electron transporting layer provided between a cathode and a light emitting layer and a hole transporting layer provided between an anode and a light emitting layer, and the like.

For example, the following structures a) to d) are specifically mentioned.

a) anode/light emitting layer/cathode

b) anode/hole transporting layer/light emitting layer/cathode

c) anode/light emitting layer/electron transporting layer/cathode

d) anode/hole transporting layer/light emitting layer/electron transporting layer/cathode

(wherein, /means adjacent lamination of layers, applicable also in the followings)

Also exemplified are structures having an interlayer layer provided adjacent to a light emitting layer between the light emitting layer and an anode in the above-described structures. That is:

a′) anode/interlayer layer/light emitting layer/cathode

b′) anode/hole transporting layer/interlayer layer/light emitting layer/cathode

c′) anode/interlayer layer/light emitting layer/electron transporting layer/cathode

d′) anode/hole transporting layer/interlayer layer/light emitting layer/electron transporting layer/cathode

When the polymer LED of the present invention contains a hole transporting layer, exemplified as the hole transporting material to be used are polyvinylcarbazole or its derivatives, polysilane or its derivatives, polysiloxane derivatives having an aromatic amine on the side chain or main chain, pyrazoline derivatives, arylamine derivatives, stilbene derivatives, triphenyldiamine derivatives, polyaniline or its derivatives, polythiophene or its derivatives, polypyrrole or its derivative, s poly(p-phenylenevinylene) or its derivatives, poly(2,5-thienylenevinylene) or its derivatives, and the like.

Specifically, exemplified as the hole transporting material are those described in JP-A Nos. 63-70257, 63-175860, 2-135359, 2-135361, 2-209988, 3-37992 and 3-152184, and the like.

Among them, preferable as the hole transporting material used in a hole transporting layer are polymer hole transporting materials such as polyvinylcarbazole or its derivatives, polsilane or its derivatives, polysiloxane derivatives having an aromatic amine compound group on the side chain or main chain, polyaniline or its derivatives, polythiophene or its derivatives, poly(p-phenylenevinylene) or its derivatives, poly(2,5-thienylenevinylene) or its derivatives, and the like, and further preferable are polyvinylcarbazole or its derivatives, polsilane or its derivatives, and polysiloxane derivatives having an aromatic amine on the side chain or main chain.

Exemplified as the hole transporting material of low molecular weight are pyrazoline derivatives, arylamine derivatives, stilbene derivatives, and triphenyldiamine derivatives. In the case of the hole transporting material of low molecular weight, it is preferably dispersed in a polymer binder in use.

The polymer binder to be mixed is preferably that which does not extremely disturb charge transportation, and those showing no strong absorption against visible ray are suitably used. Exemplified as the polymer binder are poly(N-vinylcarbazole), polyaniline or its derivatives, polythiophene or its derivatives, poly(p-phenylenevinylene) or its derivatives, poly(2,5-thienylenevinylene) or its derivatives, polycarbonate, polyacrylate, polymethyl acrylate, polymethyl methacrylate, polystyrene, polyvinyl chloride, polysiloxane and the like.

Polyvinylcarbazole or its derivative can be obtained, for example, from a vinyl monomer by cation polymerization or radical polymerization.

As the polysilane or its derivative, compounds described in Chem. Rev., vol. 89, p. 1359 (1989), GB Patent No. 2300196 publication, and the like are exemplified. Also as the synthesis method, methods described in them can be used, and particularly, the Kipping method is suitably used.

In the polysiloxane or its derivative, the siloxane skeleton structure shows little hole transporting property, thus, those having a structure of the above-mentioned hole transporting material of low molecular weight on the side chain or main chain are suitably used Particularly, those having an aromatic amine showing a hole transporting property on the side chain or main chain are exemplified.

The film formation method of a hole transporting layer is not particularly restricted, and in the case of a hole transporting material of low molecular weight, a method of film formation from a mixed solution with a polymer binder is exemplified. In the case of a hole transporting material of high molecular weight, a method of film formation from a solution is exemplified.

As the solvent used for film formation from a solution, those which can dissolve or uniformly disperse a hole transporting material are preferable. Exemplified as the solvent are chlorine-based solvents such as chloroform, methylene chloride, 1,2-dichloroethane, 1,1,2-trichloroethane, chlorobenzene, o-dichlorobenzene and the like, ether solvents such as tetrahydrofuran, dioxane and the like, aromatic hydrocarbon solvents such as toluene, xylene and the like, aliphatic hydrocarbon solvents such as cyclohexane, methylcyclohexane, n-pentane, n-hexane, n-heptane, n-octan, n-nonane, n-decane and the like, ketone solvents such as acetone, methyl ethyl ketone, cyclohexanone and the like, ester solvents such as ethyl acetate, butyl acetate, ethylcellosolve acetate and the like, polyhydric alcohols such as ethylene glycol, ethylene glycol monobutyl ether, ethylene glycol monoethyl ether, ethylene glycol monomethyl ether, dimethoxyethane, propylene glycol, diethoxymethane, triethylene glycol monoethyl ether, glycerin, 1,2-hexanediol and the like and derivatives thereof, alcohol solvents such as methanol, ethanol, propanol, isopropanol, cyclohexanol and the like, sulfoxide solvents such as dimethyl sulfoxide and the like, amide solvents such as N-methyl-2-pyrrolidone, N,N-dimethylformamide and the like. These organic solvents can be used singly or in combination of two or more.

As the method for film formation from a solution, there can be used application methods from a solution such as a spin coat method, casting method, micro gravure coat method, gravure coat method, bar coat method, roll coat method, wire bar coat method, dip coat method, spray coat method, screen printing method, flexo pringing method, offset printing method, inkjet printing method and the like.

Regarding the thickness of a hole transporting layer, the optimum value varies depending on a material to be used, and it may be advantageously selected so that the driving voltage and light emission efficiency become optimum, and a thickness at least causing no formation of pin holes is necessary, and when the thickness is too large, the driving voltage of a device increases undesirably. Therefore, the thickness of the hole transporting layer is, for example, 1 nm to 1 μm, preferably 2 nm to 500 nm, further preferably 5 nm to 200 nm.

When the polymer LED of the present invention has an electron transporting layer, known materials can be used as the electron transporting material to be used, and exemplified are oxadiazole derivatives, anthraquinodimethane or its derivatives, benzoquinone or its derivatives, naphthoquinone or its derivatives, anthraquinone or its derivatives, tetracyanoanthraquinodimethane or its derivatives, fluorenone derivatives, diphenyldicyanoethylene or its derivatives, diphenoquinone derivatives, metal complexes of 8-hydroxyquinoline or its derivatives, polyquinoline or its derivatives, polyquinoxaline or its derivatives, polyfluorene or its derivatives, and the like.

Specifically, those described in JP-A Nos. 63-70257, 63-175860, 2-135359, 2-135361, 2-209988, 3-37992, 3-152184, and the like are exemplified.

Of them, oxadiazole derivatives, benzoquinone or its derivatives, anthraquinone or its derivatives, metal complexes of 8-hydroxyquinoline or its derivative, s polyquinoline or its derivatives, polyquinoxaline or its derivatives, polyfluorene or its derivatives are preferable, and 2-(4-biphenylyl)-5-(4-t-butylphenyl)-1,3,4-oxadiazole, benzouqinone, anthraquinone, tris(8-quinolinol)aluminum and polyquinoline are further preferable.

The film formation method of an electron transporting layer is not particularly restricted, and in the case of an electron transporting material of low molecular weight, exemplified are a vacuum vapor-deposition method from powder, film formation methods from solution or melted conditions, and in the case of an electron transporting material of high molecular weight, film formation methods from solution or melted condition are exemplified, respectively. In film formation from solution or melted condition, the above-mentioned polymer binders may be used together.

As the solvent used in film formation from a solution, compounds which can dissolve or uniformly disperse an electron transporting material and/or polymer binder are preferable. Exemplified as the solvent are chlorine-based solvents such as chloroform, methylene chloride, 1,2-dichloroethane, 1,1,2-trichloroethane, chlorobenzene, o-dichlorobenzene and the like, ether solvents such as tetrahydrofuran, dioxane and the like, aromatic hydrocarbon solvents such as toluene, xylene and the like, aliphatic hydrocarbon solvents such as cyclohexane, methylcyclohexane, n-pentane, n-hexane, n-heptane, n-octan, n-nonane, n-decane and the like, ketone solvents such as acetone, methyl ethyl ketone, cyclohexanone and the like, ester solvents such as ethyl acetate, butyl acetate, ethylcellosolve acetate and the like, polyhydric alcohols such as ethylene glycol, ethylene glycol monobutyl ether, ethylene glycol monoethyl ether, ethylene glycol monomethyl ether, dimethoxyethane, propylene glycol, diethoxymethane, triethylene glycol monoethyl ether, glycerin, 1,2-hexanediol and the like and derivatives thereof, alcohol solvents such as methanol, ethanol propanol, isopropanol, cyclohexanol and the like, sulfoxide solvents such as dimethyl sulfoxide and the like, amide solvents such as N-methyl-2-pyrrolidone, N,N-dimethylformamide and the like. These organic solvents can be used singly or in combination of two or more.

As the film formation method from solution or melted condition, application methods such as a spin coat method, casting method, micro gravure coat method, gravure coat method, bar coat method, roll coat method, wire bar coat method, dip coat method, spray coat method, screen printing method, flexo pringing method, offset printing method, inkjet printing method and the like can be used.

Regarding the thickness of an electron transporting layer, the optimum value varies depending on a material to be used, and it may be advantageously selected so that the driving voltage and light emission efficiency become optimum, and a thickness at least causing no formation of pin holes is necessary, and when the thickness is too large, the driving voltage of a device increases undesirably.

Therefore, the thickness of the electron transporting layer is, for example, 1 nm to 1 μm, preferably 2 nm to 500 nm, further preferably 5 nm to 200 nm.

Among charge transporting layers provided adjacent to an electrode, those having a function of improving charge injection efficiency from an electrode and having an effect of lowering the driving voltage of a device are, in particularly, called generally a charge injection layer (hole injection layer, electron injection layer).

Further, for improving close adherence with an electrode or improving charge injection from an electron, the above-mentioned charge injection layer or an insulation layer having a thickness of 2 nm or less may be provided adjacent to the electrode, alternatively, for improving close adherence of an interface or preventing mixing, a thin buffer layer may be inserted into an interface of a charge transporting layer and a light emitting layer.

The order and number of layers to be laminated, and thickness of each layer can be appropriately determined in view of light emission efficiency and device life.

In the present invention, as the polymer LED carrying a provided charge injection layer (electron injection layer, hole injection layer), mentioned are polymer LED having a charge injection layer provided adjacent to a cathode and polymer LED having a charge injection layer provided adjacent to an anode.

For example, the following structures e) to p) are specifically mentioned.

e) anode/charge injection layer/light emitting layer/cathode

f) anode/light emitting layer/charge injection layer/cathode

g) anode/charge injection layer/light emitting layer/charge injection layer/cathode

h) anode/charge injection layer/hole transporting layer/light emitting layer/cathode

i) anode/hole transporting layer/light emitting layer/charge injection layer/cathode

j) anode/charge injection layer/hole transporting layer/light emitting layer/charge injection layer/cathode

k) anode/charge injection layer/light emitting layer/electron transporting layer/cathode

l) anode/light emitting layer/electron transporting layer/charge injection layer/cathode

m) anode/charge injection layer/light emitting layer/electron transporting layer/charge injection layer/cathode

n) anode/charge injection layer/hole transporting layer/light emitting layer/electron transporting layer/cathode

o) anode/hole transporting layer/light emitting layer/electron transporting layer/charge injection layer/cathode

p) anode/charge injection layer/hole transporting layer/light emitting layer/electron transporting layer/charge injection layer/cathode

Also exemplified are structures having an interlayer layer provided adjacent to a light emitting layer between the light emitting layer and an anode in the above-described structures. In this case, the interlayer layer may also function as a hole injection layer and/or hole transporting layer.

As specific examples of the charge injection layer, exemplified are a layer containing an electric conductive polymer, a layer provided between an anode and a hole transporting layer and containing a material having ionization potential of a value between an anode material and a hole transporting material contained in a hole transporting layer, a layer containing a material having electron affinity of a value between a cathode material and an electron transporting material contained in an electron transporting layer, and the like.

When the above-mentioned charge injection layer contains an electric conductive polymer, electric conductivity of the electric conductive polymer is preferably 10⁻⁵ S/cm or more and 10³ or less, and for decreasing leak current between light emission picture elements, more preferably 10⁻⁵ S/cm or more and 10² or less, further preferably 10⁻⁵ S/cm or more and 10¹ or less.

When the above-mentioned charge injection layer contains an electric conductive polymer, electric conductivity of the electric conductive polymer is preferably 10⁻⁵ S/cm or more and 10³ or less, and for decreasing leak current between light emission picture elements, more preferably 10⁻⁵ S/cm or more and 10² or less, further preferably 10⁻⁵ S/cm or more and 10¹ or less.

Usually, for controlling the electric conductivity of the electric conductive polymer to 10⁻⁵ S/cm or more and 10³ or less, the electric conductive polymer is doped with a suitable amount of electrons.

As the kind of ions to be doped, an anion is used in a hole injection layer and a cation is used in an electron injection layer. Examples of the anion include a polystyrenesulfonic ion, alkylbenzenesulfonic ion, camphorsulfonic ion and the like, and examples of the cation include a lithium ion, sodium ion, potassium ion, tetrabutylammonium ion and the like.

The thickness of the charge injection layer is, for example, 1 nm to 100 nm, preferably 2 nm to 50 nm.

The material used in the charge injection layer may be appropriately selected depending on a relation with materials of an electrode and an adjacent layer, and exemplified are polyaniline or its derivatives, polythiophene or its derivatives, polypyrrole and its derivatives, polyphenylenevinylene and its derivatives, polythienylenevinylene and its derivatives, polyquinoxaline and its derivatives, electric conductive polymers such as polymers containing an aromatic amine structure on the main chain or side chain, metal phthalocyanines (copper phthalocyanine and the like), carbon and the like.

An insulation layer having a thickness of 2 nm or less has a function of making charge injection easy. As the material of the above-mentioned insulation layer, a metal fluoride, metal oxide, organic insulating material and the like are mentioned. As the polymer LED carrying an insulation layer having a thickness of 2 nm or less provide thereon, there are mentioned polymer LED in which an insulation layer having a thickness of 2 nm or less is provided adjacent to a cathode, and polymer LED in which an insulation layer having a thickness of 2 nm or less is provided adjacent to an anode.

Specifically, the following structures q) to ab) are mentioned, for example.

q) anode/insulation layer having a thickness of 2 nm or less/light emitting layer/cathode

r) anode/light emitting layer/insulation layer having a thickness of 2 nm or less/cathode

s) anode/insulation layer having a thickness of 2 nm or less/light emitting layer/insulation layer having a thickness of 2 nm or less/cathode

t) anode/insulation layer having a thickness of 2 nm or less/hole injection layer/light emitting layer/cathode

u) anode/hole injection layer/light emitting layer/insulation layer having a thickness of 2 nm or less/cathode

v) anode/insulation layer having a thickness of 2 nm or less/hole transporting layer/light emitting layer/insulation layer having a thickness of 2 nm or less/cathode

w) anode/insulation layer having a thickness of 2 nm or less/light emitting layer/electron transporting layer/cathode

x) anode/light emitting layer/electron transporting layer/insulation layer having a thickness of 2 nm or less/cathode

y) anode/insulation layer having a thickness of 2 nm or less/light emitting layer/electron transporting layer/insulation layer having a thickness of 2 nm or less/cathode

z) anode/insulation layer having a thickness of 2 nm or less/hole transporting layer/light emitting layer/electron transporting layer/cathode

aa) anode/hole transporting layer/light emitting layer/electron transporting layer/insulation layer having a thickness of 2 nm or less/cathode

ab) anode/insulation layer having a thickness of 2 nm or less/hole transporting layer/light emitting layer/electron transporting layer/insulation layer having a thickness of 2 nm or less/cathode

Also exemplified are structures having an interlayer layer provided adjacent to a light emitting layer between the light emitting layer and an anode in the above-described structures. In this case, the interlayer layer may also function as a hole injection layer and/or hole transporting layer.

In structures in which an interlayer layer is applied to the above-described structures a) to ab), the interlayer layer is preferably provided between an anode and a light emitting layer and constituted of a material having intermediate ionization potential between the anode or hole injection layer or hole transporting layer, and a polymer compound constituting the light emitting layer.

As the material to be used in the interlayer layer, exemplified are polymers containing an aromatic amine such as polyvinylcarbazole or its derivatives, polyarylene derivatives having an aromatic amine on the side chain or main chain, arylamine derivatives, triphenyldiamine derivatives and the like.

The method for forming the interlayer layer is not particularly restricted, and in the case of use of, for example, a polymer material, a method of film formation from a solution is exemplified.

As the solvent used in film formation from a solution, compounds which can dissolve or uniformly disperse a hole transporting material are preferable. Exemplified as the solvent are chlorine-based solvents such as chloroform, methylene chloride, 1,2-dichloroethane, 1,1,2-trichloroethane, chlorobenzene, o-dichlorobenzene and the like, ether solvents such as tetrahydrofuran, dioxane and the like, aromatic hydrocarbon solvents such as toluene, xylene and the like, aliphatic hydrocarbon solvents such as cyclohexane, methylcyclohexane, n-pentane, n-hexane, n-heptane, n-octan, n-nonane, n-decane and the like, ketone solvents such as acetone, methyl ethyl ketone, cyclohexanone and the like, ester solvents such as ethyl acetate, butyl acetate, ethylcellosolve acetate and the like, polyhydric alcohols such as ethylene glycol, ethylene glycol monobutyl ether, ethylene glycol monoethyl ether, ethylene glycol monomethyl ether, dimethoxyethane, propylene glycol, diethoxymethane, triethylene glycol monoethyl ether, glycerin, 1,2-hexanediol and the like and derivatives thereof, alcohol solvents such as methanol, ethanol propanol, isopropanol, cyclohexanol and the like, sulfoxide solvents such as dimethyl sulfoxide and the like, amide solvents such as N-methyl-2-pyrrolidone, N,N-dimethylformamide and the like. These organic solvents can be used singly or in combination of two or more.

As the film formation method from a solution, application methods from a solution such as a spin coat method, casting method, micro gravure coat method, gravure coat method, bar coat method, roll coat method, wire bar coat method, dip coat method, spray coat method, screen printing method, flexo pringing method, offset printing method, inkjet printing method and the like can be used.

Regarding the thickness of an interlayer layer, the optimum value varies depending on a material to be used, and it may be advantageously selected so that the driving voltage and light emission efficiency become optimum. The thickness thereof is, for example, 1 nm to 1 μm, preferably 2 nm to 500 nm, further preferably 5 nm to 200 nm.

When the interlayer layer is provided adjacent to a light emitting layer, particularly when both the layers are formed by an application method, the two layers may be mixed to exert an undesirable influence on device properties and the like in some cases. When the interlayer layer is formed by an application method before formation of a light emitting layer by an application method, there is mentioned a method in which an interlayer layer is formed by an application method, then, the interlayer layer is heated to be insolubilized in an organic solvent to be used for manufacturing a light emitting layer, then, the light emitting layer is formed, as a method for reducing mixing of materials of the two layers. The heating temperature is usually about 150° C. to 300° C., and the heating time is usually about 1 minute to 1 hour. In this case, for removal of components not insolubilized in solvent by heating, the interlayer layer can be removed by rinsing with a solvent to be used for formation of a light emitting layer, after heating and before formation of the light emitting layer. When solubilization in solvent by heating is carried out sufficiently, rinsing with a solvent can be omitted. For solubilization in solvent by heating to be carried out sufficiently, it is preferable to use a compound containing at least one polymerizable group in the molecule, as a polymer compound to be used for an interlayer layer. Further, the number of polymerizable groups is preferably 5% or more based on the number of repeating units in the molecule.

The substrate which forms a polymer LED of the present invention may be that forming an electrode and which does not change in forming a layer of an organic substance, and examples thereof include substrates of glass, plastic, polymer film, silicon and the like. In the case of an opaque substrate, it is preferable that the opposite electrode is transparent or semi-transparent.

Usually, at least one of an anode and cathode contained in a polymer LED of the present invention is transparent or semi-transparent. It is preferable, that a cathode is transparent or semi-transparent.

As the material of the cathode, an electric conductive metal oxide film, semi-transparent metal thin film and the like are used. Specifically, films (NESA and the like) formed using electric conductive glass composed of indium oxide, zinc oxide, tin oxide, and composite thereof: indium•tin•oxide (ITO), indium•zinc•oxide and the like, gold, platinum, silver, copper and the like are used, and ITO, indium•zinc•oxide, tin oxide are preferable. As the manufacturing method, a vacuum vapor-deposition method, sputtering method, ion plating method, plating method and the like are mentioned. As the anode, organic transparent electric conductive films made of polyaniline or its derivative, polythiophene or its derivative, and the like may be used.

The thickness of an anode can be appropriately selected in view of light transmission and electric conductivity, and it is, for example, 10 nm to 10 μm, preferably 20 nm to 1 μm, further preferably 50 nm to 500 nm.

For making electric charge injection easy, a layer made of a phthalocyanine derivative, electric conductive polymer, carbon and the like, or a layer having an average thickness of 2 nm or less made of a metal oxide, metal fluoride, organic insulation material and the like, may be provided on an anode.

As the material of a cathode used in a polymer LED of the present invention, materials of small work function are preferable. For example, metals such as lithium, sodium, potassium, rubidium, cesium, beryllium, magnesium, calcium, strontium, barium, aluminum, scandium, vanadium, zinc, yttrium, indium, cerium, samarium, europium, terbium, ytterbium and the like, alloys of two or more of them, or alloys made of at least one of them and at least one gold, silver, platinum, copper, manganese, titanium, cobalt, nickel, tungsten and tin, graphite or graphite interlaminar compounds and the like are used. Examples of the alloy include magnesium-silver alloy, magnesium-indium alloy, magnesium-aluminum alloy, indium-silver alloy, lithium-aluminum alloy, lithium-magnesium alloy, lithium-indium alloy, calcium-aluminum alloy and the like. The cathode may take a laminated structure including two or more layers.

The thickness of a cathode can be appropriately selected in view of electric conductivity and durability, and it is, for example, 10 nm to 10 μm, preferably 20 nm to 1 μm, further preferably 50 nm to 500 nm.

As the cathode manufacturing method, a vacuum vapor-deposition method, sputtering method, lamination method of thermally press-binding a metal thin film, and the like are used. A layer made of an electric conductive polymer, or a layer having an average thickness of 2 nm or less made of a metal oxide, metal fluoride, organic insulation material and the like, may be provided between a cathode and an organic substance layer, and after manufacturing a cathode, a protective layer for protecting the polymer LED may be installed. For use of the polymer LED stably for a long period of time, it is preferable to install a protective layer and/or protective cover, for protecting a device from outside.

As the protective layer, a polymer compound, metal oxide, metal fluoride, metal boride and the like can be used. As the protective cover, a glass plate, and a plastic plate having a surface which has been subjected to low water permeation treatment, and the like can be used, and a method of pasting the cover to a device substrate with a thermosetting resin or photo-curable resin to attain sealing is suitably used. When a space is kept using a spacer, blemishing of a device can be prevented. If an inert gas such as nitrogen, argon and the like is filled in this space, oxidation of a cathode can be prevented, further, by placing a drying agent such as barium oxide and the like in this space, it becomes easy to suppress moisture adsorbed in a production process from imparting damage to the device. It is preferable to adopt one strategy among these methods.

The polymer LED of the present invention can be used as a sheet light source, a display such as a segment display, dot matrix display and the like, or back light of a liquid crystal display.

For obtaining light emission in the form of sheet using a polymer LED of the present invention, it may be advantages to place a sheet anode and a sheet cathode so as to overlap. For obtaining light emission in the form of pattern, there are a method in which a mask having a window in the form of pattern is placed on the surface of the above-mentioned sheet light emitting device, a method in which an organic substance layer in non-light emitting parts is formed with extremely large thickness to give substantially no light emission, a method in which either anode or cathode, or both electrodes are formed in the form pattern. By forming a pattern by any of these methods, and placing several electrodes so that on/off is independently possible, a display of segment type is obtained which can display digits, letters, simple marks and the like. Further, for providing a dot matrix device, it may be permissible that both an anode and a cathode are formed in the form of stripe, and placed so as to cross. By using a method in which several polymer fluorescent bodies showing different emission colors are painted separately or a method in which a color filter or a fluorescence conversion filter is used, partial color display and multi-color display are made possible. In the case of a dot matrix device, passive driving is possible, and active driving may be carried out in combination with TFT and the like. These displays can be used as a display of a computer, television, portable terminal, cellular telephone, car navigation, view finder of video camera, and the like.

Further, the above-mentioned sheet light emitting device is of self emitting and thin type, and can be suitably used as a sheet light source for back light of a liquid crystal display, or as a sheet light source for illumination. If a flexible substrate is used, it can also be used as a curved light source or display.

The present invention will be illustrated further in detail below, but the invention is not limited to them.

### (Number-Average Molecular Weight and Weight-Average Molecular Weight)

Here, as the number-average molecular weight and the weight-average molecular weight, a number-average molecular weight and a weight-average molecular weight in terms of polystyrene were measured by GPC (manufactured by Shimadzu Corp., LC-10Avp). A polymer to be measured was dissolved in tetrahydrofuran so as to give a concentration of about 0.5 wt %, and the solution was injected in an amount of 50 μL into GPC. Tetrahydrofuran was used as the mobile phase of GPC, and allowed to flow at a flow rate of 0.6 mL/min. In the column, two TSKgel Super HM-H (manufactured by Tosoh Corp.) and one TSKgel Super H2000 (manufactured by Tosoh Corp.) were connected serially. A differential refractive index detector (RID-10A: manufactured by Shimadzu Corp.) was used as a detector.

### Example 1

### Synthesis of Compound B and Compound B-1

### (Synthesis of Compound A)

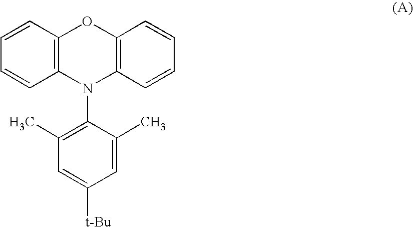

A three-necked flask was equipped with a reflux tube. Under a nitrogen atmosphere, 10.0 g of phenoxazine, 15.2 g of 1-bromo-4-t-butyl-2,6-dimethylbenzene, 21.9 g of sodium t-butoxide and 345 ml of toluene were added and stirred, then, 0.25 g of trisdibenzylideneacetonedipalladium and 0.13 g of t-butylphosphine tetrafluoroborate were added. Under reflux, the mixture was stirred for 9 hours, and cooled to room temperature. The reaction solution was filtrated through a glass filter pre-coated with alumina, and the resulting solution was washed using 3.5% hydrochloric acid, and the toluene solution was concentrated. To the resultant solid was added 5 ml of toluene and 50 ml of isopropyl alcohol and the mixture was heated, and stirred for 1 hour, then, cooled to room temperature. The generated precipitate was filtrated, and washed with isopropyl alcohol to obtain 8.3 g of compound A as pale yellow solid.

¹H-NMR (CDCl₃ 300 MHz):

d1.36 (s, 9H), 2.20 (s, 6H), 5.70-5.74 (m, 2H), 6.53-6.65 (m, 6H), 7.21 (s, 2H).

### (Synthesis of Compound B)

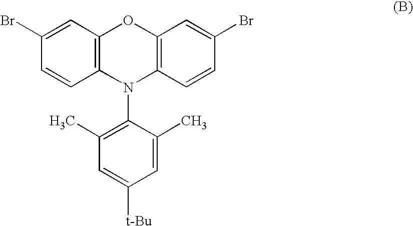

A three-necked flask was equipped with a reflux condenser. Under a nitrogen atmosphere, 8.3 g of the compound A synthesized above and 25 ml of dichloromethane were charged, and stirred at 0° C. A solution of 6.8 g of 1,3-dibromo-5,5-dimethylhydantoin in DMF (7.3 ml) was prepared, and added while stirring at 0° C. Further, 0.02 equivalent of 1,3-dibromo-5,5-dimethylhydantoine was added and stirred for 1 hour, and heated up to room temperature. 100 ml of methanol was added and the mixture was stirred, and the resultant precipitate was filtrated. To this precipitate was added 50 ml of toluene and 300 ml of methanol and the resultant mixture was stirred at 70° C. for 1 hour, then, cooled to room temperature and filtrated. Further, to the precipitate was added 100 ml of toluene and 1 g of acticated carbon and refluxing was carried out with heating, and the mixture was filtrated through a glass filted pre-coated with cerite, and the resulting solution was added to 500 ml of methanol. The generated precipitate was filtrated, and the resultant solid was re-crystallized from toluene to obtain 4.3 g of the intended compound B as pale yellow crystal.

¹H-NMR (CDCl₃ 300 MHz):

d1.35 (s, 9H), 2.16 (s, 6H), 5.58 (dd, 8.46, 1.5 Hz, 2H), 6.68 (ddd, 8.7, 2.1, 1.5 Hz, 2H), 6.79 (dd, 2.1, 1.5 Hz, 2H), 7.26 (s, 2H).

MS (APPI-Positive):

m/z calcd for [M], 499.01; found, 499 as [M₊.]. (containing two Br)

### (Synthesis of Compound B-1)

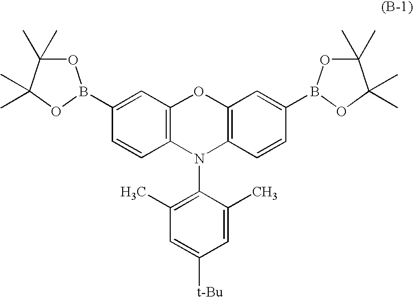

Acording to a method described in Bioorganic & Medicinal Chemistry Letters (2003), 13(18), 3059, compound B-1 can be obtained by heating in a dimethyl sulfoxide solvent in the presence of the compound B, bis(pinacolate)diborane, [1,1′-bis(diphenylphosphino)ferrocene]dichloropalladium (II) and potassium acetate.

### Synthesis Example 1

### Synthesis of Compound D

### (Synthesis of Compound C)

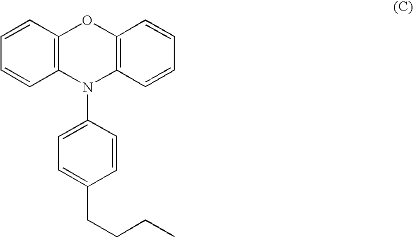

A 3 L four-necked flask was equipped with a mechanical stirrer and condenser. An atmosphere in the reaction vessel was purged with nitrogen, and 1.10 g of palladium acetate (II), 1.51 g of tris(o-tolyl)phosphine and 368 ml of toluene were added and stirred at room temperature for 30 minutes. 143 g of phenoxazine, 97.1 g of sodium t-pentoxide and 800 ml of toluene were added and the mixture was stirred, and 133.4 ml of 1-bromo-4-butylbenzene was dissolved in 60 ml of toluene and the resulting solution was dropped into the reaction vessel using a dropping funnel. The mixture was stirred at 105° C. for 5 hours, then, cooled to room temperature. The mixture was filtrated through a glass filter pre-coated with 2 cm of alumina, and the resulting solution was neutralized with 3.5% hydrochloric acid. The toluene solution was concentrated, and 30 ml of toluene was again added and the resultant mixture was stirred at 75° C. for 30 minutes, then, 700 ml of isopropanol was added slowly. After cooling to room temperature, the deposited precipitate was filtrated, and washed with isopropanol. As a result, 209 g of compound C was obtained as pale orange slid.

¹H-NMR (CDCl₃, 300 MHz)

d7.38 (d, 8.07 Hz, 2H), 7.22 (d, 8.07 Hz, 2H), 6.52-6.70 (m, 6H), 7.53 (d, 7.53 Hz, 2H), 2.69 (t, 7.53 Hz, 2H), 1.68 (m, 2H), 1.42 (m, 2H), 0.98 (t, 7.17 Hz, 3H)

### (Synthesis of Compound D)

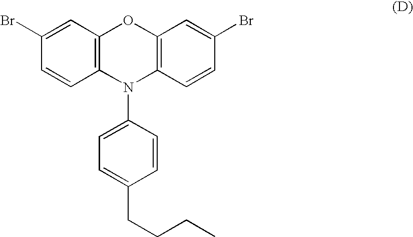

A 3 L four-necked flask was equipped with a mechanical stirrer, dropping funnel and condenser. An atmosphere in the reaction vessel was purged with nitrogen, and 209 of the compound C and 700 ml of dichloromethane were charged and stirred at room temperature. 190 g of 1,3-dibromo-5,5-dimethylhydantoin was dissolved in 200 ml of DMF. The DMF solution prepared was added at room temperature from the dropping funnel. At a stage of addition of 339 ml, the reaction was terminated. To the reaction mass, methanol was poured, and the mixture was cooled slowly to 10° C. using a water bath. After stirring for 1 hour, the deposited precipitate was filtrated, and washed with methanol, to obtain 284 g of compound D as pale white green solid.

¹H-NMR (CDCl₃, 300 MHz)

d7.38 (d, 8.07 Hz, 2H), 7.16 (d, 8.07 Hz, 2H), 6.79 (t, 1.83 Hz, 2H), 6.69 (ddd, 8.64, 1.83, 1.65 Hz, 2H), 5.76 (dd, 8.64, 1.65 Hz, 2H), 2.69 (t, 7.71 Hz, 2H), 1.67 (m, 2H), 1.41 (m, 2H), 0.97 (t, 6.03 Hz, 3H)

### Synthesis Example 2

### Synthesis of Compound F

### (Synthesis of Compound E)

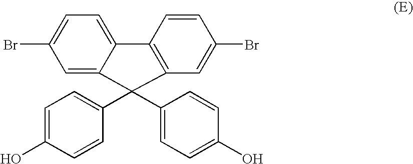

A 3 L three-necked round-bottomed flask was equipped with a mechanical stirrer and condenser, and purged with nitrogen. Then, 86.5 g of 2,7-dibromo-9-fluorenone and 500 g of phenol dissolved by heating in an oven were added. The temperature was raised up to 105° C. while stirring, and at a stage of completion dissolution of 2,7-dibromo-9-fluorenone, cooled to 65° C. 1.98 g of 3-mercaptopropane-1-sulfonic acid was weighed in a globe box, and added slowly while taking care so as not to increase the temperature in the reaction system. A catalyst was added, then, the mixture was stirred at 65° C. for 21 hours, and 722 ml of ethanol was added and dissolution was caused by heating. Thereafter, the mixture was cooled down to 45° C., and poured into 7.6 L of ion exchanged water heated at 65° C., then, the mixture was stirred for 2 hours. The deposited orange precipitate was filtrated, and washed with water, then, allowed to stand over night and day to attain drying. The resultant oragnge solid was transferred into a 3 L three-necked flask, and 400 ml of acetonitrile was added and the mixture was refluxed with heating for 1 hour. After cooling down to 50° C., filtration was carried out under heat to remove insoluble materials. The resultant acetonitrile solution was semi-concentrated, and the deposited precipitate was filtrated. The product was washed with a small amount of acetonitrile, and dried over night any day in a vacuum drying machine. As a result, 92.2 g of compound E was obtained as paled yellow solid.

¹H-NMR (CDC₁₃, 300 MHz)

d7.57 (m, 2H), 7.47 (m, 2H), 7.26 (s, 2H), 7.01 (m, 4H), 6.71 (m, 4H), 4.83 (s, 2H)

### Preparation of 3-mercaptopropane-1-sulfonic acid

Into a 500 ml eggplant-shaped flask was added 10.8 g of sodium salt of 3-mercaptopropane-1-sulfonic acid, and 101 ml of concentrated hydrochloric acid was added at room temperature. After stirring for 10 minute, the mixture was filtrated. The resultant aqueous solution was concentrated by an evaporator, to obtain 8.3 g of 3-mercaptopropane-1-sulfonic acid as colorless transparent oil.

### (Synthesis of Compound F)

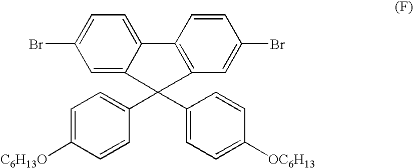

Into a 500 ml three-necked flask was added 50 g of the compound E, 55 ml of n-bromohexane, 53.6 of potassium carbonate and 238 ml of ethanol, and the mixture was stirred for 5 hours unde reflux with heating. 512 ml of ethanol was added and the mixture was cooled down to 50° C. Into a 1 L beaker was added 584 ml of ion exchanged water, and the reaction solution was poured. After stirring for 1 hour, an aqueous layer was removed by decantation. To this was added 487 ml of ion exchanged water and the mixture was further stirred for 1 hour, then, an aqueous layer was removed by decantation. To this was added 292 ml of ethanol and the mixture was stirred for 1 hour. The resultant crystal was filtrated, and washed with ethanol and water, to obtain 60.1 g of compound F as while solid.

¹H-NMR (CDCl₃, 300 MHz)

d 7.56 (m, 2H), 7.47 (s, 2H), 7.45 (m, 2H), 7.04 (d, 4H), 6.76 (d, 4H), 3.90 (t, 4H), 1.70-1.80 (m, 4H), 1.25-1.50 (m, 12H), 0.89 (t, 6H)

### Synthesis Example 3

### Synthesis of Compound H

### (Synthesis of Compound G)

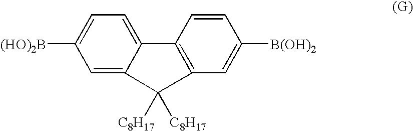

A 3 L four-necked flask was purged with nitrogen, and 80 g (0.15 mol) of 2,7-dibromo-9,9-dioctylfluorene was weighed and dissolved in 1.08 L of methyl-t-butyl ether. After cooling down to −78° C., 240 ml (0.38 mol) of n-BuLi was dropped slowly over a period of 20 minutes. After completion of dropping, the mixture was stirred at 0° C. for 2 hours, and cooled again down to −78° C. Subsequently, 71.33 g (0.38 mol) of B(OiPr)₃ was dropped over a period of 20 minutes and the mixture was heated up to room temperature, and allowed to stand overnight. The reaction solution was cooled to 0° C., then, ion exchanged water (300 ml) was dropped over a period of 30 minute while stirring. After dropping, the mixture was stirred for 30 minute and allowed to stand still for 30 minutes, and a solvent was distilled off under reduced pressure at 30° C. The residue was cooled down to 0° C., and a hydrochloric acid aqueous solution prepared by diluting 80 ml of 35%-HCl with 1 L of ion exchanged water was poured to cause hydrolysis, and the mixture was extracted with toluene. An organic layer was dried over magnesium sulfate, and filtrated, then, a solvent was distilled off under reduced pressure at 30° C. The resultant residue (compound G: 39.37 g) was in the form of gel containing toluene. Without effecting further purification, the charging amount was determined from theoretical yield and used in the subsequently process.

### (Synthesis of Compound H)

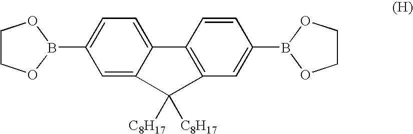

A 3 L four-necked flask was purged with nitrogen, and 39.37 g of the compound G synthesized above was dissolved in 800 ml of toluene, and 164.06 g (0.341 mol) was magnesium sulfate was added. Thereafter, 51.08 g (0.823 mol) of ethylene glycol was dropped over a period of 10 minutes. The reaction solution was stirred at room temperature for 2 hours. After completion of the reaction, MgSO₄ was removed by filtration, and a solvent was distilled off under reduced pressure at 45° C., to obtain 21.58 g of a coarse product as viscous liquid. Re-crystallization was carried out from hexane/acetonitrile, to obtain compound H.

¹H-NMR (CDCl₃, 300 MHz)

d7.83˜7.74 (m, 6H), 4.43 (s, 8H), 2.03˜1.97 (m, 4H), 1.26˜1.00 (m, 20H), 0.81 (t, 6H), 0.54 (brs, 4H)

### Example 2

Synthesis of Polymer Compound A

Into a 300 ml four-necked flask was placed 0.86 g of Aliquat 336, 0.43 g of the compound B, 3.29 g the compound F and 3.10 g of the compound H, and an atmosphere was purged with nitrogen. Argon-bubbled toluene 50 was added, and further, bubbling was carried out for 30 minutes while stirring. 4.5 mg of dichlorobis(triphenylphosphine)palladium (II) and 12 ml of 2 M sodium carbonate aqueous solution were added, and the mixture was stirred for 7 hours at a bath temperature of 105° C., then, 0.52 g of phenylboric acid was dissolved in 20 ml of toluene and 25 ml of tetrahydrofuran at a bath temperature of 105° C. and added to this, and the mixture was stirred for 3 horus. An aqueous solution prepared by dissolving 5 g of sodium N,N-diethyldithiocarbamate in 40 ml of water was added, and the mixture was further stirred for 3 hours at a bath temperature of 90° C. 400 ml of toluene was added, the reaction solution was partitioned, then, an organic phase was washed with 250 ml of water four times, then, dropped into 2.5 L of methanol, to cause re-precipiration of a polymer. After filtration and drying under reduced pressure, the mixture was dissolved in 150 ml of toluene, and passed through a silica gel-alumina column, and washed with 350 ml of toluene. The resultant toluene solution was dropped into 2.5 L of methanol, to cause re-precipiration of a polymer. After filtration and drying under reduced pressure, the mixture was dissolved in 150 ml of toluene, and dropped into 2.5 L of methanol, to cause re-precipiration of a polymer. Filtration and drying under reduced pressure were performed to obtain 4.08 g of polymer compound A.

Mn=98,000 Mw=210,000

### Synthesis Example 4

### Synthesis of Polymer Compound 2

The same operation was carried out using 0.41 g of the compound D instead of the compound B, to obtain 4.04 g of polymer compound B

Mn=81,000 Mw=200,000

### (Fluorescent Spectrum)

Fluorescent spectrum was measured according to the following method. A 0.8 wt % toluene solution of a polymer was spin-coated on quartz to form a thin film of the polymer. This thin film was excited at a wavelength of 350 nm, and fluorescent spectrum was measured using a fluorescence spectrophotometer (Fluorolog manufactured by Horiba, Ltd.). For obtaining relative fluorescence intensity in the thin film, fluorescent spectrum plotted against wave number was integrated in the spectrum measuring range utilizing the intensity of Raman line of water as a standard, and measurement was performed using a spectrophotometer (Cary 5E, manufactured Varian), obtaining a value allocated to the absorbance at the excited wavelength.

TABLE 1

| | Fluorescence peak | | Fluorescence |
|---|---|---|---|
| Polymer compound | wavelength (nm) | CIE(x, y) | intensity |
| Polymer compound | 458 | (0.144, 0.182) | 1.4 |
| 1(Example 2) | | | |
| Polymer compound | 460 | (0.144, 0.196) | 1.4 |
| 2(Synthesis | | | |
| Example 4) | | | |

As shown in Table 1, the polymer compound 1 of the example shows a smaller y value of C.I.E. chromatic coordinate as compared with the comparative example, thus, manifests high color purity as a blue light emitting material.

### Example 3

### Preparation of Solution

The polymer compound 1 obtained above was dissolved in xylene, to produce a xylene solution having a polymer concentration of 1.5 wt %.

### Manufacturing of EL Device

On a glass substrate carrying thereon an ITO film with a thickness of 150 nm formed by a sputtering method, a solution prepared by filtrating a suspension of poly(3,4)ethylenedioxythiophene/polystyrenesulfonic acid (manufactured by Bayer, BaytronP AI4083) through a 0.2 μm membrane filter was spin-coated to form a thin film with a thickness of 70 nm, and dried on a hot plate at 200° C. for 10 minutes. Next, the xylene solution obtained above was spin-coated at a rotation rate of 2100 rpm to form a film. The thickness after film formation was about 86 nm. Further, this was dried at 80° C. under reduced pressure for 1 hour, then, lithium fluoride was vapor-deposited with a thickness of about 3 nm, then, as a cathode, calcium was vapor-deposited with a thickness of about 10 nm and then aluminum was vapor-deposited with a thickness of about 80 nm, to manufacture an EL device. After the degree of vacuum reached 1×10⁻⁴ Pa or less, vapor-deposition of a metal was initiated.

### Ability of EL Device

By applying voltage on the resultant device, EL light emission showing a peak at 465 nm was obtained from this device. C.I.E. color coordinate values of EL light emission color in application of 6.0 V were x=0.145 and y=0.224. The intensity of EL light emission was in approximate proportion to the current density. This device showed initiation of light emission from 3.0 V, and the maximum light emission efficiency was 3.05 cd/A.

### Measurement of Life

The EL device obtained above was driven at a current value set so that the initial luminance was 400 cd/m², and change by time of luminance was measured, as a result, this device showed a luminance half life of 34 hours. The voltage necessary for driving was 4.41 V at initial and 4.60 V after luminance half life, thus, change in voltage during driving was 0.19 V. The voltage increase ratio was calculated from this converted half life, to find a value of 5.6 mV/hour.

### Comparative Example 1

### Preparation of Solution

The polymer compound 2 obtained above was dissolved in xylene, to produce a xylene solution having a polymer concentration of 1.5 wt %.

### Manufacturing of EL Device

An EL device was manufactured in utterly the same manner as in Example 1 excepting that the xylene solution obtained above was used. In this procedure, the spin coat revolution was 1800 rpm, and the film thickness of the resultant polymer was 88 nm.

### Ability of EL Device

By applying voltage on the resultant device, EL light emission showing a peak at 480 nm was obtained from this device. C.I.E. color coordinate values of EL light emission color in application of 6.0 V were x=0.147 and y=0.266. The intensity of EL light emission was in approximate proportion to the current density. This device showed initiation of light emission from 3.0 V, and the maximum light emission efficiency was 2.03 cd/A.

### Measurement of Life

The EL device obtained above was driven at a current value set so that the initial luminance was 400 cd/m², and change by time of luminance was measured, as a result, this device showed a luminance half life of 19 hours. The voltage necessary for driving was 4.19 V at initial and 4.89 V after luminance half life, thus, change in voltage during driving was 0.70 V. The voltage increase ratio was calculated from this converted half life, to find a value of 36.8 mV/hour.

As shown in the results of Example 3 and Comparative Example 1, the polymer compound 1 of the example shows a smaller y value of C.I.E. chromatic coordinate as compared with the comparative example, thus, manifests high color purity as a blue light emitting material. When driven as an EL device at constant current from an initial luminance of 400 cd/m², the life of the device of Example 3 is longer than the device of Comparative Example 1.

### Example 4

### Synthesis of Compound J and Compound J-1

### (Synthesis of Compound I)

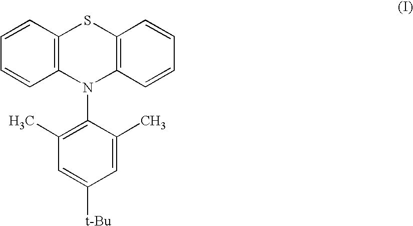

A three-necked flask was equipped with a reflux tube and thermo couple. Under a nitrogen atmosphere, phenothiazine (10.0 g), 1-bromo-4-t-butyl-2,6-dimethylbenzene (14.0 g), sodium-t-butoxide (20.1 g) and toluene (318 ml) were chared, then, trisdibenzylideneacetonepalladium (0.23 g) and tri(t-butyl)phosphine tetrafluoroborate (0.12 g) were charged. While refluxing under heat, the mixture was stirred for 10 hours, and cooled to room temperature. The reaction solution was filtrated through a glss filter pre-coated with alumina, and the resultant solution was washed with 3.5% hydrochloric acid, and the toluene solution was concentrated. To the resultant solid was added toluene and isopropyl alcohol and the mixture was heated to 75° C., and stirred for 1 hour, then, cooled to room temperature. The resultant precipitate was filtrated, and washed with isopropyl alcohol (100 ml) to obtain 16 g of compound I (10-(4-tert-Butyl-2,6-dimethyl-phenyl)-10H-phenothiazine) as pale yellow solid.

¹H-NMR (THF-d₈, 300 MHz):

d7.36 (s, 2H), 6.87 (d, 6.9 Hz, 2H), 6.68-6.78 (m, 4H), 5.87 (d, 8.1 Hz, 2H), 2.20 (s, 6H), 1.40 (s, 9H).

### (Synthesis of Compound J)

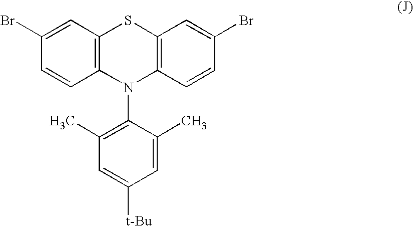

A three-necked flask was equipped with a reflux tube and thermo couple. Under a nitrogen atmosphere, the compound I (10-(4-tert-Butyl-2,6-dimethyl-phenyl)-10H-phenothiazine) (16.0 g) and dichloromethane (47 ml) were charged, and stirred at 0° C. A solution of 1,3-dibromo-5,5-dimethylhydantoine (12.8 g) in DMF (13.7 ml) was prepared and dropped at 0° C. 94.5 ml of methanol was added and the mixture was stirred, and the resultant precipitate was filtrated. This precipitate was dissolved in 30 ml of toluene, and 300 ml of methanol was added and the mixture was stirred at 70° C. for 1 hour, then, cooled to room temperature, and the generated crystal was filtrated. This crystal was re-crystallized from toluene (10 ml)/methanol (100 ml) three times, to obtain 9.76 g of the intended compound J (3,7-Dibromo-10-(4-tert-butyl-2,6-dimethyl-phenyl)-10H-pheno-thiazine) as a pale yellow green crystal.

(Synthesis of compound J-1)

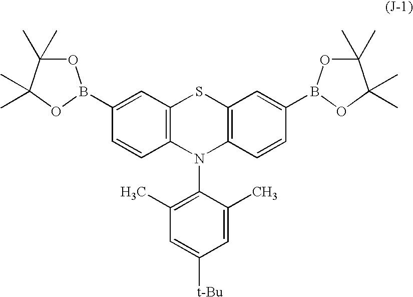

According to a method described in Bioorganic & Medicinal Chemistry Letters (2003), 13(18), 3059, compound J-1 can be obtained by heating in a dimethyl sulfoxide solvent in the presence of the compound B, bis(pinacolate)diborane, [1,1′-bis(diphenylphosphino)ferrocene]dichloropalladium (II) and potassium acetate.

### Example 5

For a monomer of a compound, a value of (1−A)×√\vec{}B was calculated using a molecular orbital calculation program, WinMOPAC 3.0 Professional (MOPAC2000 V1.3). The calculation was carried out while optimizing the structure by the AM1 method. Calculation of a value of the sum of squares of the atom orbital factor, is performed with 3 significant digits. The most stable conformation of a compound for calculating solid angle summation φf and molecular orbital, and the atom orbital factor of the highest occupied molecular orbital were obtained by optimizing the structured by a semi-empiriral molecular orbital method, AM1 method (Dewar, M. J. S. et al., J. Am. Chem. Soc., 107, 3902 (1985)).

TABLE 2

| monomer | K | L | M | N | O | P |
|---|---|---|---|---|---|---|
| (1-A) × \sqrt{B} | 0.051 | 0.027 | 0.120 | 0.103 | 0.092 | 0.120 |

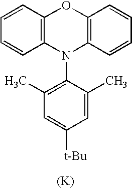

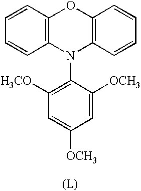

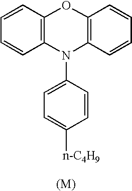

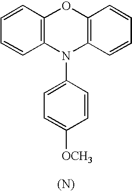

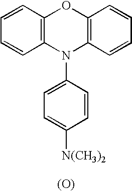

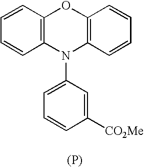

### INDUSTRIAL APPLICABILITY

The polymer compound of the present invention is useful as a light emitting material or charge transporting material, and when used in a polymer light emitting device, shows short light emission wavelength, and excellent in device properties such as chromaticity when used as a blue light emitting material, and life when used as a light emitting material of blue, green, red, white and the like. Therefore, a polymer LED containing the polymer compound of the present invention can be used for backlight of liquid crystal displays, or curved or plane light sources for illumination, segment type displays, dot matrix type flat panel displays, and the like.

1. A polymer compound comprising a residue of a compound of the following formula (1): 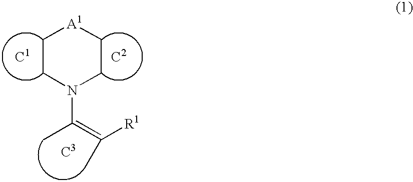
wherein, a ring C¹, ring C² and ring C³ represent each independently an aromatic hydrocarbon ring or hetero ring; A¹ represents a di-valent group containing one or more atoms selected from a boron atom, carbon atom, nitrogen atom, oxygen atom, phosphorus atom, sulfur atom and selenium atom; and R¹ represents an alkyl group, alkoxy group, alkylthio group, aryl group, aryloxy group, arylthio group, arylalkyl group, arylalkyloxy group, arylalkylthio group, alkenyl group, alkynyl group, di-substituted amino group, tri-substituted silyl group, acyl group, acyloxy group, imine residue, amide group, acid imide group, mono-valent heterocyclic group, substituted carboxyl group, heteroaryloxy group or heteroarylthio group, or is connected to an atom adjacent to an atom on the ring C³ to which R¹ is connected, to form a ring.

2. A polymer compound comprising a structural unit of the following formula (2): 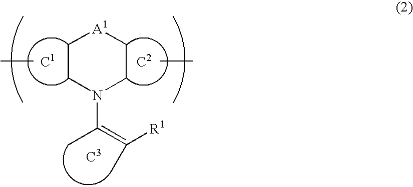
wherein, a ring C¹, ring C², ring C³, A¹ and R¹ have the same meanings as described above.

3. The polymer compound according to claim 1, wherein the ring C¹ and the ring C² are a benzene ring or monocyclic hetero ring.

4. The polymer compound according to claim 1, wherein the ring C³ is an aromatic hydrocarbon ring.

5. The polymer compound according to claim 1, wherein the ring C³ is represented by the following formula (3): 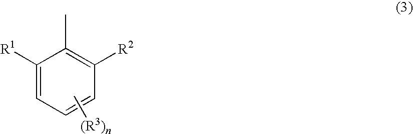
wherein, R¹ has the same meaning as described above; R² and R³ represent each independently an alkyl group, alkoxy group, alkylthio group, aryl group, aryloxy group, arylthio group, arylalkyl group, arylalkyloxy group, arylalkylthio group, alkenyl group, alkynyl group, di-substituted amino group, tri-substituted silyl group, acyl group, acyloxy group, imine residue, amide group, acid imide group, mono-valent heterocyclic group, substituted carboxyl group, heteroaryloxy group or heteroarylthio group, and when R³ is connected to a carbon atom adjacent to a carbon atom to which R¹ or R² is connected, the R³ may be connected to R¹ or R² to form a ring; n represents 0, 1, 2 or 3; and when n is 2 or more, plural R³s may be the same or different.

6. The polymer compound according to claim 1, wherein A¹ is an oxygen atom, sulfur atom, —S(═O)—, —S(═O)₂—, selenium atom, —Se(═O)— or —Se(═O)₂—.

7. The polymer compound according to claim 1, wherein A¹ is an oxygen atom, sulfur atom or selenium atom.

8. The polymer compound according to claim 1, satisfying the following formula (11):
(1−A)×√B≦0.070 (11)
wherein, A represents the shielding ratio of a nitrogen atom connected to the ring C³, and B represents the electron density of a nitrogen atom connected to the ring C³.

9. The polymer compound according to claim 1, further comprising a repeating unit of the following formula (4): 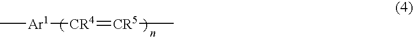
wherein, Ar¹ represents an arylene group, di-valent heterocyclic group or di-valent group having a metal complex structure; R⁴ and R⁵ represent each independently a hydrogen atom, alkyl group, aryl group, mono-valent heterocyclic group or cyano group; and n represents 0 or 1.

10. The polymer compound according to claim 9, wherein n is 0, in said formula (4).

11. The polymer compound according to claim 9, wherein Ar¹ is an arylene group, in said formula (4).

12. The polymer compound according to claim 1, further comprising a repeating unit of the following formula (5): 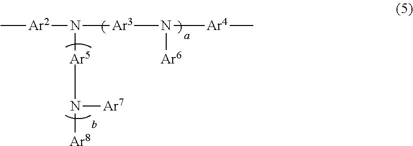
wherein, Ar², Ar³, Ar⁴ and Ar⁵ represent each independently an arylene group or di-valent heterocyclic group; Ar⁶, Ar⁷ and Ar⁸ represent each independently an aryl group or mono-valent heterocyclic group; and a and b represent each independently 0 or a positive integer.

13. The polymer compound according to claim 1, comprising a structural unit containing a residue of a compound of said formula (1) in an amount of 0.1 mol % or more and 40 mol % or less based on all structural units.

14. The polymer compound according to claim 1, wherein the polystyrene-reduced number average molecular weight is 10³ to 10⁸.

15. A method for producing a polymer compound of said formula (2) comprising polymerizing a compound of the following formula (6) as a raw material: 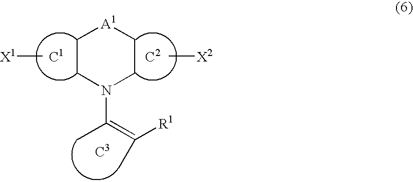
wherein, a ring C¹, ring C², ring C³, A¹ and R¹ have the same meanings as described above; and X¹ and X² represent each independently a substituent correlatable with polymerization.

16. The production method according to claim 15, wherein X¹ and X² represent each independently —B(OH)₂, borate group, magnesium halide, stannyl group, halogen atom, alkyl sulfonate group, aryl sulfonate group or aryl alkyl sulfonate group.

17. The production method according to claim 15, wherein X¹ and X² represent each independently —B(OH)₂, borate group, halogen atom, alkyl sulfonate group, aryl sulfonate group or aryl alkyl sulfonate group.

18. A compound of the following formula (7): 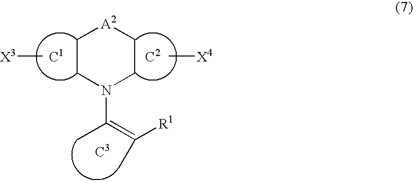
wherein, a ring C¹, ring C², ring C³ and R¹ have the same meanings as described above; A² represents a di-valent group represented by —BR′—, —C(R′)₂—, —NR′—, —O—, —PR′—, —P(═O)R¹—, —Se—, —Se(═O)— or —Se(═O)₂—; R′s represent each independently an alkyl group, alkoxy group, alkylthio group, aryl group, aryloxy group, arylthio group, arylalkyl group, arylalkyloxy group, arylalkylthio group, alkenyl group, alkynyl group, di-substituted amino group, tri-substituted silyl group, acyl group, acyloxy group, mono-valent heterocyclic group, substituted carboxyl group, heteroaryloxy group or heteroarylthio group; and X³ and X⁴ represent each independently a halogen atom, alkyl sulfonate group, aryl sulfonate group, aryl alkyl sulfonate group, borate group, sulfoniummethyl group, phosphoniummethyl group, phosphonatemethyl group, methyl monohalide group, magnesium halide group, substituted silyl group, stannyl group, —B(OH)₂, formyl group, cyano group or vinyl group.

19. The compound according to claim 18, wherein the ring C¹ and the ring C² are a benzene ring or monocyclic hetero ring, in said formula (7).

20. The compound according to claim 18, wherein the ring C³ is an aromatic hydrocarbon ring, in said formula (7).

21. The compound according to claim 18, wherein the ring C³ is represented by said formula (3), in said formula (7).

22. The compound according to claim 18, wherein A² is an oxygen atom, selenium atom, —Se(═O)— or —Se(═O)₂—, in said formula (7).

23. The compound according to claim 18, wherein X³ and X⁴ represent each independently —B(OH)₂, borate group, halogen atom, alkyl sulfonate group, aryl sulfonate group or aryl alkyl sulfonate group, in said formula (7).

24. A compound of the following formula (8): 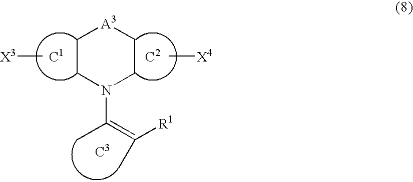
wherein, a ring C¹, ring C², ring C³, R¹, X³ and X⁴ have the same meanings as described above; and A³ represents a di-valent group containing a boron atom, carbon atom, nitrogen atom, oxygen atom, phosphorus atom, sulfur atom or selenium atom and forming a 7-membered ring or 8-membered ring together with the ring C¹, N atom and ring C².

25. The compound according to claim 24, wherein the ring C¹ and the ring C² are a benzene ring or monocyclic hetero ring, in said formula (8).

26. The compound according to claim 24, wherein the ring C³ is an aromatic hydrocarbon ring, in said formula (8).

27. The compound according to claim 24, wherein the ring C³ is represented by said formula (3), in said formula (8).

28. The compound according to claim 24, wherein X³ and X⁴ represent each independently —B(OH)₂, borate group, halogen atom, alkyl sulfonate group, aryl sulfonate group or aryl alkyl sulfonate group, in said formula (8).

29. A compound of the following formula (9): 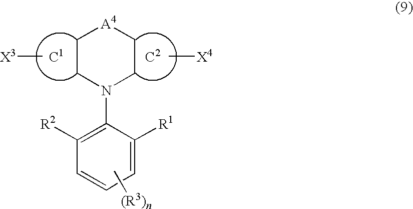
wherein, a ring C¹, ring C², R¹, R², R³, n, X³ and X⁴ have the same meanings as described above; and A⁴ represents a di-valent group represented by —C(═O)—, —C(═CR′₂)—, —S—, —S(═O)— or —S(═O)₂.

30. The compound according to claim 29, wherein the ring C¹ and the ring C² are a benzene ring or monocyclic hetero ring, in said formula (9).

31. The compound according to claim 29, wherein A⁴ is —S—, —S(═O)— or —S(═O)₂—, in said formula (9).

32. The compound according to claim 30, wherein X³ and X⁴ represent each independently —B(OH)₂, borate group, halogen atom, alkyl sulfonate group, aryl sulfonate group or aryl alkyl sulfonate group, in said formula (9).

33. A solution comprising the polymer compound as described in claim 1.

34. A light emitting thin film comprising the polymer compound as described in claim 1.

35. An electric conductive thin film comprising the polymer compound as described in claim 1.

36. An organic transistor comprising the polymer compound as described in claim 1.

37. A method for forming the thin film as described in claim 34, using an inkjet method.

38. A polymer light emitting device having an organic layer between electrodes composed of an anode and a cathode, wherein the organic layer contains the polymer compound as described in claim 1.

39. A sheet light source using the polymer light emitting device as described in claim 38.

40. A display using the polymer light emitting device as described in claim 38.
